(19) United States
(12) Patent Application Publication
(10) Pub. No.: US 2021/0320251 A1
(43) Pub. Date: Oct. 14, 2021

(54) ORGANIC ELECTROLUMINESCENCE DEVICE AND ELECTRONIC APPLIANCE

(71) Applicant: IDEMITSU KOSAN CO.,LTD., Tokyo (JP)

(72) Inventors: Yuki NAKANO, Sodegaura-shi (JP); Taro YAMAKI, Sodegaura-shi (JP); Satomi TASAKI, Sodegaura-shi (JP); Tetsuya MASUDA, Sodegaura-shi (JP)

(73) Assignee: IDEMITSU KOSAN CO.,LTD., Tokyo (JP)

(21) Appl. No.: 17/265,303

(22) PCT Filed: Aug. 2, 2019
(86) PCT No.: PCT/JP2019/030493

§ 371 Date: Feb. 2, 2021

(30) Foreign Application Priority Data
Aug. 3, 2018 (JP) 2018-147160

(51) Int. Cl. H01L 51/00; C07D 307/91; C09K 11/06

(52) U.S. Cl. CPC H01L 51/006; C07D 307/91; H01L 51/5016; H01L 51/0061; C09K 11/06

(57) ABSTRACT

An organic electroluminescence device, comprising: a cathode; an anode; and an emitting layer disposed between the cathode and the anode, wherein the emitting layer comprises: a compound represented by the following formula (1) and a compound represented by the following formula (11) (at least one of Ar₁₀₁ and Ar₁₀₂ is a monovalent group represented by the formula (12)).

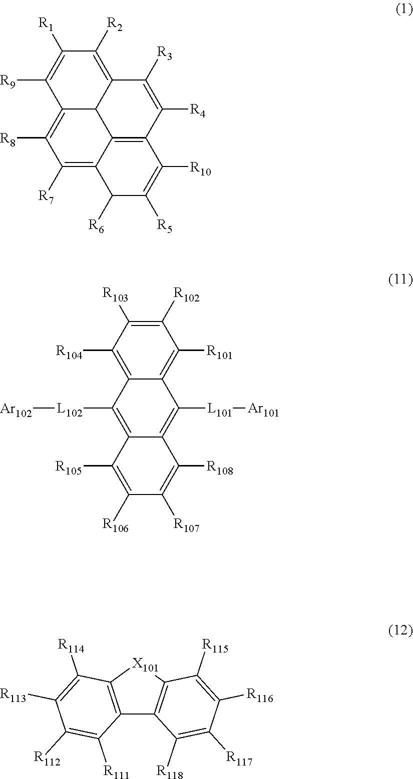

### TECHNICAL FIELD

The invention relates to an organic electroluminescence device and an electronic appliance.

### BACKGROUND ART

When voltage is applied to an organic electroluminescence device (hereinafter, referred to as an organic EL device), holes and electrons are injected into an emitting layer from an anode and a cathode, respectively. Then, thus injected holes and electrons are recombined in the emitting layer, and excitons are formed therein.

The organic EL device includes the emitting layer between the anode and the cathode. Further, the organic EL device has a stacked structure including an organic layer such as a hole-injecting layer, a hole-transporting layer, an electron-injecting layer, and an electron-transporting layer in several cases.

Patent Document 1 discloses a specific aromatic amine derivative as a material for an organic EL device.

### RELATED ART DOCUMENTS

### Patent Documents

[Patent Document 1] WO 2013/077405 A1

### SUMMARY OF THE INVENTION

It is an object of the invention to provide an organic electroluminescence device and an electronic appliance with reduced drive voltages while maintaining higher luminous efficiency.

According to an aspect of the invention, the following organic electroluminescence device is provided.

An organic electroluminescence device comprising: a cathode, an anode, and an emitting layer disposed between the cathode and the anode, wherein the emitting layer comprises a compound represented by the following formula (1) and a compound represented by the following formula (11):

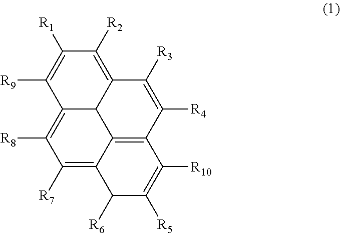

wherein in the formula (1), at least one of R₁ to R₁₀ is a monovalent group represented by the following formula (2);

R₁ to R₁₀ which are not the monovalent group represented by the following formula (2) are independently

a hydrogen atom, a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 carbon atoms that form a ring (hereinafter referred to as “ring carbon atoms”),

—Si(R₉₀₁)(R₉₀₂)(R₉₀₃),

—O—(R₉₀₄),

—S—(R₉₀₅),

—N(R₉₀₆)(R₉₀₇),

a substituted or unsubstituted aryl group including 6 to 50 ring carbon atoms, or
a substituted or unsubstituted monovalent heterocyclic group including 5 to 50 atoms that form a ring (hereinafter referred to as “ring atoms”);

adjacent two or more among R₁ to R₁₀ do not form a ring by bonding with each other;

R₉₀₁ to R₉₀₇ are independently

a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and

when two or more of each of R₉₀₁ to R₉₀₇ are present, the two or more of each of R₉₀₁ to R₉₀₇ may be the same or different;

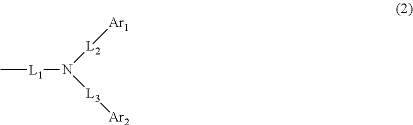

wherein in the formula (2), at least one of Ar₁ and Ar₂ is a group represented by the following formula (3);

Ar₁ or Ar₂ which is not the monovalent group represented by the following formula (3) is a substituted or unsubstituted aryl group including 6 to 50 ring carbon atoms, or

a substituted or unsubstituted monovalent heterocyclic group including 5 to 50 ring atoms;

L₁, L₂, and L₃ are independently a single bond,

a substituted or unsubstituted arylene group including 6 to 30 ring carbon atoms, or
a substituted or unsubstituted divalent heterocyclic group including 5 to 30 ring atoms; and

when two or more of each of Ar₁, Ar₂, L₁, L₂, and L₃ are present, the two or more of each of Ar₁, Ar₂, L₁, L₂, and L₃ may be the same or different;

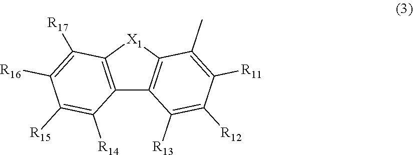

wherein in the formula (3), R₁ is a substituted or unsubstituted alkyl group including 1 to 50 carbon atoms,

a substituted or unsubstituted aryl group including 6 to 50 ring carbon atoms, or
a substituted or unsubstituted monovalent heterocyclic group including 5 to 50 ring atoms;

one or more sets of adjacent two or more among R₁₂ to R₁₇ form a substituted or unsubstituted, saturated or unsaturated ring by bonding with each other, or do not form a substituted or unsubstituted, saturated or unsaturated ring;

R₁₂ to R₁₇ which do not form a substituted or unsubstituted, saturated or unsaturated ring are independently

a hydrogen atom, a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a substituted or unsubstituted aryl group including 6 to 50 ring carbon atoms, or
a substituted or unsubstituted monovalent heterocyclic group including 5 to 50 ring atoms;

R₉₀₁ to R₉₀₇ are as defined in the formula (1); and

X₁ is an oxygen atom or a sulfur atom;

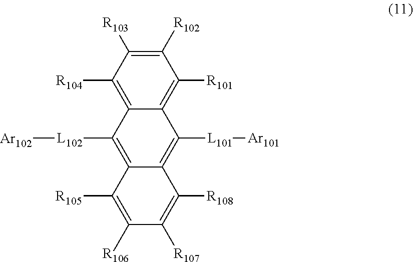

wherein in the formula (11), R₁₀₁ to R₁₀₈ are independently

a hydrogen atom, a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a substituted or unsubstituted aryl group including 6 to 50 ring carbon atoms, or
a substituted or unsubstituted monovalent heterocyclic group including 5 to 50 ring atoms;

adjacent two or more among R₁₀₁ to R₁₀₄, and adjacent two or more among R₁₀₅ to R₁₀₈ do not form a ring by bonding with each other;

R₉₀₁ to R₉₀₇ are as defined in the formula (1);

L₁₀₁ and L₁₀₂ are independently

a single bond,
a substituted or unsubstituted arylene group including 6 to 30 ring carbon atoms, or

a substituted or unsubstituted divalent heterocyclic group including 5 to 30 ring atoms; at least one of Ar₁₀₁ and Ar₁₀₂ is a monovalent group represented by the following formula (12);

Ar₁₀₁ or Ar₁₀₂ which is not the monovalent group represented by the following formula (12) is a substituted or unsubstituted aryl group including 6 to 50 ring carbon atoms, or

a substituted or unsubstituted monovalent heterocyclic group including 5 to 50 ring atoms; and

when both Ar₁₀₁ and Ar₁₀₂ are the monovalent groups represented by the formula (12), Ar₁₀₁ and Ar₁₀₂ which are the monovalent groups represented by the following formula (12) may be the same as or different from each other;

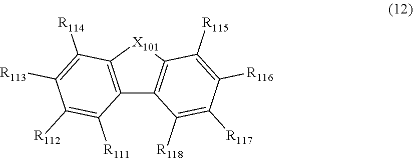

wherein in the formula (12),

X₁₀₁ is an oxygen atom or a sulfur atom; and

one or more sets of adjacent two or more of R₁₁₁ to R₁₁₈ form an unsaturated ring represented by the following formula (20) by bonding with each other, or do not form the unsubstituted ring represented by the following formula (20);

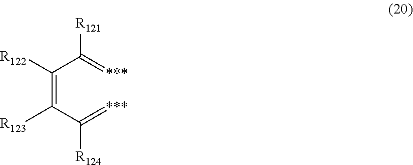

wherein in the formula (20), “***” indicates a position bonding to adjacent two of R₁₁₁ to R₁₁₈; when one or more sets of adjacent two of R₁₁₁ to R₁₁₈ form the unsaturated ring represented by the formula (20) by bonding with each other, one of R₁₁₁ to R₁₁₈ which do not form the unsaturated ring represented by the formula (20), and one of R₁₂₁ to R₁₂₄ is a single bond bonding with L₁₀₁ or L₁₀₂;

when two or more of the unsaturated rings represented by the formula (20) are formed, a plurality of each of R₁₂₁ to R₁₂₄ may be the same as or different from each other;

when one or more sets of adjacent two of R₁₁₁ to R₁₁₈ do not form the unsaturated ring represented by the formula (20), one of R₁₁₁ to R₁₁₈ is a single bond bonding with L₁₀₁ or L₁₀₂;

when the unsaturated ring represented by the formula (20) is formed and when the unsaturated ring represented by the formula (20) is not formed, one or more sets of adjacent two of R₁₁₁ to R₁₁₈ which do not form the unsaturated ring represented by the formula (20) and are not a single bond bonding with L₁₀₁ or L₁₀₂ form a substituted or unsubstituted, saturated or unsaturated ring other than the unsaturated ring represented by the formula (20) by bonding with each other, or do not form a substituted or unsubstituted, saturated or unsaturated ring;

R₁₁₁ to R₁₁₈ which do not form the unsaturated ring represented by the formula (20), do not form a substituted or unsubstituted, saturated or unsaturated ring other than the unsaturated ring represented by the formula (20), and are not a single bond bonding with L₁₀₁ or L₁₀₂, and R₁₂₁ to R₁₂₄ which are not a single bond bonding with L₁₀₁ or L₁₀₂ are independently

a hydrogen atom, a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a substituted or unsubstituted aryl group including 6 to 50 ring carbon atoms, or
a substituted or unsubstituted monovalent heterocyclic group including 5 to 50 ring atoms; and

R₉₀₁ to R₉₀₇ are as defined in the formula (1).

Another aspect of the invention provides an electronic appliance provided with the organic electroluminescence device.

The invention provides an organic electroluminescence device and an electronic appliance with reduced drive voltages while maintaining higher luminous efficiency.

### BRIEF DESCRIPTION OF THE DRAWINGS

FIG. 1 is a diagram showing a schematic configuration of one embodiment of an organic EL device of the invention.

### MODE FOR CARRYING OUT THE INVENTION

### Definition

In the this specification, a hydrogen atom means an atom including isotopes different in the number of neutrons, namely, a protium, a deuterium and a tritium.

In the this specification, to a bondable position in which a symbol such as “R”, or “D” representing a deuterium atom is not specified in a chemical formula, a hydrogen atom, that is, a light hydrogen atom, a deuterium atom, or a tritium atom is bonded thereto.

In the this specification, a term “ring carbon atoms” represents the number of carbon atoms among atoms forming a subject ring itself of a compound having a structure in which atoms are bonded in a ring form (for example, a monocyclic compound, a fused ring compound, a cross-linked compound, a carbocyclic compound or a heterocyclic compound). When the subject ring is substituted by a substituent, the carbon contained in the substituent is not included in the number of ring carbon atoms. The same shall apply to the “ring carbon atoms” described below, unless otherwise noted. For example, a benzene ring has 6 ring carbon atoms, a naphthalene ring has 10 ring carbon atoms, a pyridine ring has 5 ring carbon atoms, and a furan ring has 4 ring carbon atoms. Further, for example, a 9,9-diphenylfluorenyl group has 13 ring carbon atoms, and a 9,9′-spirobifluorenyl group has 25 ring carbon atoms.

Further, when the benzene ring or the naphthalene ring is substituted by an alkyl group as a substituent, for example, the number of carbon atoms of the alkyl group is not included in the ring carbon atoms.

In the this specification, a term “ring atoms” represents the number of atoms forming a subject ring itself of a compound having a structure in which atoms are bonded in a ring form (for example, a monocycle, a fused ring and a ring assembly) (for example, a monocyclic compound, a fused ring compound, a cross-linked compound, a carbocyclic compound or a heterocyclic compound). The term “ring atoms” does not include atoms which do not form the ring (for example, a hydrogen atom which terminates a bond of the atoms forming the ring) or atoms contained in a substituent when the ring is substituted by the substituent. The same shall apply to the “ring atoms” described below, unless otherwise noted. For example, a pyridine ring has 6 ring atoms, a quinazoline ring has 10 ring atoms, and a furan ring has 5 ring atoms. A hydrogen atom bonded with a carbon atom of the pyridine ring or the quinazoline ring or an atom forming the substituent is not included in the number of the ring atoms.

In the this specification, a term “XX to YY carbon atoms” in an expression of “substituted or unsubstituted ZZ group including XX to YY carbon atoms” represents the number of carbon atoms when the ZZ group is unsubstituted. The number of carbon atoms of a substituent when the ZZ group is substituted is not included. Here, “YY” is larger than “XX”, and “XX” and “YY” each mean an integer of 1 or more.

In the this specification, a term “XX to YY atoms” in an expression of “substituted or unsubstituted ZZ group including XX to YY atoms” represents the number of atoms when the ZZ group is unsubstituted. The number of atoms of a substituent when the group is substituted is not included. Here, “YY” is larger than “XX”, and “XX” and “YY” each mean an integer of 1 or more.

A term “unsubstituted” in the case of “substituted or unsubstituted ZZ group” means that the ZZ group is not substituted by a substituent, and a hydrogen atom is bonded therewith. Alternatively, a term “substituted” in the case of “substituted or unsubstituted ZZ group” means that one or more hydrogen atoms in the ZZ group are substituted by a substituent. Similarly, a term “substituted” in the case of “BB group substituted by an AA group” means that one or more hydrogen atoms in the BB group are substituted by the AA group.

Hereinafter, the substituent described herein will be described.

The number of the ring carbon atoms of the “unsubstituted aryl group” described herein is 6 to 50, preferably 6 to 30, and more preferably 6 to 18, unless otherwise specified.

The number of the ring carbon atoms of the “unsubstituted heterocyclic group” described herein is 5 to 50, preferably 5 to 30, and more preferably 5 to 18, unless otherwise specified.

The number of the carbon atoms of the “unsubstituted alkyl group” described herein is 1 to 50, preferably 1 to 20, and more preferably 1 to 6, unless otherwise specified.

The number of the carbon atoms of the “unsubstituted alkenyl group” described herein is 2 to 50, preferably 2 to 20, and more preferably 2 to 6, unless otherwise specified.

The number of the carbon atoms of the “unsubstituted alkynyl group” described herein is 2 to 50, preferably 2 to 20, and more preferably 2 to 6, unless otherwise specified.

The number of the ring carbon atoms of the “unsubstituted cycloalkyl group” described herein is 3 to 50, preferably 3 to 20, and more preferably 3 to 6, unless otherwise specified.

The number of the ring carbon atoms of the “unsubstituted arylene group” described herein is 6 to 50, preferably 6 to 30, and more preferably 6 to 18, unless otherwise specified.

The number of the ring atoms of the “unsubstituted divalent heterocyclic group” described herein is 5 to 50, preferably 5 to 30, and more preferably 5 to 18, unless otherwise specified.

The number of the carbon atoms of the “unsubstituted alkylene group” described herein is 1 to 50, preferably 1 to 20, and more preferably 1 to 6, unless otherwise specified.

Specific examples (specific example group G1) of the “substituted or unsubstituted aryl group” described herein include an unsubstituted aryl group and a substituted aryl group described below. (Here, a term “unsubstituted aryl group” refers to a case where the “substituted or unsubstituted aryl group” is the “unsubstituted aryl group,” and a term “substituted aryl group” refers to a case where the “substituted or unsubstituted aryl group” is the “substituted aryl group”. Hereinafter, a case of merely “aryl group” includes both the “unsubstituted aryl group” and the “substituted aryl group”.

The “substituted aryl group” refers to a case where the “unsubstituted aryl group” has a substituent, and specific examples thereof include a group in which the “unsubstituted aryl group” has the substituent, and a substituted aryl group described below. It should be noted that examples of the “unsubstituted aryl group” and examples of the “substituted aryl group” listed herein are only one example, and the “substituted aryl group” described herein also includes a group in which a group in which “unsubstituted aryl group” has a substituent further has a substituent, and a group in which “substituted aryl group” further has a substituent, and the like.

An unsubstituted aryl group:

a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group,
a phenalenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group, and
a perylenyl group.

A substituted aryl group:

an o-tolyl group,
a m-tolyl group,
a p-tolyl group,
a p-xylyl group,
a m-xylyl group,
an o-xylyl group,
a p-isopropyl phenyl group,
a m-isopropyl phenyl group,
an o-isopropyl phenyl group,
a p-t-butylphenyl group,
a m-t-butylphenyl group,
an o-t-butylphenyl group,
a 3,4,5-trimethylphenyl group,
a 9,9-dimethylfluorenyl group,
a 9,9-diphenylfluorenyl group
a 9,9-di(4-methylphenyl)fluorenyl group,
a 9,9-di(4-isopropylphenyl)fluorenyl group,
a 9,9-di(4-t-butylphenyl)fluorenyl group,
a cyanophenyl group,
a triphenylsilylphenyl group,
a trimethylsilylphenyl group,
a phenylnaphthyl group, and
a naphthylphenyl group.

The “heterocyclic group” described herein is a ring group including at least one hetero atom in the ring atom. Specific examples of the hetero atom include a nitrogen atom, an oxygen atom, a sulfur atom, a silicon atom, a phosphorus atom and a boron atom.

The “heterocyclic group” described herein may be a monocyclic group, or a fused ring group.

The “heterocyclic group” described herein may be an aromatic heterocyclic group, or an aliphatic heterocyclic group.

Specific examples (specific example group G2) of the “substituted or unsubstituted heterocyclic group” include an unsubstituted heterocyclic group and a substituted heterocyclic group described below. (Here, the unsubstituted heterocyclic group refers to a case where the “substituted or unsubstituted heterocyclic group” is the “unsubstituted heterocyclic group,” and the substituted heterocyclic group refers to a case where the “substituted or unsubstituted heterocyclic group” is the “substituted heterocyclic group”. Hereinafter, the case of merely “heterocyclic group” includes both the “unsubstituted heterocyclic group” and the “substituted heterocyclic group”.

The “substituted heterocyclic group” refers to a case where the “unsubstituted heterocyclic group” has a substituent, and specific examples thereof include a group in which the “unsubstituted heterocyclic group” has a substituent, and a substituted heterocyclic group described below. It should be noted that examples of the “unsubstituted heterocyclic group” and examples of the “substituted heterocyclic group” listed herein are merely one example, and the “substituted heterocyclic group” described herein also includes a group in which “unsubstituted heterocyclic group” which has a substituent further has a substituent, and a group in which “substituted heterocyclic group” further has a substituent, and the like.

An unsubstituted heterocyclic group including a nitrogen atom:

a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolyl group,
a phthalazinyl group,
a quinazolinyl group,
a quinoxalinyl group,
a benzimidazolyl group,
an indazolyl group,
a phenanthrolinyl group,
a phenanthridinyl group
an acridinyl group,
a phenazinyl group,
a carbazolyl group,
a benzocarbazolyl group,
a morpholino group,
a phenoxazinyl group,
a phenothiazinyl group,
an azacarbazolyl group, and
a diazacarbazolyl group.

An unsubstituted heterocyclic group including an oxygen atom:

a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

An unsubstituted heterocyclic group including a sulfur atom:

a thienyl group,
a thiazolyl group,
an isothiazolyl group,
a thiadiazolyl group,
a benzothiophenyl group,
an isobenzothiophenyl group,
a dibenzothiophenyl group,
a naphthobenzothiophenyl group,
a benzothiazolyl group,
a benzisothiazolyl group,
a phenothiazinyl group,
a dinaphthothiophenyl group,
an azadibenzothiophenyl group,
a diazadibenzothiophenyl group,
an azanaphthobenzothiophenyl group, and
a diazanaphthobenzothiophenyl group.

A substituted heterocyclic group including a nitrogen atom:

a (9-phenyl)carbazolyl group,
a (9-biphenylyl)carbazolyl group,
a (9-phenyl)phenylcarbazolyl group,
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenylyltriazinyl group,
a diphenyltriazinyl group,
a phenylquinazolinyl group, and
a biphenylylquinazolinyl group.

A substituted heterocyclic group including an oxygen atom:

a phenyldibenzofuranyl group,
a methyldibenzofuranyl group,
a t-butyldibenzofuranyl group, and
a monovalent residue of spiro[9H-xanthene-9,9′-[9H]fluorene].

A substituted heterocyclic group including a sulfur atom:

a phenyldibenzothiophenyl group,
a methyldibenzothiophenyl group,
a t-butyldibenzothiophenyl group, and
a monovalent residue of spiro[9H-thioxantene-9,9′-[9H]fluorene].

A monovalent group derived from the following unsubstituted heterocyclic ring containing at least one of a nitrogen atom, an oxygen atom and a sulfur atom by removal of one hydrogen atom bonded to the ring atoms thereof, and a monovalent group in which a monovalent group derived from the following unsubstituted heterocyclic ring has a substituent by removal of one hydrogen atom bonded to the ring atoms thereof:

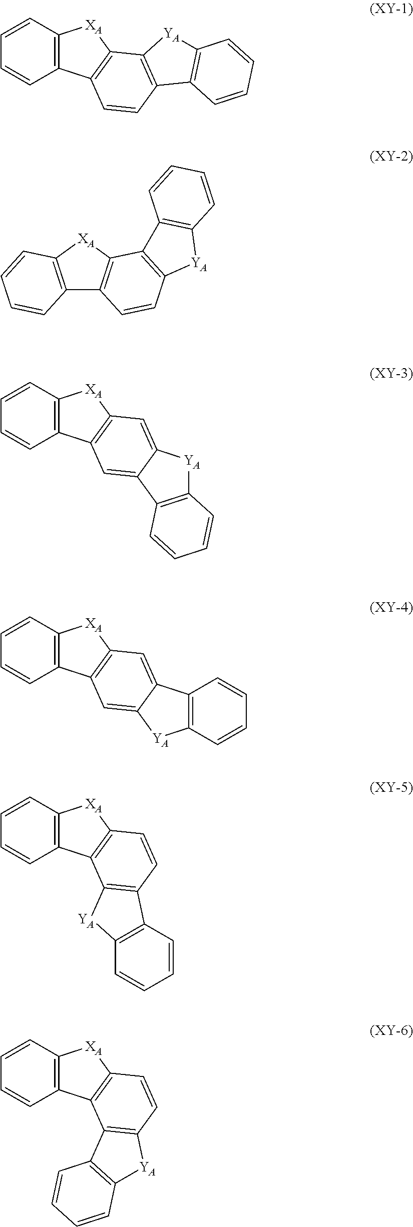

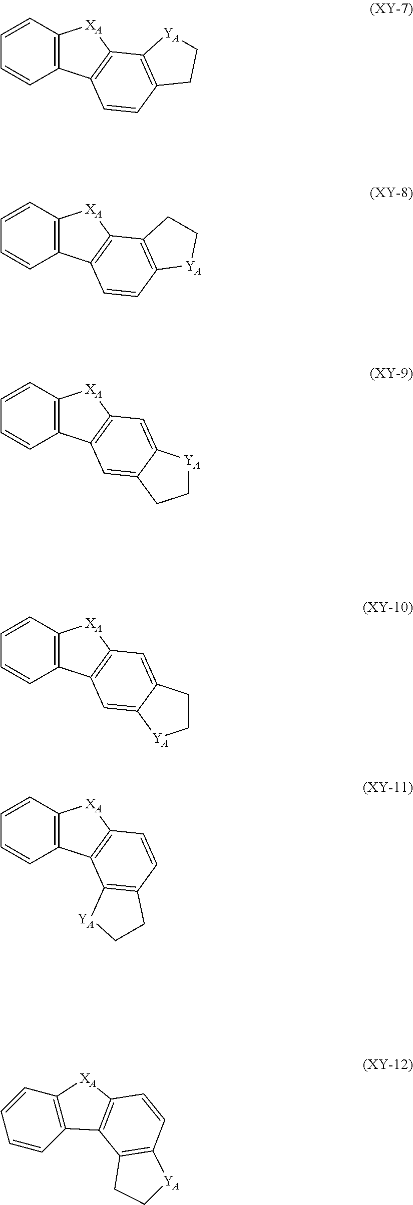

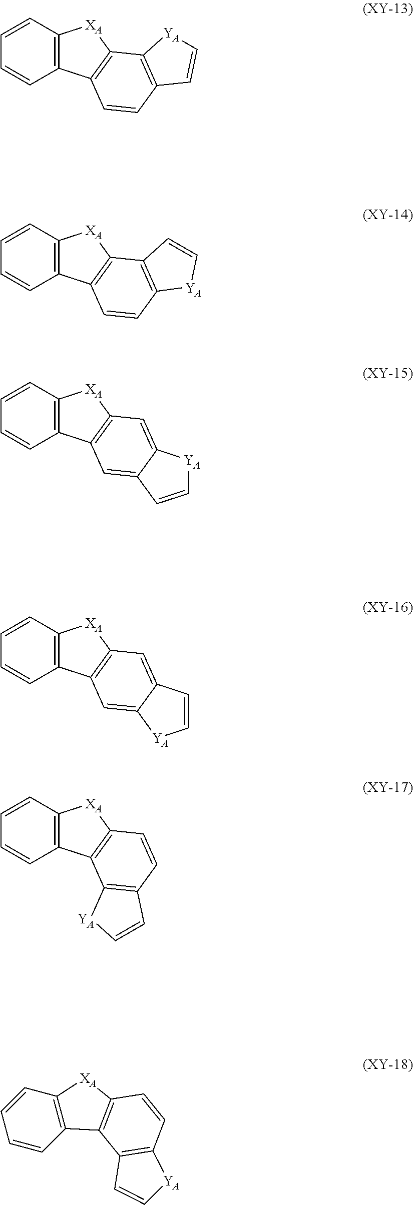

In the formulas (XY-1) to (XY-18), X_{A} and Y_{A} are independently an oxygen atom, a sulfur atom, NH or CH₂. However, at least one of X_{A} and Y_{A} is an oxygen atom, a sulfur atom or NH.

The heterocyclic ring represented by the formulas (XY-1) to (XY-18) becomes a monovalent heterocyclic group including a bond at an arbitrary position.

An expression “the monovalent group derived from the unsubstituted heterocyclic ring represented by the formulas (XY-1) to (XY-18) has a substituent” refers to a case where the hydrogen atom bonded with the carbon atom which constitutes a skeleton of the formulas is substituted by a substituent, or a state in which X_{A} or Y_{A} is NH or CH₂, and the hydrogen atom in the NH or CH₂ is replaced with a substituent.

Specific examples (specific example group G3) of the “substituted or unsubstituted alkyl group” include an unsubstituted alkyl group and a substituted alkyl group described below. (Here, the unsubstituted alkyl group refers to a case where the “substituted or unsubstituted alkyl group” is the “unsubstituted alkyl group,” and the substituted alkyl group refers to a case where the “substituted or unsubstituted alkyl group” is the “substituted alkyl group”). Hereinafter, the case of merely “alkyl group” includes both the “unsubstituted alkyl group” and the “substituted alkyl group”.

The “substituted alkyl group” refers to a case where the “unsubstituted alkyl group” has a substituent, and specific examples thereof include a group in which the “unsubstituted alkyl group” has a substituent, and a substituted alkyl group described below. It should be noted that examples of the “unsubstituted alkyl group” and examples of the “substituted alkyl group” listed herein are merely one example, and the “substituted alkyl group” described herein also includes a group in which “unsubstituted alkyl group” has a substituent further has a substituent, a group in which “substituted alkyl group” further has a substituent, and the like.

An unsubstituted alkyl group:

a methyl group,
an ethyl group,
a n-propyl group,
an isopropyl group,
a n-butyl group,
an isobutyl group,
a s-butyl group, and
a t-butyl group.

A substituted alkyl group:

a heptafluoropropyl group (including an isomer),
a pentafluoroethyl group,
a 2,2,2-trifluoroethyl group, and
a trifluoromethyl group.

Specific examples (specific example group G4) of the “substituted or unsubstituted alkenyl group” include an unsubstituted alkenyl group and a substituted alkenyl group described below. (Here, the unsubstituted alkenyl group refers to a case where the “substituted or unsubstituted alkenyl group” is the “unsubstituted alkenyl group,” and the substituted alkenyl group refers to a case where the “substituted or unsubstituted alkenyl group” is the “substituted alkenyl group”). Hereinafter, the case of merely “alkenyl group” includes both the “unsubstituted alkenyl group” and the “substituted alkenyl group”.

The “substituted alkenyl group” refers to a case where the “unsubstituted alkenyl group” has a substituent, and specific examples thereof include a group in which the “unsubstituted alkenyl group” has a substituent, and a substituted alkenyl group described below. It should be noted that examples of the “unsubstituted alkenyl group” and examples of the “substituted alkenyl group” listed herein are merely one example, and the “substituted alkenyl group” described herein also includes a group in which “unsubstituted alkenyl group” has a substituent further has a substituent, a group in which “substituted alkenyl group” further has a substituent, and the like.

An unsubstituted alkenyl group and a substituted alkenyl group:

a vinyl group,
an allyl group,
a 1-butenyl group,
a 2-butenyl group,
a 3-butenyl group,
a 1,3-butanedienyl group,
a 1-methylvinyl group,
a 1-methylallyl group,
a 1,1-dimethylallyl group,
a 2-methylallyl group, and
a 1,2-dimethylallyl group.

Specific examples (specific example group G5) of the “substituted or unsubstituted alkynyl group” include an unsubstituted alkynyl group described below. (Here, the unsubstituted alkynyl group refers to a case where the “substituted or unsubstituted alkynyl group” is the “unsubstituted alkynyl group”). Hereinafter, a case of merely “alkynyl group” includes both the “unsubstituted alkynyl group” and the “substituted alkynyl group”.

The “substituted alkynyl group” refers to a case where the “unsubstituted alkynyl group” has a substituent, and specific examples thereof include a group in which the “unsubstituted alkynyl group” described below has a substituent.

An unsubstituted alkynyl group:

an ethynyl group.

Specific examples (specific example group G6) of the “substituted or unsubstituted cycloalkyl group” described herein include an unsubstituted cycloalkyl group and a substituted cycloalkyl group described below. (Here, the unsubstituted cycloalkyl group refers to a case where the “substituted or unsubstituted cycloalkyl group” is the “unsubstituted cycloalkyl group,” and the substituted cycloalkyl group refers to a case where the “substituted or unsubstituted cycloalkyl group” is the “substituted cycloalkyl group”). Hereinafter, a case of merely “cycloalkyl group” includes both the “unsubstituted cycloalkyl group” and the “substituted cycloalkyl group”.

The “substituted cycloalkyl group” refers to a case where the “unsubstituted cycloalkyl group” a the substituent, and specific examples thereof include a group in which the “unsubstituted cycloalkyl group” has a substituent, and a substituted cycloalkyl group described below. It should be noted that examples of the “unsubstituted cycloalkyl group” and examples of the “substituted cycloalkyl group” listed herein are merely one example, and the “substituted cycloalkyl group” described herein also includes a group in which “unsubstituted cycloalkyl group” has a substituent further has a substituent, a group in which “substituted cycloalkyl group” further has a substituent, and the like.

An unsubstituted aliphatic ring group:

a cyclopropyl group,
a cyclobutyl group,
a cyclopentyl group,
a cyclohexyl group,
a 1-adamantyl group,
a 2-adamantyl group,
a 1-norbornyl group, and
a 2-norbornyl group.

A substituted cycloalkyl group:

a 4-methylcyclohexyl group.

Specific examples (specific example group G7) of the group represented by —Si(R₉₀₁)(R₉₀₂)(R₉₀₃) described herein include

—Si(G1)(G1)(G1),

—Si(G1)(G2)(G2),

—Si(G1)(G1)(G2),

—Si(G2)(G2)(G2),

—Si(G3)(G3)(G3),

—Si(G5)(G5)(G5) and

—Si(G6)(G6)(G6).

In which,

G1 is the “aryl group” described in the specific example group G1.

G2 is the “heterocyclic group” described in the specific example group G2.

G3 is the “alkyl group” described in the specific example group G3.

G5 is the “alkynyl group” described in the specific example group G5.

G6 is the “cycloalkyl group” described in the specific example group G6.

Specific examples (specific example group G8) of the group represented by —O—(R₉₀₄) described herein include

—O(G1),

—O(G2),

—O(G3) and

—O(G6).

In which,

G1 is the “aryl group” described in the specific example group G1.

G2 is the “heterocyclic group” described in the specific example group G2.

G3 is the “alkyl group” described in the specific example group G3.

G6 is the “cycloalkyl group” described in the specific example group G6.

Specific examples (specific example group G9) of the group represented by —S—(R₉₀₅) described herein include

—S(G1),

—S(G2),

—S(G3) and

—S(G6).

In which,

G1 is the “aryl group” described in the specific example group G1.

G2 is the “heterocycle group” described in the specific example group G2.

G3 is the “alkyl group” described in the specific example group G3.

G6 is the “cycloalkyl group” described in the specific example group G6.

Specific examples (specific example group G10) of the group represented by —N(R₉₀₆)(R₉₀₇) described herein include

—N(G1)(G1),

—N(G2)(G2),

—N(G1)(G2),

—N(G3)(G3) and

—N(G6)(G6).

In which,

G1 is the “aryl group” described in the specific example group G1.

G2 is the “heterocycle group” described in the specific example group G2.

G3 is the “alkyl group” described in the specific example group G3.

G6 is the “cycloalkyl group” described in the specific example group G6.

Specific examples (specific example group G11) of the “halogen atom” described herein include a fluorine atom, a chlorine atom, a bromine atom and an iodine atom.

Specific examples of the “alkoxy group” described herein include a group represented by —O(G3), where G3 is the “alkyl group” described in the specific example group G3. The number of carbon atoms of the “unsubstituted alkoxy group” are 1 to 50, preferably 1 to 30, and more preferably 1 to 18, unless otherwise specified.

Specific examples of the “alkylthio group” described herein include a group represented by —S(G3), where G3 is the “alkyl group” described in the specific example group G3. The number of carbon atoms of the “unsubstituted alkylthio group” are 1 to 50, preferably 1 to 30, and more preferably 1 to 18, unless otherwise specified.

Specific examples of the “aryloxy group” described herein include a group represented by —O(G1), where G1 is the “aryl group” described in the specific example group G1. The number of ring carbon atoms of the “unsubstituted aryloxy group” are 6 to 50, preferably 6 to 30, and more preferably 6 to 18, unless otherwise specified.

Specific examples of the “arylthio group” described herein include a group represented by —S(G1), where G1 is the “aryl group” described in the specific example group G1. The number of ring carbon atoms of the “unsubstituted arylthio group” are 6 to 50, preferably 6 to 30, and more preferably 6 to 18, unless otherwise specified.

Specific examples of the “aralkyl group” described herein include a group represented by -(G3)-(G1), where G3 is the “alkyl group” described in the specific example group G3, and G1 is the “aryl group” described in the specific example group G1. Accordingly, the “aralkyl group” is one embodiment of the “substituted alkyl group” substituted by the “aryl group”. The number of carbon atoms of the “unsubstituted aralkyl group,” which is the “unsubstituted alkyl group” substituted by the “unsubstituted aryl group,” are 7 to 50, preferably 7 to 30, and more preferably 7 to 18, unless otherwise specified.

Specific example of the “aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, and a 2-β-naphthylisopropyl group.

The substituted or unsubstituted aryl group described herein is, unless otherwise specified, preferably a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, a 9,9-diphenylfluorenyl group, or the like.

The substituted or unsubstituted heterocyclic group described herein is, unless otherwise specified, preferably a pyridyl group, a pyrimidinyl group, a triazinyl group, a quinolyl group, an isoquinolyl group, a quinazolinyl group, a benzimidazolyl group, a phenanthrolinyl group, a carbazolyl group (a 1-carbazolyl group, a 2-carbazolyl group, a 3-carbazolyl group, a 4-carbazolyl group, or a 9-carbazolyl group), a benzocarbazolyl group, an azacarbazolyl group, a diazacarbazolyl group, a dibenzofuranyl group, a naphthobenzofuranyl group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazole-9-yl group, a phenylcarbazol-9-yl group, a phenyltriazinyl group, a biphenylyltriazinyl group, diphenyltriazinyl group, a phenyldibenzofuranyl group, a phenyldibenzothiophenyl group, an indrocarbazolyl group, a pyrazinyl group, a pyridazinyl group, a quinazolinyl group, a cinnolinyl group, a phthalazinyl group, a quinoxalinyl group, a pyrrolyl group, an indolyl group, a pyrrolo[3,2,1-jk]carbazolyl group, a furanyl group, a benzofuranyl group, a thiophenyl group, a benzothiophenyl group, a pyrazolyl group, an imidazolyl group, a benzimidazolyl group, a triazolyl group, an oxazolyl group, a benzoxazolyl group, a thiazolyl group, a benzothiazolyl group, an isothiazolyl group, a benzisothiazolyl group, a thiadiazolyl group, an isoxazolyl group, a benzisoxazolyl group, a pyrrolidinyl group, a piperidinyl group, a piperazinyl group, an imidazolidinyl group, an indro[3,2,1-jk]carbazolyl group, a dibenzothiophenyl group, or the like.

The dibenzofuranyl group and the dibenzothiophenyl group as described above are specifically any group described below, unless otherwise specified.

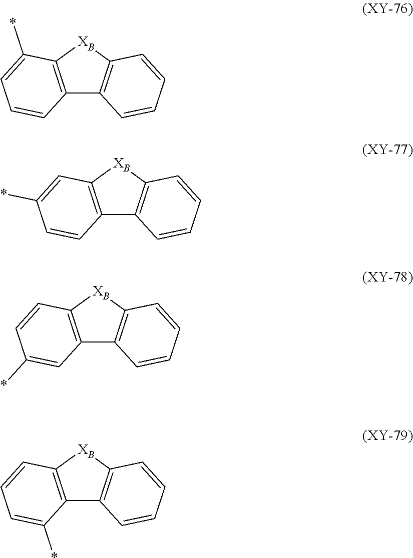

In the formulas (XY-76) to (XY-79), X_{B} is an oxygen atom or a sulfur atom.

The substituted or unsubstituted alkyl group described herein is, unless otherwise specified, preferably a methyl group, an ethyl group, a propyl group, an isopropyl group, a n-butyl group, an isobutyl group, a t-butyl group, or the like.

The “substituted or unsubstituted arylene group” descried herein refers to a group in which the above-described “aryl group” is converted into divalence, unless otherwise specified. Specific examples (specific example group G12) of the “substituted or unsubstituted arylene group” include a group in which the “aryl group” described in the specific example group G1 is converted into divalence. Namely, specific examples (specific example group G12) of the “substituted or unsubstituted arylene group” refer to a group derived from the “aryl group” described in specific example group G1 by removal of one hydrogen atom bonded to the ring carbon atoms thereof.

Specific examples (specific example group G13) of the “substituted or unsubstituted divalent heterocyclic group” include a group in which the “heterocyclic group” described in the specific example group G2 is converted into divalence. Namely, specific examples (specific example group G13) of the “substituted or unsubstituted divalent heterocyclic group” refer to a group derived from the “heterocyclic group” described in specific example group G2 by removal of one hydrogen atom bonded to the ring atoms thereof.

Specific examples (specific example group G14) of the “substituted or unsubstituted alkylene group” include a group in which the “alkyl group” described in the specific example group G3 is converted into divalence. Namely, specific examples (specific example group G14) of the “substituted or unsubstituted alkylene group” refer to a group derived from the “alkyl group” described in specific example group G3 by removal of one hydrogen atom bonded to the carbon atoms constituting the alkane structure thereof.

The substituted or unsubstituted arylene group described herein is any group described below, unless otherwise specified.

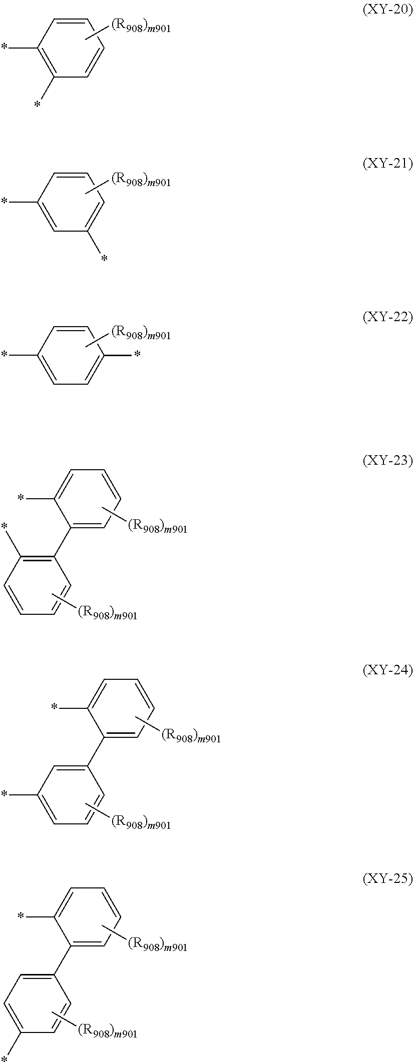

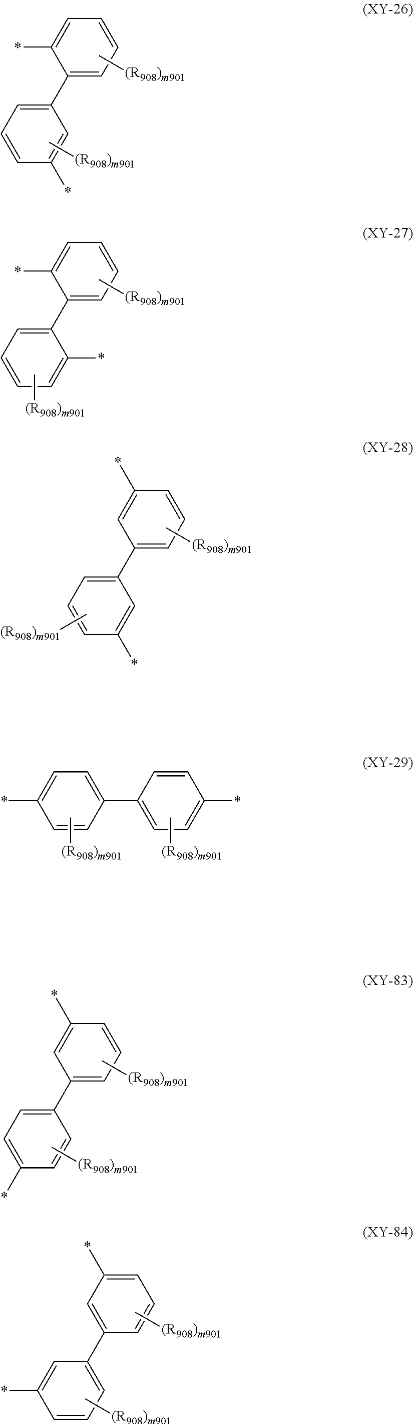

In the formulas (XY-20) to (XY-29), (XY-83) and (XY-84), R₉₀₈ is a substituent.

Then, m901 is an integer of 0 to 4, and when m901 is 2 or more, a plurality of R₉₀₈ may be the same with or different from each other.

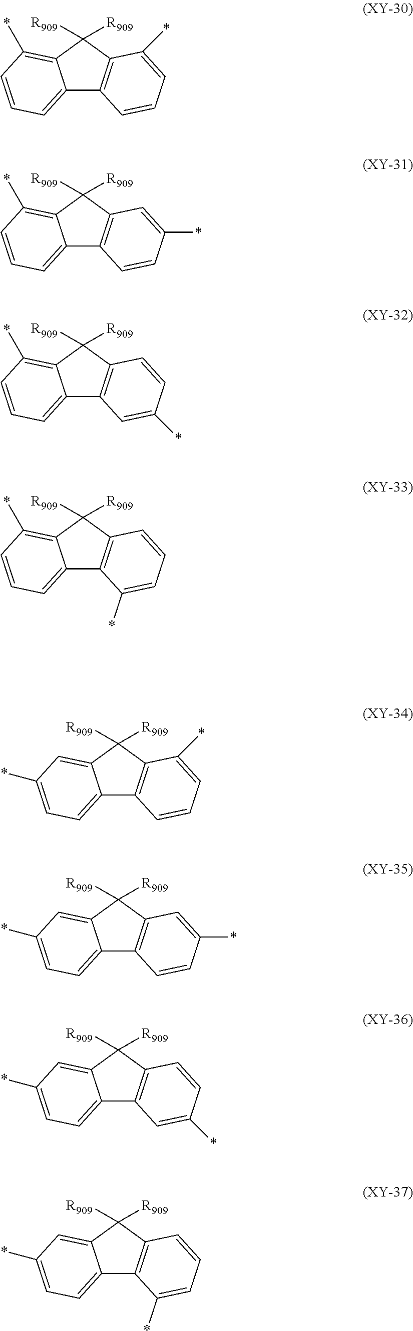

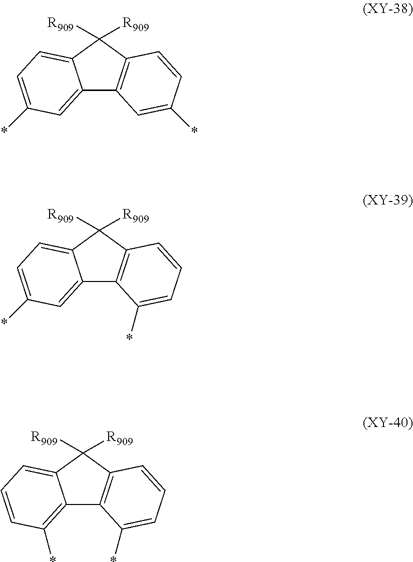

In the formulas (XY-30) to (XY-40), R₉₀₉ is independently a hydrogen atom or a substituent. Two of R₉₀₉ may form a ring by bonding with each other through a single bond.

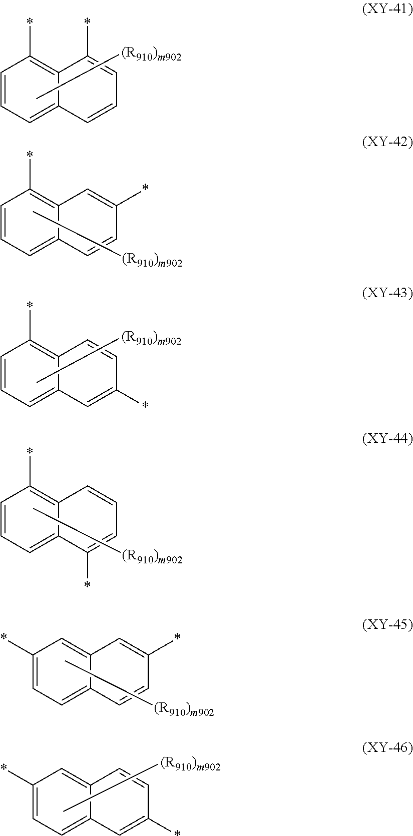

In the formulas (XY-41) to (XY-46), R₉₁₀ is a substituent.

Then, m902 is an integer of 0 to 6. When m902 is 2 or more, a plurality of R₉₁₀ may be the same with or different from each other.

The substituted or unsubstituted divalent heterocyclic group described herein is preferably any group described below, unless otherwise specified.

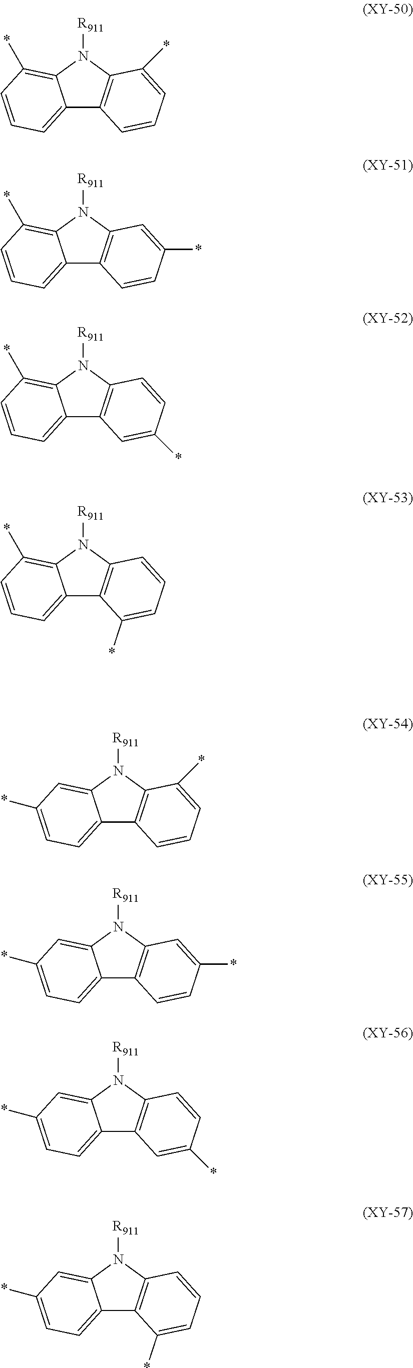

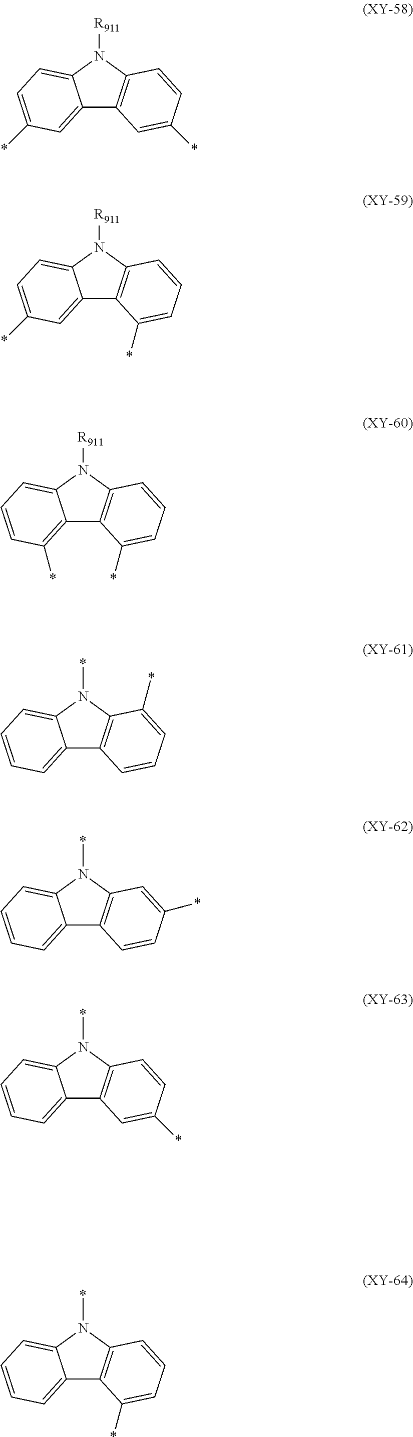

In the formulas (XY-50) to (XY-60), R₉₁₁ is a hydrogen atom or a substituent.

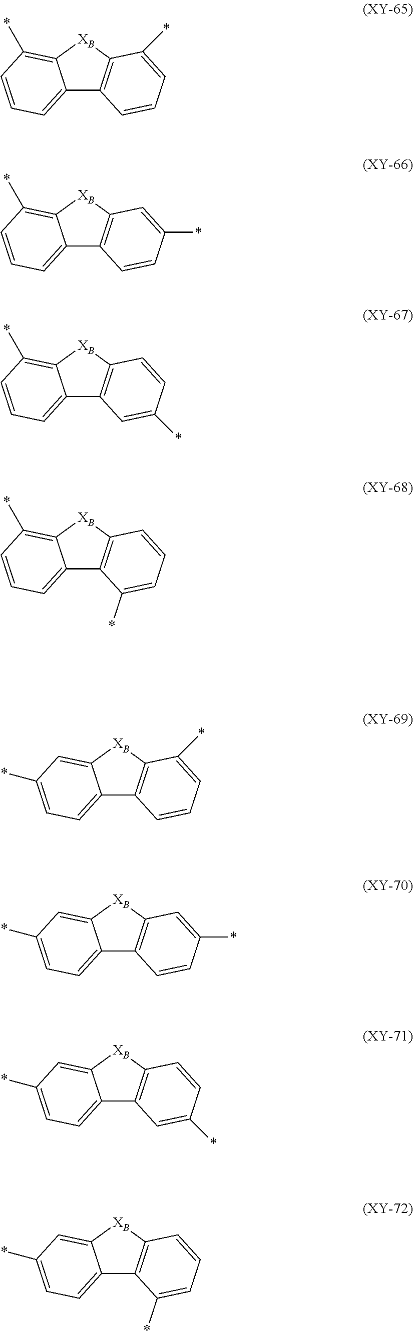

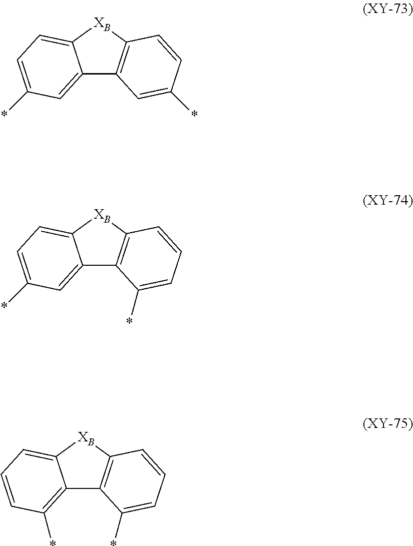

In the formulas (XY-65) to (XY-75), X_{B} is an oxygen atom or a sulfur atom.

Herein, a case where “one or more sets of two or more groups adjacent to each other form a substituted or unsubstituted and saturated or unsaturated ring by bonding with each other” will be described by taking, as an example, a case of an anthracene compound represented by the following formula (XY-80) in which a mother skeleton is an anthracene ring.

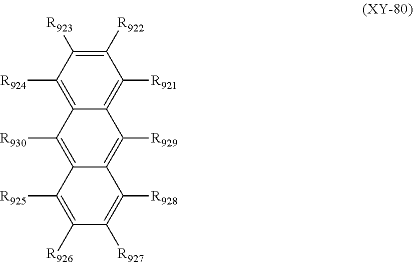

For example, two adjacent to each other into one set when “one or more sets of two or more groups adjacent to each other form the ring by bonding with each other” among R₉₂₁ to R₉₃₀ include R₉₂₁ and R₉₂₂, R₉₂₂ and R₉₂₃, R₉₂₃ and R₉₂₄, R₉₂₄ and R₉₃₀, R₉₃₀ and R₉₂₅, R₉₂₅ and R₉₂₆, R₉₂₆ and R₉₂₇, R₉₂₇ and R₉₂₈, R₉₂₈ and R₉₂₉, and R₉₂₉ and R₉₂₁.

The above-described “one or more sets” means that two or more sets of two groups adjacent to each other may simultaneously form the ring. For example, a case where R₉₂₁ and R₉₂₂ form a ring A by bonding with each other, and simultaneously R₉₂₅ and R₉₂₆ form a ring B by bonding with each other is represented by the following formula (XY-81).

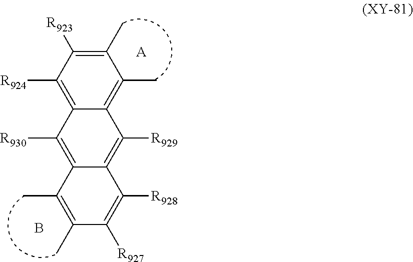

A case where “two or more groups adjacent to each other” form a ring means that, for example, R₉₂₁ and R₉₂₂ form a ring A by bonding with each other, and R₉₂₂ and R₉₂₃ form a ring C by bonding with each other. A case where the ring A and ring C sharing R₉₂₂ are formed, in which the ring A and the ring C are fused to the anthracene mother skeleton by three of R₉₂₁ to R₉₂₃ adjacent to each other, is represented by the following (XY-82).

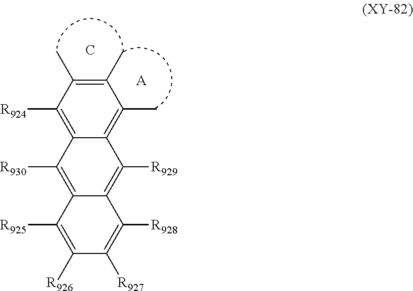

The rings A to C formed in the formulas (XY-81) and (XY-82) are a saturated or unsaturated ring.

A term “unsaturated ring” means an aromatic hydrocarbon ring or an aromatic heterocyclic ring. A term “saturated ring” means an aliphatic hydrocarbon ring or an aliphatic heterocyclic ring.

For example, the ring A formed by R₉₂₁ and R₉₂₂ being bonded with each other, represented by the formula (XY-81), means a ring formed by a carbon atom of the anthracene skeleton bonded with R₉₂₁, a carbon atom of the anthracene skeleton bonded with R₉₂₂, and one or more arbitrary elements. Specific examples include, when the ring A is formed by R₉₂₁ and R₉₂₂, a case where an unsaturated ring is formed of a carbon atom of an anthracene skeleton bonded with R₉₂₁, a carbon atom of the anthracene skeleton bonded with R₉₂₂, and four carbon atoms, in which a ring formed by R₉₂₁ and R₉₂₂ is formed into a benzene ring. Further, when a saturated ring is formed, the ring is formed into a cyclohexane ring.

Here, “arbitrary elements” are preferably a C element, a N element, an O element and a S element. In the arbitrary elements (for example, a case of the C element or the N element), the bond(s) that is(are) not involved in the formation of the ring may be terminated by a hydrogen atom, or may be substituted by an arbitrary substituent. When the ring contains the arbitrary elements other than the C element, the ring to be formed is a heterocyclic ring.

The number of “one or more arbitrary elements” forming the saturated or unsaturated ring is preferably 2 or more and 15 or less, more preferably 3 or more and 12 or less, and further preferably 3 or more and 5 or less.

As specific examples of the aromatic hydrocarbon ring, a structure in which the aryl group described in specific example group G1 is terminated with a hydrogen atom may be mentioned.

As specific examples of the aromatic heterocyclic ring, a structure in which the aromatic heterocyclic group described in specific example group G2 is terminated with a hydrogen atom may be mentioned.

As specific examples of the aliphatic hydrocarbon ring, a structure in which the cycloalkyl group described in specific example group G6 is terminated with a hydrogen atom may be mentioned.

When the above-described “saturated or unsaturated ring” has a substituent, the substituent is an “arbitrary substituent” as described below, for example. When the above-mentioned “saturated or unsaturated ring” has a substituent, specific examples of the substituent refer to the substituents described in above-mentioned “the substituent described herein”.

In one embodiment of the this specification, the substituent (hereinafter, referred to as an “arbitrary substituent” in several cases) in the case of the “substituted or unsubstituted” is a group selected from the group consisting of

an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,

—Si(R₉₀₁)(R₉₀₂)(R₉₀₃),

—O—(R₉₀₄),

—S—(R₉₀₅)

—N(R₉₀₆)(R₉₀₇)

wherein,
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and when
two or more of R₉₀₁ to R₉₀₇ exist, two or more of R₉₀₁ to R₉₀₇ may be the same with or different from each other,
a halogen atom, a cyano group, a nitro group,
an unsubstituted aryl group including 6 to 50 ring carbon atoms, and
an unsubstituted monovalent heterocyclic group including 5 to 50 ring atoms.

In one embodiment, the substituent in the case of “substituted or unsubstituted” is a group selected from the group consisting of

an alkyl group including 1 to 50 carbon atoms,
an aryl group including 6 to 50 ring carbon atoms, and
a monovalent heterocyclic group including 5 to 50 ring atoms.

In one embodiment, the substituent in the case of “substituted or unsubstituted” is a group selected from the group consisting of

an alkyl group including 1 to 18 carbon atoms,
an aryl group including 6 to 18 ring carbon atoms, and
a monovalent heterocyclic group including 5 to 18 ring atoms.

Specific examples of each group of the arbitrary substituent described above are as described above.

Herein, unless otherwise specified, the saturated or unsaturated ring (preferably substituted or unsubstituted and saturated or unsaturated five-membered or six-membered ring, more preferably a benzene ring) may be formed by the arbitrary substituents adjacent to each other.

Herein, unless otherwise specified, the arbitrary substituent may further have the substituent. Specific examples of the substituent that the arbitrary substituent further has include to the ones same as the arbitrary substituent described above.

### [Organic Electroluminescence Device]

An organic electroluminescence device according to the first aspect of the invention containing:

a cathode;
an anode; and
an emitting layer disposed between the cathode and the anode,

wherein the emitting layer contains a compound represented by the following formula (1) and a compound represented by the following formula (11).

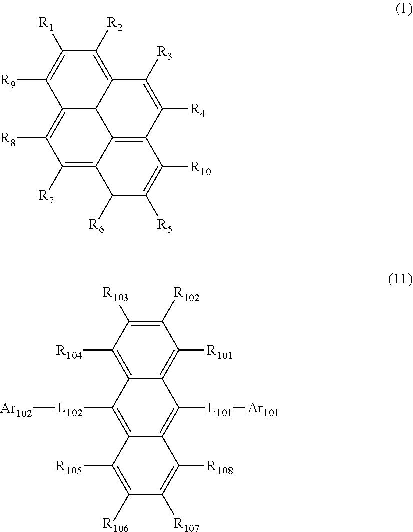

Each substituent in the formulas (1) and (11) will be described later.

The inventors have found that, by the use of a diaminopyrene compound represented by the formula (1) (dopant material), which has a sterically hindered group and an anthracene compound represented by the above formula (11) (host material) in combination in an emitting layer, a driving voltage could be further reduced while maintaining high luminous efficiency properties of the obtained organic EL device.

Next, a compound represented by the formula (1) will be described.

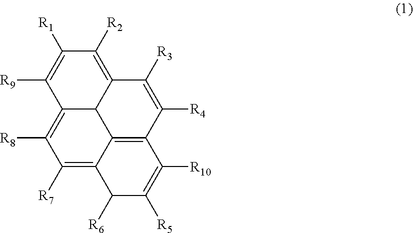

In the formula (1), at least one of R₁ to R₁₀ is a monovalent group represented by the following formula (2).

R₁ to R₁₀ which are not the monovalent group represented by the following formula (2) are independently

a hydrogen atom, a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a substituted or unsubstituted aryl group including 6 to 50 ring carbon atoms, or
a substituted or unsubstituted monovalent heterocyclic group including 5 to 50 ring atoms.

Adjacent two or more among R₁ to R₁₀ do not form a ring by bonding with each other.

R₉₀₁ to R₉₀₇ are independently

a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms.

When two or more of each of R₉₀₁ to R₉₀₇ are present, the two or more of each of R₉₀₁ to R₉₀₇ may be the same or different.

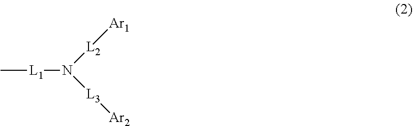

In the formula (2), at least one of Ar₁ and Ar₂ is a group represented by the following formula (3).

Ar₁ or Ar₂ which is not the monovalent group represented by the following formula (3) is a substituted or unsubstituted aryl group including 6 to 50 ring carbon atoms, or a substituted or unsubstituted monovalent heterocyclic group including 5 to 50 ring atoms.

L₁, L₂, and L₃ are independently a single bond,

a substituted or unsubstituted arylene group including 6 to 30 ring carbon atoms, or
a substituted or unsubstituted divalent heterocyclic group including 5 to 30 ring atoms.

When two or more of each of Ar₁, Ar₂, L₁, L₂, and L₃ are present, the two or more of each of Ar₁, Ar₂, L₁, L₂, and L₃ may be the same or different.

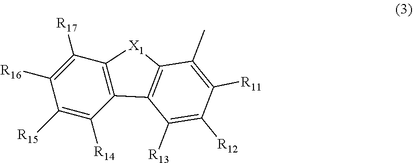

In the formula (3), R₁ is a substituted or unsubstituted alkyl group including 1 to 50 carbon atoms,

a substituted or unsubstituted aryl group including 6 to 50 ring carbon atoms, or
a substituted or unsubstituted monovalent heterocyclic group including 5 to 50 ring atoms.

One or more sets of adjacent two or more among R₁₂ to R₁₇ form a substituted or unsubstituted, saturated or unsaturated ring by bonding with each other, or do not form a substituted or unsubstituted, saturated or unsaturated ring.

R₁₂ to R₁₇ which do not form a substituted or unsubstituted, saturated or unsaturated ring are independently

a hydrogen atom, a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a substituted or unsubstituted aryl group including 6 to 50 ring carbon atoms, or
a substituted or unsubstituted monovalent heterocyclic group including 5 to 50 ring atoms.

R₉₀₁ to R₉₀₇ are as defined in the formula (1).

X₁ is an oxygen atom or a sulfur atom.

The compound represented by the formula (1) is used for an emitting layer to obtain an organic EL device having higher luminous efficiency.

It is preferable that X₁ in the formula (3) be an oxygen atom.

It is preferable that L₁ in the formula (2) be a single bond.

It is preferable that Ar₁ in the formula (2) be a group represented by the formula (3) and Ar₂ in the formula (2) be a substituted or unsubstituted aryl group including 6 to 50 ring carbon atoms.

It is preferable that L₂ and L₃ in the formula (2) be single bonds.

It is preferable that two among R₁ to R₁₀ in the formula (1) be the monovalent group represented by the formula (2).

It is preferable that one or more sets of adjacent two or more among R₁₂ to R₁₇ in the formula (3) do not form a substituted or unsubstituted, saturated or unsaturated ring by bonding with each other.

It is preferable that the compound represented by the formula (1) be a compound represented by the following formula (4A).

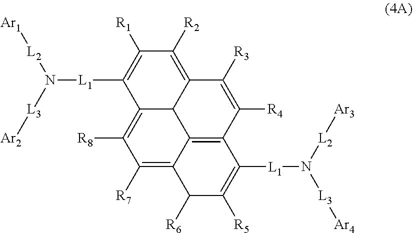

In the formula (4A),

R₁ to R₈ are as defined in the formula (1).

Ar₁, Ar₂, L₁, L₂, and L₃ are as defined in the formula (2).

Ar₃ and Ar₄ are independently a substituted or unsubstituted aryl group including 6 to 50 ring carbon atoms, a substituted or unsubstituted heteroaryl group including 5 to 50 ring atoms, or a group represented by the formula (3);

provided that at least one of Ar₃ and Ar₄ is a group represented by the formula (3).

It is preferable that the compound represented by the formula (1) be a compound represented by the following formula (4B).

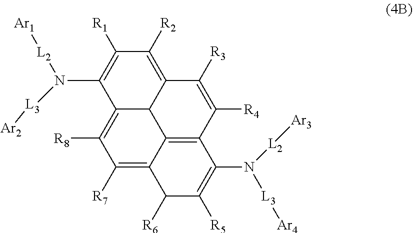

In the formula (4B),

R₁ to R₈ are as defined in the formula (1).

Ar₁, Ar₂, L₂, and L₃ are as defined in the formula (2).

Ar₃ and Ar₄ are independently a substituted or unsubstituted aryl group including 6 to 50 ring carbon atoms, a substituted or unsubstituted heteroaryl group including 5 to 50 ring atoms, or a group represented by the formula (3);

provided that at least one of Ar₃ and Ar₄ is a group represented by the formula (3).

It is preferable that the compound represented by the formula (1) be a compound represented by the following formula (4).

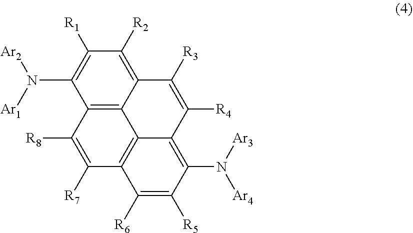

In the formula (4),

R₁ to R₈ is as defined in the formula (1).

Ar₁ and Ar₂ are as defined in the formula (2).

Ar₃ and Ar₄ are independently a substituted or unsubstituted aryl group including 6 to 50 ring carbon atoms, a substituted or unsubstituted heteroaryl group including 5 to 50 ring atoms, or a group represented by the formula (3);

provided that at least one (or both) of Ar₃ and Ar₄ is a group represented by the formula (3).

It is preferable that R₁ to R₈ in the formula (1) be independently a hydrogen atom, or a substituted or unsubstituted alkyl group including 1 to 50 carbon atoms;

Ar₁ in the formula (2), and Ar₃ in the formulas (4A), (4B) and (4) be independently a group represented by the formula (3);

Ar₂ in the formula (2), and Ar₄ in the formulas (4A), (4B) and (4) be independently a substituted or unsubstituted aryl group including 6 to 50 ring carbon atoms;

R₁₁ in the formula (3) be independently a substituted or unsubstituted alkyl group including 1 to 50 carbon atoms, or a substituted or unsubstituted aryl group including 6 to 50 ring carbon atoms; and

R₁₂ to R₁₇ in the formula (3) be independently a hydrogen atom, a substituted or unsubstituted alkyl group including 1 to 50 carbon atoms, or a substituted or unsubstituted aryl group including 6 to 50 ring carbon atoms.

It is preferable that R₁ to R₈ in the formula (1) be independently a hydrogen atom, or a substituted or unsubstituted alkyl group including 1 to 18 carbon atoms;

Ar₁ in the formula (2), and Ar₃ in the formulas (4A), (4B) and (4) be independently a group represented by the formula (3);

Ar₂ in the formula (2), and Ar₄ in the formulas (4A), (4B) and (4) be independently a substituted or unsubstituted aryl group including 6 to 18 ring carbon atoms;

R₁₁ in the formula (3) be independently a substituted or unsubstituted alkyl group including 1 to 18 carbon atoms, or a substituted or unsubstituted aryl group including 6 to 18 ring carbon atoms; and

R₁₂ to R₁₇ in the formula (3) be independently a hydrogen atom, a substituted or unsubstituted alkyl group including 1 to 18 carbon atoms, or a substituted or unsubstituted aryl group including 6 to 18 ring carbon atoms.

It is preferable that Ar₂ in the formula (2) and Ar₄ in the formulas (4A), (4B) and (4) be independently a substituted or unsubstituted phenyl group, a substituted or unsubstituted biphenyl group, or a substituted or unsubstituted naphthyl group.

When Ar₂ and Ar₄ have a substituent, an alkyl group including 1 to 18 (preferably 1 to 8) carbon atoms is preferable as the substituent, and Ar₂ and Ar₄ may be substituted by 1 or 2 or more alkyl groups. Examples of Ar₂ and Ar₄ in the case of having a substituent include a methylphenyl group, a dimethylphenyl group, a methylbiphenyl group, and the like.

It is preferable that R₁ in the formula (3) be a substituted or unsubstituted alkyl group including 1 to 18 carbon atoms. It is more preferable that R₁ be a substituted or unsubstituted alkyl group including 1 to 8 carbon atoms.

It is preferable that R₁ to R₈ in the formula (1) be hydrogen atoms or a substituted or unsubstituted alkyl group including 1 to 18 (preferably 1 to 8) carbon atoms.

In one embodiment, R₁ to R₈ in the formula (1) are hydrogen atoms.

In one embodiment, at least one of R₁ to R₈ in the formula (1) is a substituted or unsubstituted alkyl group including 1 to 18 (preferably 1 to 8) carbon atoms, and R₁ to R₈ which are not the substituted or unsubstituted alkyl group including 1 to 18 carbon atoms are hydrogen atoms.

In one embodiment, at least two of R₁ to R₈ in the formula (1) are substituted or unsubstituted alkyl groups including 1 to 18 (preferably 1 to 8) carbon atoms, and R₁ to R₈ which are not the substituted or unsubstituted alkyl group including 1 to 18 carbon atoms are hydrogen atoms.

For example, R₂ and R₆ among R₁ to R₈ in the formula (1) are an unsubstituted alkyl group including 1 to 8 carbon atoms, and R₁, R₃ to R₅, R₇, and R₈ are hydrogen atoms.

For example, R₂ and R₆ among R₁ to R₈ in the formula (1) are an unsubstituted alkyl group including 1 to 4 carbon atoms, and R₁, R₃ to R₅, R₇, and R₈ are hydrogen atoms.

R₁₂ to R₁₇ in the formula (3) are preferably a hydrogen atom or a substituted or unsubstituted alkyl group including 1 to 18 (preferably 1 to 8) carbon atoms. R₁₇ among R₁₂ to R₁₇ may be a substituted or unsubstituted alkyl group including 1 to 18 (preferably 1 to 8) carbon atoms, and R₁₂ to R₁₆ may be hydrogen atoms.

The compound represented by the formula (1) is preferably a compound represented by any one of the following formulas (5A) to (5D).

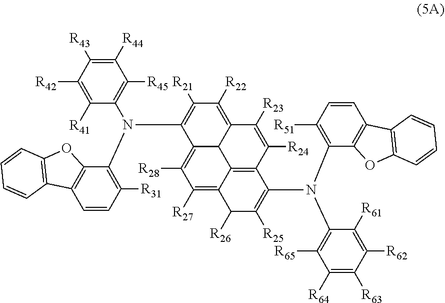

In the formula (5A), R₂₁ to R₂₈ are independently a hydrogen atom or a substituted or unsubstituted alkyl group including 1 to 50 carbon atoms.

R₃₁ is a substituted or unsubstituted alkyl group including 1 to 50 carbon atoms.

R₄₁ to R₄₅ are independently a hydrogen atom or a substituted or unsubstituted alkyl group including 1 to 50 carbon atoms.

R₅₁ is a substituted or unsubstituted alkyl group including 1 to 50 carbon atoms.

R₆₁ to R₆₅ are independently a hydrogen atom or a substituted or unsubstituted alkyl group including 1 to 50 carbon atoms.

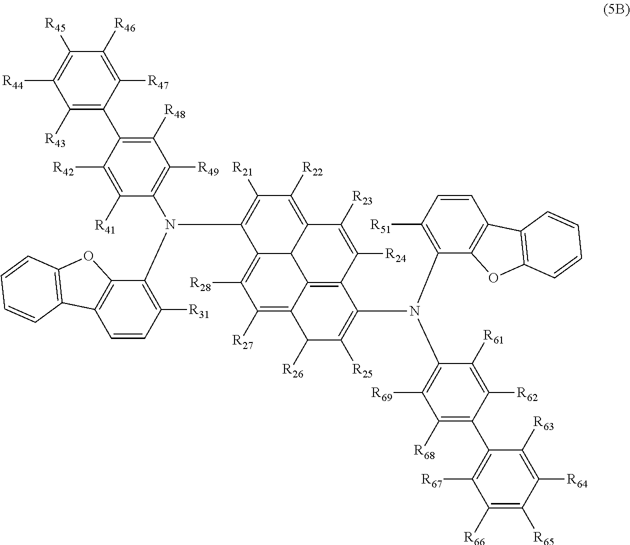

In the formula (5B), R₂₁ to R₂₈ are independently a hydrogen atom or a substituted or unsubstituted alkyl group including 1 to 50 carbon atoms.

R₃₁ is a substituted or unsubstituted alkyl group including 1 to 50 carbon atoms.

R₄₁ to R₄₉ are independently a hydrogen atom or a substituted or unsubstituted alkyl group including 1 to 50 carbon atoms.

R₅₁ is a substituted or unsubstituted alkyl group including 1 to 50 carbon atoms.

R₆₁ to R₆₉ are independently a hydrogen atom or a substituted or unsubstituted alkyl group including 1 to 50 carbon atoms.

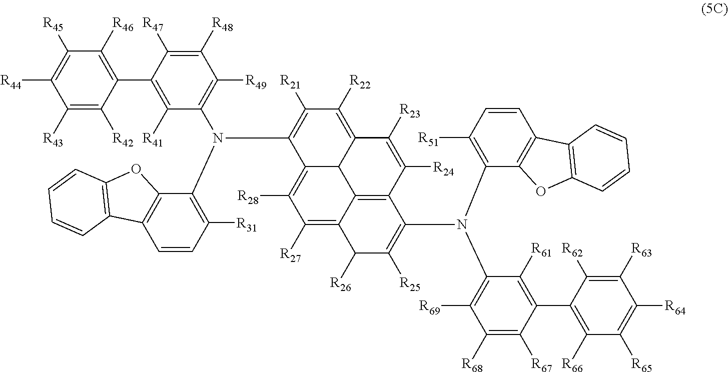

In the formula (5C), R₂₁ to R₂₈ are independently a hydrogen atom or a substituted or unsubstituted alkyl group including 1 to 50 carbon atoms.

R₃₁ is a substituted or unsubstituted alkyl group including 1 to 50 carbon atoms.

R₄₁ to R₄₉ are independently a hydrogen atom or a substituted or unsubstituted alkyl group including 1 to 50 carbon atoms.

R₅₁ is a substituted or unsubstituted alkyl group including 1 to 50 carbon atoms.

R₆₁ to R₆₉ are independently a hydrogen atom or a substituted or unsubstituted alkyl group including 1 to 50 carbon atoms.

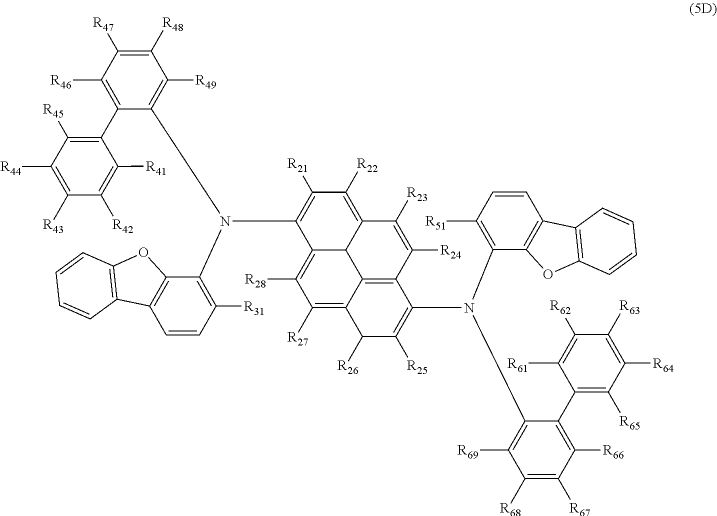

In the formula (5D), R₂₁ to R₂₈ are independently a hydrogen atom or a substituted or unsubstituted alkyl group including 1 to 50 carbon atoms.

R₃₁ is a substituted or unsubstituted alkyl group including 1 to 50 carbon atoms.

R₄₁ to R₄₉ are independently a hydrogen atom or a substituted or unsubstituted alkyl group including 1 to 50 carbon atoms.

R₅₁ is a substituted or unsubstituted alkyl group including 1 to 50 carbon atoms.

R₆₁ to R₆₉ are independently a hydrogen atom or a substituted or unsubstituted alkyl group including 1 to 50 carbon atoms.

The carbon number of the alkyl groups for R₂₁ to R₂₈, R₃₁, R₄₁ to R₄₉, R₅₁, and R₆₁ to R₆₉ are preferably 1 to 18, and more preferably 1 to 8.

Details of the substituents in the formulas (1), (2), (3), (4A), (4B), (4) and (5A) to (5D), and the substituent in the case of “a substituted or unsubstituted” are as defined in the [Definition] part of this specification.

The compound represented by the formula (1) can be synthesized with reference to the reactions described in Synthesis Examples by using known alternative reactions or raw materials tailored to the target compound.

Specific examples of the compound represented by the formula (1) will be described below, but these are merely examples, and the compound represented by the formula (1) is not limited to the following specific examples.

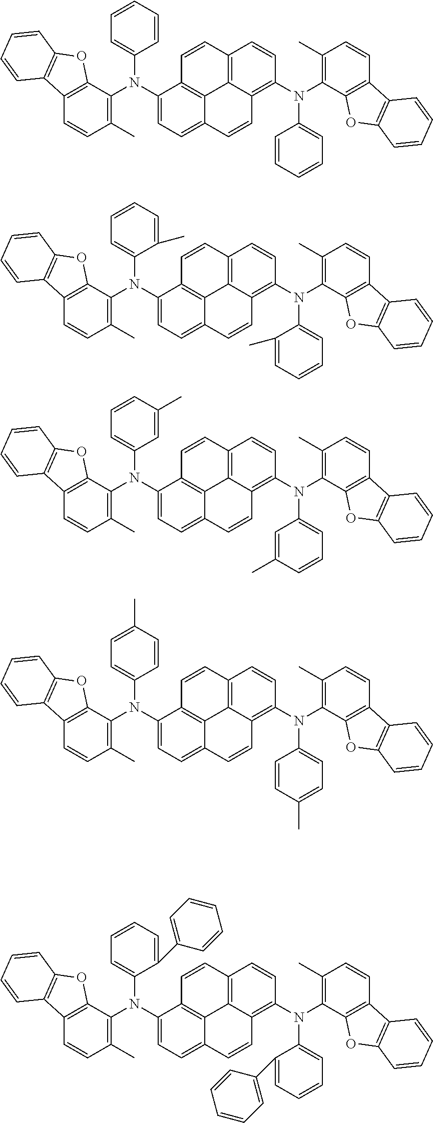

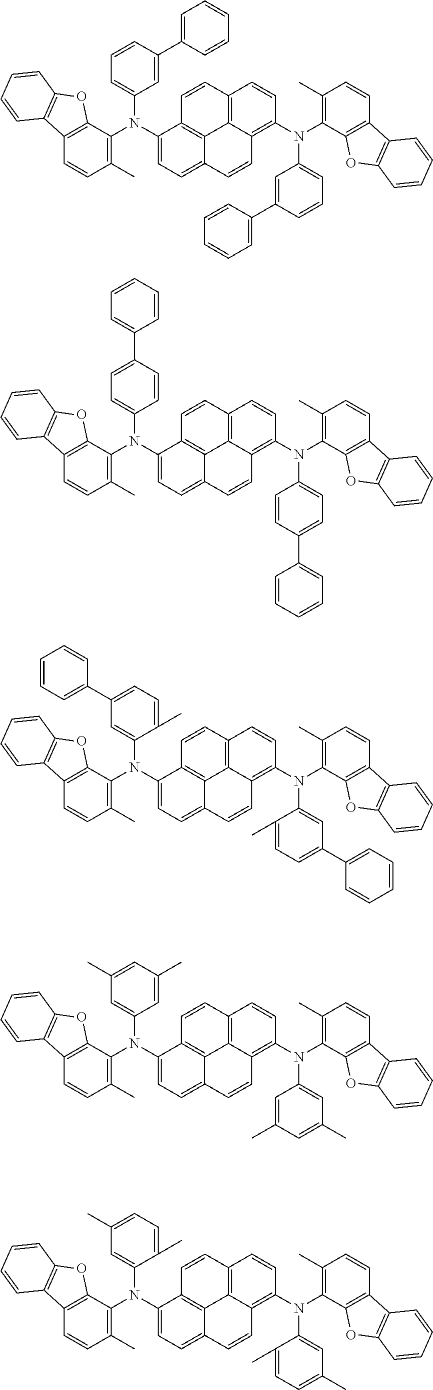

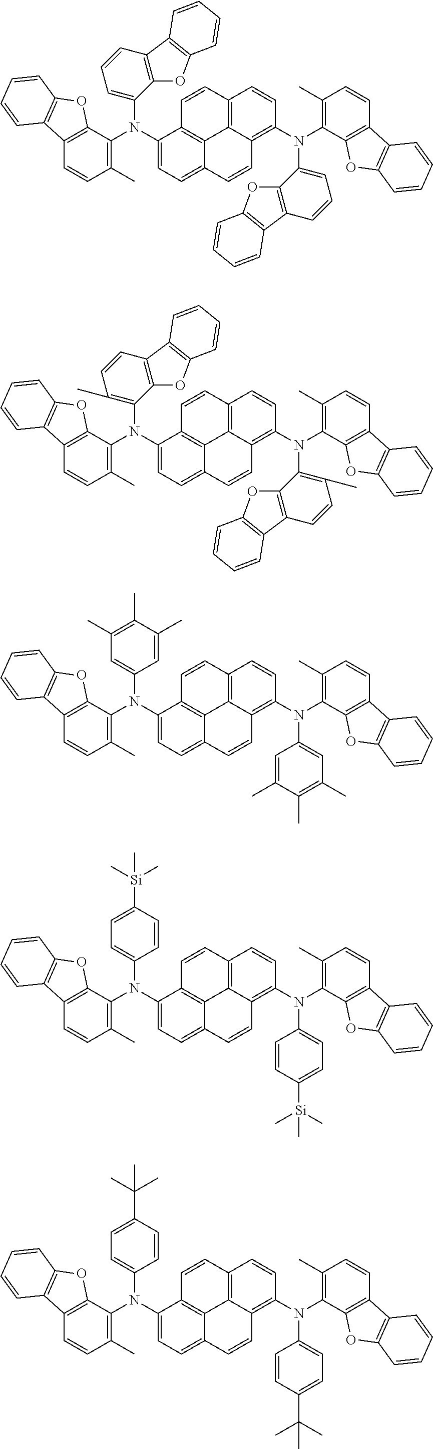

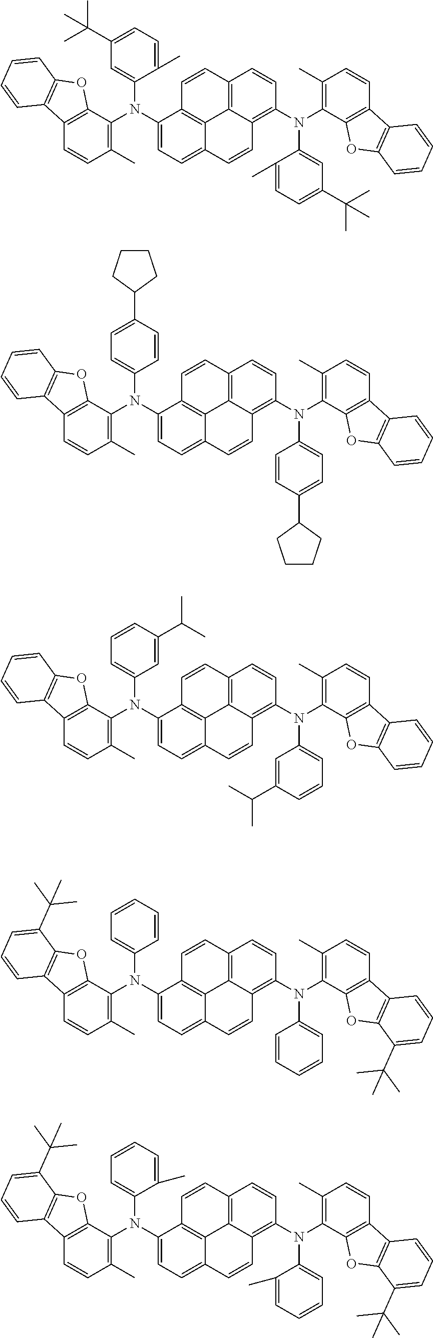

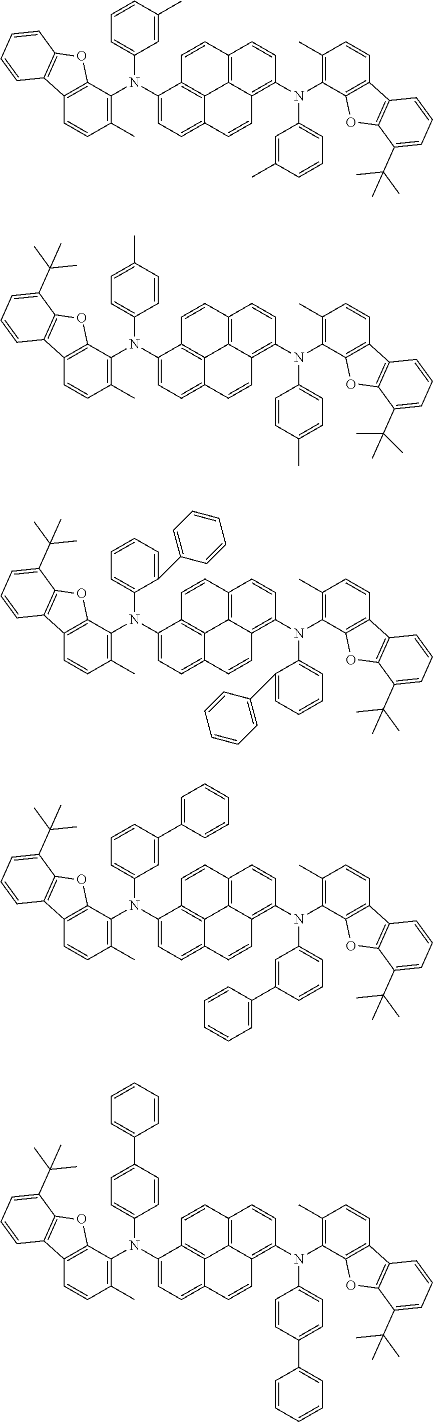

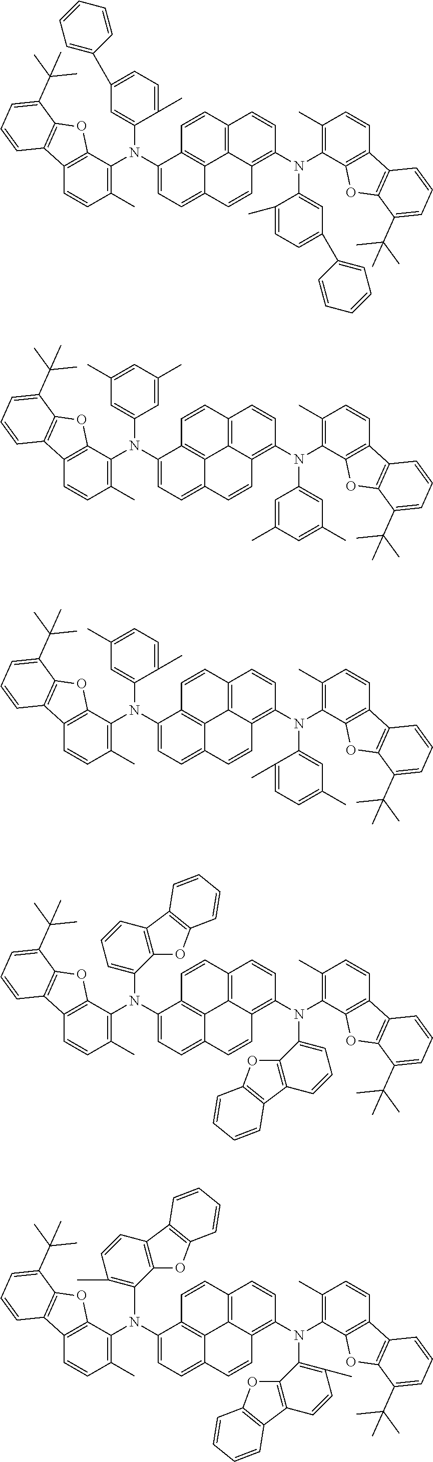

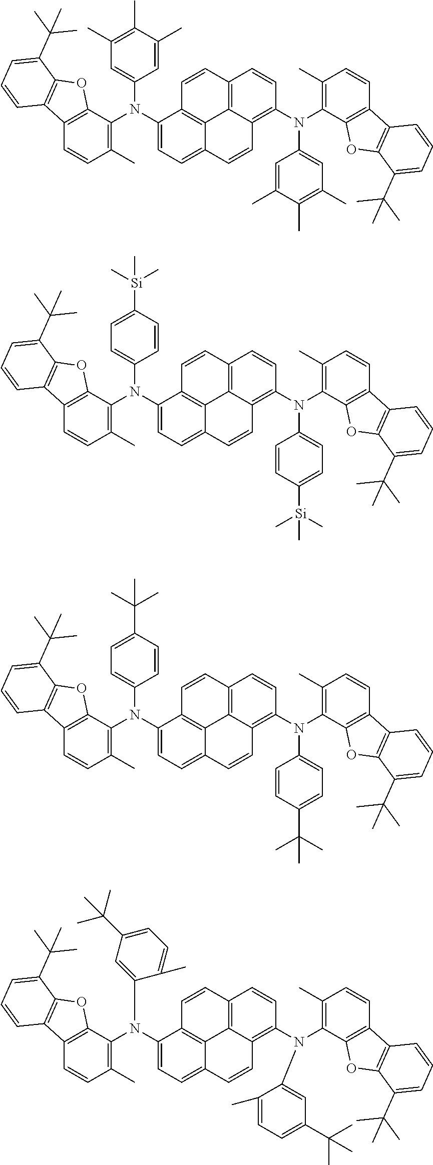

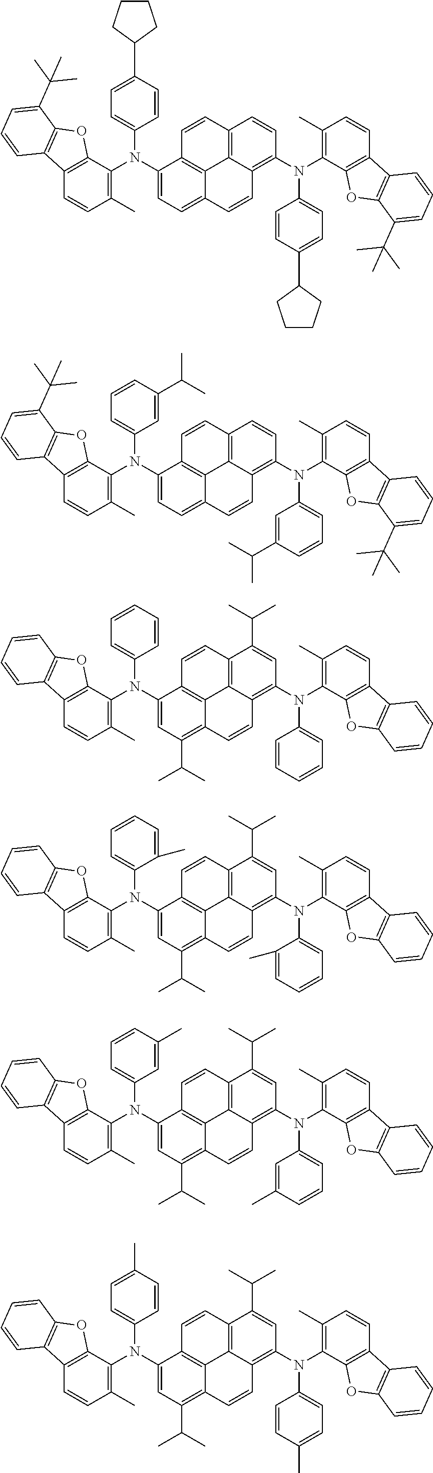

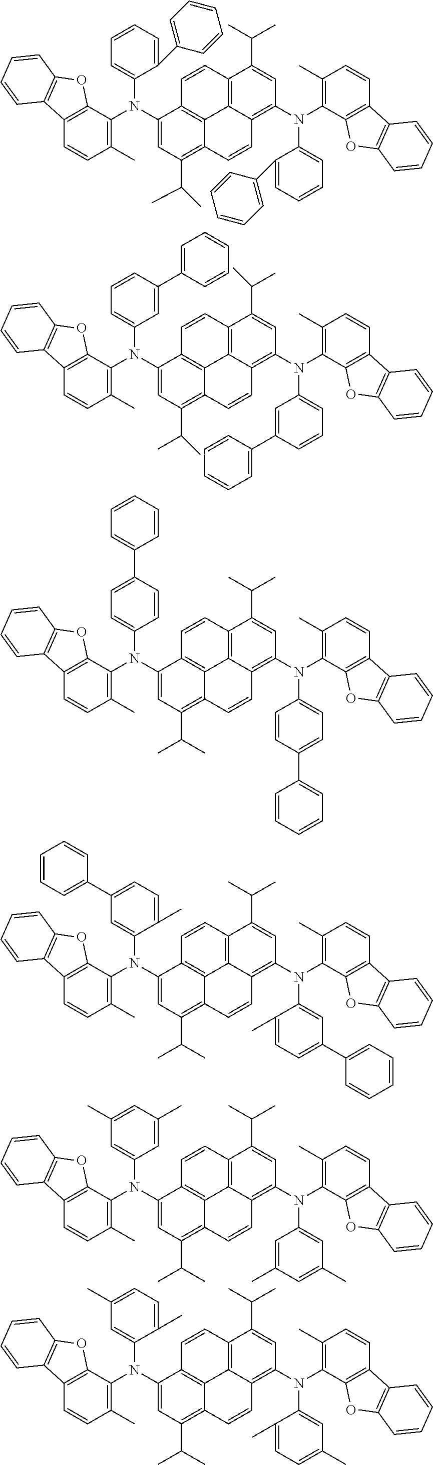

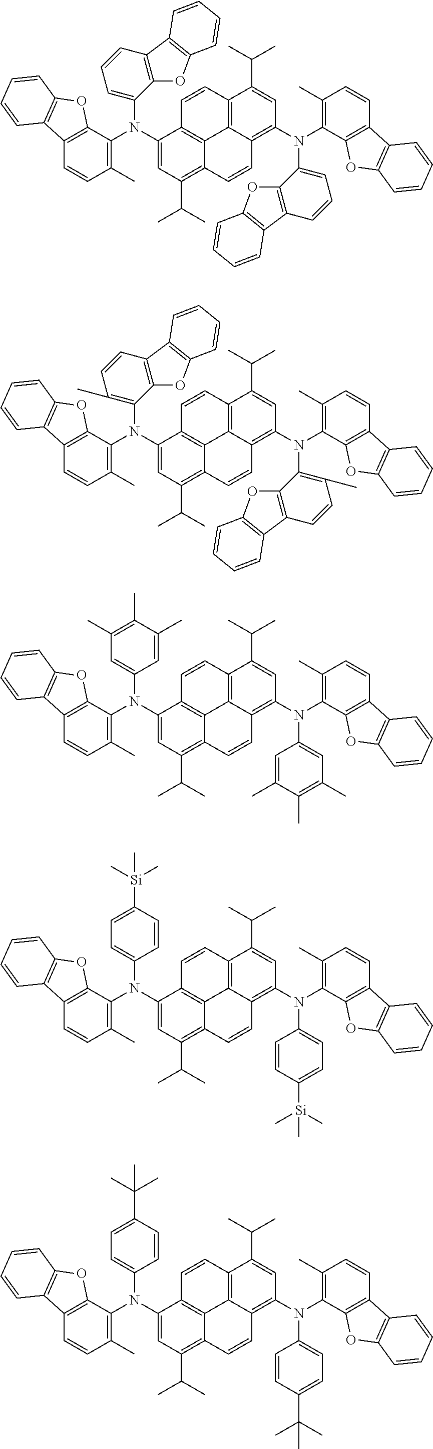

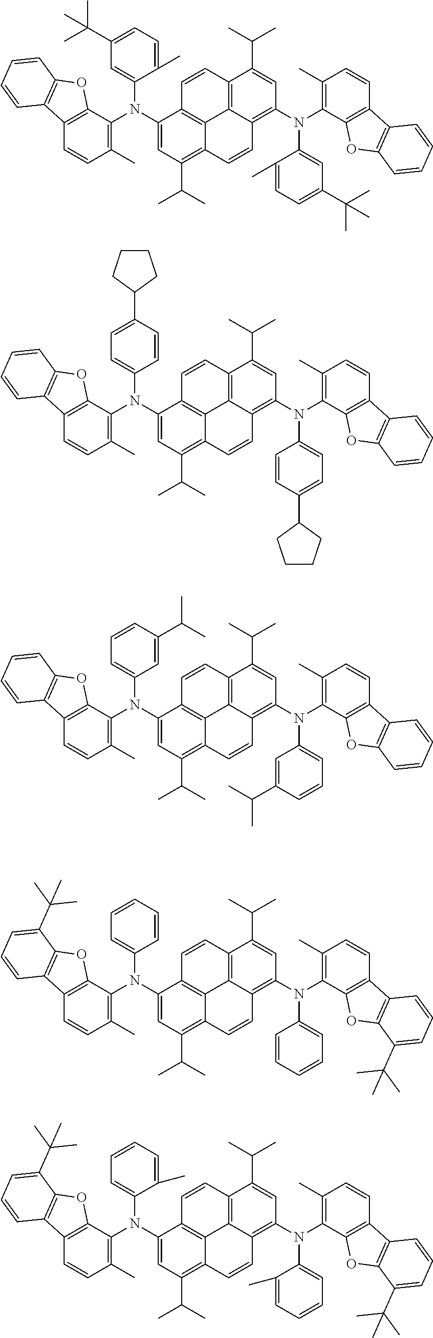

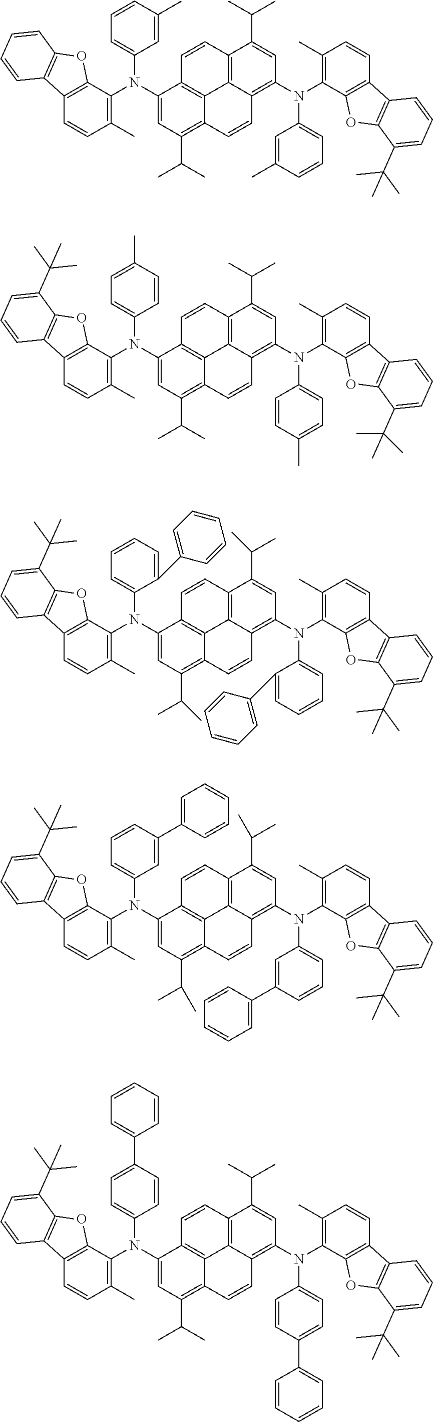

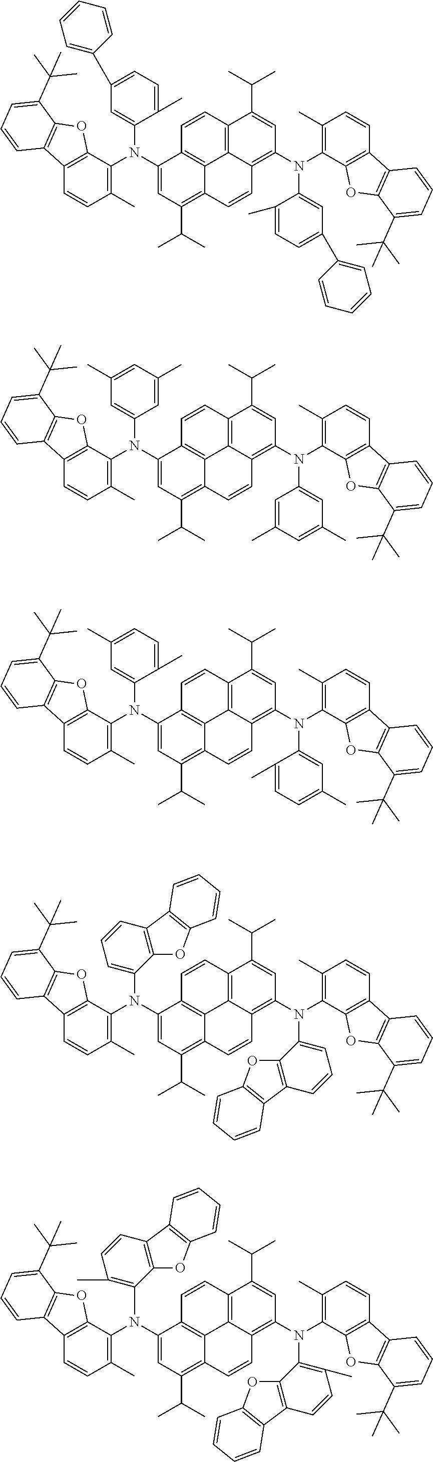

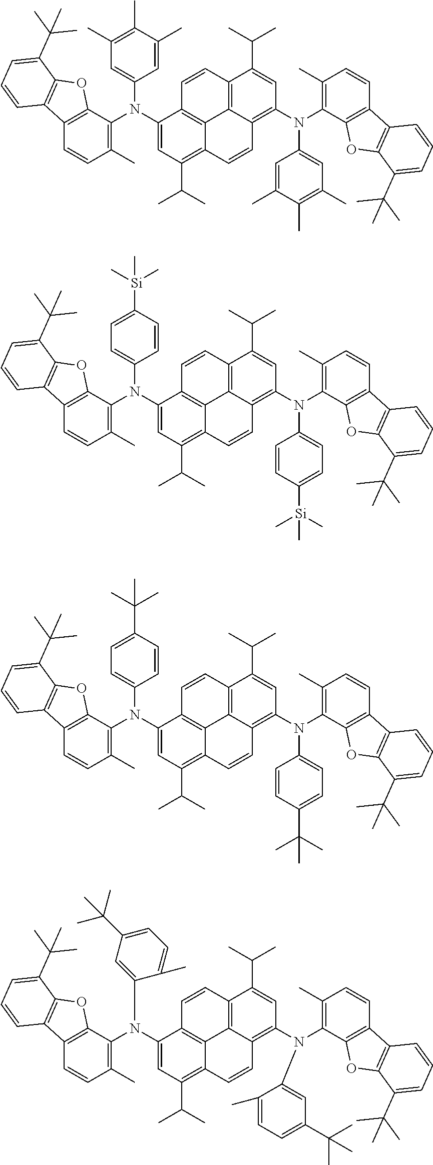

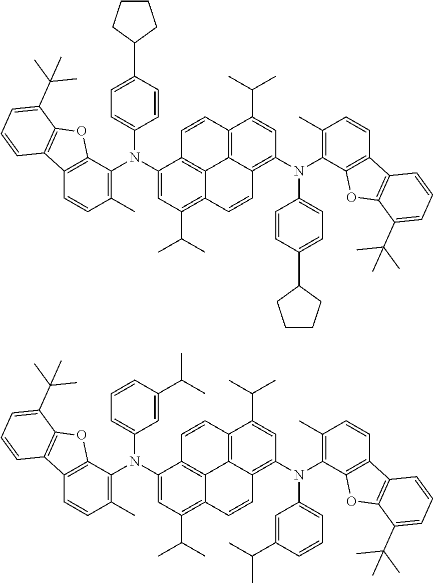 [SMILES: Cc1ccc2c(oc3c(C(C)(C)C)cccc32)c1N(c1ccc(C2CCCC2)cc1)c1cc(C(C)C)c2ccc3c(N(c4ccc(C5CCCC5)cc4)c4c(C)ccc5c4oc4c(C(C)(C)C)cccc45)cc(C(C)C)c4ccc1c2c43 ; Cc1ccc2c(oc3c(C(C)(C)C)cccc32)c1N(c1cccc(C(C)C)c1)c1cc(C(C)C)c2ccc3c(N(c4cccc(C(C)C)c4)c4c(C)ccc5c4oc4c(C(C)(C)C)cccc45)cc(C(C)C)c4ccc1c2c43]

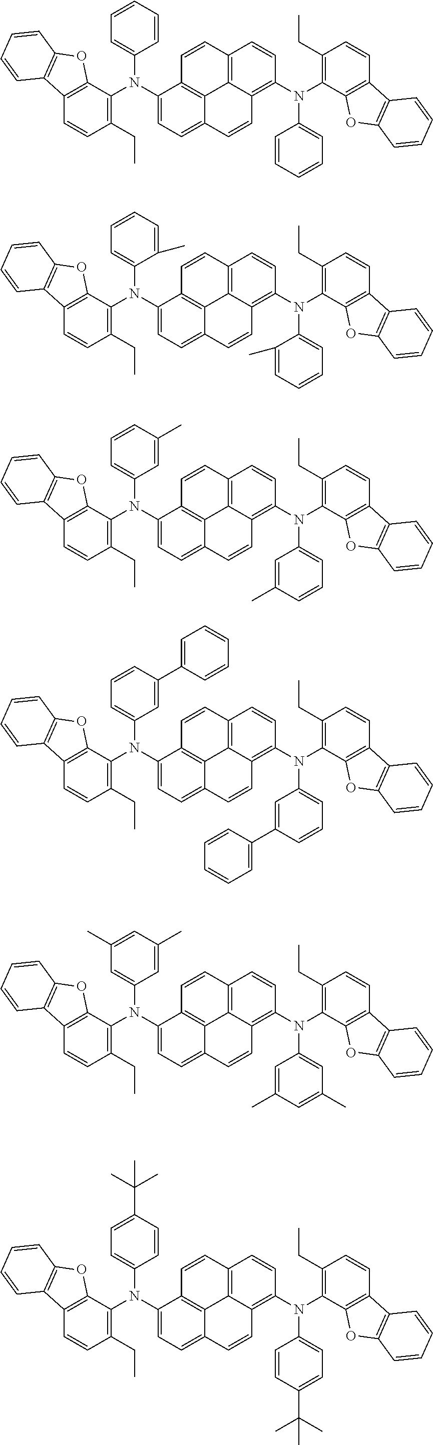

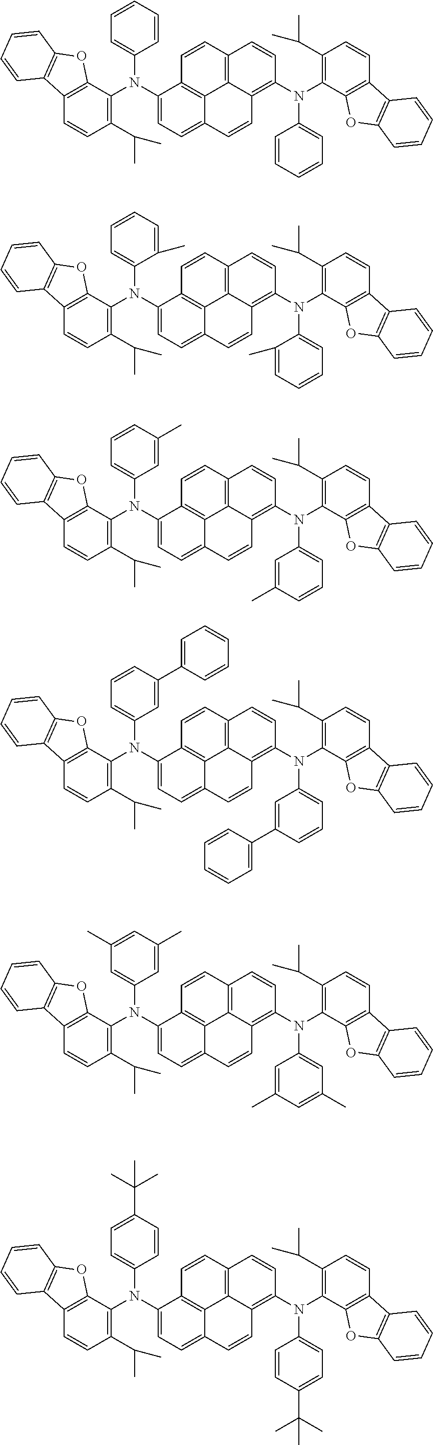

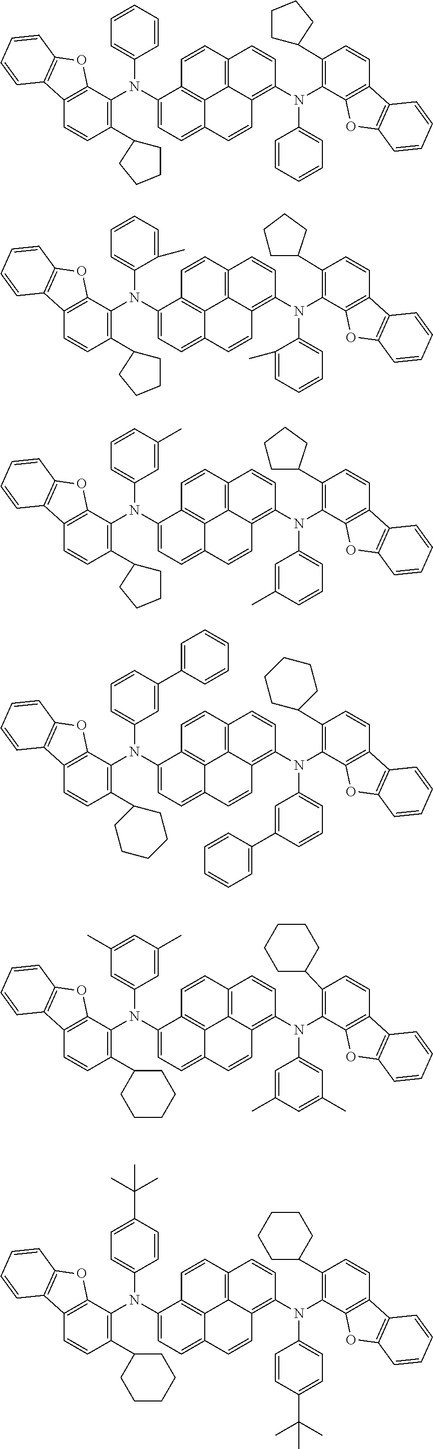

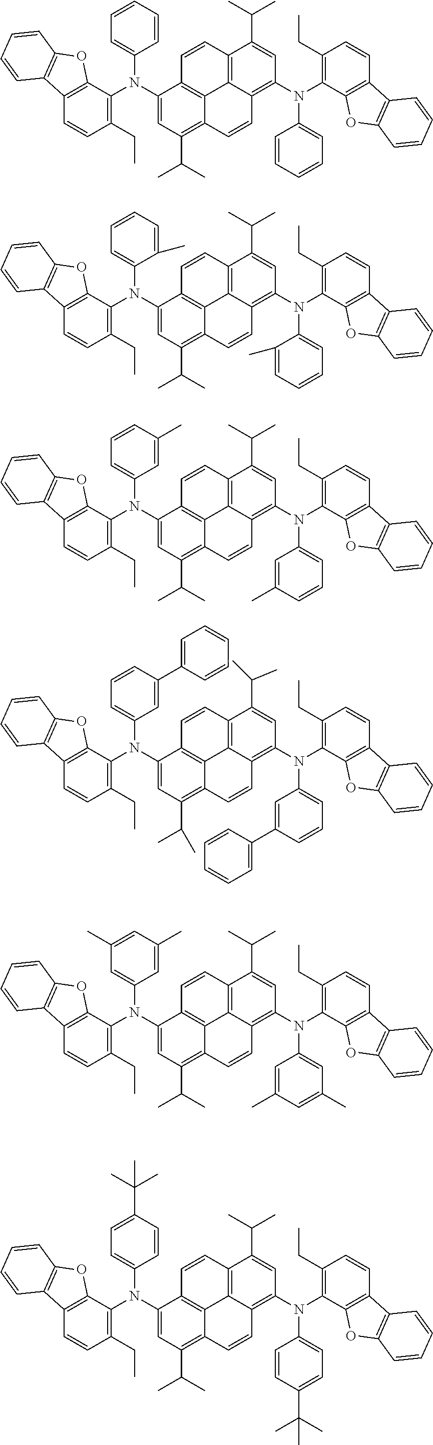

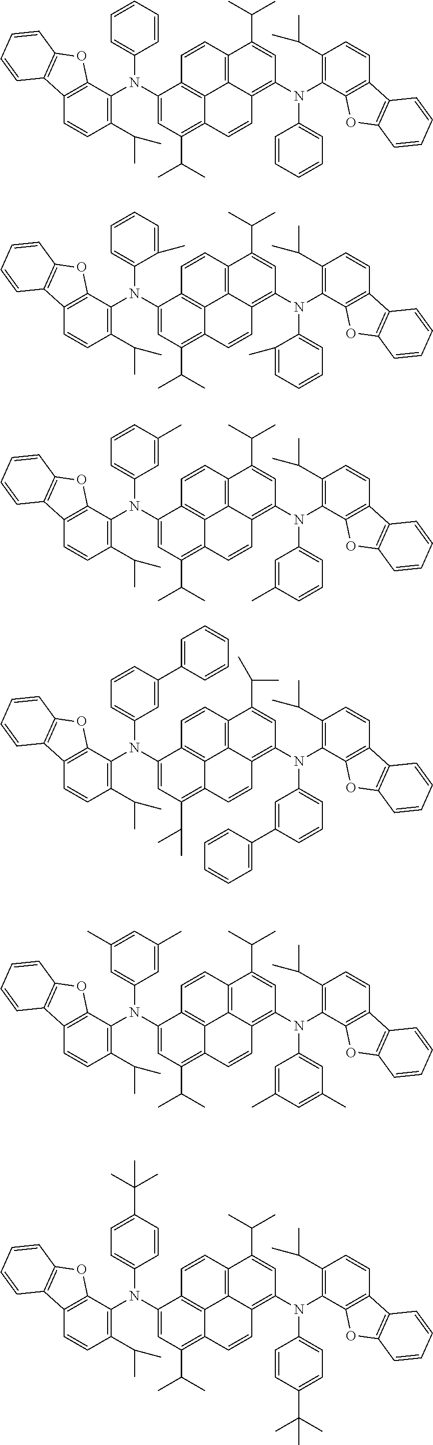

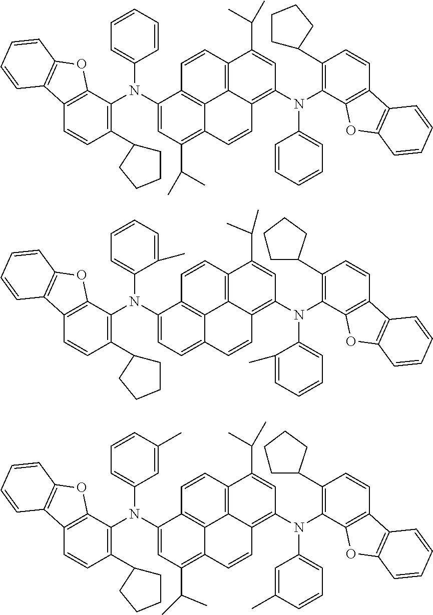

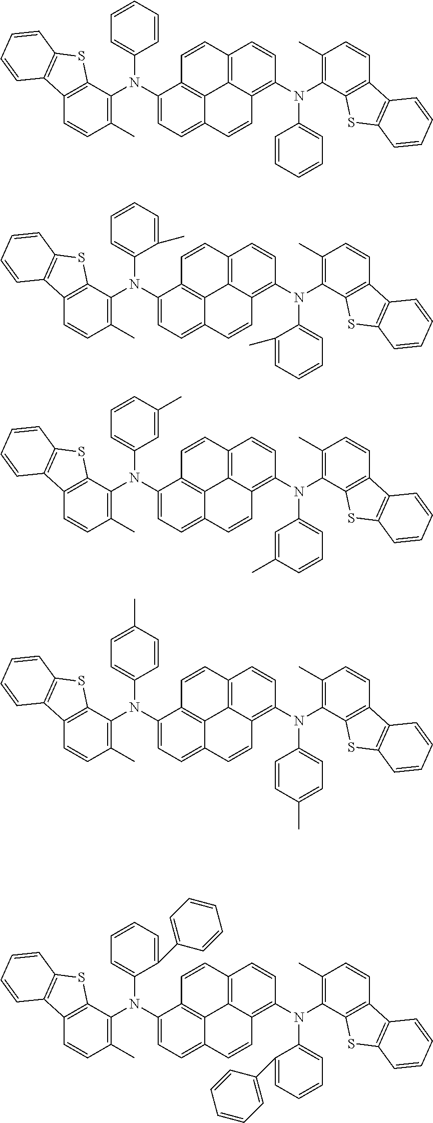

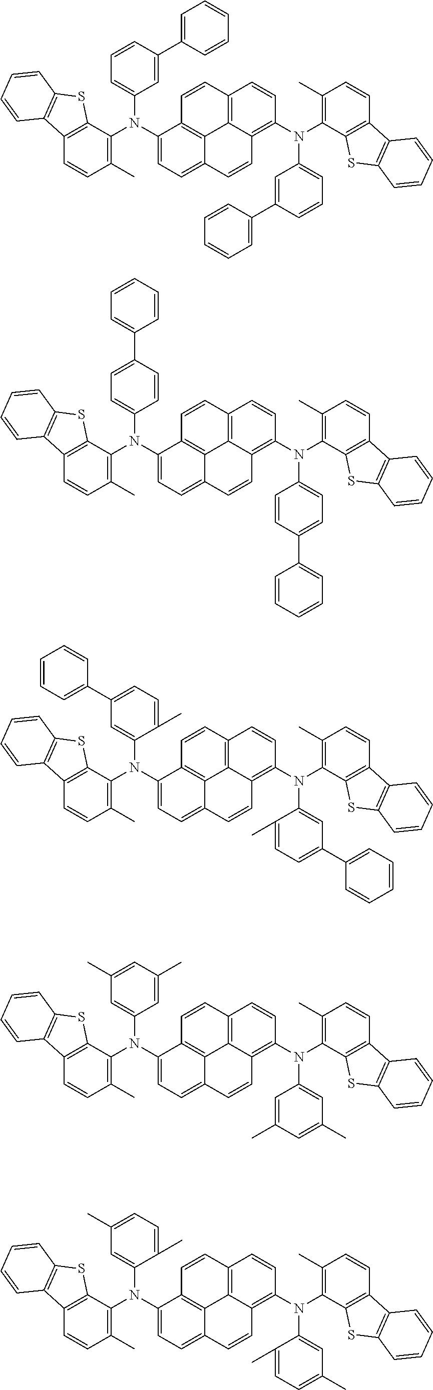

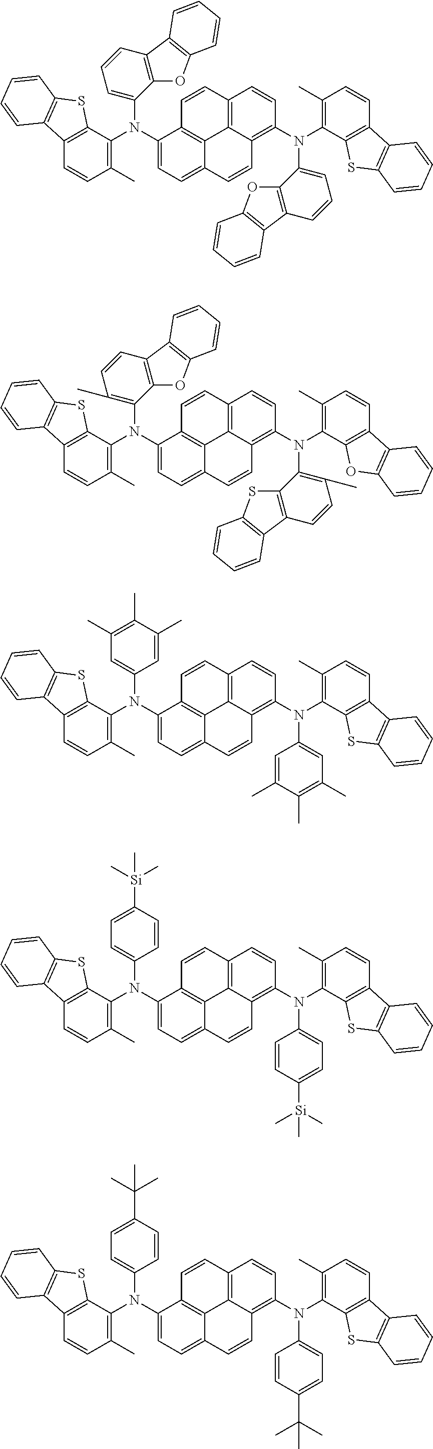

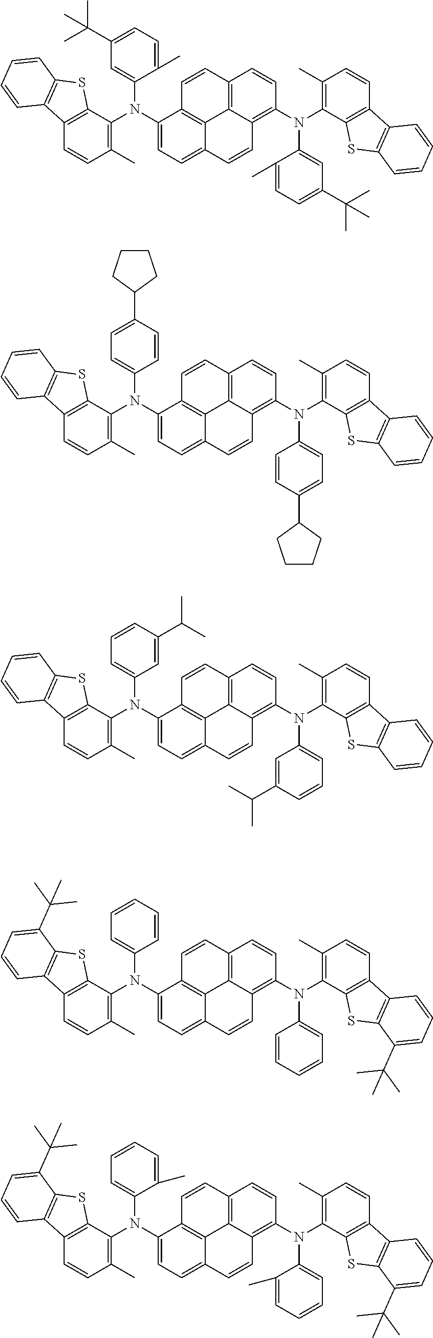

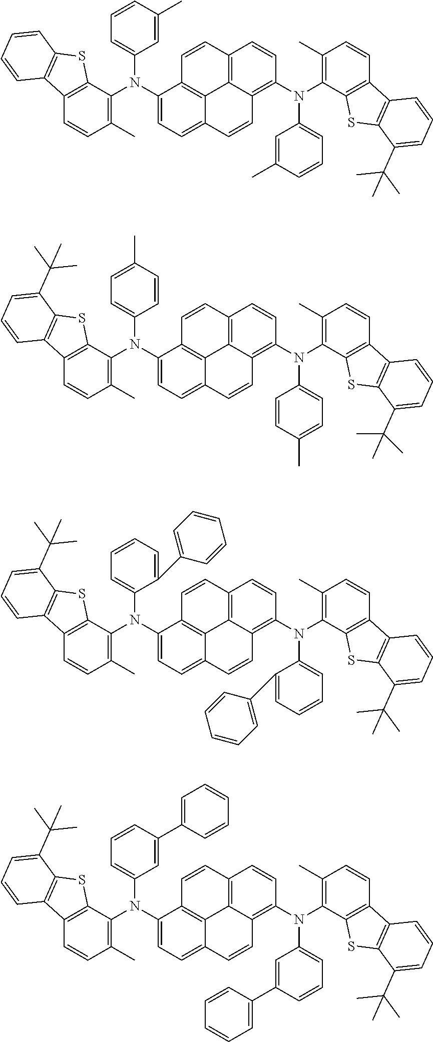 [SMILES: Cc1cccc(N(c2ccc3ccc4c(N(c5cccc(C)c5)c5c(C)ccc6c5sc5c(C(C)(C)C)cccc56)ccc5ccc2c3c54)c2c(C)ccc3c2sc2ccccc23)c1 ; Cc1ccc(N(c2ccc3ccc4c(N(c5ccc(C)cc5)c5c(C)ccc6c5sc5c(C(C)(C)C)cccc56)ccc5ccc2c3c54)c2c(C)ccc3c2sc2c(C(C)(C)C)cccc23)cc1 ; Cc1ccc2c(sc3c(C(C)(C)C)cccc32)c1N(c1ccccc1-c1ccccc1)c1ccc2ccc3c(N(c4ccccc4-c4ccccc4)c4c(C)ccc5c4sc4c(C(C)(C)C)cccc45)ccc4ccc1c2c43 ; Cc1ccc2c(sc3c(C(C)(C)C)cccc32)c1N(c1cccc(-c2ccccc2)c1)c1ccc2ccc3c(N(c4cccc(-c5ccccc5)c4)c4c(C)ccc5c4sc4c(C(C)(C)C)cccc45)ccc4ccc1c2c43]

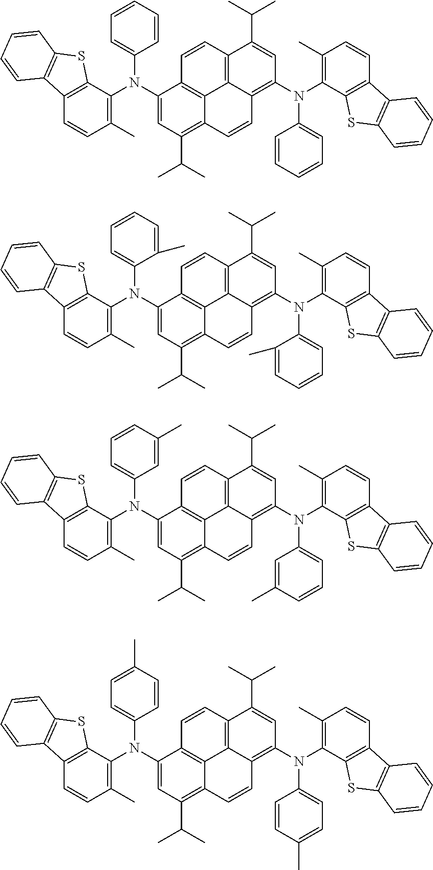 [SMILES: Cc1ccc2c(sc3ccccc32)c1N(c1ccccc1)c1cc(C(C)C)c2ccc3c(N(c4ccccc4)c4c(C)ccc5c4sc4ccccc45)cc(C(C)C)c4ccc1c2c43 ; Cc1ccccc1N(c1cc(C(C)C)c2ccc3c(N(c4ccccc4C)c4c(C)ccc5c4sc4ccccc45)cc(C(C)C)c4ccc1c2c43)c1c(C)ccc2c1sc1ccccc12 ; Cc1cccc(N(c2cc(C(C)C)c3ccc4c(N(c5cccc(C)c5)c5c(C)ccc6c5sc5ccccc56)cc(C(C)C)c5ccc2c3c54)c2c(C)ccc3c2sc2ccccc23)c1 ; Cc1ccc(N(c2cc(C(C)C)c3ccc4c(N(c5ccc(C)cc5)c5c(C)ccc6c5sc5ccccc56)cc(C(C)C)c5ccc2c3c54)c2c(C)ccc3c2sc2ccccc23)cc1]

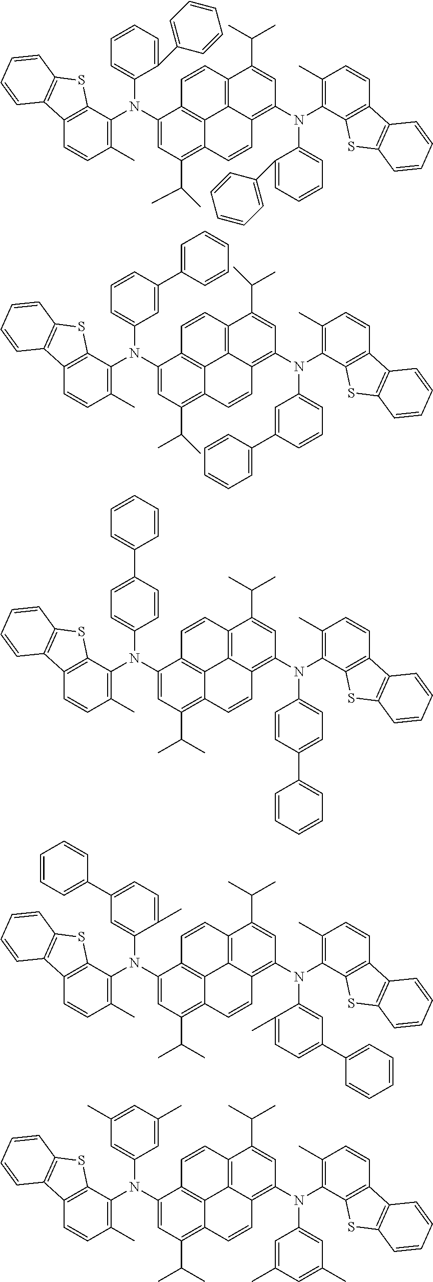

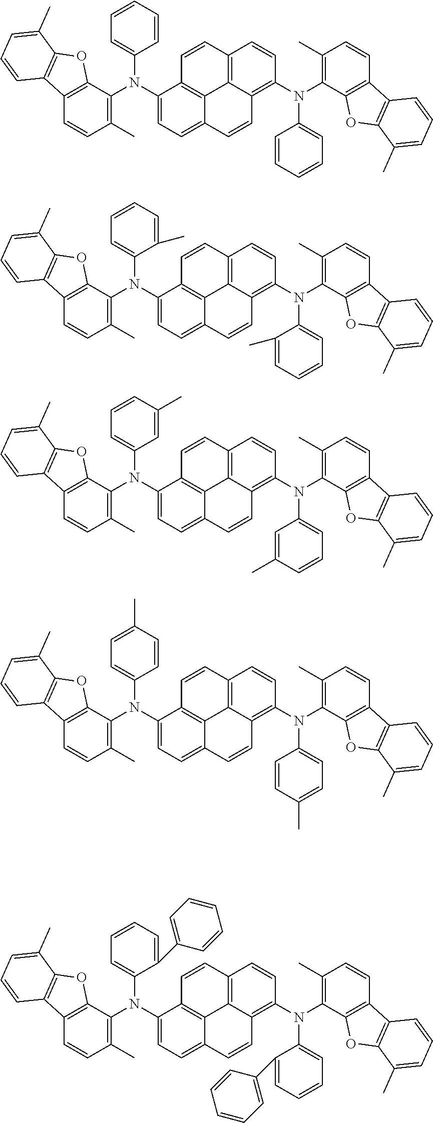

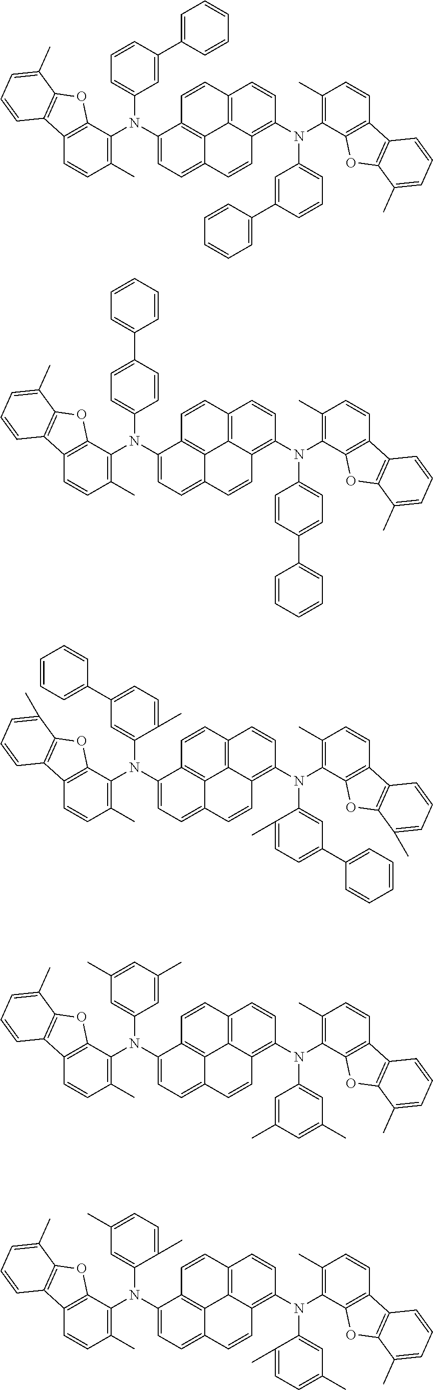

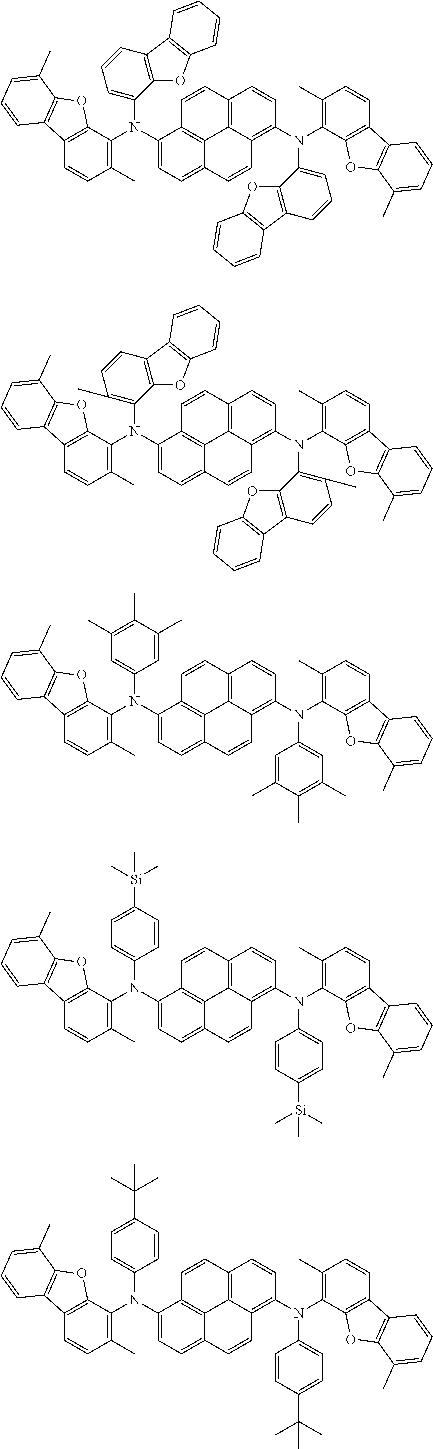

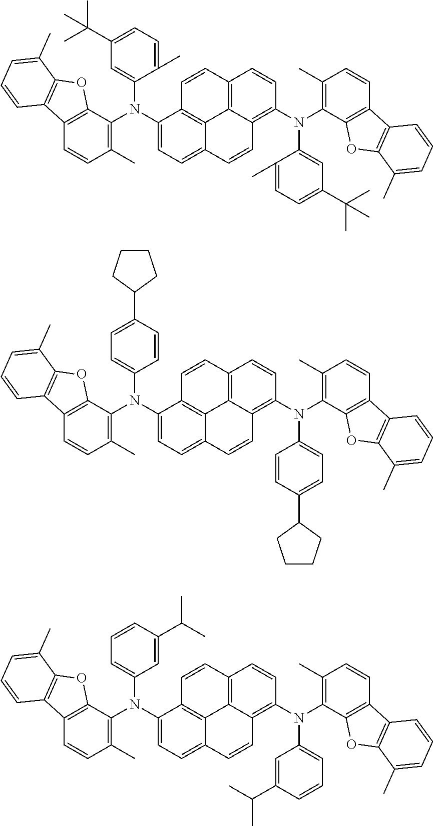 [SMILES: Cc1ccc(C(C)(C)C)cc1N(c1ccc2ccc3c(N(c4cc(C(C)(C)C)ccc4C)c4c(C)ccc5c4oc4c(C)cccc45)ccc4ccc1c2c43)c1c(C)ccc2c1oc1c(C)cccc12 ; Cc1ccc2c(oc3c(C)cccc32)c1N(c1ccc(C2CCCC2)cc1)c1ccc2ccc3c(N(c4ccc(C5CCCC5)cc4)c4c(C)ccc5c4oc4c(C)cccc45)ccc4ccc1c2c43 ; Cc1ccc2c(oc3c(C)cccc32)c1N(c1cccc(C(C)C)c1)c1ccc2ccc3c(N(c4cccc(C(C)C)c4)c4c(C)ccc5c4oc4c(C)cccc45)ccc4ccc1c2c43]

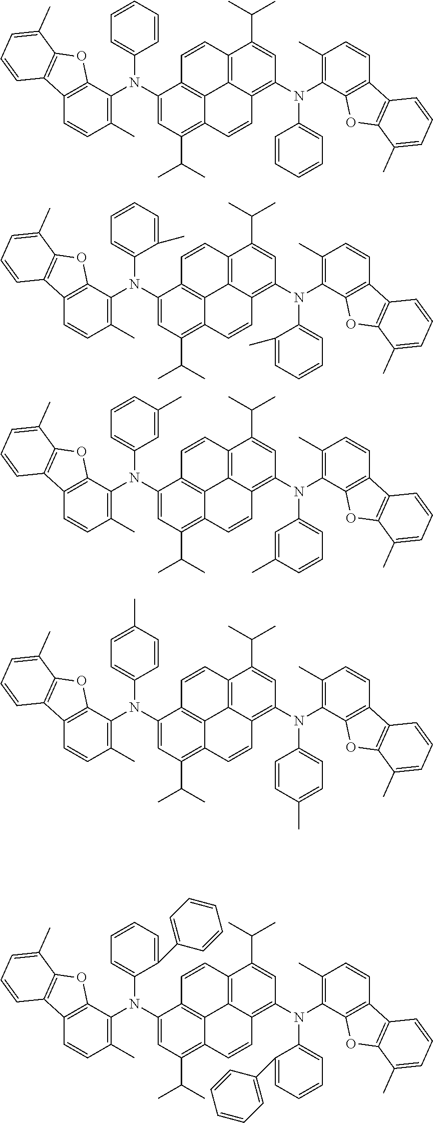

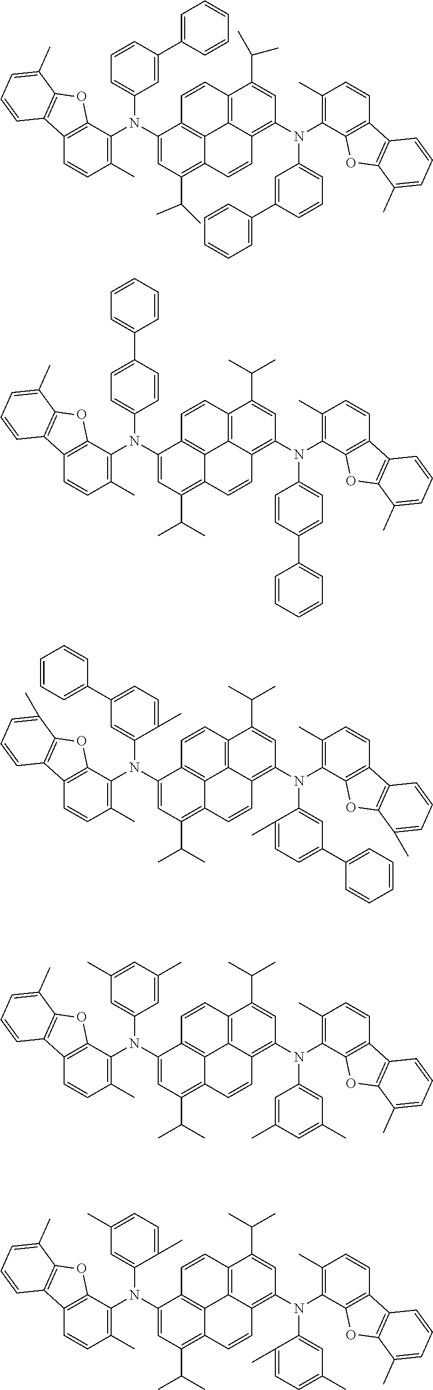

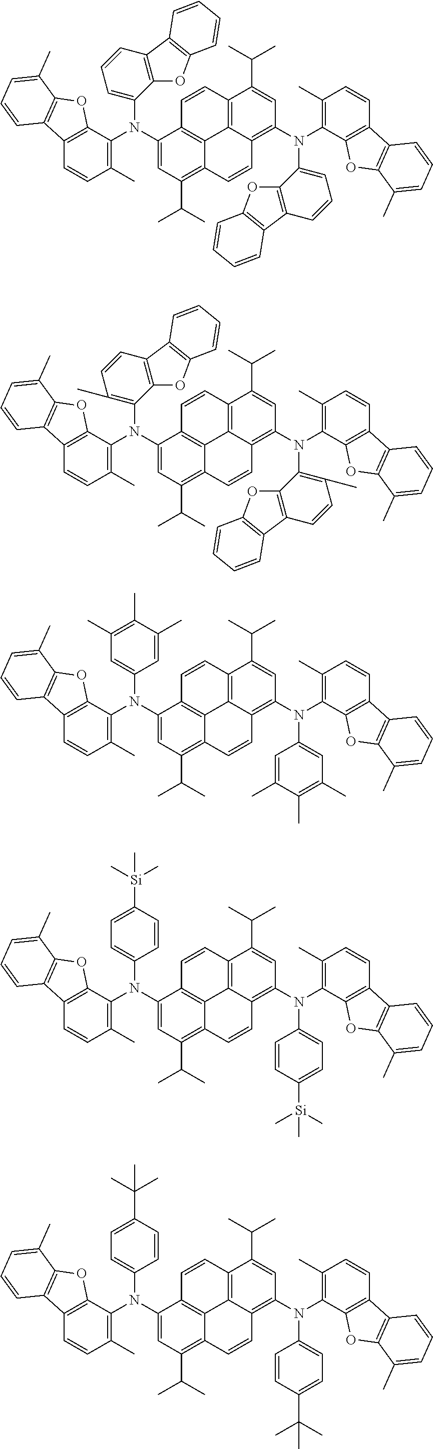

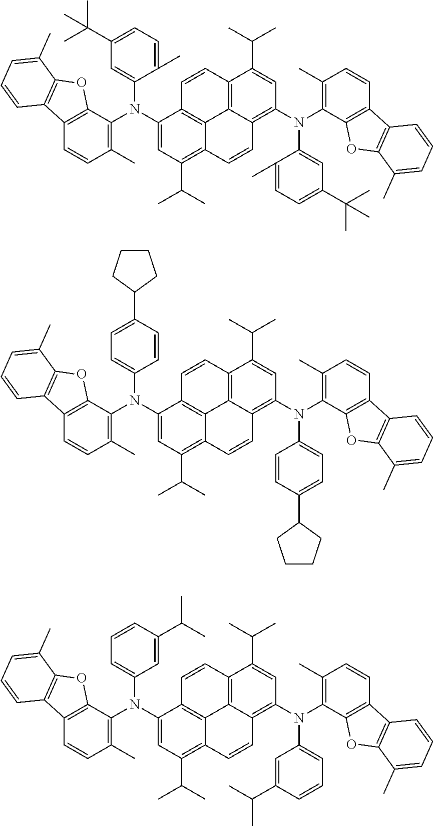 [SMILES: Cc1ccc(C(C)(C)C)cc1N(c1cc(C(C)C)c2ccc3c(N(c4cc(C(C)(C)C)ccc4C)c4c(C)ccc5c4oc4c(C)cccc45)cc(C(C)C)c4ccc1c2c43)c1c(C)ccc2c1oc1c(C)cccc12 ; Cc1ccc2c(oc3c(C)cccc32)c1N(c1ccc(C2CCCC2)cc1)c1cc(C(C)C)c2ccc3c(N(c4ccc(C5CCCC5)cc4)c4c(C)ccc5c4oc4c(C)cccc45)cc(C(C)C)c4ccc1c2c43 ; Cc1ccc2c(oc3c(C)cccc32)c1N(c1cccc(C(C)C)c1)c1cc(C(C)C)c2ccc3c(N(c4cccc(C(C)C)c4)c4c(C)ccc5c4oc4c(C)cccc45)cc(C(C)C)c4ccc1c2c43]

The organic EL device of an aspect of the invention includes: a cathode; an anode; an emitting layer disposed between the cathode and the anode; and the emitting layer contains a compound represented by the formula (1).

An organic EL device having high luminous efficiency can be obtained by using the compound represented by the formula (1) for an emitting layer.

In the organic EL device of an aspect of the invention, the emitting layer further contains a compound represented by the following formula (11). An organic EL device having high luminous efficiency and reduced driving voltages can be obtained by using the compound represented by the formula (1) and the compound represented by the following formula (11) for an emitting layer.

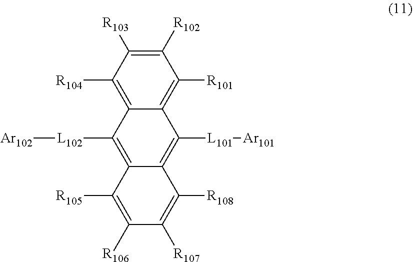

In the formula (11), R₁₀₁ to R₁₀₈ are independently

a hydrogen atom, a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a substituted or unsubstituted aryl group including 6 to 50 ring carbon atoms, or
a substituted or unsubstituted monovalent heterocyclic group including 5 to 50 ring atoms.

Adjacent two or more among R₁₀₁ to R₁₀₄, and adjacent two or more among R₁₀₅ to R₁₀₈ do not form a ring by bonding with each other.

R₉₀₁ to R₉₀₇ are as defined in the formula (1).

L₁₀₁ and L₁₀₂ are independently

a single bond,
a substituted or unsubstituted arylene group including 6 to 30 ring carbon atoms, or
a substituted or unsubstituted divalent heterocyclic group including 5 to 30 ring atoms.

At least one of Ar₁₀₁ and Ar₁₀₂ is a monovalent group represented by the following formula (12).

Ar₁₀₁ and Ar₁₀₂ which are not the monovalent group represented by the following formula (12) are

a substituted or unsubstituted aryl group including 6 to 50 ring carbon atoms, or
a substituted or unsubstituted monovalent heterocyclic group including 5 to 50 ring atoms.

When both Ar₁₀₁ and Ar₁₀₂ are the monovalent group represented by the formula (12), Ar₁₀₁ and Ar₁₀₂ which are the monovalent group represented by the following formula (12) may be the same as or different from each other.

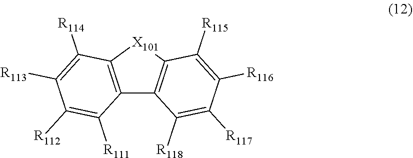

In the formula (12),

X₁₀₁ is an oxygen atom or a sulfur atom.

One or more sets of adjacent two of R₁₁₁ to R₁₁₈ form an unsaturated ring represented by the following formula (20) by bonding with each other, or do not form the unsubstituted ring represented by the following formula (20).

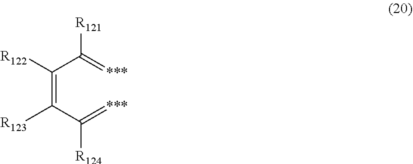

In the formula (20), “***” indicates a position bonding to adjacent two of R₁₁₁ to R₁₁₈.

When one or more sets of adjacent two of R₁₁₁ to R₁₁₈ form the unsaturated ring represented by the formula (20) by bonding with each other, one of R₁₁₁ to R₁₁₈ which do not form the unsaturated ring represented by the formula (20), and one of R₁₂₁ to R₁₂₄ is a single bond bonding with L₁₀₁ or L₁₀₂.

When two or more of the unsaturated rings represented by the formula (20) are formed, a plurality of each of R₁₂₁ to R₁₂₄ may be the same as or different from each other.

When one or more sets of adjacent two of R₁₁₁ to R₁₁₈ do not form the unsaturated ring represented by the formula (20), one of R₁₁₁ to R₁₁₈ is a single bond bonding with L₁₀₁ or L₁₀₂.

When the unsaturated ring represented by the formula (20) is formed and when the unsaturated ring represented by the formula (20) is not formed, one or more sets of adjacent two of R₁₁₁ to R₁₁₈ which do not form the unsaturated ring represented by the formula (20) and are not a single bond bonding with L₁₀₁ or L₁₀₂ form a substituted or unsubstituted, saturated or unsaturated ring other than the unsaturated ring represented by the formula (20) by bonding with each other, or do not form a substituted or unsubstituted, saturated or unsaturated ring.

R₁₁₁ to R₁₁₈ which do not form the unsaturated ring represented by the formula (20), do not form a substituted or unsubstituted, saturated or unsaturated ring other than the unsaturated ring represented by the formula (20), and are not a single bond bonding with L₁₀₁ or L₁₀₂, and R₁₂₁ to R₁₂₄ which are not a single bond bonding with L₁₀₁ or L₁₀₂ are independently

a hydrogen atom, a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a substituted or unsubstituted aryl group including 6 to 50 ring carbon atoms, or
a substituted or unsubstituted monovalent heterocyclic group including 5 to 50 ring atoms.

R₉₀₁ to R₉₀₇ are as defined in the formula (1).

In one embodiment, one of Ar₁₀₁ and Ar₁₀₂ in the formula (11) is a monovalent group represented by the formula (12).

In one embodiment, the compound represented by the formula (11) is a compound represented by the following formula (13).

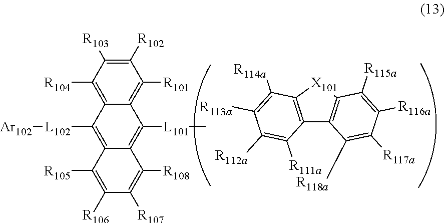

In the formula (13),

R₁₀₁ to R₁₀₈, L₁₀₁, and L₁₀₂ are as defined in the formula (11).

Ar₁₀₂ is a monovalent group represented by the formula (12),

a substituted or unsubstituted aryl group including 6 to 50 ring carbon atoms, or
a substituted or unsubstituted monovalent heterocyclic group including 5 to 50 ring atoms.

X₁₀₁ is as defined in the formula (12).

One of R₁₁₁ₐ and R₁₁₂ₐ is a single bond bonding with L₁₀₁.

One or more sets of adjacent two of R₁₁₁ₐ or R₁₁₂ₐ which is not a single bond bonding with L₁₀₁, and R₁₁₃ₐ to R₁₁₈ₐ form an unsaturated ring represented by the formula (20) by bonding with each other, or do not form the unsaturated ring represented by the formula (20).

R₁₁₁ₐ or R₁₁₂ₐ which is not a single bond bonding with L₁₀₁ and which does not form the unsaturated ring represented by the formula (20), and R₁₁₃ₐ to R₁₁₈ₐ which do not form the unsaturated ring represented by the formula (20) are independently

a hydrogen atom, a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a substituted or unsubstituted aryl group including 6 to 50 ring carbon atoms, or
a substituted or unsubstituted monovalent heterocyclic group including 5 to 50 ring atoms.

R₉₀₁ to R₉₀₇ are as defined in the formula (1).

In one embodiment, the compound represented by the formula (11) is a compound represented by the following formula (14).

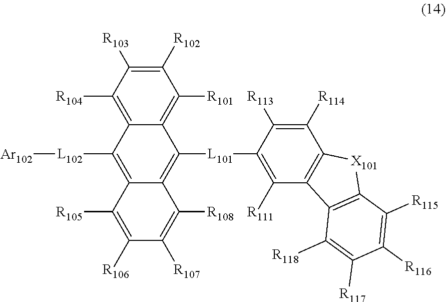

In the formula (14),

R₁₀₁ to R₁₀₈, L₁₀₁, L₁₀₂, X₁₀₁, R₁₁₁, and R₁₁₃ to R₁₁₈ are as defined in the formula (11) and (12).

Ar₁₀₂ is a monovalent group represented by the formula (12),

a substituted or unsubstituted aryl group including 6 to 50 ring carbon atoms, or
a substituted or unsubstituted monovalent heterocyclic group including 5 to 50 ring atoms.

In one embodiment, the compound represented by the formula (11) is a compound represented by the following formula (14a).

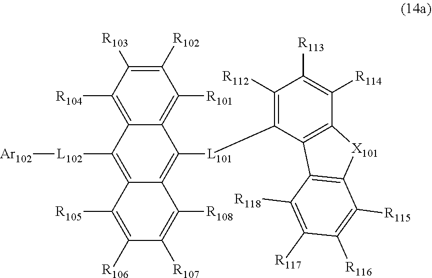

In the formula (14a), R₁₀₁ to R₁₀₈, L₁₀₁, L₁₀₂, X₁₀₁, R₁₁₂ to R₁₁₈ are as defined in the formula (11) and (12).

Ar₁₀₂ is a monovalent group represented by the formula (12),

a substituted or unsubstituted aryl group including 6 to 50 ring carbon atoms, or
a substituted or unsubstituted monovalent heterocyclic group including 5 to 50 ring atoms.

In one embodiment, the monovalent group represented by the formula (12) is selected from the monovalent group represented by any of the following formulas (12A) to (12F).

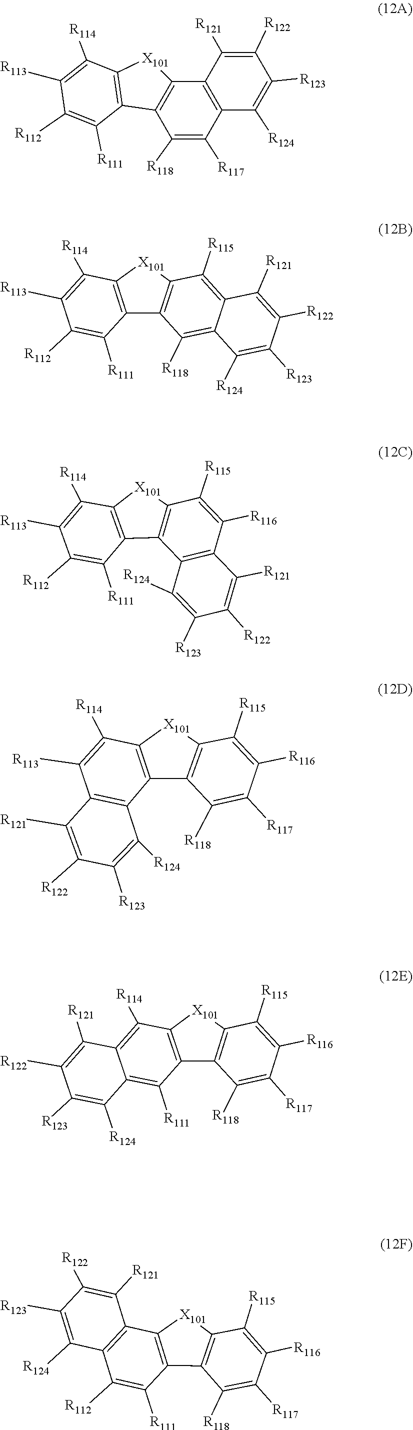

In the formula (12A) to (12F), X₁₀₁, R₁₁₁ to R₁₁₁, and R₁₂₁ to R₁₂₄ are as defined in the formula (12).

In one embodiment, one of Ar₁₀₁ and Ar₁₀₂ in the formula (11) is the monovalent group represented by the formula (12), and the other is a substituted or unsubstituted aryl group including 6 to 50 ring carbon atoms.

In one embodiment, Ar₁₀₁ or Ar₁₀₂ in the formula (11) which is not the monovalent group represented by the formula (12) is selected from groups represented by any of the following formulas (a1) to (a4).

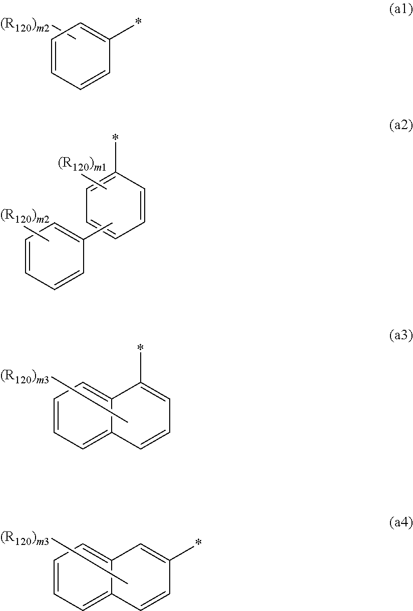

In the formulas (a1) to (a4),

“*” is a single bond bonding with L₁₀₁ or L₁₀₂.

R₁₂₀ is

a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a substituted or unsubstituted aryl group including 6 to 50 ring carbon atoms, or
a substituted or unsubstituted monovalent heterocyclic group including 5 to 50 ring atoms.

R₉₀₁ to R₉₀₇ are as defined in the formula (1).

m1 is an integer of 0 to 4.

m2 is an integer of 0 to 5.
m3 is an integer of 0 to 7.

When each of m1 to m3 is 2 or more, a plurality of R₁₂₀'s may be the same as or different from each other.

When each of m1 to m3 is 2 or more, a plurality of adjacent R₁₂₀'s form a substituted or unsubstituted, saturated or unsaturated ring by bonding with each other, or do not form a substituted or unsubstituted saturated or unsaturated ring.

In one embodiment, L₁₀₁ and L₁₀₂ in the formula (11) are independently a substituted or unsubstituted arylene group including 6 to 14 ring carbon atoms.

In one embodiment, when L₁₀₁ and L₁₀₂ in the formula (11) are a divalent linking groups, the linking groups are independently selected from groups represented by any of the following formulas (b1) to (b17).

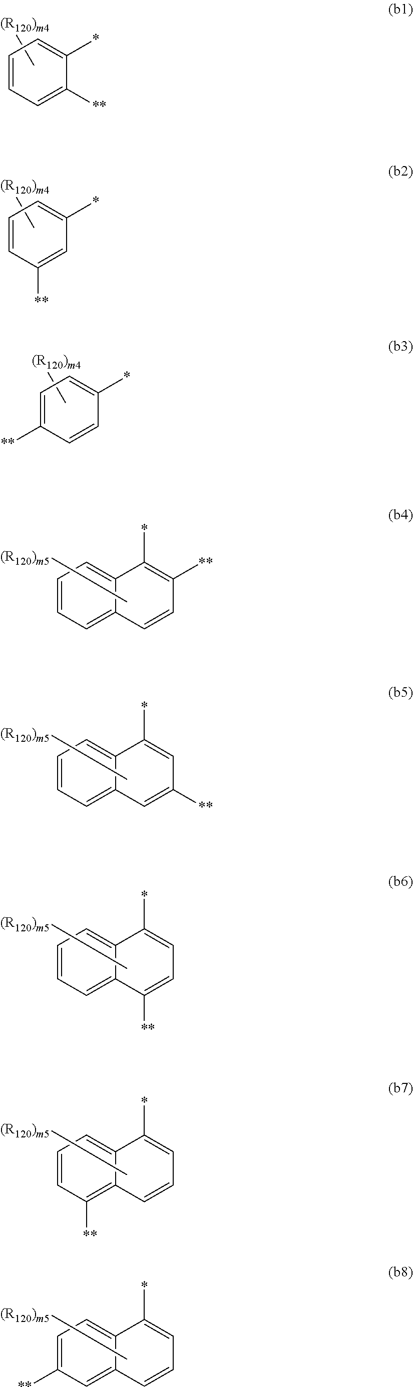

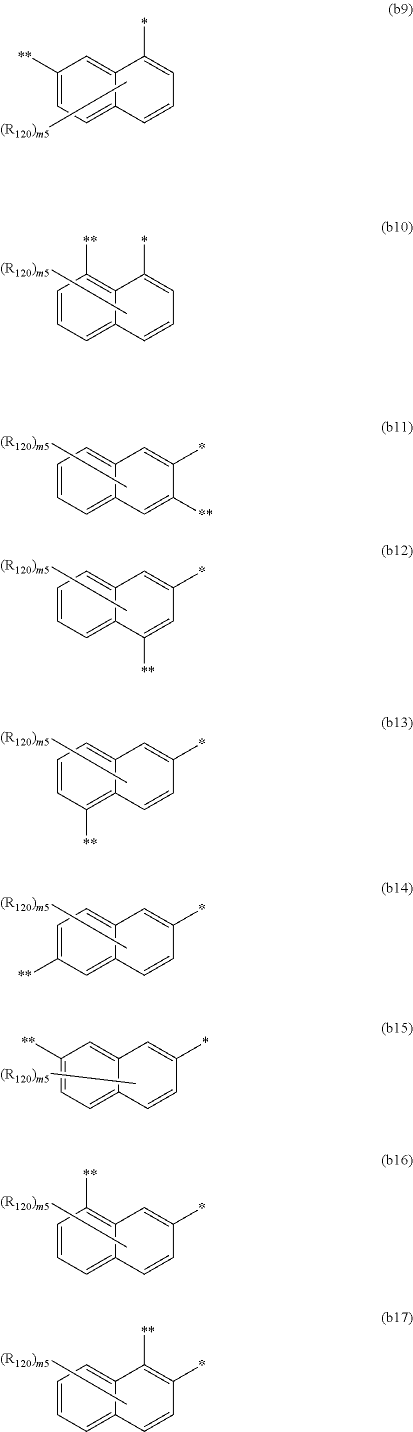

In the formulas (b1) to (b17), “*” is a single bond bonding with Ar₁₀₁ or Ar₁₀₂.

“**” is a single bond bonding with the anthracene skeleton in the formula (11).

R₁₂₀ is

a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a substituted or unsubstituted aryl group including 6 to 50 ring carbon atoms, or
a substituted or unsubstituted monovalent heterocyclic group including 5 to 50 ring atoms;

R₉₀₁ to R₉₀₇ are as defined in the formula (1).

m4 is an integer of 0 to 4.

m5 is an integer of 0 to 6.

When m4 and m5 are each 2 or more, a plurality of R₁₂₀'s may be the same as or different from each other.

When m4 and m5 are each 2 or more, a plurality of adjacent R₁₂₀'s form a saturated or unsaturated ring by bonding with each other or do not form a saturated or unsaturated ring.

In one embodiment, X₁₀₁ in the formula (12) is an oxygen atom.

In one embodiment, the compound represented by the formula (11) is a compound represented by the following formula (15).

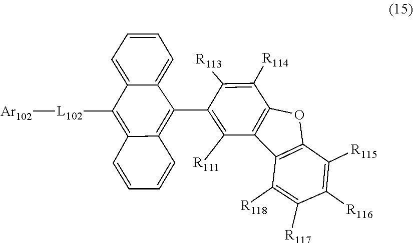

In the formula (15), L₁₀₂ is as defined in the formula (11).

Ar₁₀₂ is the monovalent group represented by the formula (12),

a substituted or unsubstituted aryl group including 6 to 50 ring carbon atoms, or
a substituted or unsubstituted monovalent heterocyclic group including 5 to 50 ring atoms.

R₁₁₁ and R₁₁₃ to R₁₁₈ are as defined in the formula (12).

In one embodiment, the compound represented by the formula (11) is a compound represented by the following formula (15a).

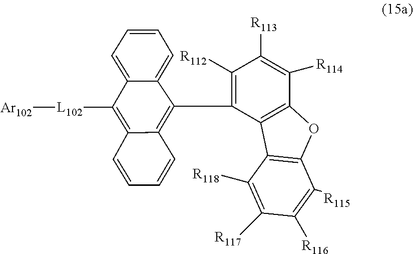

In the formula (15a), L₁₀₂ is as defined in the formula (11).

Ar₁₀₂ is the monovalent group represented by the formula (12),

a substituted or unsubstituted aryl group including 6 to 50 ring carbon atoms, or
a substituted or unsubstituted monovalent heterocyclic group including 5 to 50 ring atoms.

R₁₁₂ to R₁₁₈ are as defined in the formula (12).

In one embodiment, the compound represented by the formula (11) is a compound represented by the following formula (16).

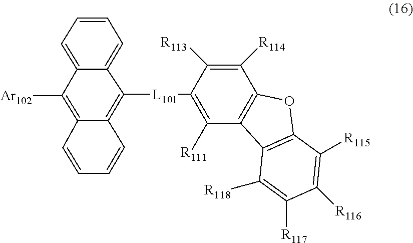

In the formula (16), L₁₀₁ is as defined in the formula (11).

Ar₁₀₂ is the monovalent group represented by the formula (12),

a substituted or unsubstituted aryl group including 6 to 50 ring carbon atoms, or
a substituted or unsubstituted monovalent heterocyclic group including 5 to 50 ring atoms.

R₁₁₁ and R₁₁₃ to R₁₁₈ are as defined in the formula (12).

In one embodiment, the compound represented by the formula (11) is a compound represented by the following formula (16a).

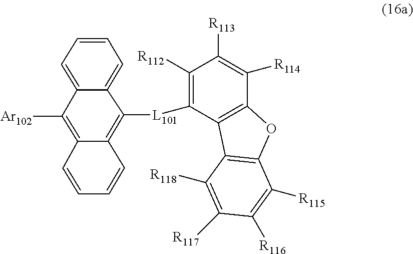

In the formula (16a), L₁₀₁ is as defined in the formula (11).

Ar₁₀₂ is the monovalent group represented by the formula (12),

a substituted or unsubstituted aryl group including 6 to 50 ring carbon atoms, or
a substituted or unsubstituted monovalent heterocyclic group including 5 to 50 ring atoms.

R₁₁₂ to R₁₁₈ are as defined in the formula (12).

In one embodiment, the compound represented by the formula (11) is a compound represented by the following formula (17).

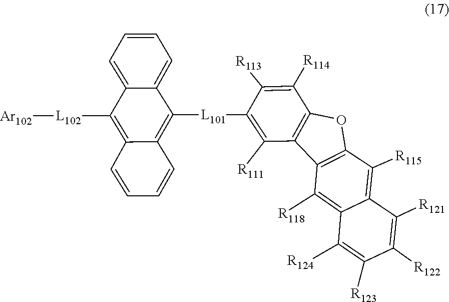

In the formula (17),

L₁₀₁ and L₁₀₂ are as defined in the formula (11).

Ar₁₀₂ is the monovalent group represented by the formula (12), a substituted or unsubstituted aryl group including 6 to 50 ring carbon atoms, or a substituted or unsubstituted monovalent heterocyclic group including 5 to 50 ring atoms.

R₁₁₁, R₁₁₃ to R₁₁₅, R₁₁₈, and R₁₂₁ to R₁₂₄ are as defined in the formula (12).

In one embodiment, R₁₀₁ to R₁₀₈ in the formula (11) are hydrogen atoms.

In one embodiment, R₁₁₁ to R₁₁₈ in the formula (12) which are not a single bond bonding with L₁₀₁, and R₁₂₁ to R₁₂₄ which are not a single bond bonding with L₁₀₁ are hydrogen atoms.

In one embodiment, the compound represented by the formula (11) is a compound represented by the following formula (18).

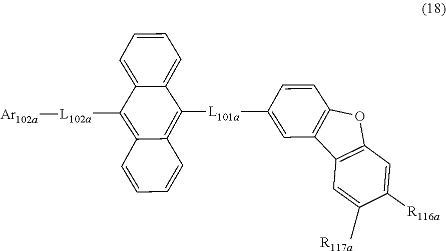

In the formula (18),

L₁₀₁ₐ and L₁₀₂ₐ are independently

a single bond, or
a substituted or unsubstituted arylene group including 6 to 10 ring carbon atoms.

Ar₁₀₂ₐ is a substituted or unsubstituted aryl group including 6 to 10 ring carbon atoms.

R₁₁₆ₐ and R₁₁₇ₐ form a saturated or unsaturated ring by bonding with each other, or do not form the saturated or unsaturated ring.

R₁₁₆ₐ and R₁₁₇ₐ which do not form the saturated or unsaturated ring by bonding with each other are independently

a hydrogen atom, a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅), or

—N(R₉₀₆)(R₉₀₇).

R₉₀₁ to R₉₀₇ are as defined in the formula (1).

In one embodiment, the compound represented by the formula (11) is a compound represented by the following formula (18a).

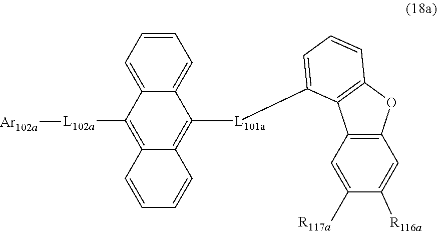

In the formula (18a),

L₁₀₁ₐ and L₁₀₂ₐ are independently

a single bond, or
a substituted or unsubstituted arylene group including 6 to 10 ring carbon atoms.

Ar₁₀₂ₐ is a substituted or unsubstituted aryl group including 6 to 10 ring carbon atoms.

R₁₁₆ₐ and R₁₁₇ₐ form a saturated or unsaturated ring by bonding with each other, or do not form the saturated or unsaturated ring.

R₁₁₆ₐ and R₁₁₇ₐ which do not form a saturated or unsaturated ring by bonding with each other are independently

a hydrogen atom, a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅), or

—N(R₉₀₆)(R₉₀₇).

R₉₀₁ to R₉₀₇ are as defined in the formula (1).

Details of the substituents in the formulas (11), (12), (12A) to (12F), (20), (13) to (18), (13a) to (16a), (18a), (a1) to (a4) and (b1) to (b17), and the substituents in the case of “a substituted or unsubstituted” are as defined in the [Definition] part of this specification.

Specific examples of the compound represented by the formula (11) include compounds shown below.

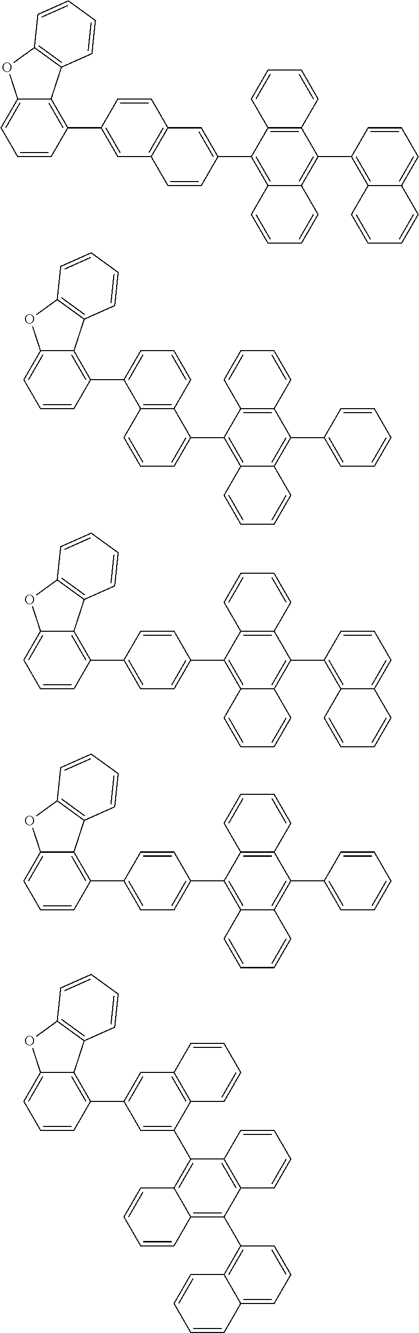 [SMILES: c1ccc2c(-c3c4ccccc4c(-c4ccc5cc(-c6cccc7oc8ccccc8c67)ccc5c4)c4ccccc34)cccc2c1 ; c1ccc(-c2c3ccccc3c(-c3cccc4c(-c5cccc6oc7ccccc7c56)cccc34)c3ccccc23)cc1 ; c1ccc2c(-c3c4ccccc4c(-c4ccc(-c5cccc6oc7ccccc7c56)cc4)c4ccccc34)cccc2c1 ; c1ccc(-c2c3ccccc3c(-c3ccc(-c4cccc5oc6ccccc6c45)cc3)c3ccccc23)cc1 ; c1ccc2c(-c3c4ccccc4c(-c4cc(-c5cccc6oc7ccccc7c56)cc5ccccc45)c4ccccc34)cccc2c1]

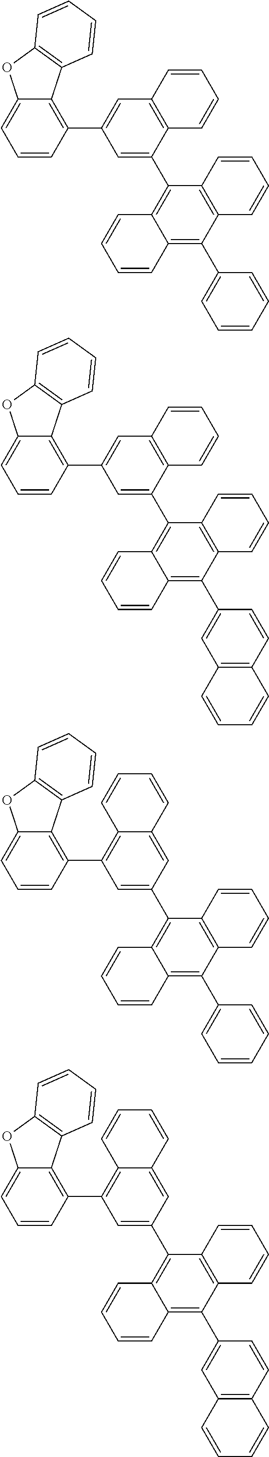 [SMILES: c1ccc(-c2c3ccccc3c(-c3cc(-c4cccc5oc6ccccc6c45)cc4ccccc34)c3ccccc23)cc1 ; c1ccc2cc(-c3c4ccccc4c(-c4cc(-c5cccc6oc7ccccc7c56)cc5ccccc45)c4ccccc34)ccc2c1 ; c1ccc(-c2c3ccccc3c(-c3cc(-c4cccc5oc6ccccc6c45)c4ccccc4c3)c3ccccc23)cc1 ; c1ccc2cc(-c3c4ccccc4c(-c4cc(-c5cccc6oc7ccccc7c56)c5ccccc5c4)c4ccccc34)ccc2c1]

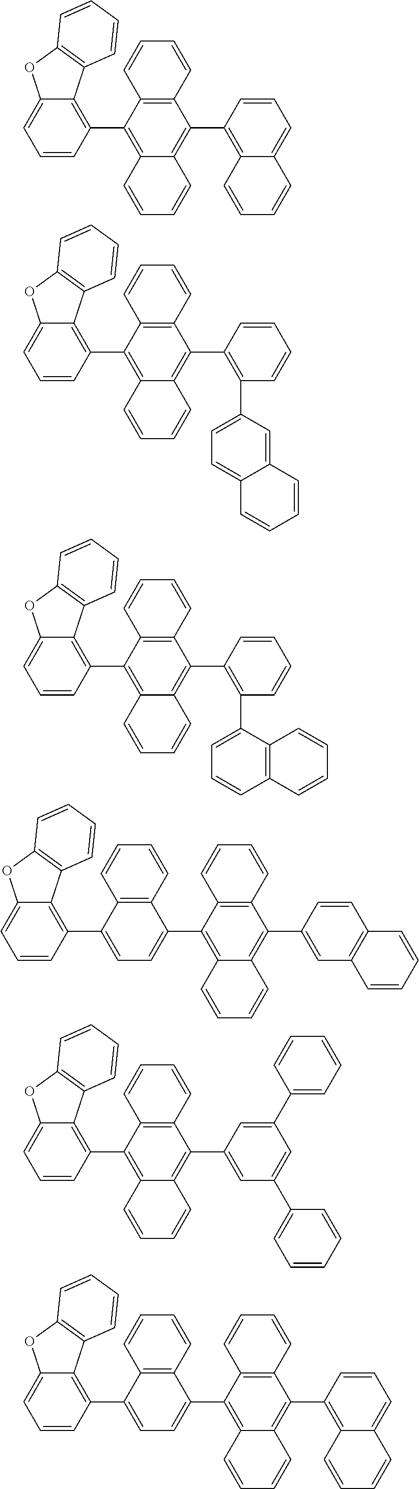

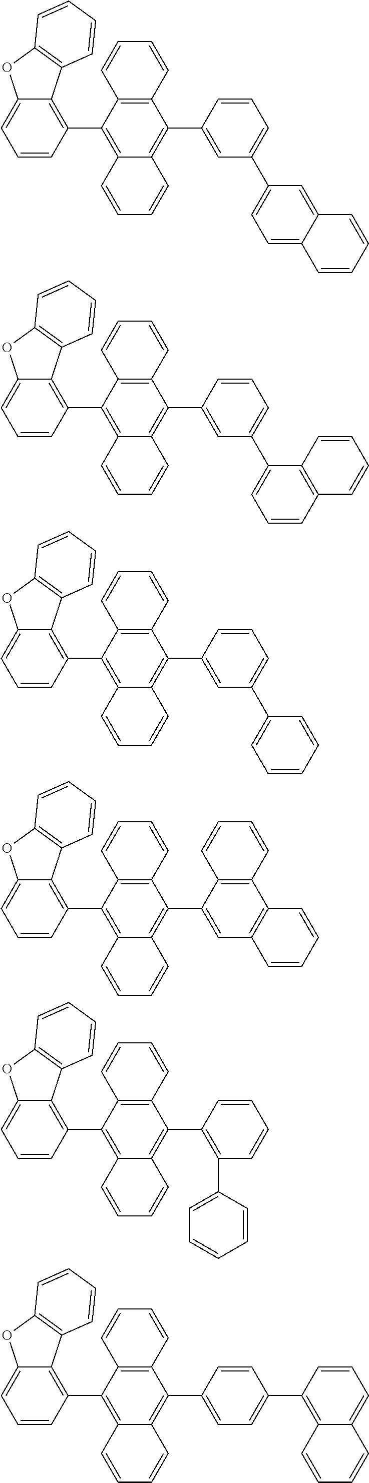

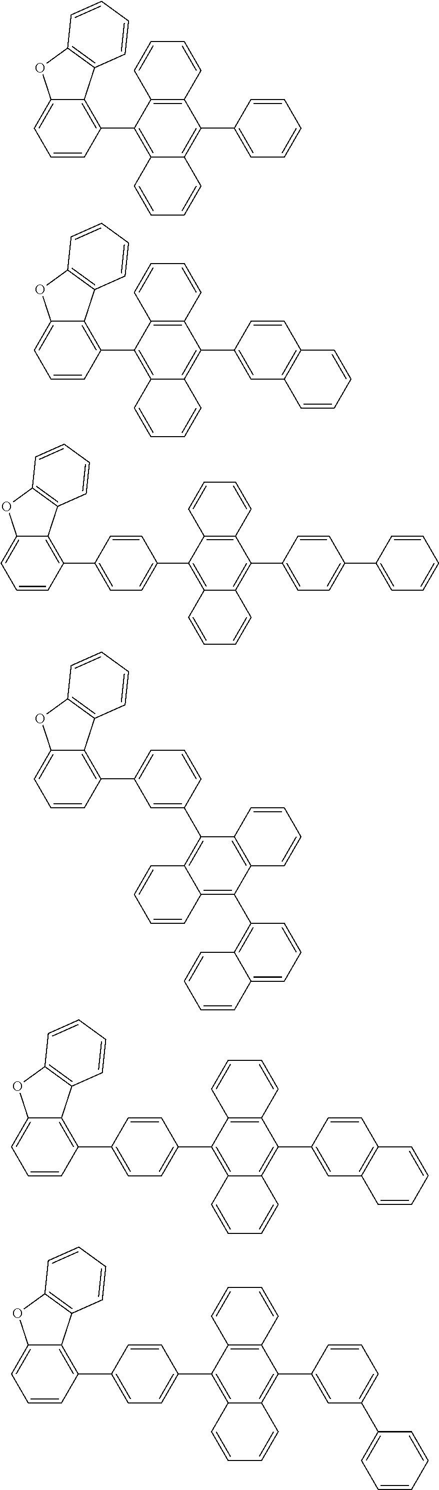

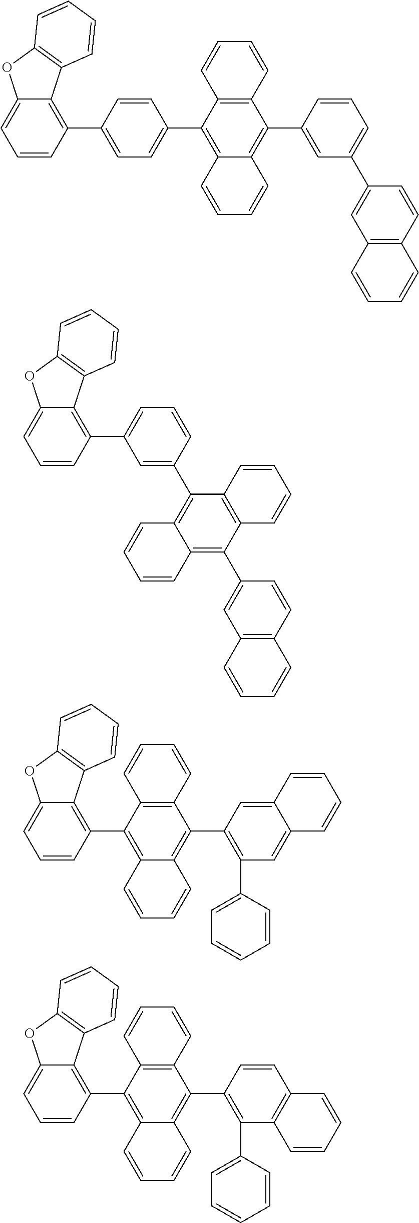 [SMILES: c1cc(-c2ccc3ccccc3c2)cc(-c2c3ccccc3c(-c3ccc(-c4cccc5oc6ccccc6c45)cc3)c3ccccc23)c1 ; c1cc(-c2c3ccccc3c(-c3ccc4ccccc4c3)c3ccccc23)cc(-c2cccc3oc4ccccc4c23)c1 ; c1ccc(-c2cc3ccccc3cc2-c2c3ccccc3c(-c3cccc4oc5ccccc5c34)c3ccccc23)cc1 ; c1ccc(-c2c(-c3c4ccccc4c(-c4cccc5oc6ccccc6c45)c4ccccc34)ccc3ccccc23)cc1]

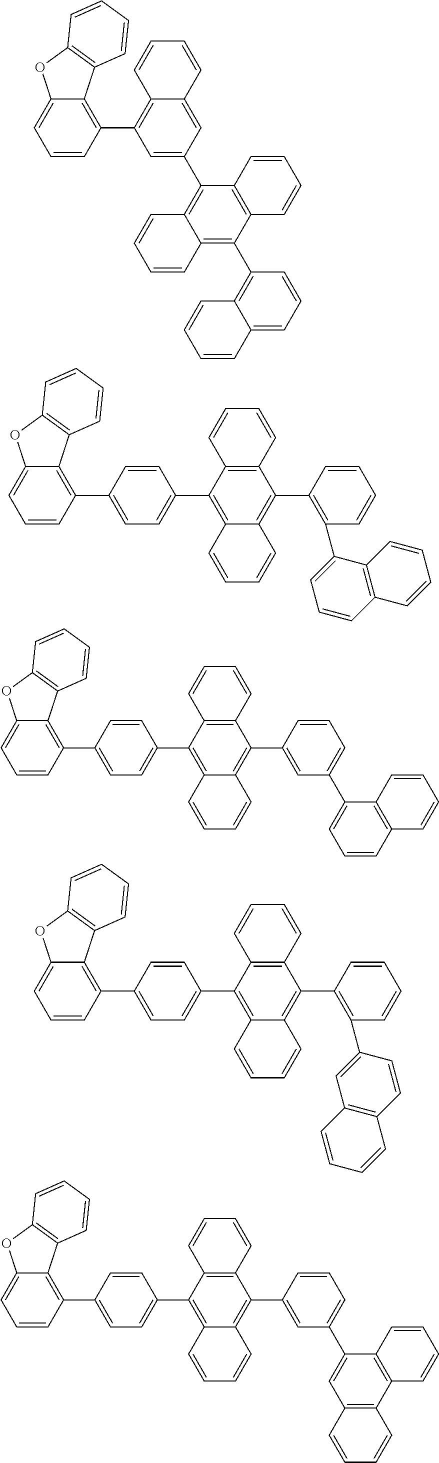 [SMILES: c1ccc2c(-c3c4ccccc4c(-c4cc(-c5cccc6oc7ccccc7c56)c5ccccc5c4)c4ccccc34)cccc2c1 ; c1ccc(-c2c3ccccc3c(-c3ccc(-c4cccc5oc6ccccc6c45)cc3)c3ccccc23)c(-c2cccc3ccccc23)c1 ; c1cc(-c2cccc3ccccc23)cc(-c2c3ccccc3c(-c3ccc(-c4cccc5oc6ccccc6c45)cc3)c3ccccc23)c1 ; c1ccc(-c2c3ccccc3c(-c3ccc(-c4cccc5oc6ccccc6c45)cc3)c3ccccc23)c(-c2ccc3ccccc3c2)c1 ; c1cc(-c2c3ccccc3c(-c3ccc(-c4cccc5oc6ccccc6c45)cc3)c3ccccc23)cc(-c2cc3ccccc3c3ccccc23)c1]

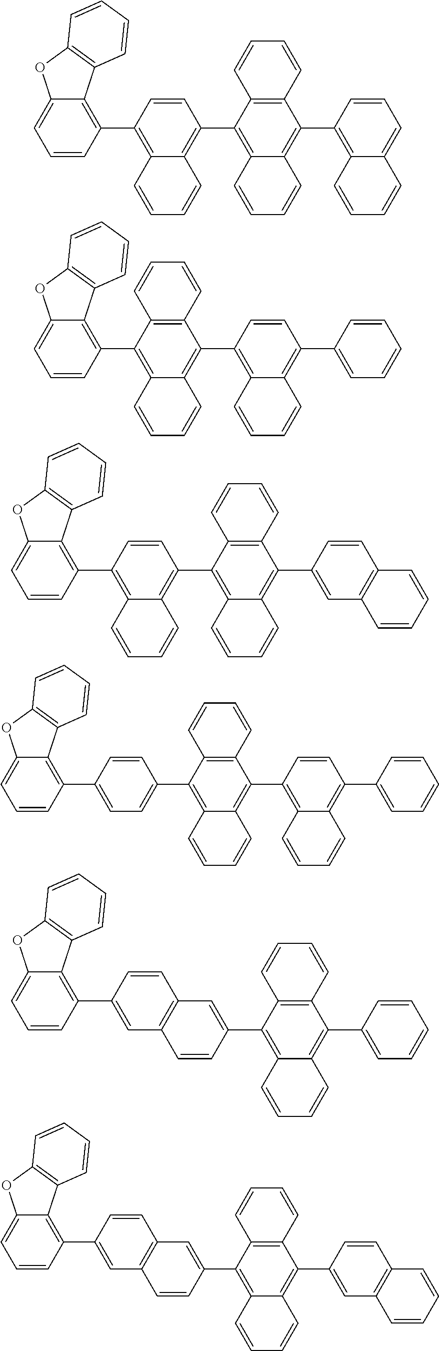

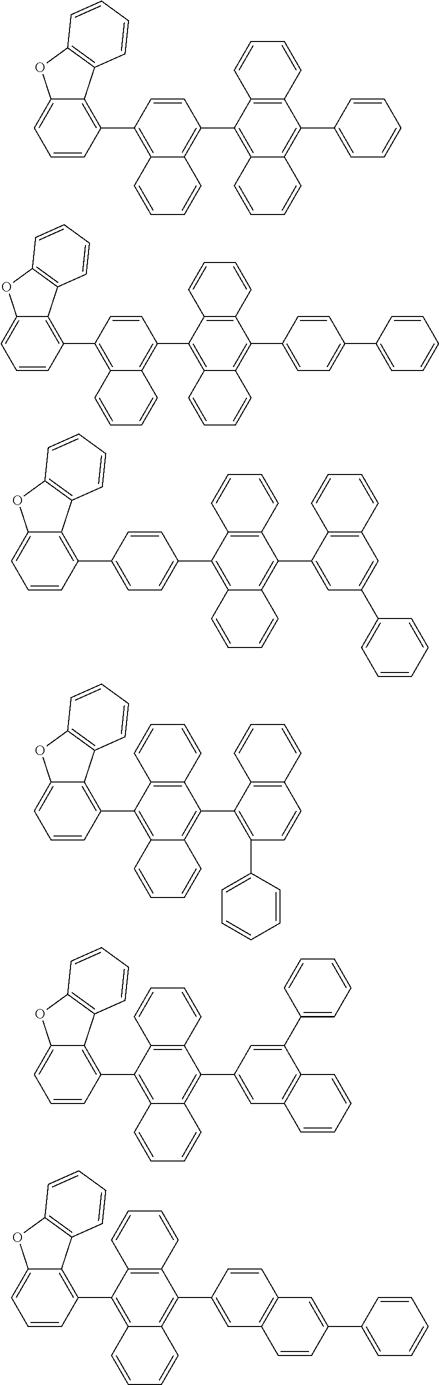

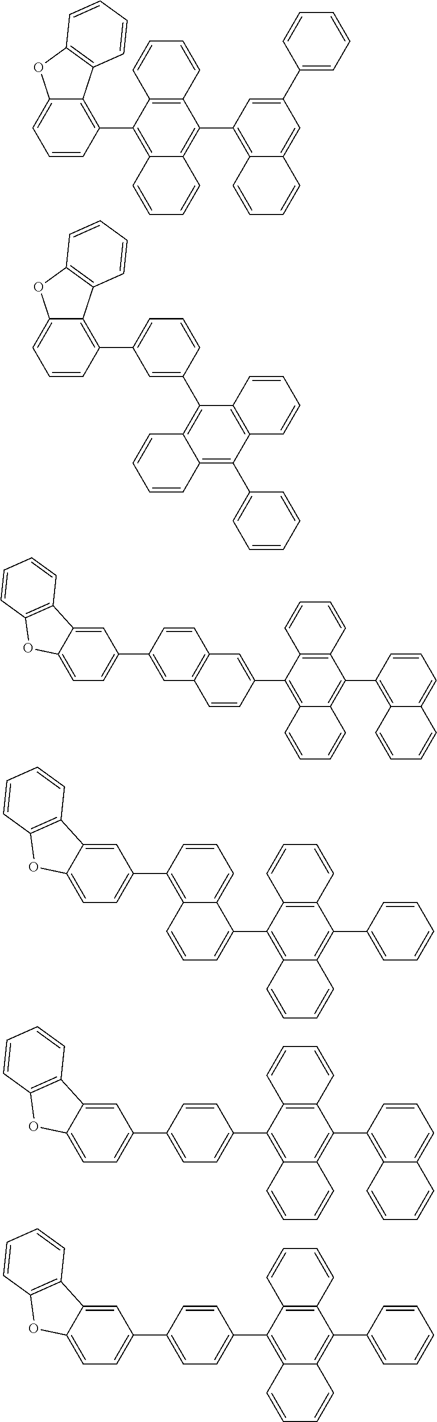

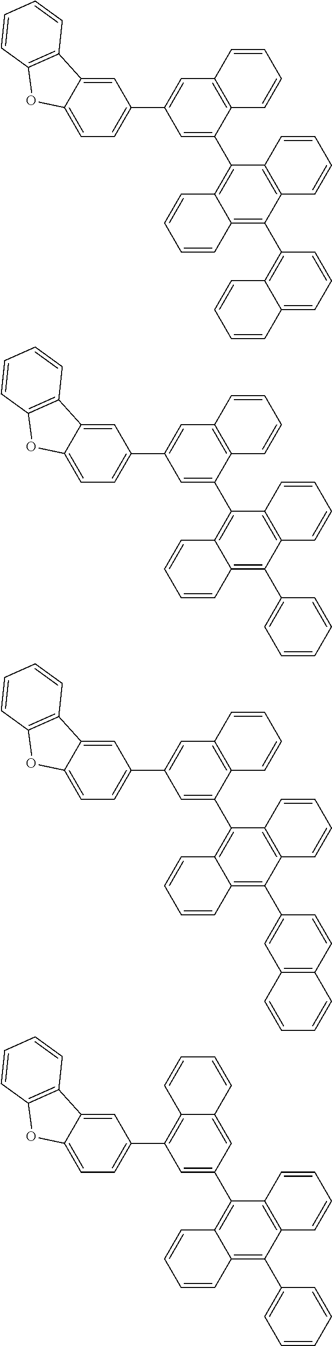 [SMILES: c1ccc2c(-c3c4ccccc4c(-c4cc(-c5ccc6oc7ccccc7c6c5)cc5ccccc45)c4ccccc34)cccc2c1 ; c1ccc(-c2c3ccccc3c(-c3cc(-c4ccc5oc6ccccc6c5c4)cc4ccccc34)c3ccccc23)cc1 ; c1ccc2cc(-c3c4ccccc4c(-c4cc(-c5ccc6oc7ccccc7c6c5)cc5ccccc45)c4ccccc34)ccc2c1 ; c1ccc(-c2c3ccccc3c(-c3cc(-c4ccc5oc6ccccc6c5c4)c4ccccc4c3)c3ccccc23)cc1]

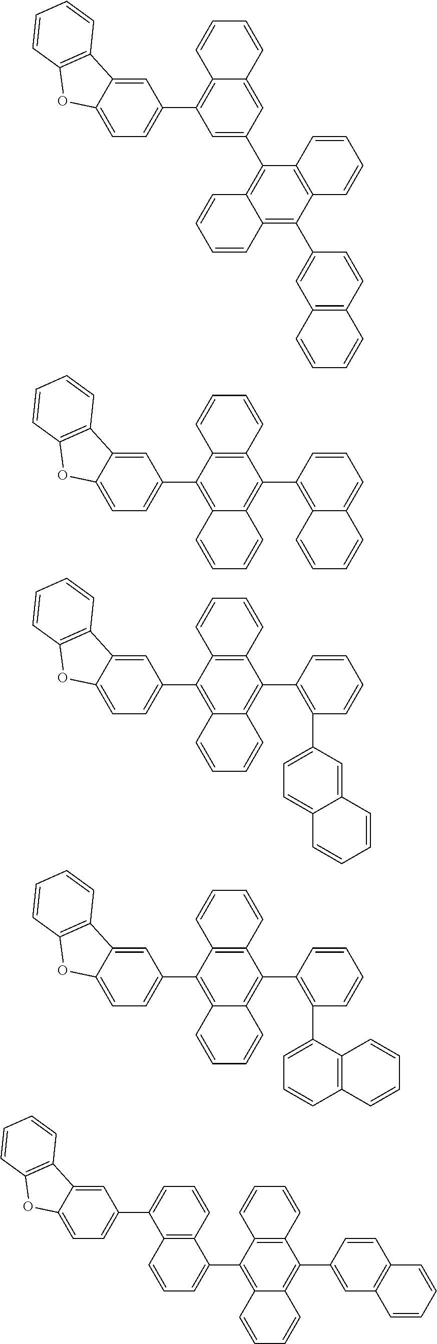

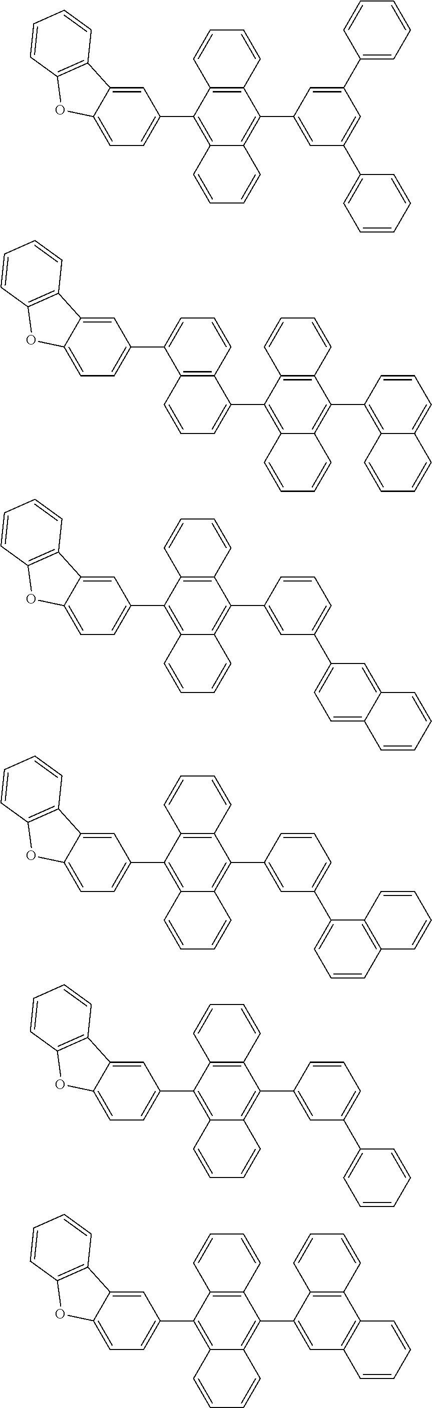

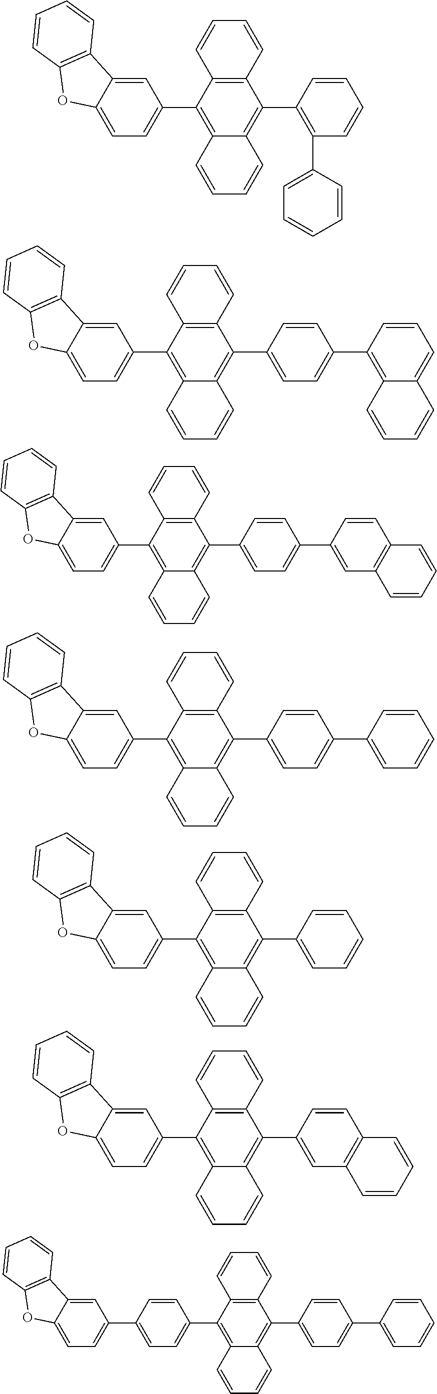

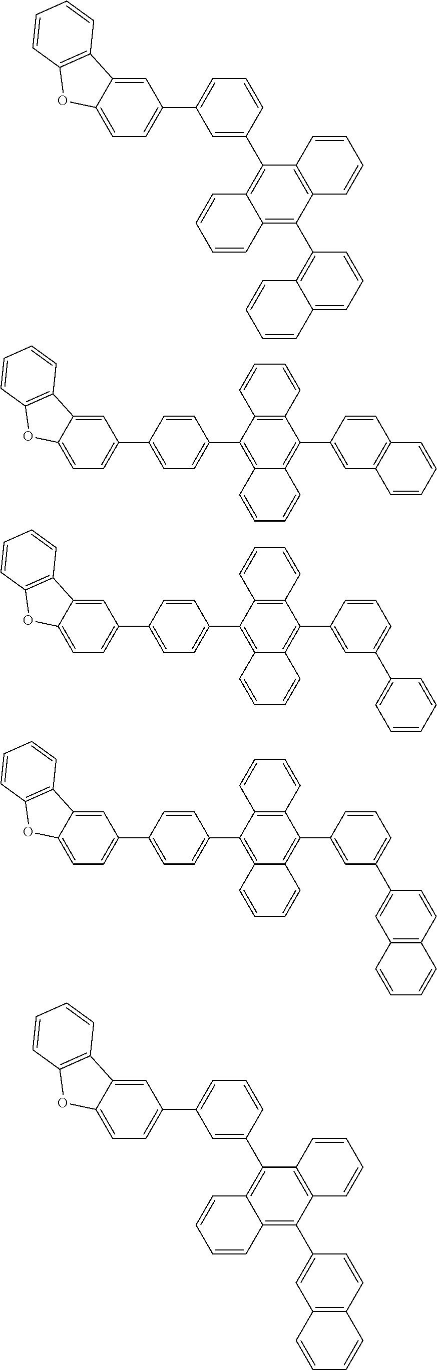

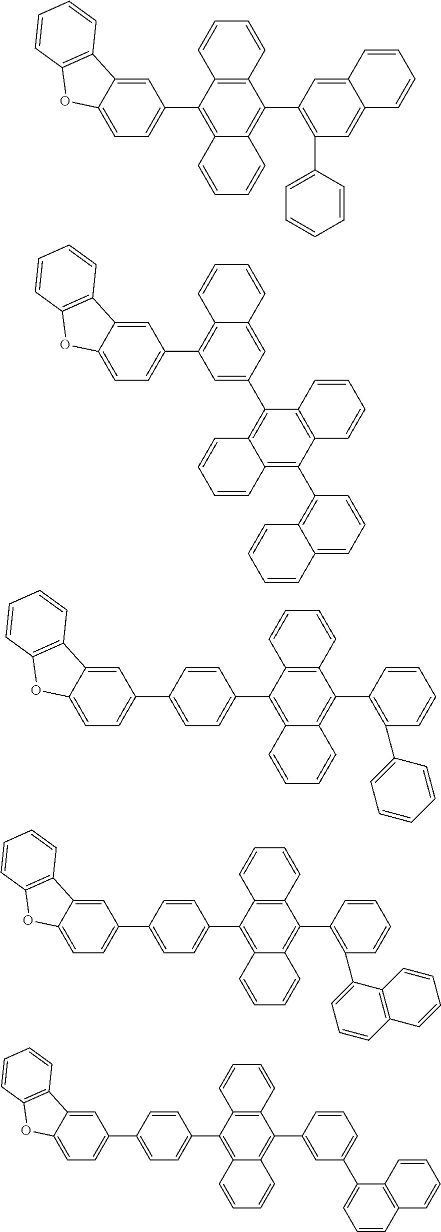

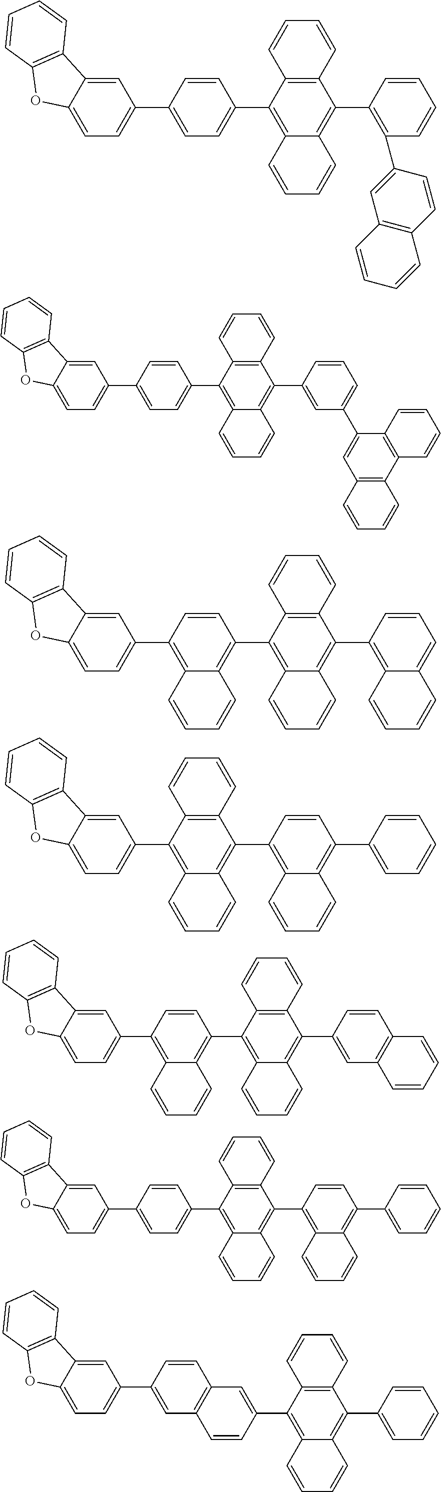

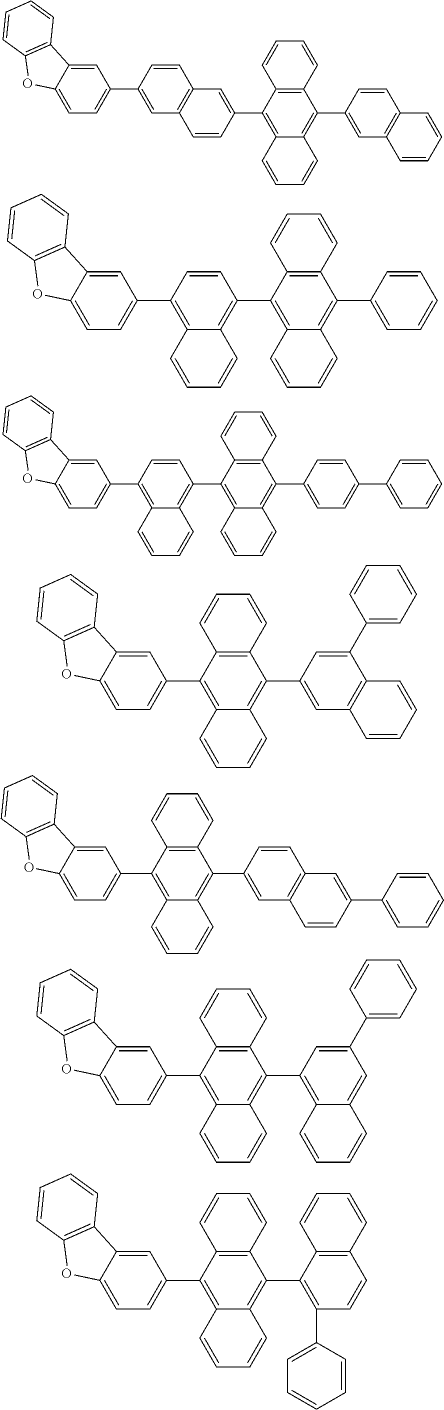

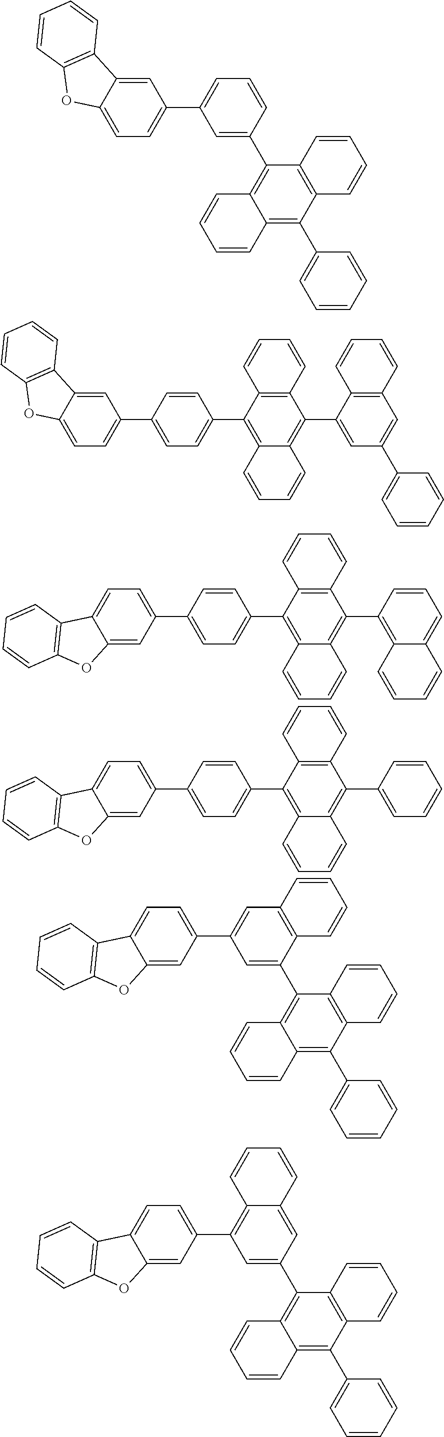

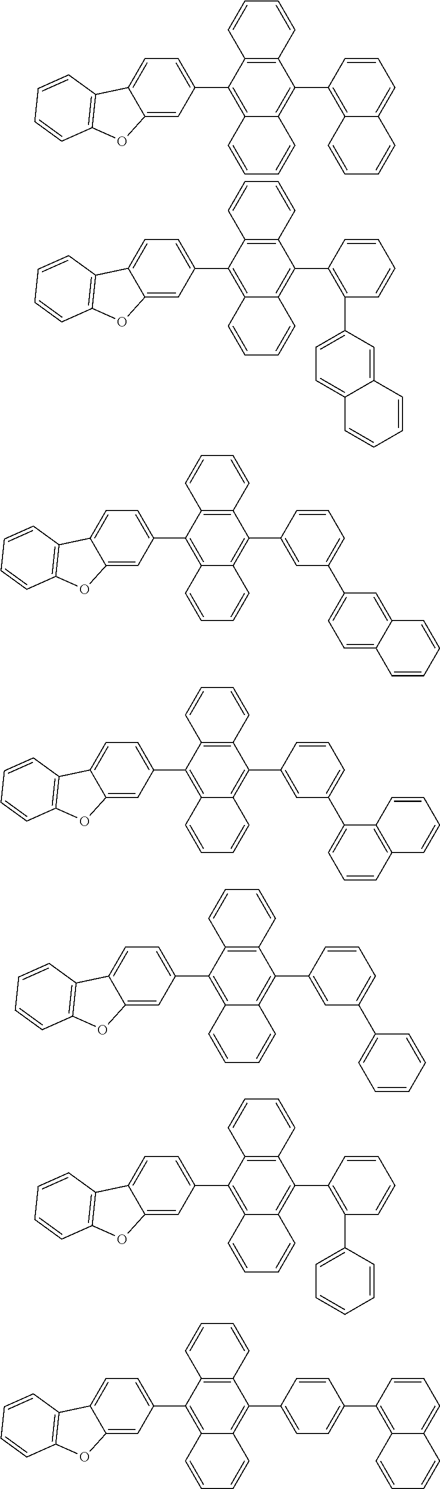

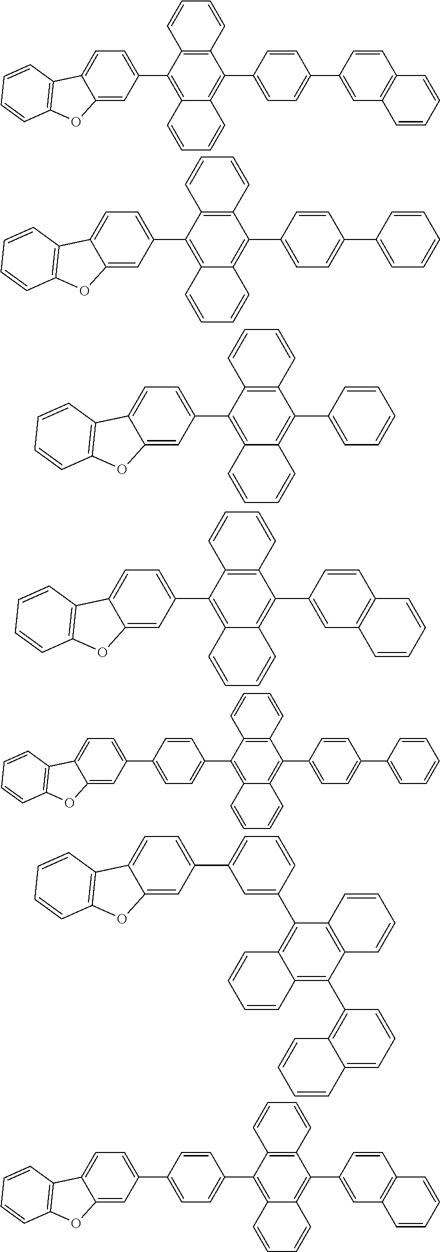

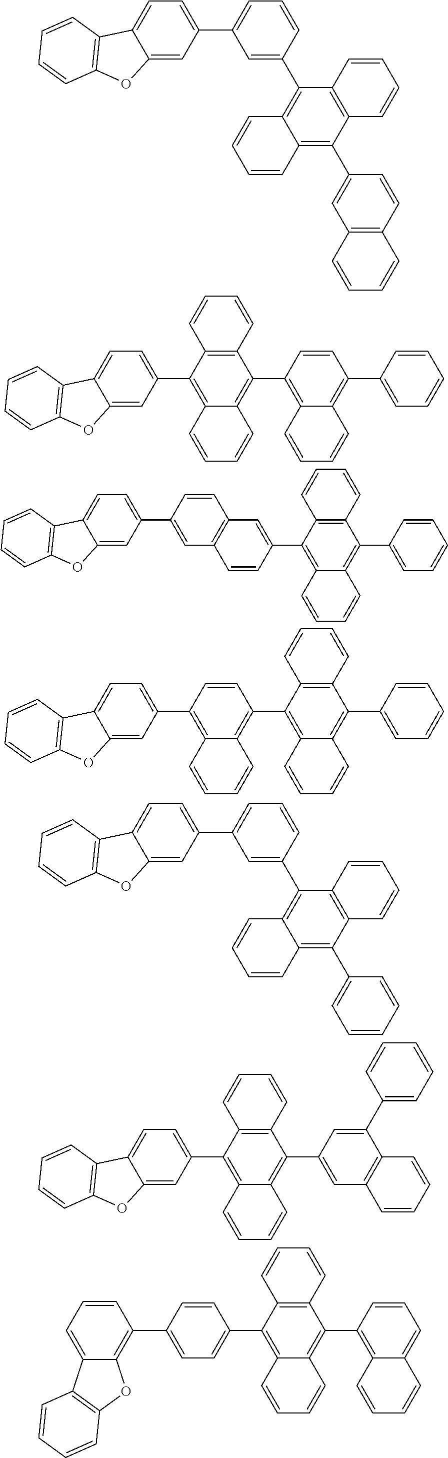

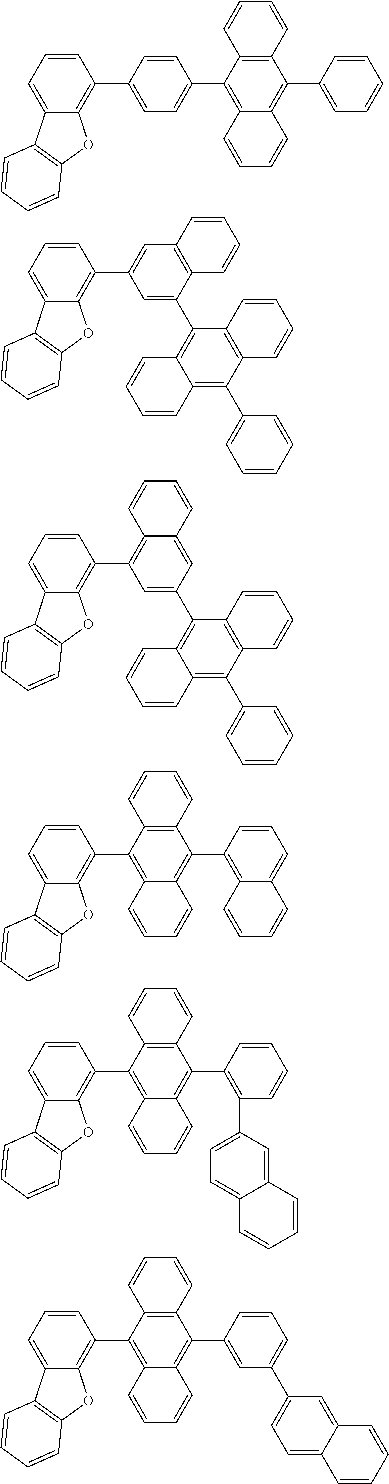

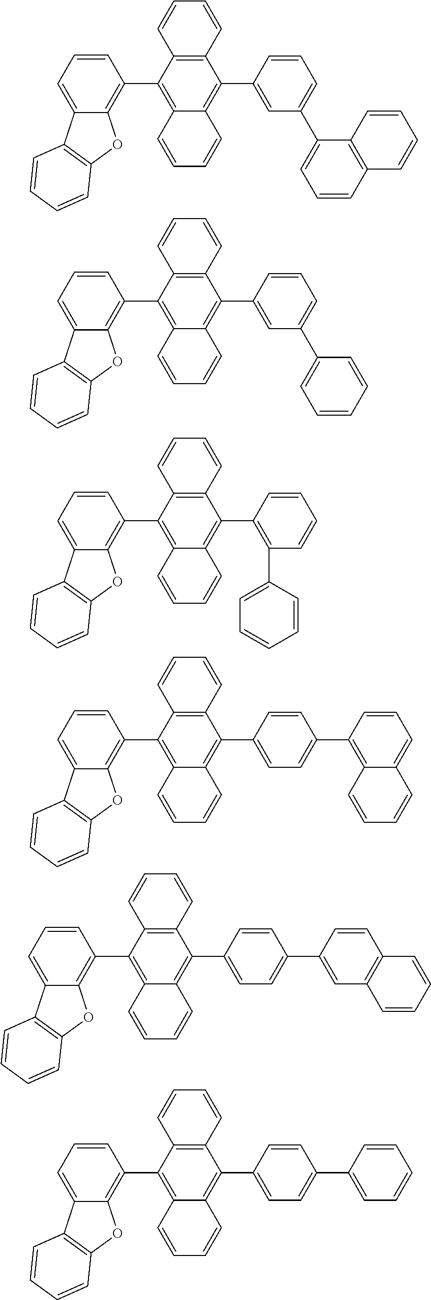

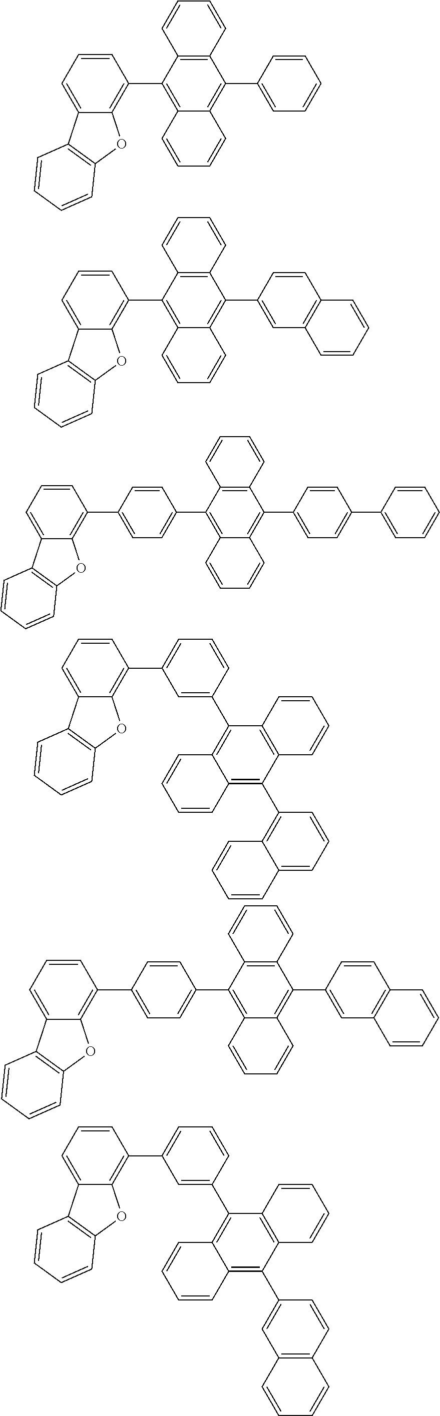

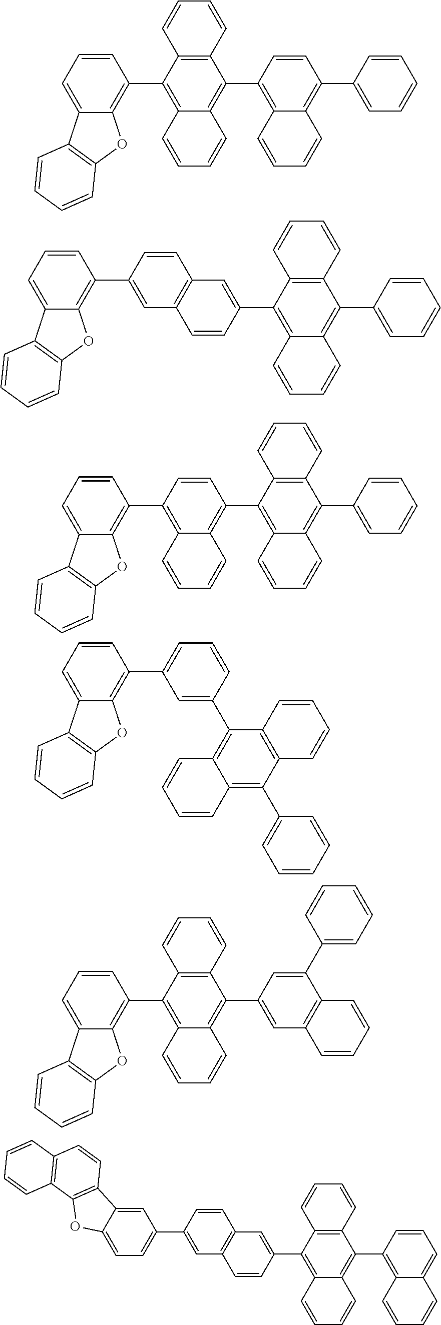

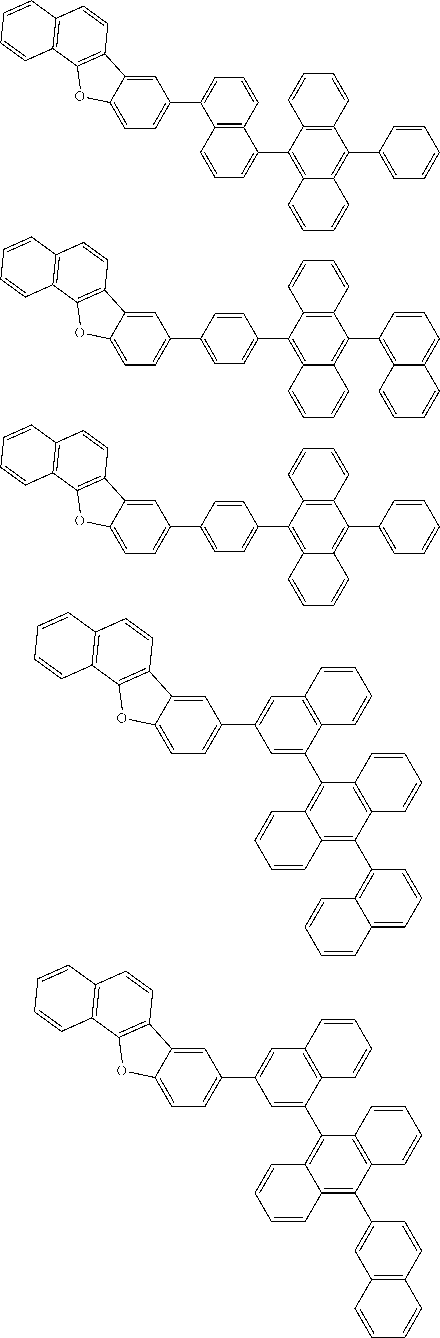

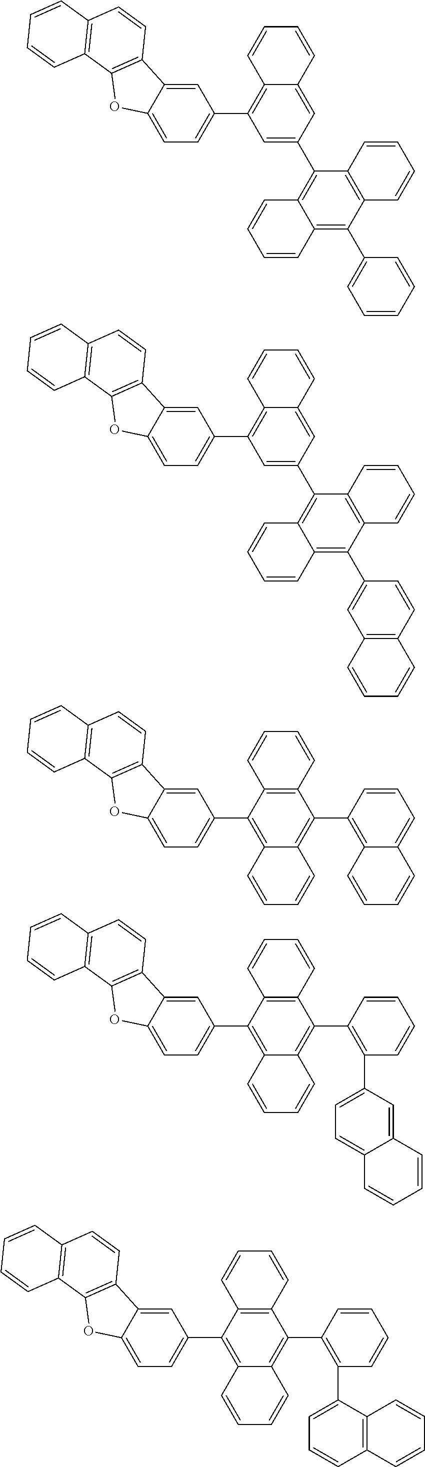

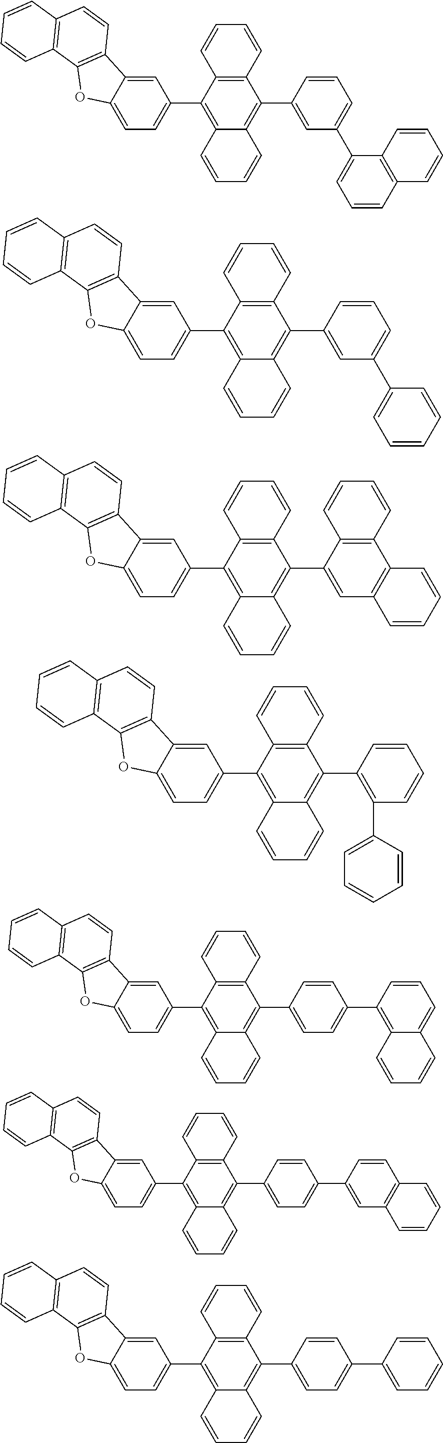

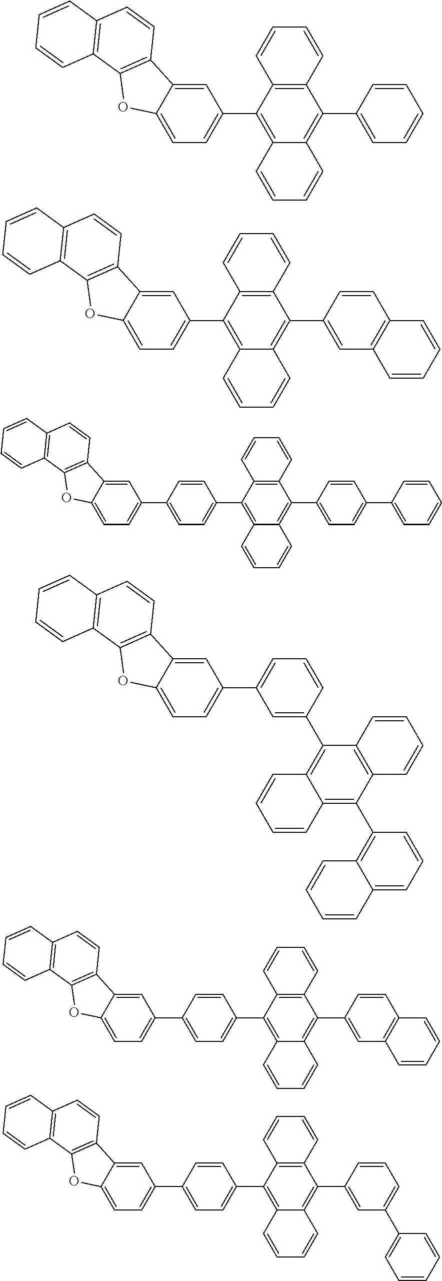

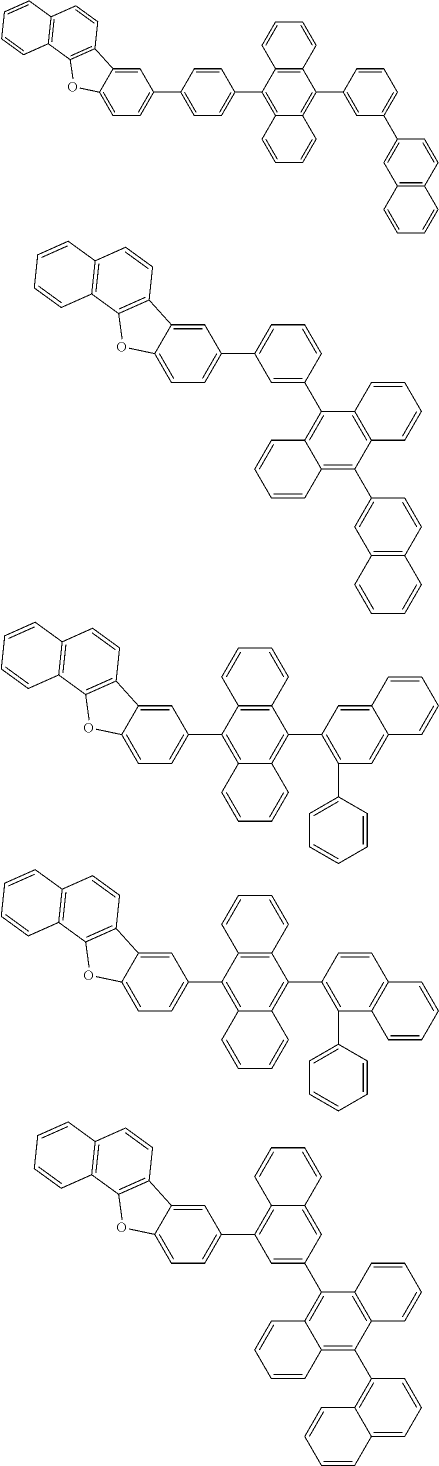 [SMILES: c1cc(-c2ccc3ccccc3c2)cc(-c2c3ccccc3c(-c3ccc(-c4ccc5oc6c7ccccc7ccc6c5c4)cc3)c3ccccc23)c1 ; c1cc(-c2ccc3oc4c5ccccc5ccc4c3c2)cc(-c2c3ccccc3c(-c3ccc4ccccc4c3)c3ccccc23)c1 ; c1ccc(-c2cc3ccccc3cc2-c2c3ccccc3c(-c3ccc4oc5c6ccccc6ccc5c4c3)c3ccccc23)cc1 ; c1ccc(-c2c(-c3c4ccccc4c(-c4ccc5oc6c7ccccc7ccc6c5c4)c4ccccc34)ccc3ccccc23)cc1 ; c1ccc2c(-c3ccc4oc5c6ccccc6ccc5c4c3)cc(-c3c4ccccc4c(-c4cccc5ccccc45)c4ccccc34)cc2c1]

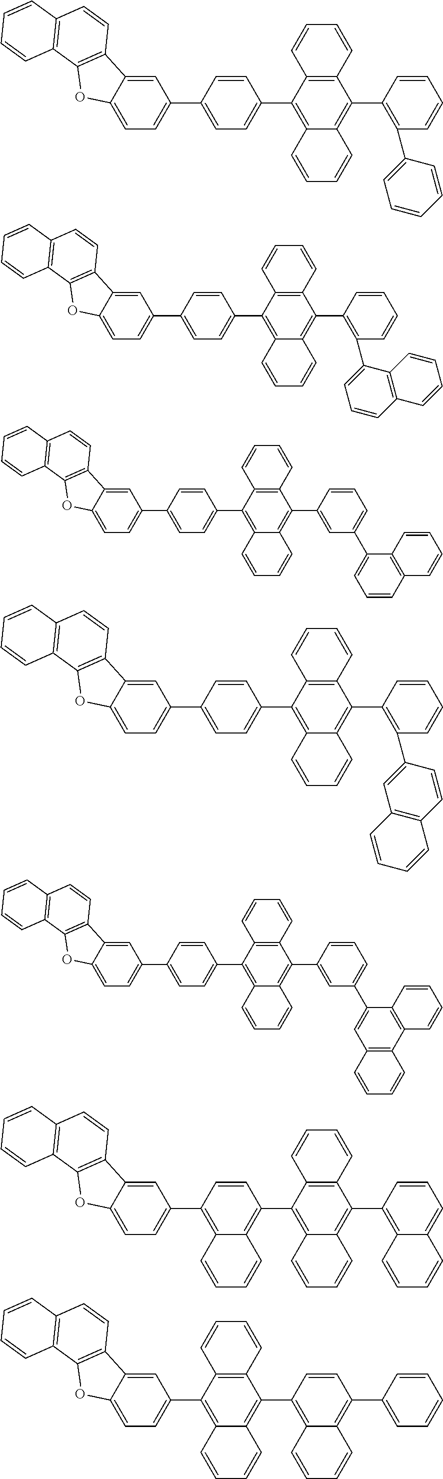

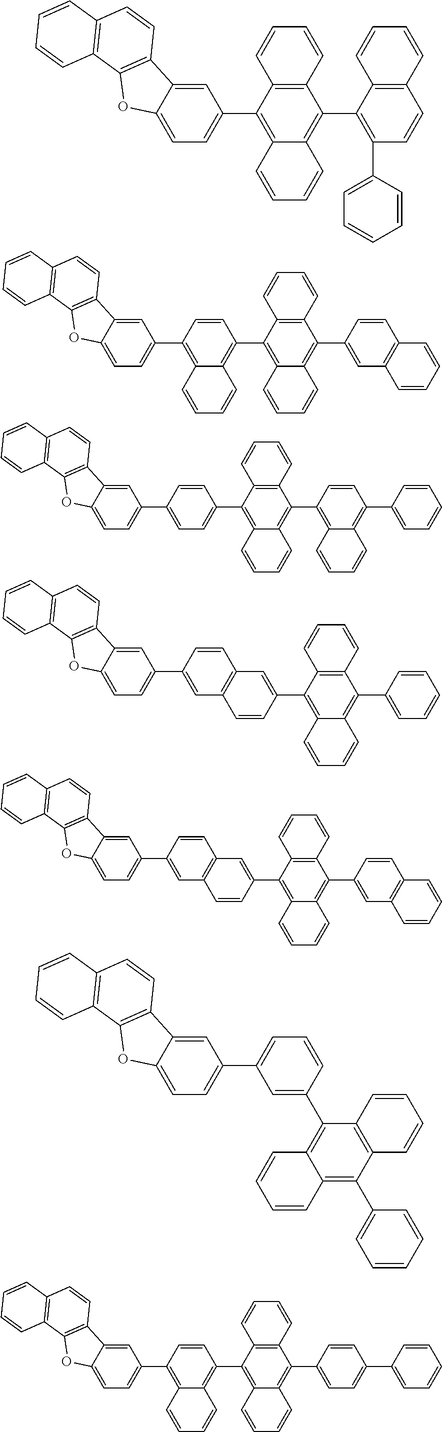

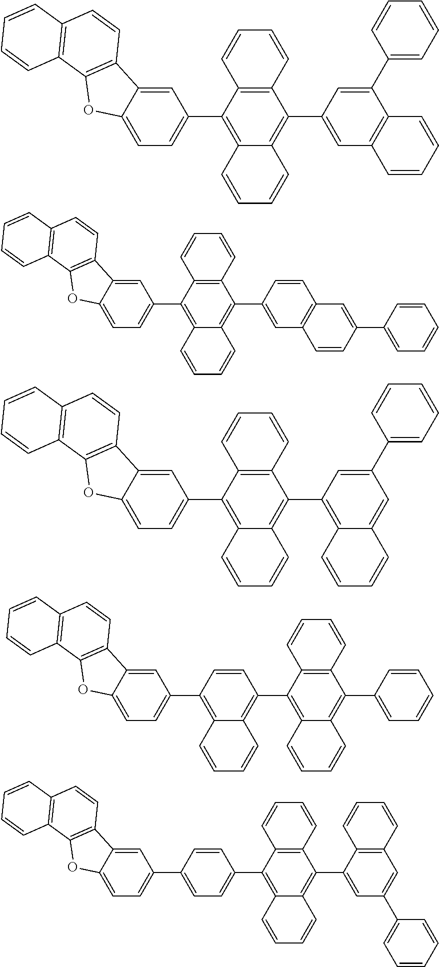

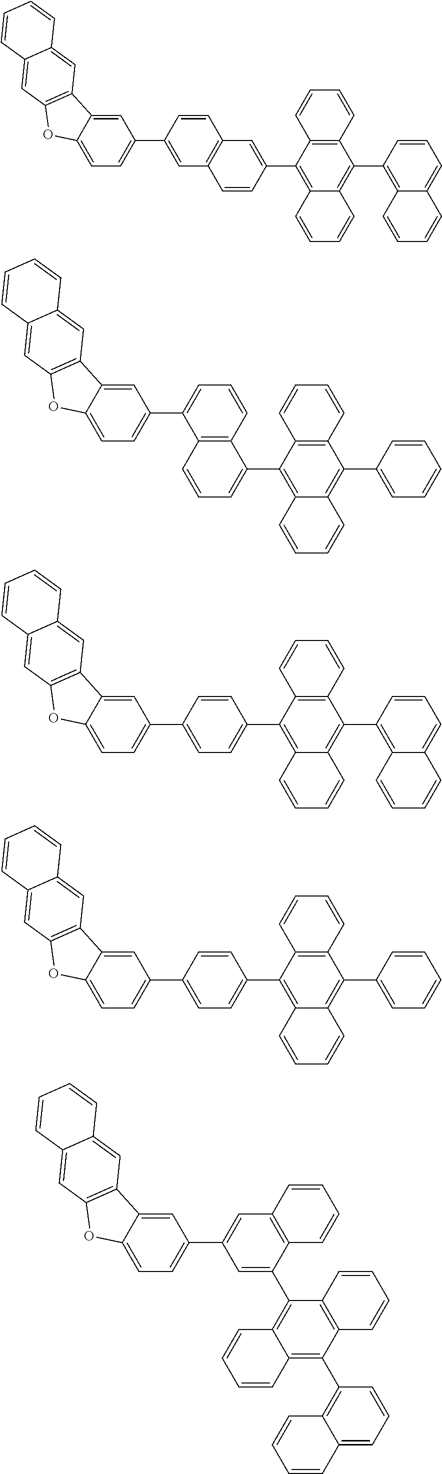 [SMILES: c1ccc2cc3c(cc2c1)oc1ccc(-c2ccc4cc(-c5c6ccccc6c(-c6cccc7ccccc67)c6ccccc56)ccc4c2)cc13 ; c1ccc(-c2c3ccccc3c(-c3cccc4c(-c5ccc6oc7cc8ccccc8cc7c6c5)cccc34)c3ccccc23)cc1 ; c1ccc2cc3c(cc2c1)oc1ccc(-c2ccc(-c4c5ccccc5c(-c5cccc6ccccc56)c5ccccc45)cc2)cc13 ; c1ccc(-c2c3ccccc3c(-c3ccc(-c4ccc5oc6cc7ccccc7cc6c5c4)cc3)c3ccccc23)cc1 ; c1ccc2cc3c(cc2c1)oc1ccc(-c2cc(-c4c5ccccc5c(-c5cccc6ccccc56)c5ccccc45)c4ccccc4c2)cc13]

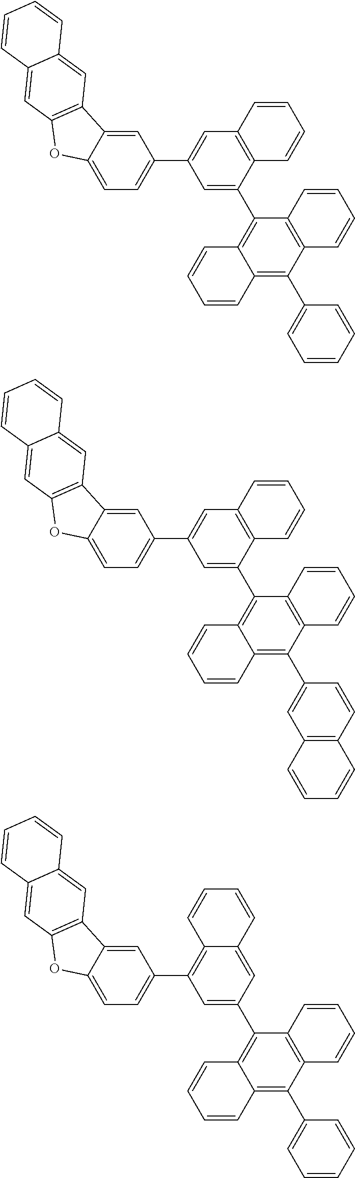 [SMILES: c1ccc(-c2c3ccccc3c(-c3cc(-c4ccc5oc6cc7ccccc7cc6c5c4)cc4ccccc34)c3ccccc23)cc1 ; c1ccc2cc(-c3c4ccccc4c(-c4cc(-c5ccc6oc7cc8ccccc8cc7c6c5)cc5ccccc45)c4ccccc34)ccc2c1 ; c1ccc(-c2c3ccccc3c(-c3cc(-c4ccc5oc6cc7ccccc7cc6c5c4)c4ccccc4c3)c3ccccc23)cc1]

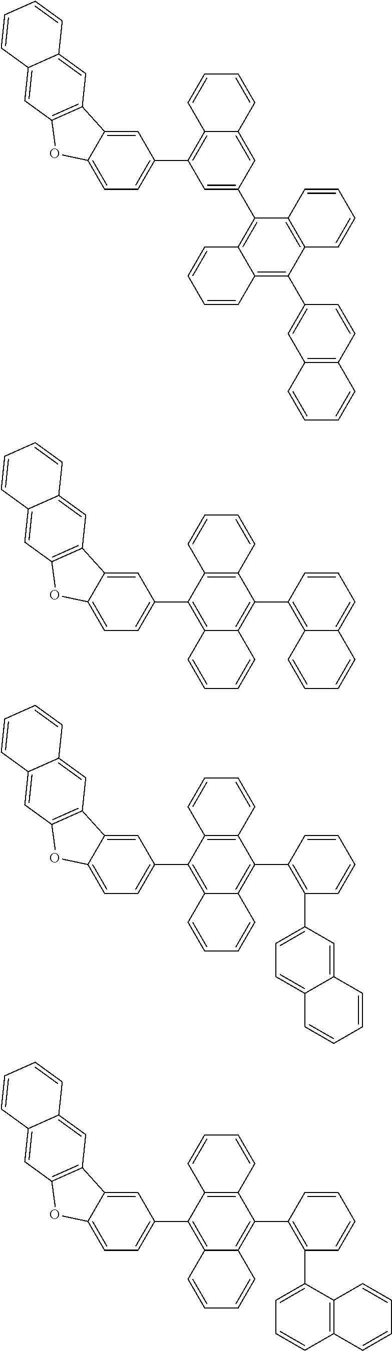 [SMILES: c1ccc2cc(-c3c4ccccc4c(-c4cc(-c5ccc6oc7cc8ccccc8cc7c6c5)c5ccccc5c4)c4ccccc34)ccc2c1 ; c1ccc2cc3c(cc2c1)oc1ccc(-c2c4ccccc4c(-c4cccc5ccccc45)c4ccccc24)cc13 ; c1ccc(-c2c3ccccc3c(-c3ccc4oc5cc6ccccc6cc5c4c3)c3ccccc23)c(-c2ccc3ccccc3c2)c1 ; c1ccc(-c2c3ccccc3c(-c3ccc4oc5cc6ccccc6cc5c4c3)c3ccccc23)c(-c2cccc3ccccc23)c1]

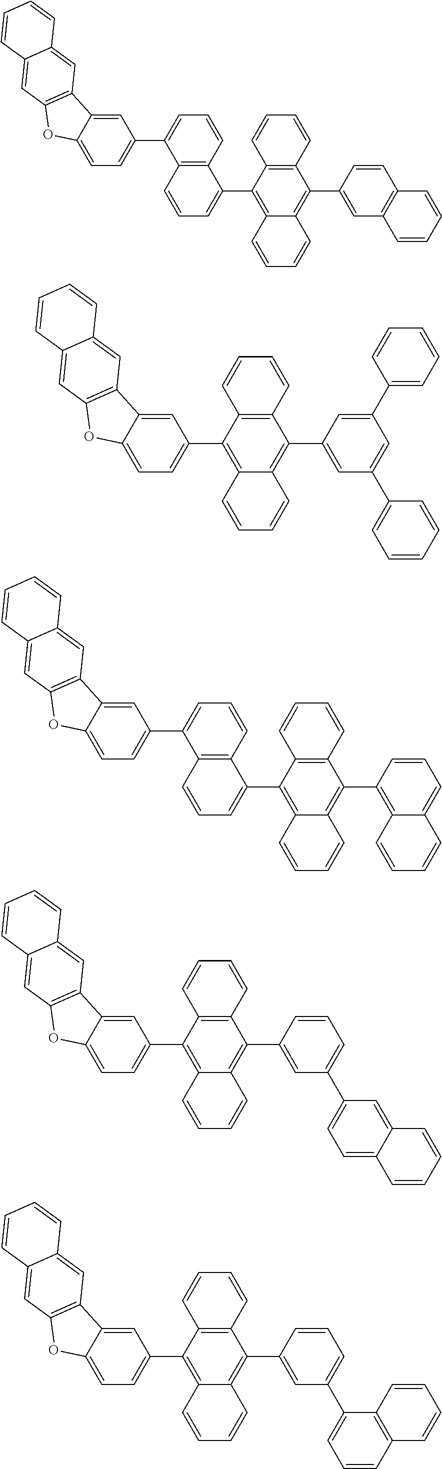 [SMILES: c1ccc2cc(-c3c4ccccc4c(-c4cccc5c(-c6ccc7oc8cc9ccccc9cc8c7c6)cccc45)c4ccccc34)ccc2c1 ; c1ccc(-c2cc(-c3ccccc3)cc(-c3c4ccccc4c(-c4ccc5oc6cc7ccccc7cc6c5c4)c4ccccc34)c2)cc1 ; c1ccc2cc3c(cc2c1)oc1ccc(-c2cccc4c(-c5c6ccccc6c(-c6cccc7ccccc67)c6ccccc56)cccc24)cc13 ; c1cc(-c2ccc3ccccc3c2)cc(-c2c3ccccc3c(-c3ccc4oc5cc6ccccc6cc5c4c3)c3ccccc23)c1 ; c1cc(-c2cccc3ccccc23)cc(-c2c3ccccc3c(-c3ccc4oc5cc6ccccc6cc5c4c3)c3ccccc23)c1]

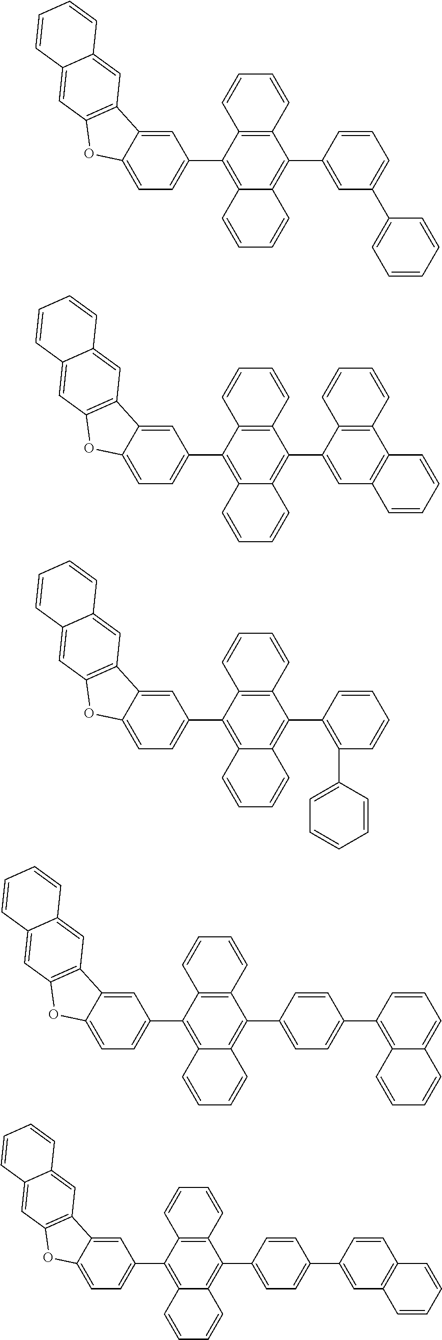 [SMILES: c1ccc(-c2cccc(-c3c4ccccc4c(-c4ccc5oc6cc7ccccc7cc6c5c4)c4ccccc34)c2)cc1 ; c1ccc2cc3c(cc2c1)oc1ccc(-c2c4ccccc4c(-c4cc5ccccc5c5ccccc45)c4ccccc24)cc13 ; c1ccc(-c2ccccc2-c2c3ccccc3c(-c3ccc4oc5cc6ccccc6cc5c4c3)c3ccccc23)cc1 ; c1ccc2cc3c(cc2c1)oc1ccc(-c2c4ccccc4c(-c4ccc(-c5cccc6ccccc56)cc4)c4ccccc24)cc13 ; c1ccc2cc(-c3ccc(-c4c5ccccc5c(-c5ccc6oc7cc8ccccc8cc7c6c5)c5ccccc45)cc3)ccc2c1]

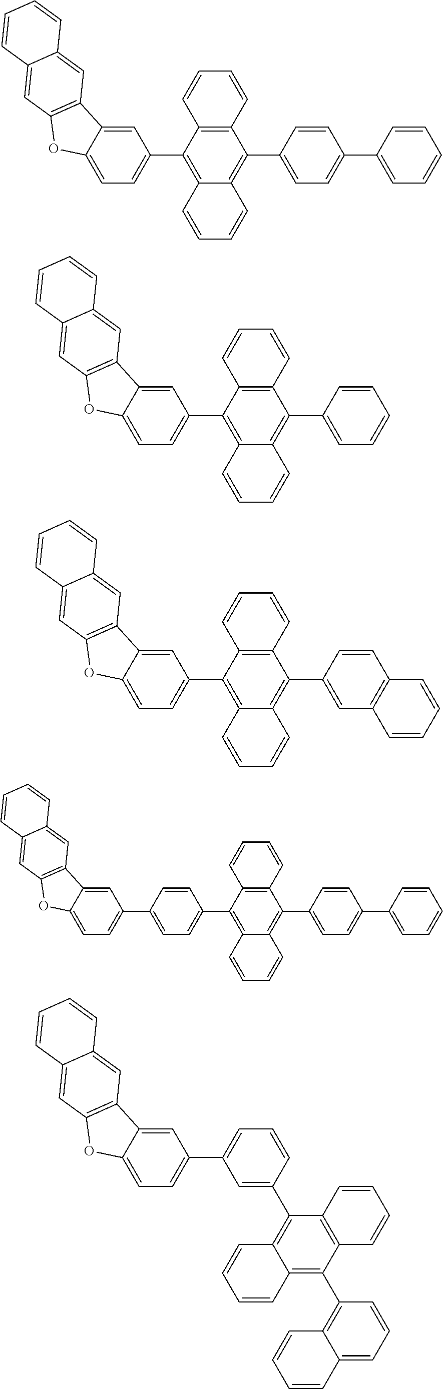 [SMILES: c1ccc(-c2ccc(-c3c4ccccc4c(-c4ccc5oc6cc7ccccc7cc6c5c4)c4ccccc34)cc2)cc1 ; c1ccc(-c2c3ccccc3c(-c3ccc4oc5cc6ccccc6cc5c4c3)c3ccccc23)cc1 ; c1ccc2cc(-c3c4ccccc4c(-c4ccc5oc6cc7ccccc7cc6c5c4)c4ccccc34)ccc2c1 ; c1ccc(-c2ccc(-c3c4ccccc4c(-c4ccc(-c5ccc6oc7cc8ccccc8cc7c6c5)cc4)c4ccccc34)cc2)cc1 ; c1cc(-c2ccc3oc4cc5ccccc5cc4c3c2)cc(-c2c3ccccc3c(-c3cccc4ccccc34)c3ccccc23)c1]

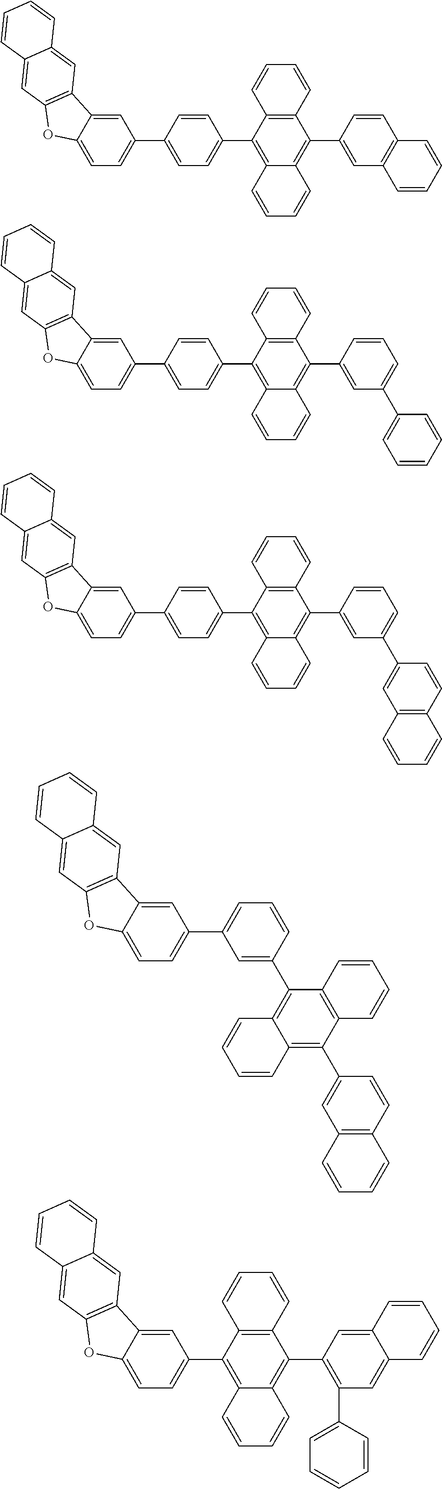 [SMILES: c1ccc2cc(-c3c4ccccc4c(-c4ccc(-c5ccc6oc7cc8ccccc8cc7c6c5)cc4)c4ccccc34)ccc2c1 ; c1ccc(-c2cccc(-c3c4ccccc4c(-c4ccc(-c5ccc6oc7cc8ccccc8cc7c6c5)cc4)c4ccccc34)c2)cc1 ; c1cc(-c2ccc3ccccc3c2)cc(-c2c3ccccc3c(-c3ccc(-c4ccc5oc6cc7ccccc7cc6c5c4)cc3)c3ccccc23)c1 ; c1cc(-c2ccc3oc4cc5ccccc5cc4c3c2)cc(-c2c3ccccc3c(-c3ccc4ccccc4c3)c3ccccc23)c1 ; c1ccc(-c2cc3ccccc3cc2-c2c3ccccc3c(-c3ccc4oc5cc6ccccc6cc5c4c3)c3ccccc23)cc1]

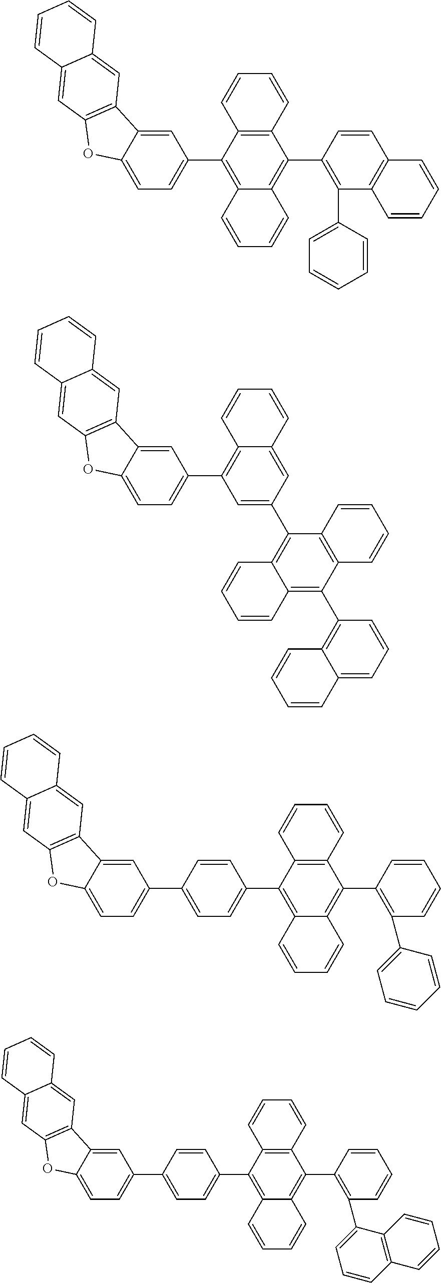 [SMILES: c1ccc(-c2c(-c3c4ccccc4c(-c4ccc5oc6cc7ccccc7cc6c5c4)c4ccccc34)ccc3ccccc23)cc1 ; c1ccc2cc3c(cc2c1)oc1ccc(-c2cc(-c4c5ccccc5c(-c5cccc6ccccc56)c5ccccc45)cc4ccccc24)cc13 ; c1ccc(-c2ccccc2-c2c3ccccc3c(-c3ccc(-c4ccc5oc6cc7ccccc7cc6c5c4)cc3)c3ccccc23)cc1 ; c1ccc(-c2c3ccccc3c(-c3ccc(-c4ccc5oc6cc7ccccc7cc6c5c4)cc3)c3ccccc23)c(-c2cccc3ccccc23)c1]

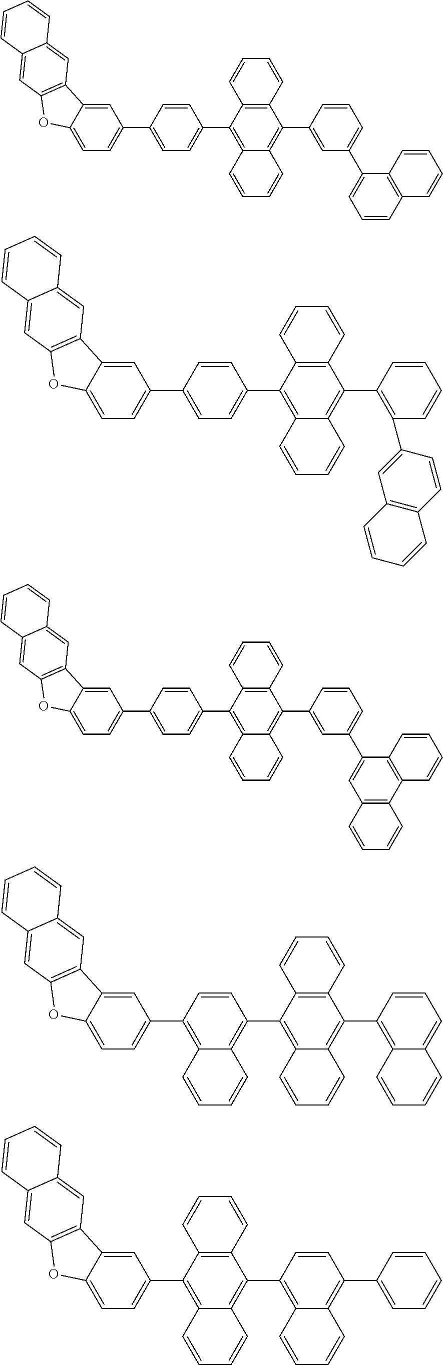 [SMILES: c1cc(-c2cccc3ccccc23)cc(-c2c3ccccc3c(-c3ccc(-c4ccc5oc6cc7ccccc7cc6c5c4)cc3)c3ccccc23)c1 ; c1ccc(-c2c3ccccc3c(-c3ccc(-c4ccc5oc6cc7ccccc7cc6c5c4)cc3)c3ccccc23)c(-c2ccc3ccccc3c2)c1 ; c1cc(-c2c3ccccc3c(-c3ccc(-c4ccc5oc6cc7ccccc7cc6c5c4)cc3)c3ccccc23)cc(-c2cc3ccccc3c3ccccc23)c1 ; c1ccc2cc3c(cc2c1)oc1ccc(-c2ccc(-c4c5ccccc5c(-c5cccc6ccccc56)c5ccccc45)c4ccccc24)cc13 ; c1ccc(-c2ccc(-c3c4ccccc4c(-c4ccc5oc6cc7ccccc7cc6c5c4)c4ccccc34)c3ccccc23)cc1]

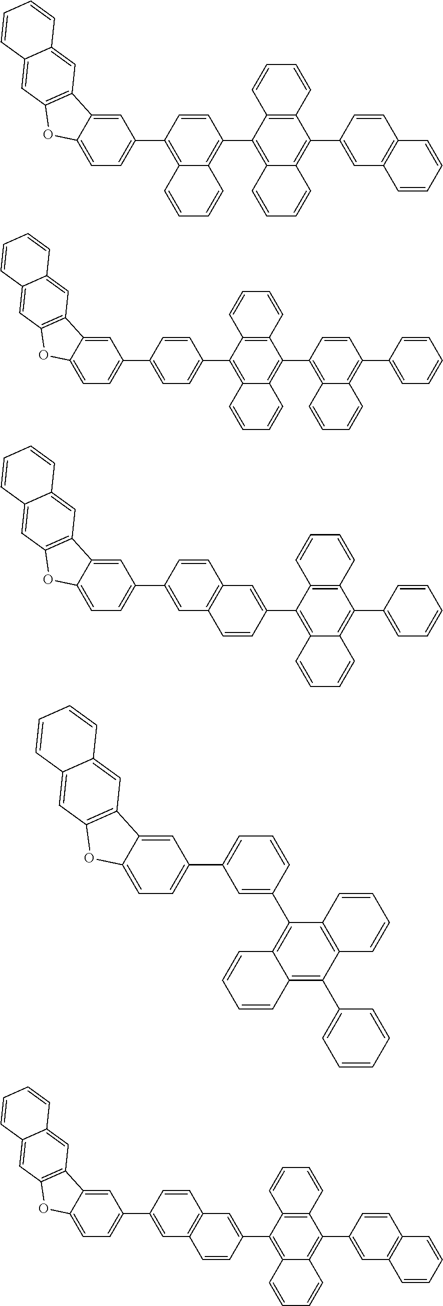 [SMILES: c1ccc2cc(-c3c4ccccc4c(-c4ccc(-c5ccc6oc7cc8ccccc8cc7c6c5)c5ccccc45)c4ccccc34)ccc2c1 ; c1ccc(-c2ccc(-c3c4ccccc4c(-c4ccc(-c5ccc6oc7cc8ccccc8cc7c6c5)cc4)c4ccccc34)c3ccccc23)cc1 ; c1ccc(-c2c3ccccc3c(-c3ccc4cc(-c5ccc6oc7cc8ccccc8cc7c6c5)ccc4c3)c3ccccc23)cc1 ; c1ccc(-c2c3ccccc3c(-c3cccc(-c4ccc5oc6cc7ccccc7cc6c5c4)c3)c3ccccc23)cc1 ; c1ccc2cc(-c3c4ccccc4c(-c4ccc5cc(-c6ccc7oc8cc9ccccc9cc8c7c6)ccc5c4)c4ccccc34)ccc2c1]

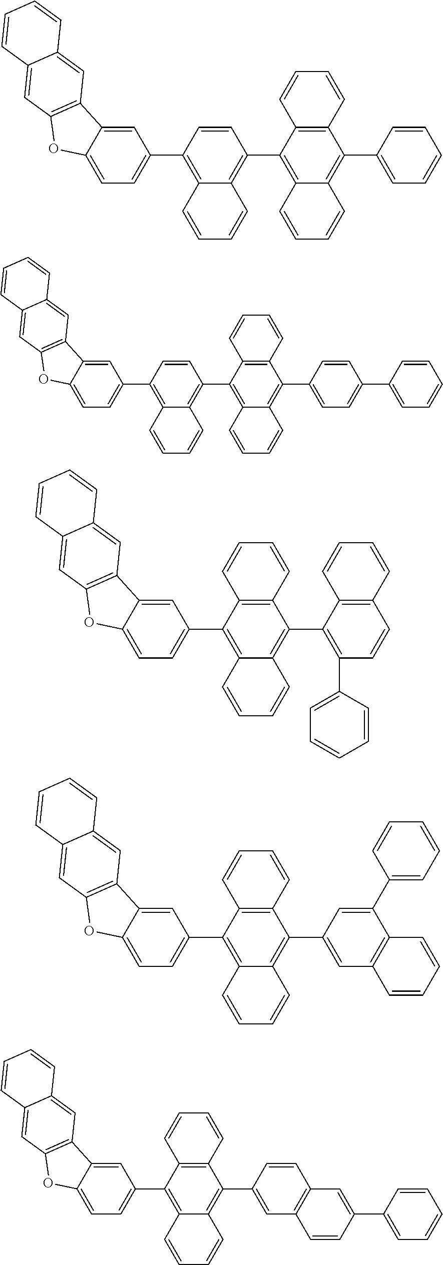 [SMILES: c1ccc(-c2c3ccccc3c(-c3ccc(-c4ccc5oc6cc7ccccc7cc6c5c4)c4ccccc34)c3ccccc23)cc1 ; c1ccc(-c2ccc(-c3c4ccccc4c(-c4ccc(-c5ccc6oc7cc8ccccc8cc7c6c5)c5ccccc45)c4ccccc34)cc2)cc1 ; c1ccc(-c2ccc3ccccc3c2-c2c3ccccc3c(-c3ccc4oc5cc6ccccc6cc5c4c3)c3ccccc23)cc1 ; c1ccc(-c2cc(-c3c4ccccc4c(-c4ccc5oc6cc7ccccc7cc6c5c4)c4ccccc34)cc3ccccc23)cc1 ; c1ccc(-c2ccc3cc(-c4c5ccccc5c(-c5ccc6oc7cc8ccccc8cc7c6c5)c5ccccc45)ccc3c2)cc1]

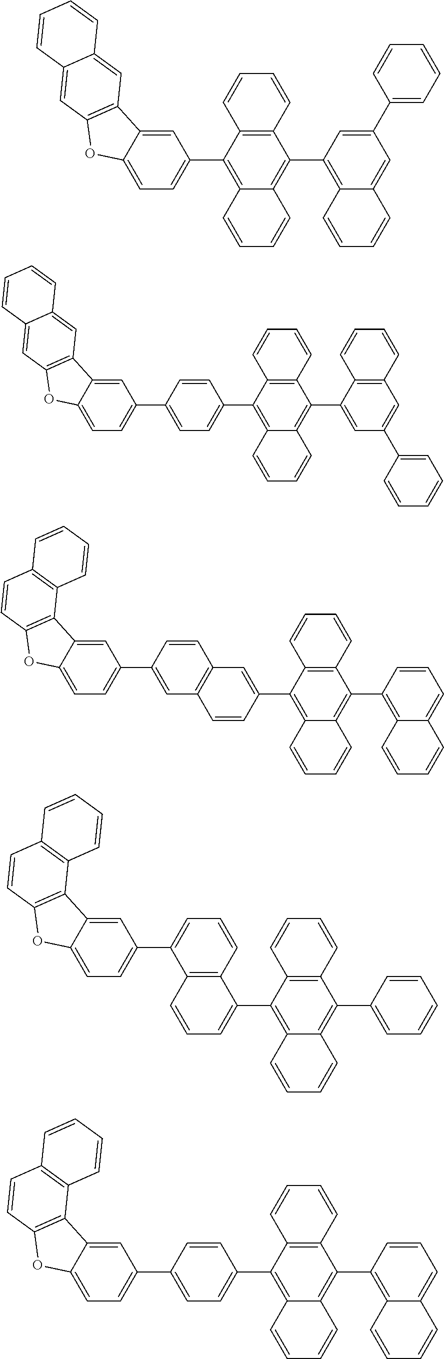 [SMILES: c1ccc(-c2cc(-c3c4ccccc4c(-c4ccc5oc6cc7ccccc7cc6c5c4)c4ccccc34)c3ccccc3c2)cc1 ; c1ccc(-c2cc(-c3c4ccccc4c(-c4ccc(-c5ccc6oc7cc8ccccc8cc7c6c5)cc4)c4ccccc34)c3ccccc3c2)cc1 ; c1ccc2c(-c3c4ccccc4c(-c4ccc5cc(-c6ccc7oc8ccc9ccccc9c8c7c6)ccc5c4)c4ccccc34)cccc2c1 ; c1ccc(-c2c3ccccc3c(-c3cccc4c(-c5ccc6oc7ccc8ccccc8c7c6c5)cccc34)c3ccccc23)cc1 ; c1ccc2c(-c3c4ccccc4c(-c4ccc(-c5ccc6oc7ccc8ccccc8c7c6c5)cc4)c4ccccc34)cccc2c1]

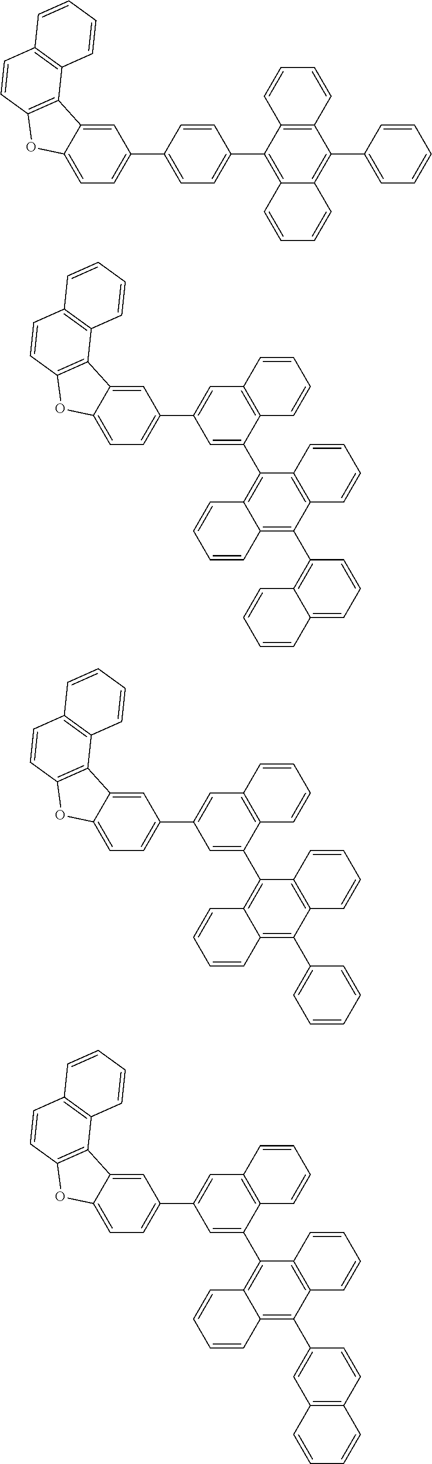 [SMILES: c1ccc(-c2c3ccccc3c(-c3ccc(-c4ccc5oc6ccc7ccccc7c6c5c4)cc3)c3ccccc23)cc1 ; c1ccc2c(-c3c4ccccc4c(-c4cc(-c5ccc6oc7ccc8ccccc8c7c6c5)cc5ccccc45)c4ccccc34)cccc2c1 ; c1ccc(-c2c3ccccc3c(-c3cc(-c4ccc5oc6ccc7ccccc7c6c5c4)cc4ccccc34)c3ccccc23)cc1 ; c1ccc2cc(-c3c4ccccc4c(-c4cc(-c5ccc6oc7ccc8ccccc8c7c6c5)cc5ccccc45)c4ccccc34)ccc2c1]

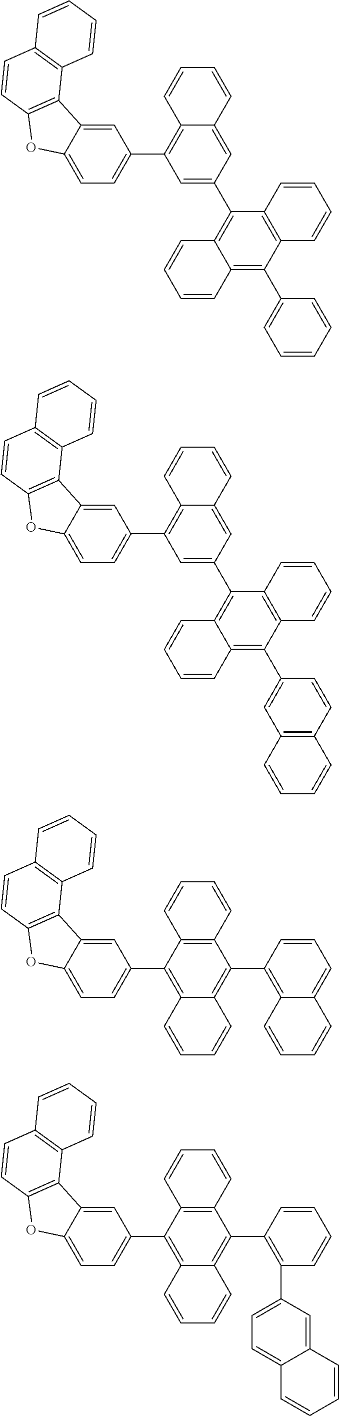 [SMILES: c1ccc(-c2c3ccccc3c(-c3cc(-c4ccc5oc6ccc7ccccc7c6c5c4)c4ccccc4c3)c3ccccc23)cc1 ; c1ccc2cc(-c3c4ccccc4c(-c4cc(-c5ccc6oc7ccc8ccccc8c7c6c5)c5ccccc5c4)c4ccccc34)ccc2c1 ; c1ccc2c(-c3c4ccccc4c(-c4ccc5oc6ccc7ccccc7c6c5c4)c4ccccc34)cccc2c1 ; c1ccc(-c2c3ccccc3c(-c3ccc4oc5ccc6ccccc6c5c4c3)c3ccccc23)c(-c2ccc3ccccc3c2)c1]

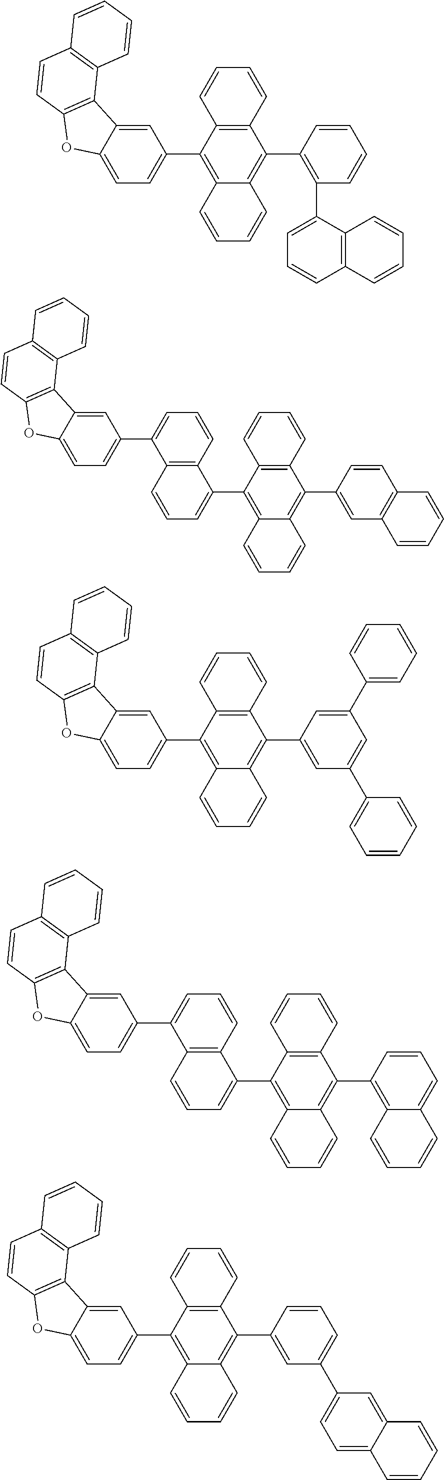 [SMILES: c1ccc(-c2c3ccccc3c(-c3ccc4oc5ccc6ccccc6c5c4c3)c3ccccc23)c(-c2cccc3ccccc23)c1 ; c1ccc2cc(-c3c4ccccc4c(-c4cccc5c(-c6ccc7oc8ccc9ccccc9c8c7c6)cccc45)c4ccccc34)ccc2c1 ; c1ccc(-c2cc(-c3ccccc3)cc(-c3c4ccccc4c(-c4ccc5oc6ccc7ccccc7c6c5c4)c4ccccc34)c2)cc1 ; c1ccc2c(-c3c4ccccc4c(-c4cccc5c(-c6ccc7oc8ccc9ccccc9c8c7c6)cccc45)c4ccccc34)cccc2c1 ; c1cc(-c2ccc3ccccc3c2)cc(-c2c3ccccc3c(-c3ccc4oc5ccc6ccccc6c5c4c3)c3ccccc23)c1]

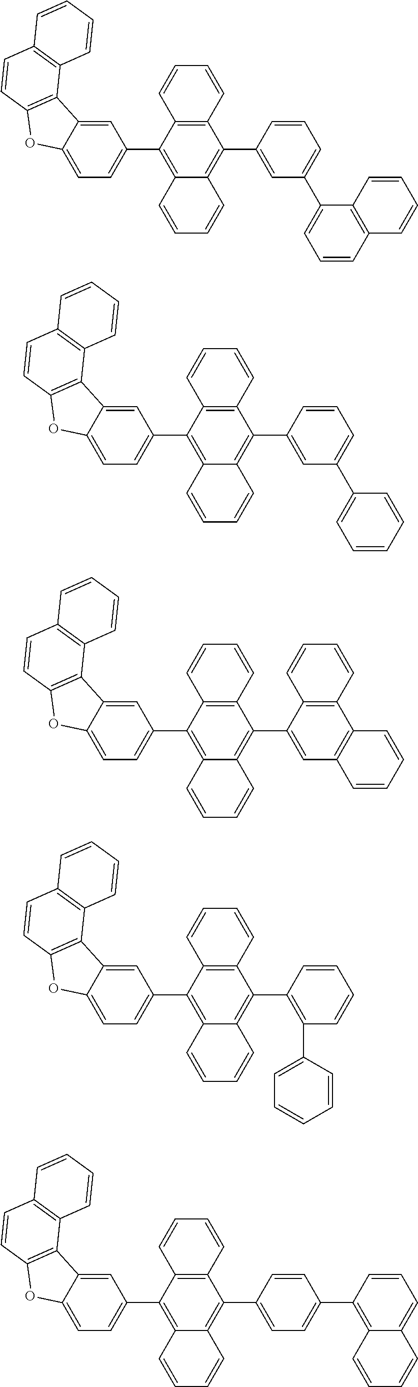 [SMILES: c1cc(-c2cccc3ccccc23)cc(-c2c3ccccc3c(-c3ccc4oc5ccc6ccccc6c5c4c3)c3ccccc23)c1 ; c1ccc(-c2cccc(-c3c4ccccc4c(-c4ccc5oc6ccc7ccccc7c6c5c4)c4ccccc34)c2)cc1 ; c1ccc2c(c1)cc(-c1c3ccccc3c(-c3ccc4oc5ccc6ccccc6c5c4c3)c3ccccc13)c1ccccc12 ; c1ccc(-c2ccccc2-c2c3ccccc3c(-c3ccc4oc5ccc6ccccc6c5c4c3)c3ccccc23)cc1 ; c1ccc2c(-c3ccc(-c4c5ccccc5c(-c5ccc6oc7ccc8ccccc8c7c6c5)c5ccccc45)cc3)cccc2c1]

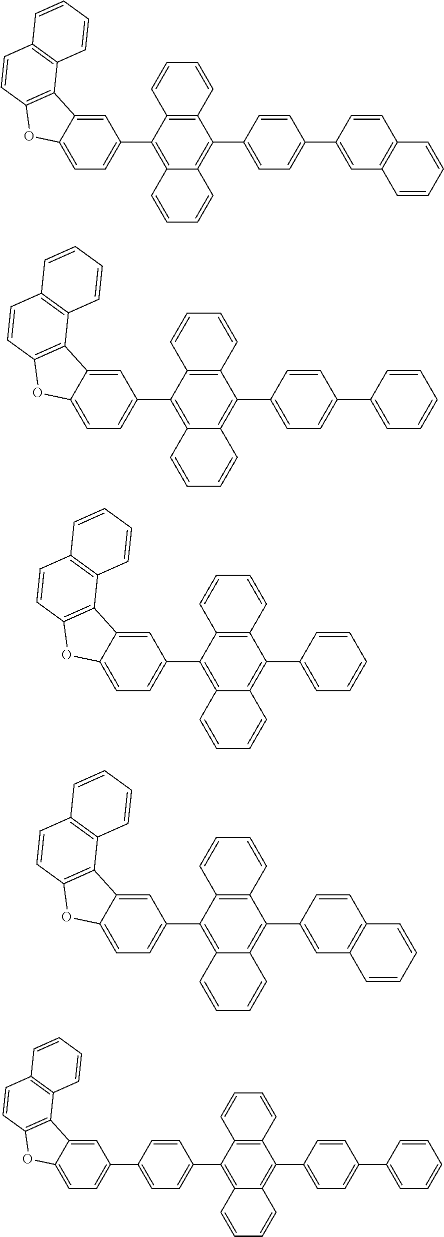 [SMILES: c1ccc2cc(-c3ccc(-c4c5ccccc5c(-c5ccc6oc7ccc8ccccc8c7c6c5)c5ccccc45)cc3)ccc2c1 ; c1ccc(-c2ccc(-c3c4ccccc4c(-c4ccc5oc6ccc7ccccc7c6c5c4)c4ccccc34)cc2)cc1 ; c1ccc(-c2c3ccccc3c(-c3ccc4oc5ccc6ccccc6c5c4c3)c3ccccc23)cc1 ; c1ccc2cc(-c3c4ccccc4c(-c4ccc5oc6ccc7ccccc7c6c5c4)c4ccccc34)ccc2c1 ; c1ccc(-c2ccc(-c3c4ccccc4c(-c4ccc(-c5ccc6oc7ccc8ccccc8c7c6c5)cc4)c4ccccc34)cc2)cc1]

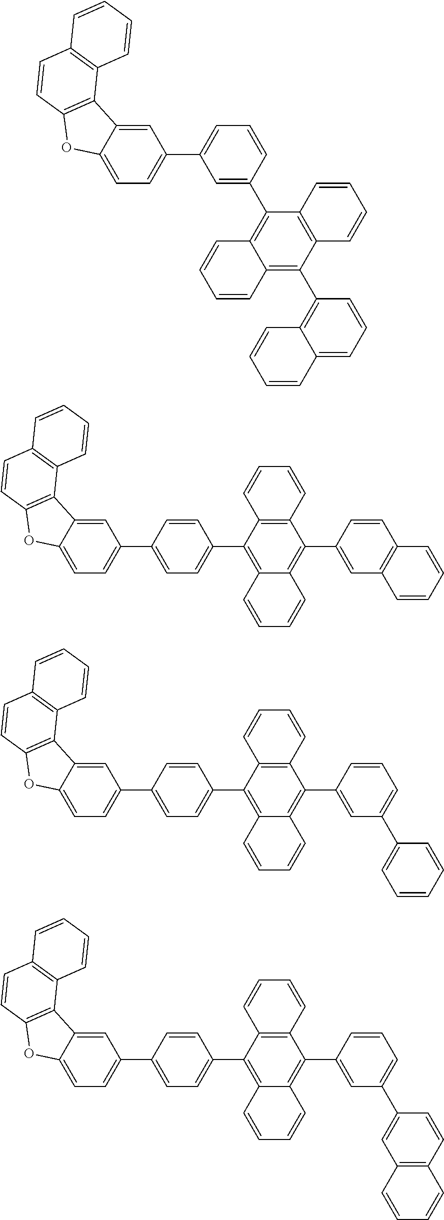 [SMILES: c1cc(-c2ccc3oc4ccc5ccccc5c4c3c2)cc(-c2c3ccccc3c(-c3cccc4ccccc34)c3ccccc23)c1 ; c1ccc2cc(-c3c4ccccc4c(-c4ccc(-c5ccc6oc7ccc8ccccc8c7c6c5)cc4)c4ccccc34)ccc2c1 ; c1ccc(-c2cccc(-c3c4ccccc4c(-c4ccc(-c5ccc6oc7ccc8ccccc8c7c6c5)cc4)c4ccccc34)c2)cc1 ; c1cc(-c2ccc3ccccc3c2)cc(-c2c3ccccc3c(-c3ccc(-c4ccc5oc6ccc7ccccc7c6c5c4)cc3)c3ccccc23)c1]

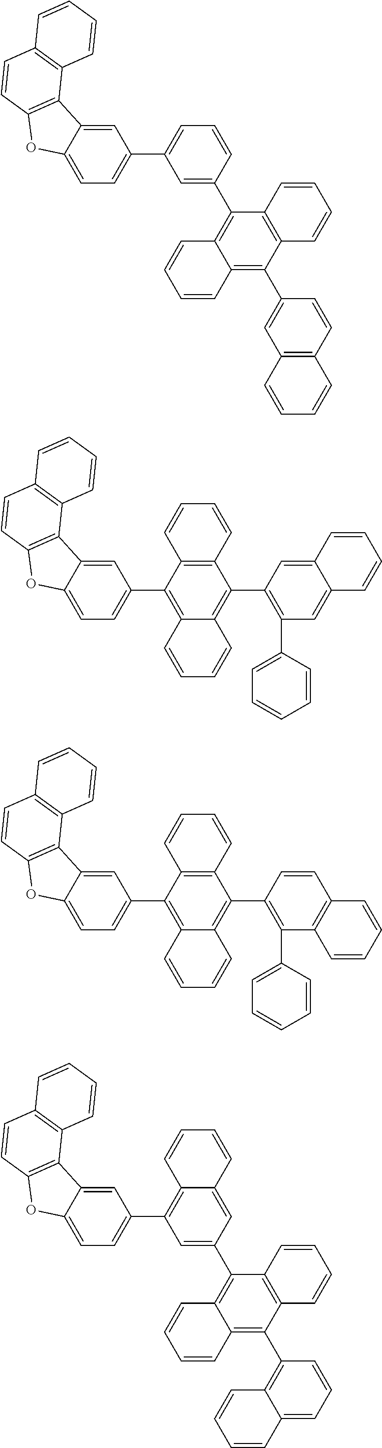 [SMILES: c1cc(-c2ccc3oc4ccc5ccccc5c4c3c2)cc(-c2c3ccccc3c(-c3ccc4ccccc4c3)c3ccccc23)c1 ; c1ccc(-c2cc3ccccc3cc2-c2c3ccccc3c(-c3ccc4oc5ccc6ccccc6c5c4c3)c3ccccc23)cc1 ; c1ccc(-c2c(-c3c4ccccc4c(-c4ccc5oc6ccc7ccccc7c6c5c4)c4ccccc34)ccc3ccccc23)cc1 ; c1ccc2c(-c3ccc4oc5ccc6ccccc6c5c4c3)cc(-c3c4ccccc4c(-c4cccc5ccccc45)c4ccccc34)cc2c1]

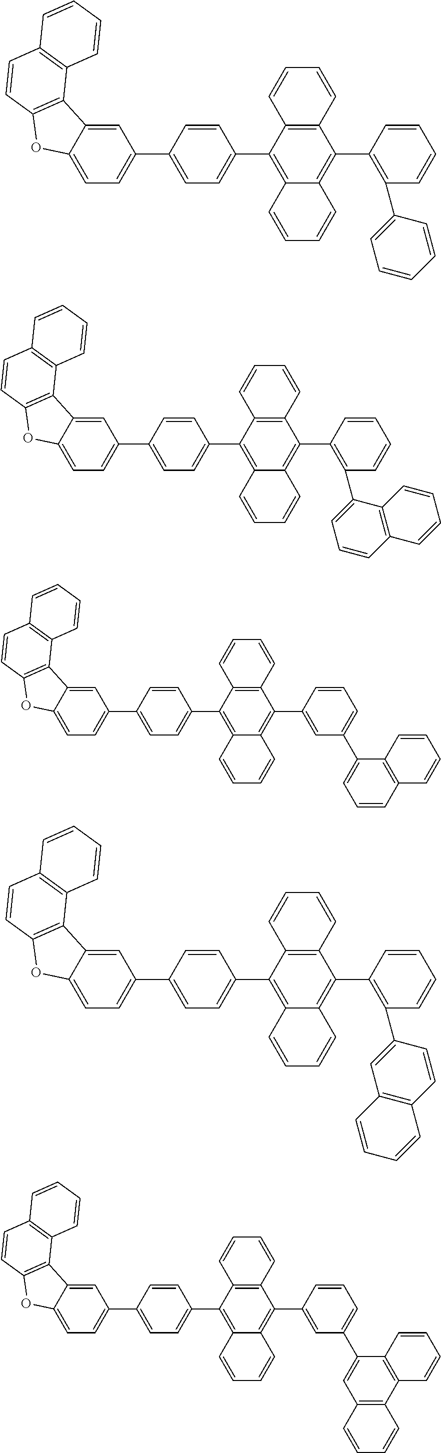 [SMILES: c1ccc(-c2ccccc2-c2c3ccccc3c(-c3ccc(-c4ccc5oc6ccc7ccccc7c6c5c4)cc3)c3ccccc23)cc1 ; c1ccc(-c2c3ccccc3c(-c3ccc(-c4ccc5oc6ccc7ccccc7c6c5c4)cc3)c3ccccc23)c(-c2cccc3ccccc23)c1 ; c1cc(-c2cccc3ccccc23)cc(-c2c3ccccc3c(-c3ccc(-c4ccc5oc6ccc7ccccc7c6c5c4)cc3)c3ccccc23)c1 ; c1ccc(-c2c3ccccc3c(-c3ccc(-c4ccc5oc6ccc7ccccc7c6c5c4)cc3)c3ccccc23)c(-c2ccc3ccccc3c2)c1 ; c1cc(-c2c3ccccc3c(-c3ccc(-c4ccc5oc6ccc7ccccc7c6c5c4)cc3)c3ccccc23)cc(-c2cc3ccccc3c3ccccc23)c1]

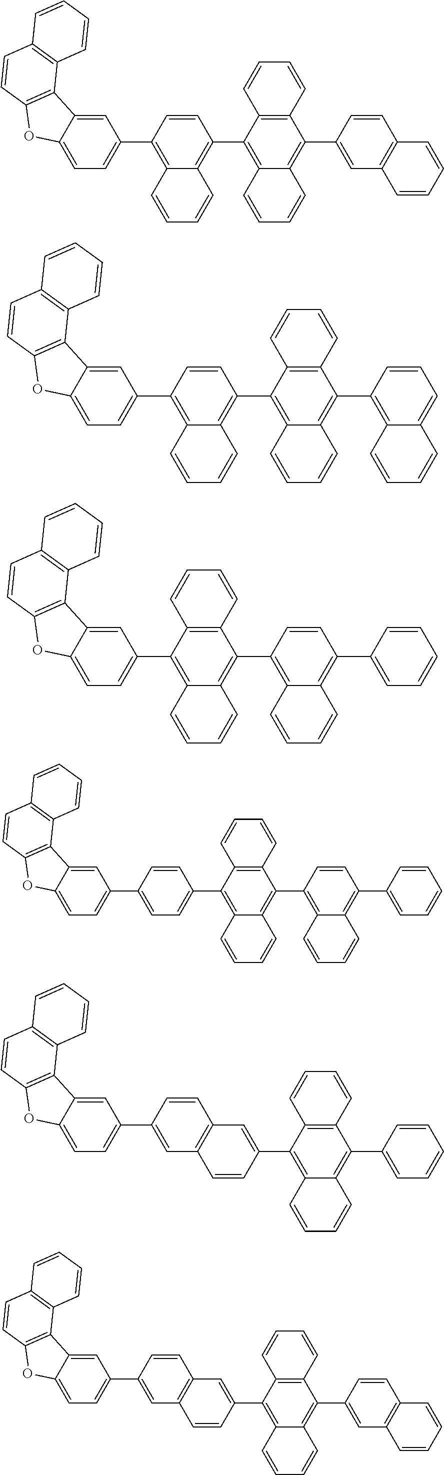 [SMILES: c1ccc2cc(-c3c4ccccc4c(-c4ccc(-c5ccc6oc7ccc8ccccc8c7c6c5)c5ccccc45)c4ccccc34)ccc2c1 ; c1ccc2c(-c3c4ccccc4c(-c4ccc(-c5ccc6oc7ccc8ccccc8c7c6c5)c5ccccc45)c4ccccc34)cccc2c1 ; c1ccc(-c2ccc(-c3c4ccccc4c(-c4ccc5oc6ccc7ccccc7c6c5c4)c4ccccc34)c3ccccc23)cc1 ; c1ccc(-c2ccc(-c3c4ccccc4c(-c4ccc(-c5ccc6oc7ccc8ccccc8c7c6c5)cc4)c4ccccc34)c3ccccc23)cc1 ; c1ccc(-c2c3ccccc3c(-c3ccc4cc(-c5ccc6oc7ccc8ccccc8c7c6c5)ccc4c3)c3ccccc23)cc1 ; c1ccc2cc(-c3c4ccccc4c(-c4ccc5cc(-c6ccc7oc8ccc9ccccc9c8c7c6)ccc5c4)c4ccccc34)ccc2c1]

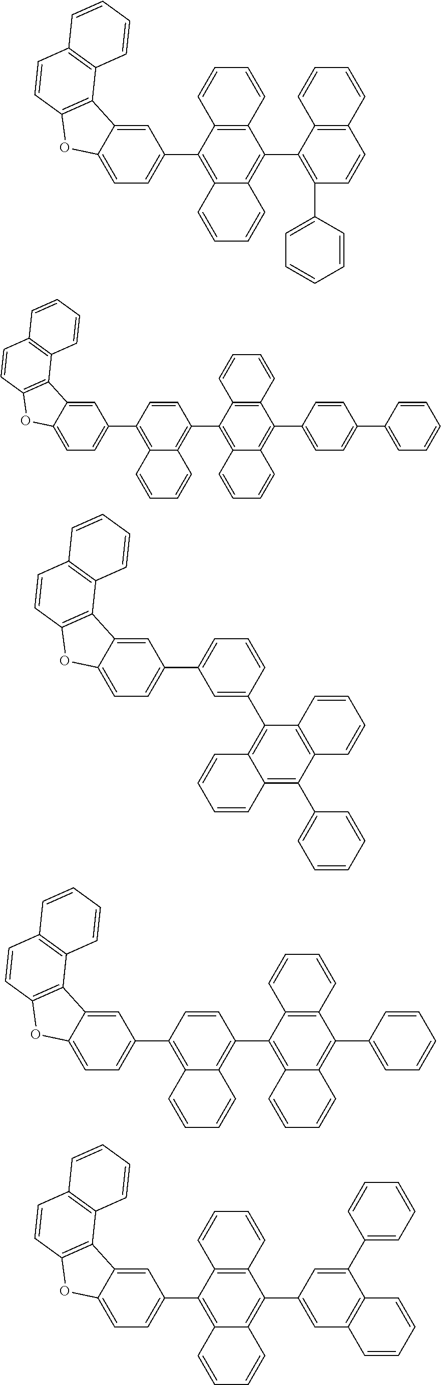

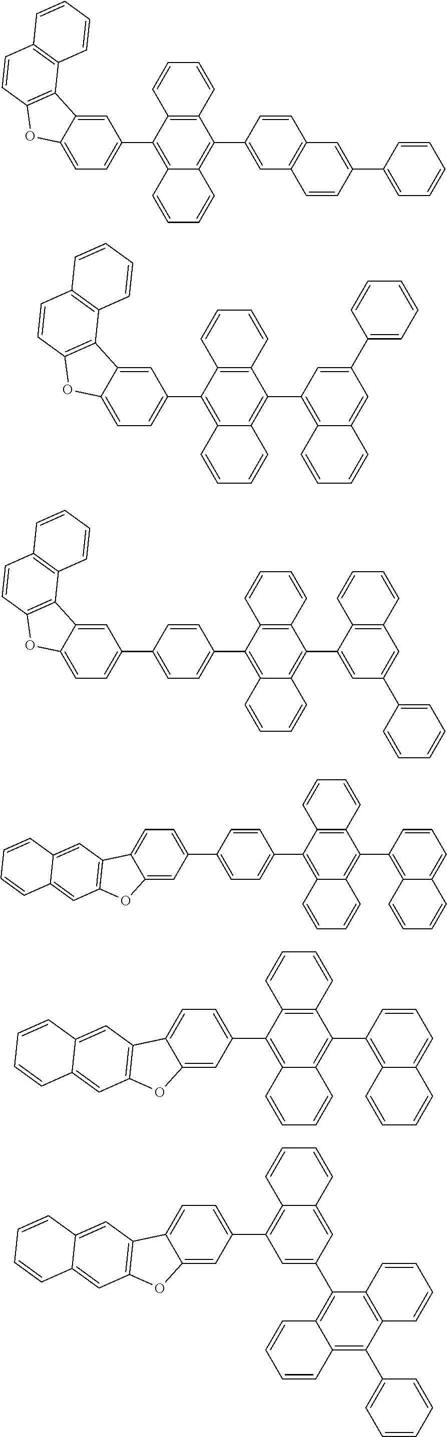

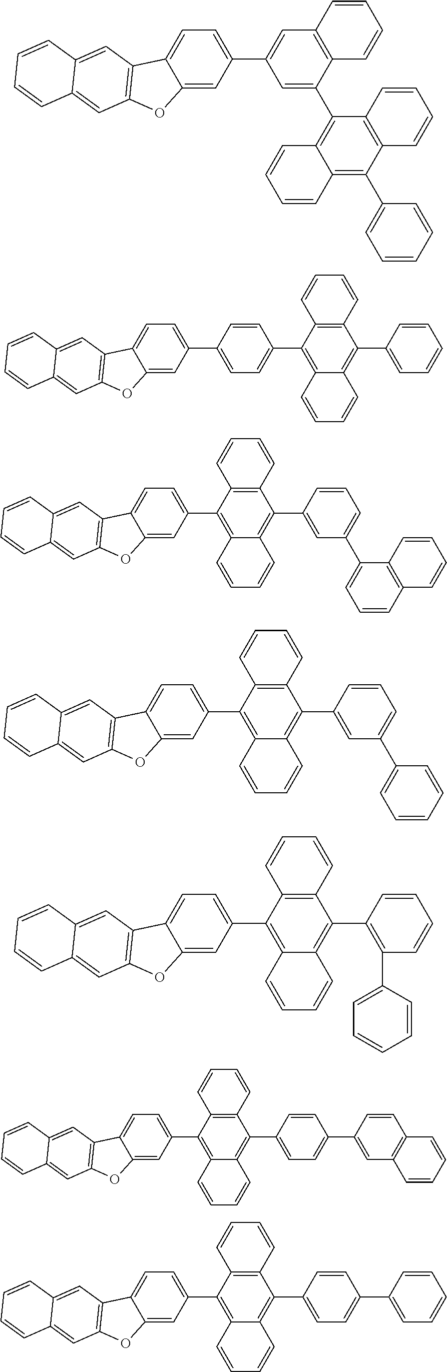

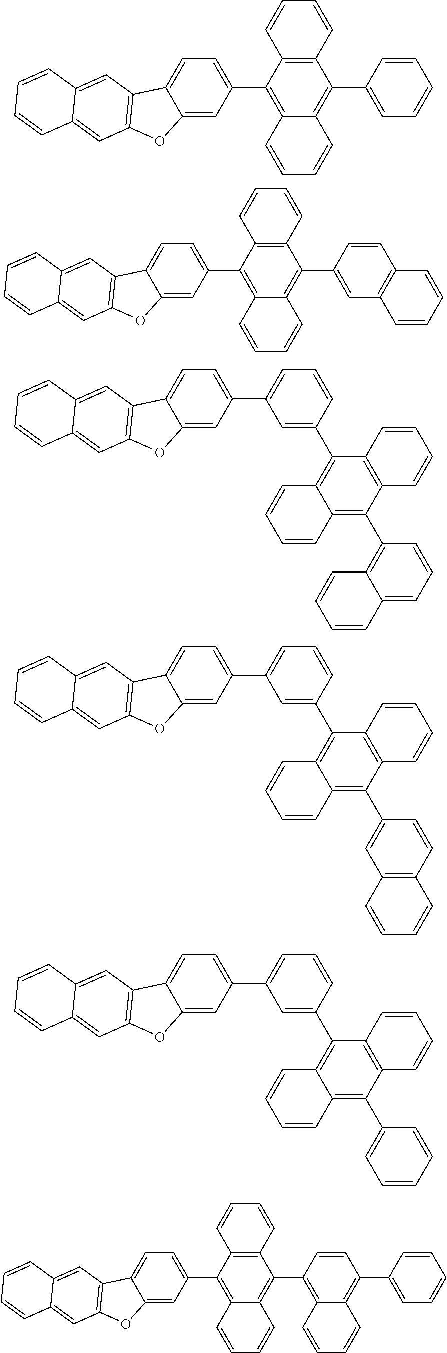

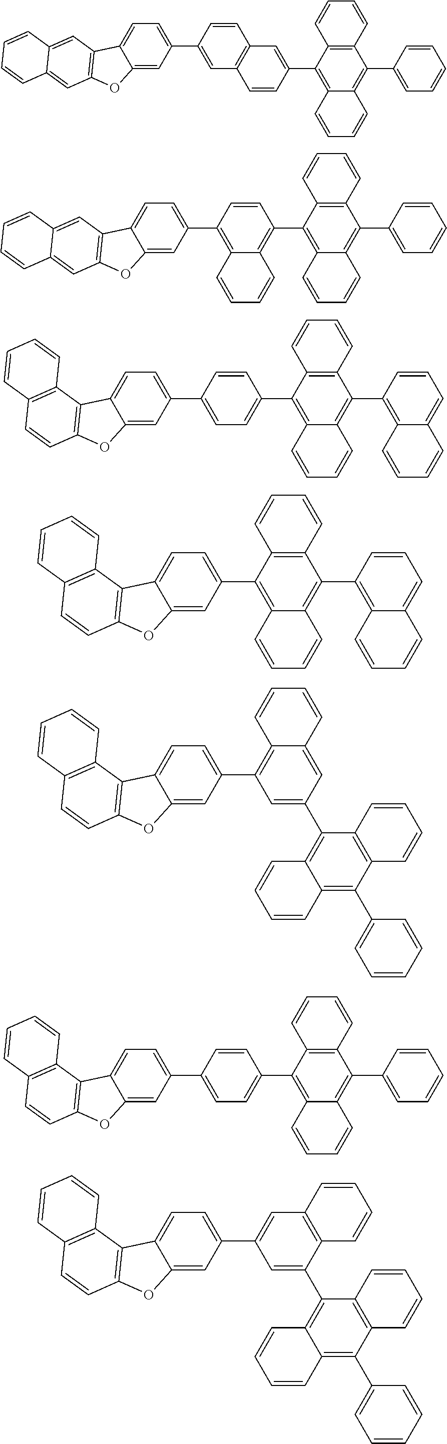

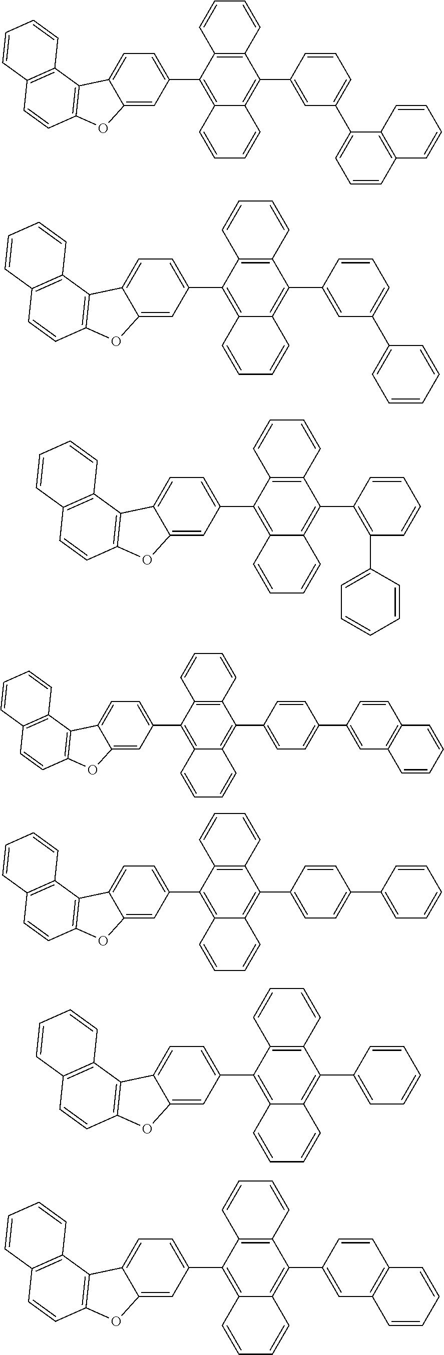

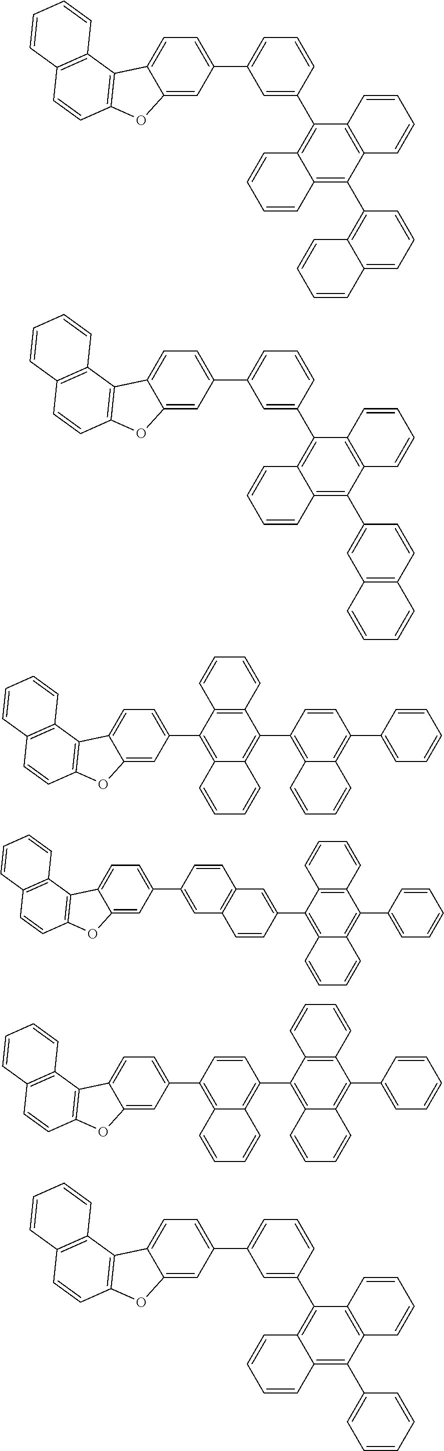

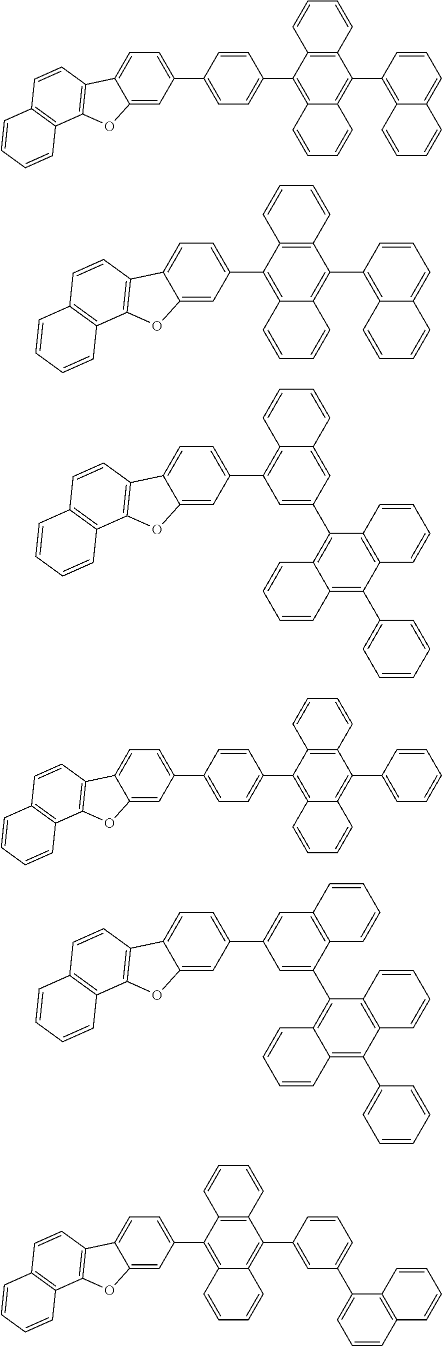

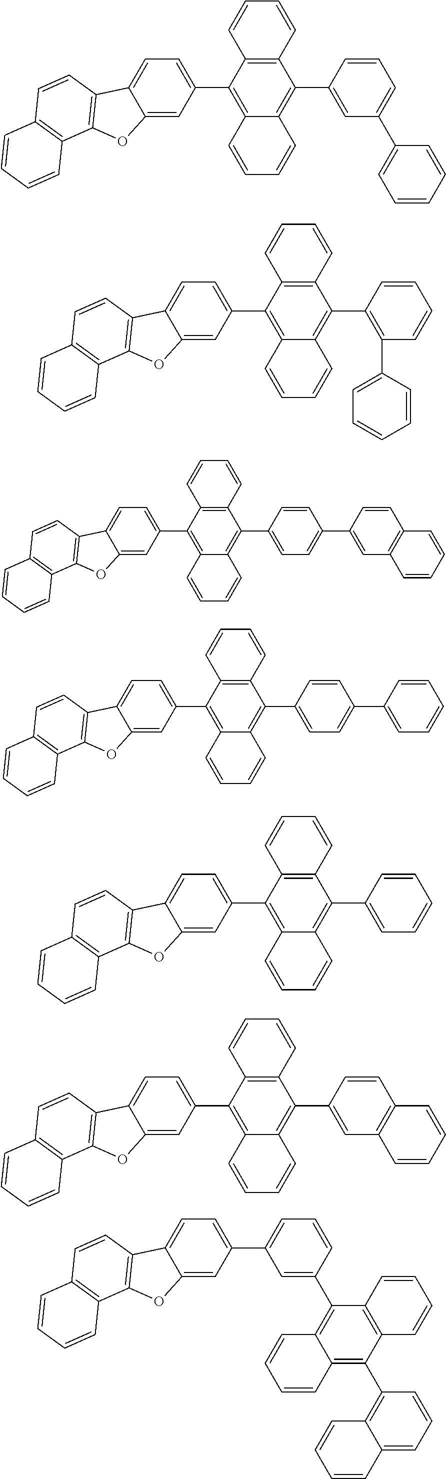

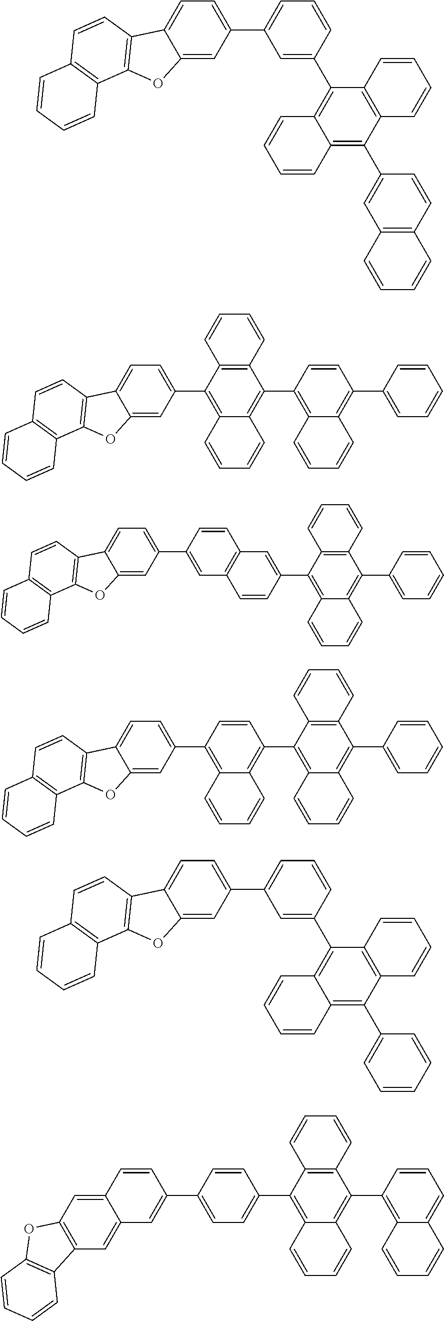

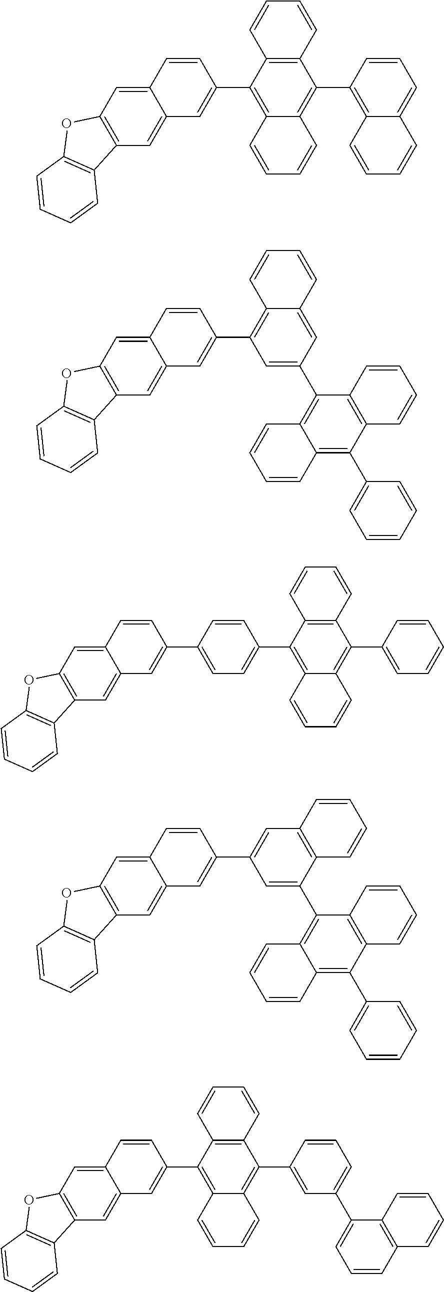

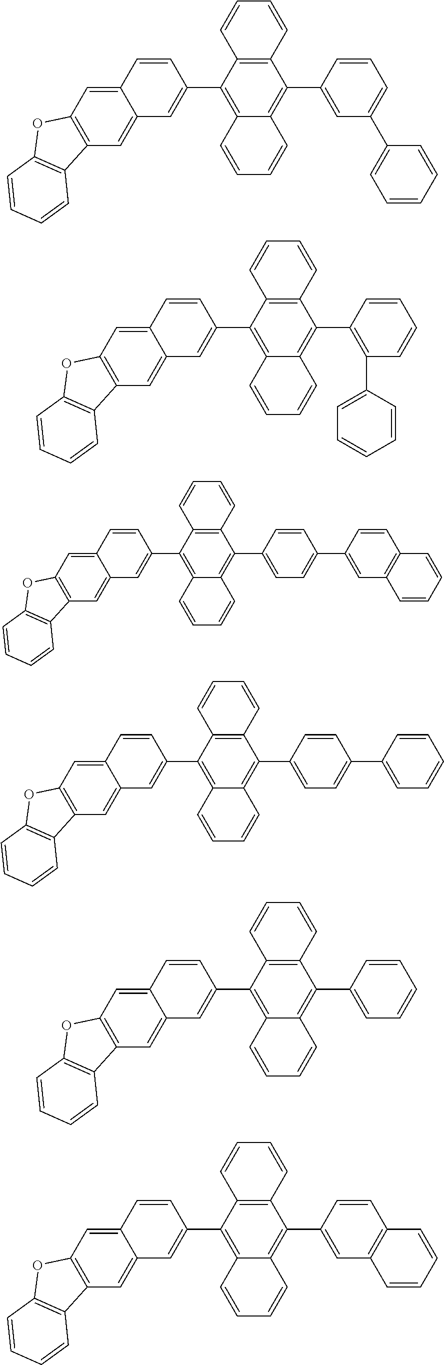 [SMILES: c1ccc(-c2cccc(-c3c4ccccc4c(-c4ccc5cc6oc7ccccc7c6cc5c4)c4ccccc34)c2)cc1 ; c1ccc(-c2ccccc2-c2c3ccccc3c(-c3ccc4cc5oc6ccccc6c5cc4c3)c3ccccc23)cc1 ; c1ccc2cc(-c3ccc(-c4c5ccccc5c(-c5ccc6cc7oc8ccccc8c7cc6c5)c5ccccc45)cc3)ccc2c1 ; c1ccc(-c2ccc(-c3c4ccccc4c(-c4ccc5cc6oc7ccccc7c6cc5c4)c4ccccc34)cc2)cc1 ; c1ccc(-c2c3ccccc3c(-c3ccc4cc5oc6ccccc6c5cc4c3)c3ccccc23)cc1 ; c1ccc2cc(-c3c4ccccc4c(-c4ccc5cc6oc7ccccc7c6cc5c4)c4ccccc34)ccc2c1]

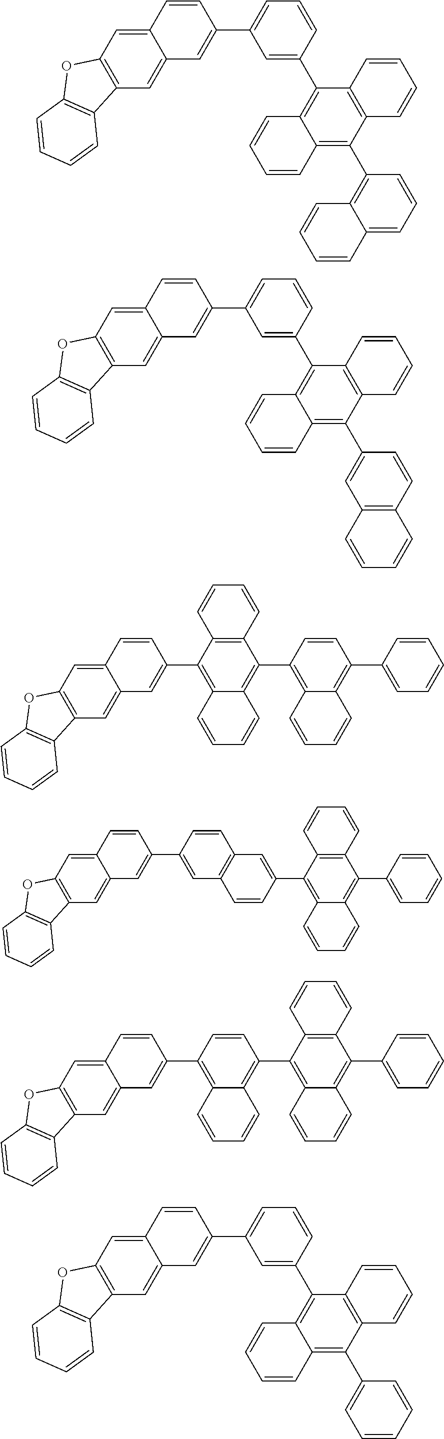

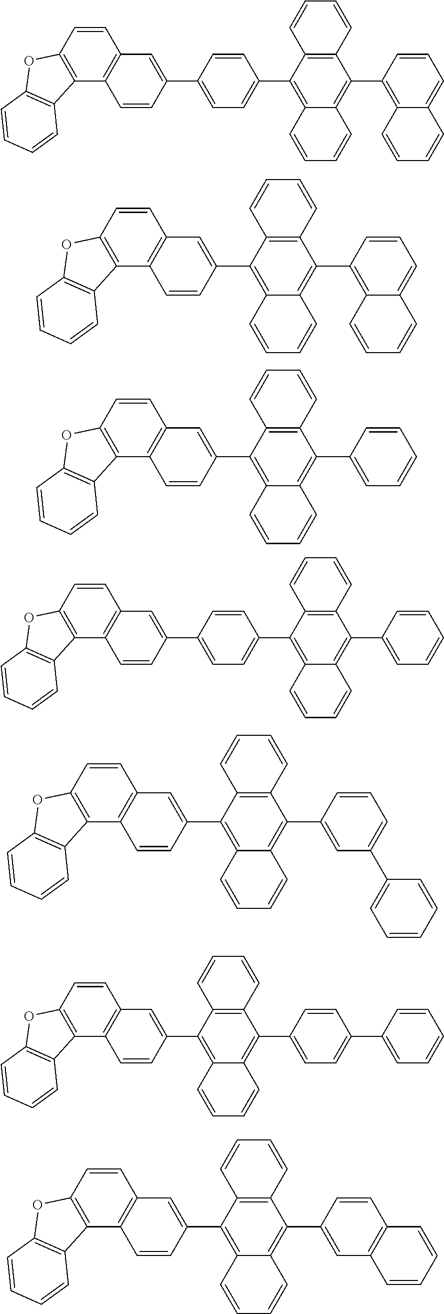

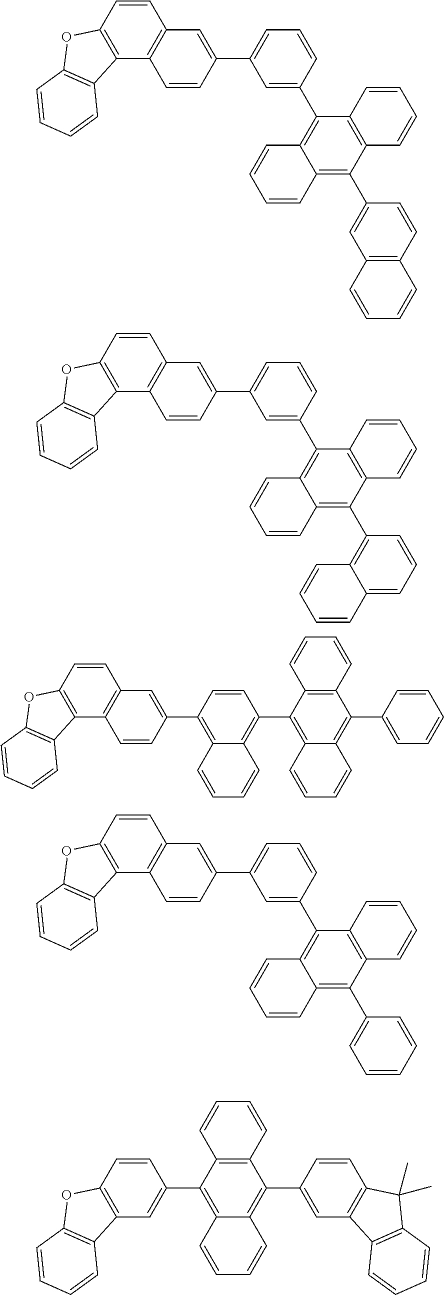

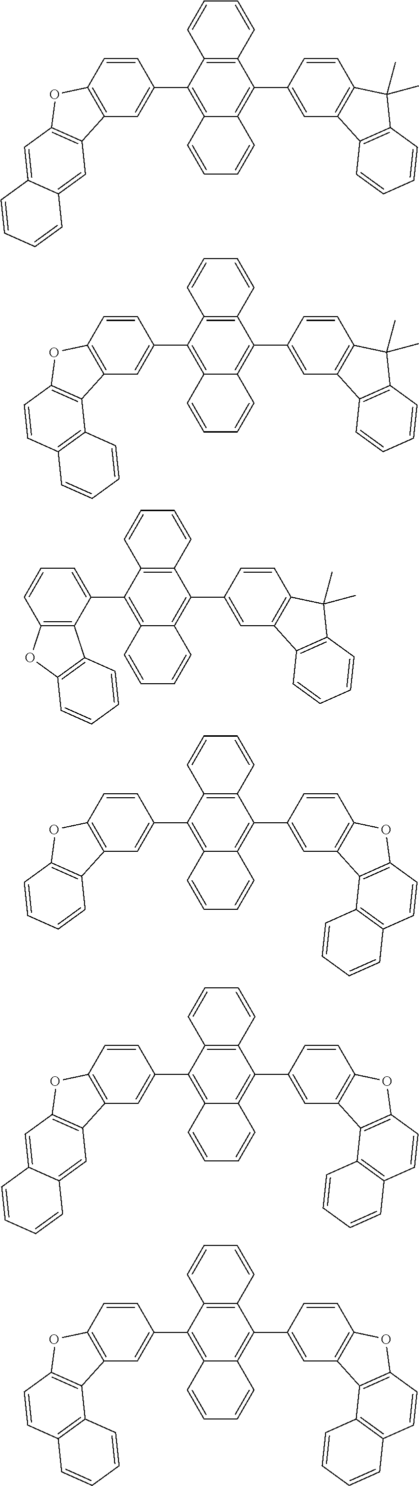

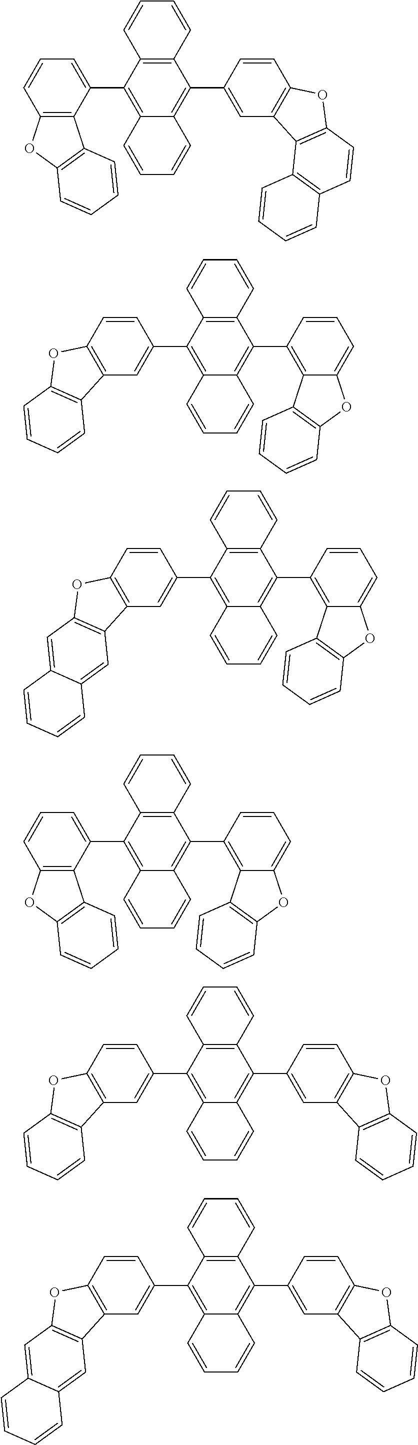

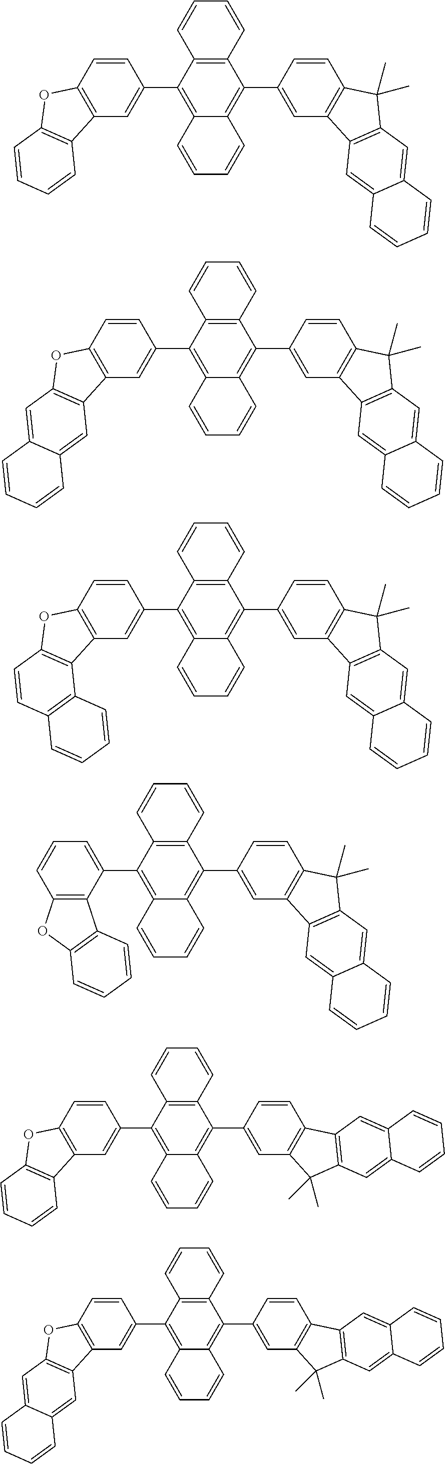

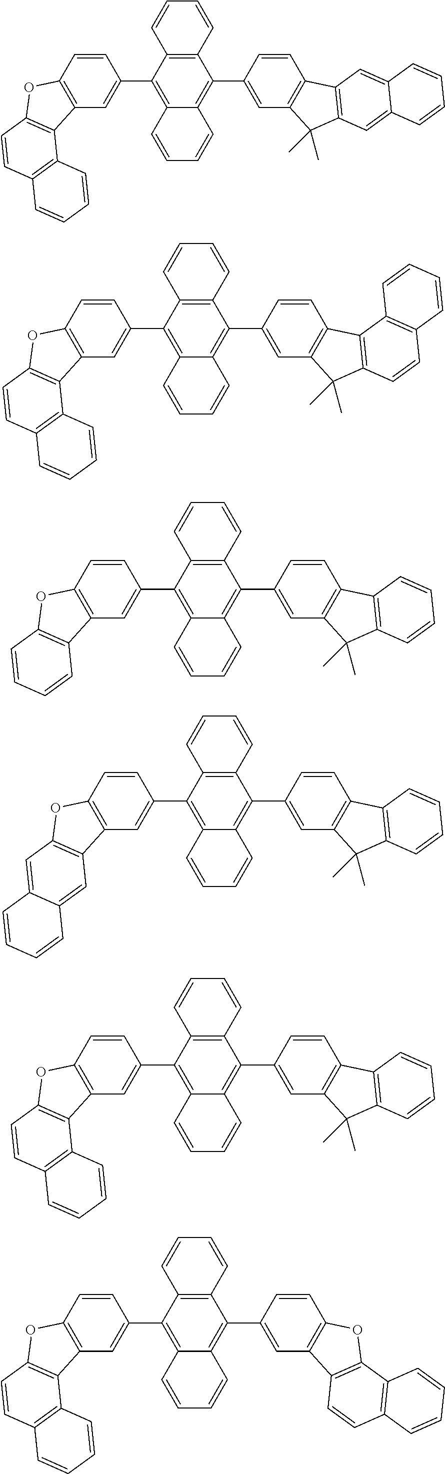

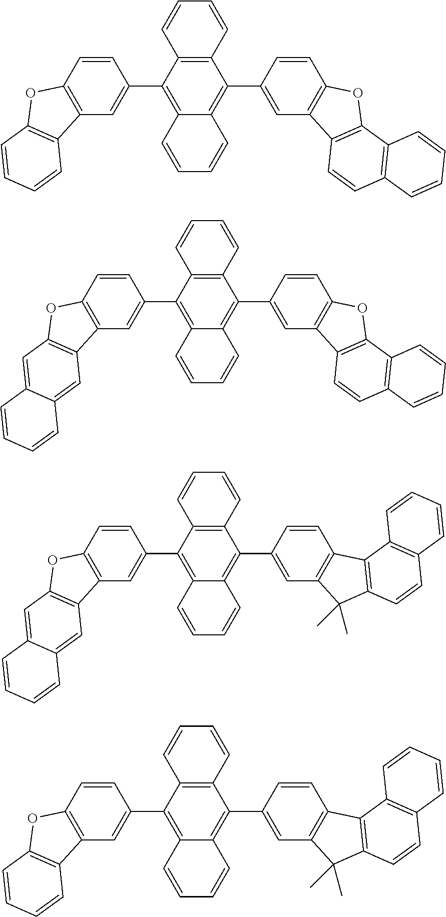 [SMILES: c1ccc2c(c1)ccc1c3cc(-c4c5ccccc5c(-c5ccc6oc7ccccc7c6c5)c5ccccc45)ccc3oc21 ; c1ccc2cc3c(cc2c1)oc1ccc(-c2c4ccccc4c(-c4ccc5oc6c7ccccc7ccc6c5c4)c4ccccc24)cc13 ; CC1(C)c2cc(-c3c4ccccc4c(-c4ccc5oc6cc7ccccc7cc6c5c4)c4ccccc34)ccc2-c2c1ccc1ccccc21 ; CC1(C)c2cc(-c3c4ccccc4c(-c4ccc5oc6ccccc6c5c4)c4ccccc34)ccc2-c2c1ccc1ccccc21]

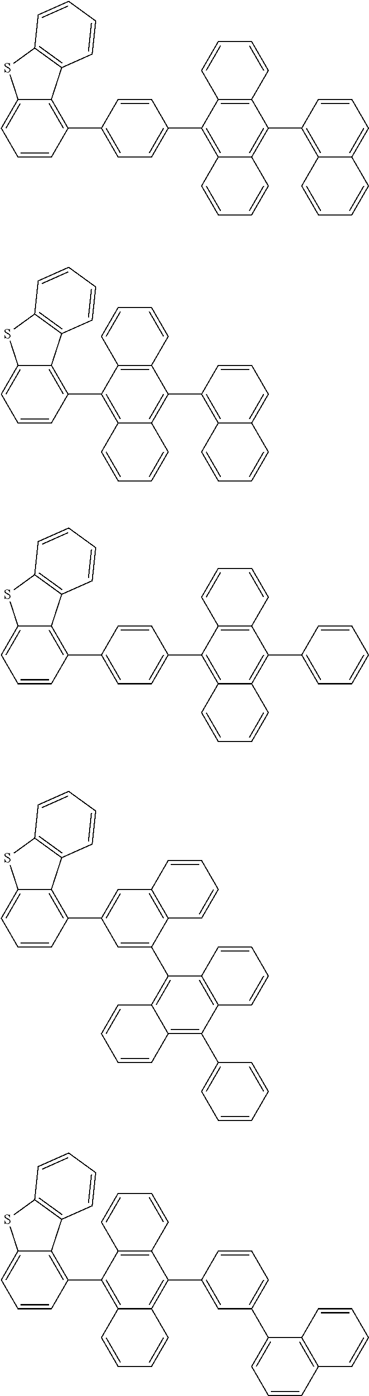 [SMILES: c1ccc2c(-c3c4ccccc4c(-c4ccc(-c5cccc6sc7ccccc7c56)cc4)c4ccccc34)cccc2c1 ; c1ccc2c(-c3c4ccccc4c(-c4cccc5sc6ccccc6c45)c4ccccc34)cccc2c1 ; c1ccc(-c2c3ccccc3c(-c3ccc(-c4cccc5sc6ccccc6c45)cc3)c3ccccc23)cc1 ; c1ccc(-c2c3ccccc3c(-c3cc(-c4cccc5sc6ccccc6c45)cc4ccccc34)c3ccccc23)cc1 ; c1cc(-c2cccc3ccccc23)cc(-c2c3ccccc3c(-c3cccc4sc5ccccc5c34)c3ccccc23)c1]

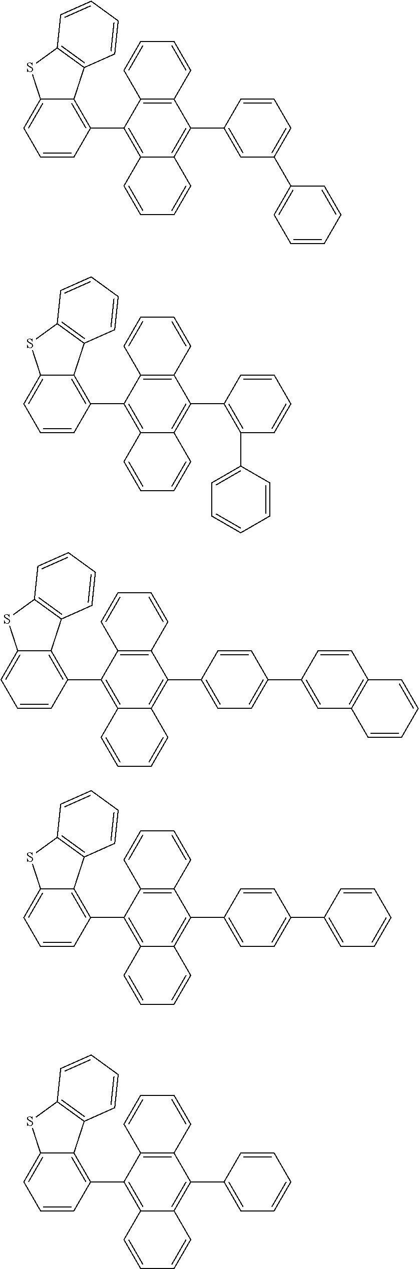

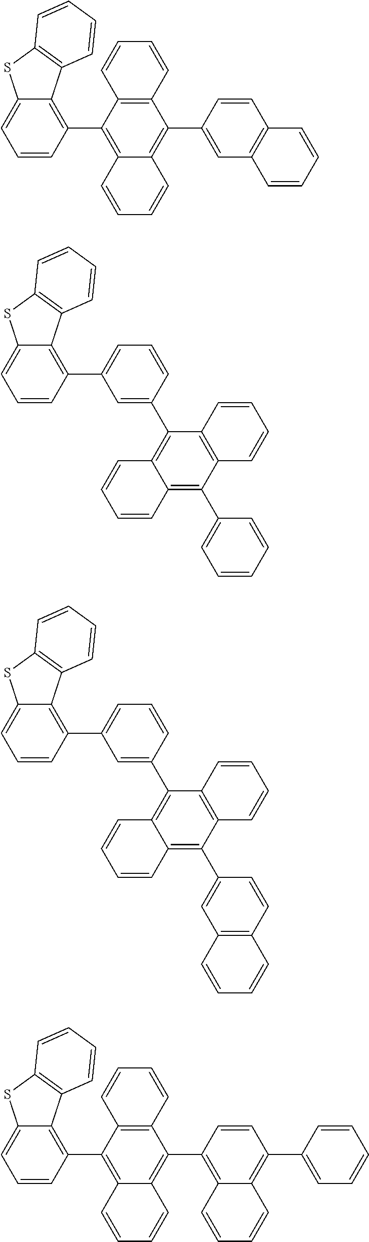 [SMILES: c1ccc2cc(-c3c4ccccc4c(-c4cccc5sc6ccccc6c45)c4ccccc34)ccc2c1 ; c1ccc(-c2c3ccccc3c(-c3cccc(-c4cccc5sc6ccccc6c45)c3)c3ccccc23)cc1 ; c1cc(-c2c3ccccc3c(-c3ccc4ccccc4c3)c3ccccc23)cc(-c2cccc3sc4ccccc4c23)c1 ; c1ccc(-c2ccc(-c3c4ccccc4c(-c4cccc5sc6ccccc6c45)c4ccccc34)c3ccccc23)cc1]

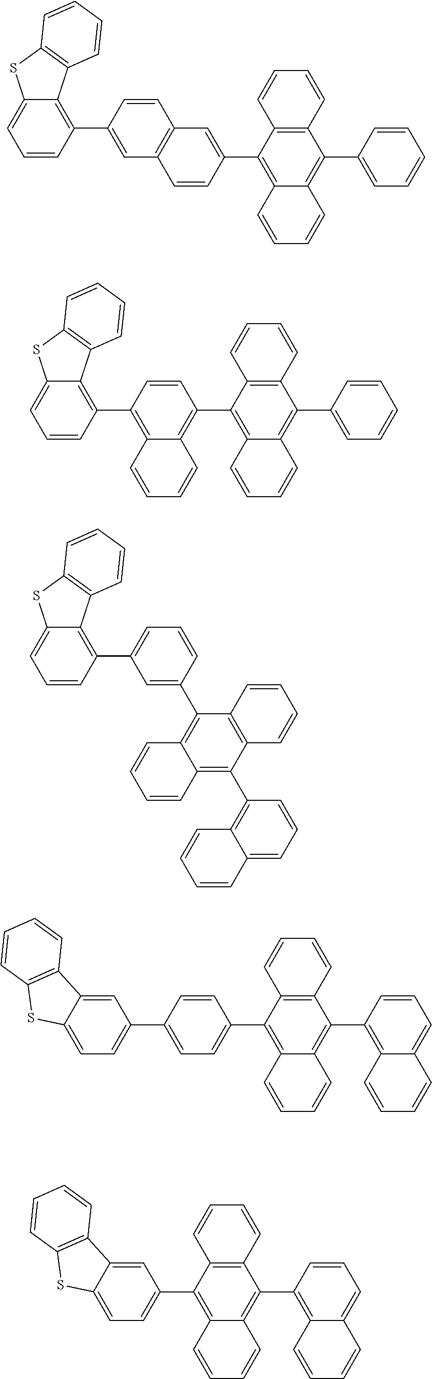 [SMILES: c1ccc(-c2c3ccccc3c(-c3ccc4cc(-c5cccc6sc7ccccc7c56)ccc4c3)c3ccccc23)cc1 ; c1ccc(-c2c3ccccc3c(-c3ccc(-c4cccc5sc6ccccc6c45)c4ccccc34)c3ccccc23)cc1 ; c1cc(-c2c3ccccc3c(-c3cccc4ccccc34)c3ccccc23)cc(-c2cccc3sc4ccccc4c23)c1 ; c1ccc2c(-c3c4ccccc4c(-c4ccc(-c5ccc6sc7ccccc7c6c5)cc4)c4ccccc34)cccc2c1 ; c1ccc2c(-c3c4ccccc4c(-c4ccc5sc6ccccc6c5c4)c4ccccc34)cccc2c1]

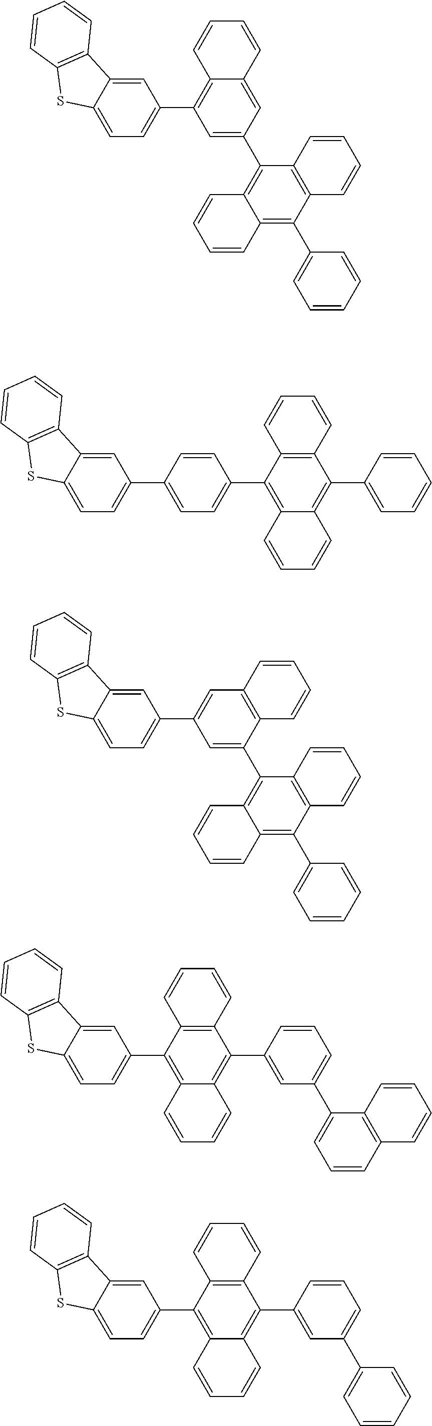 [SMILES: c1ccc(-c2c3ccccc3c(-c3cc(-c4ccc5sc6ccccc6c5c4)c4ccccc4c3)c3ccccc23)cc1 ; c1ccc(-c2c3ccccc3c(-c3ccc(-c4ccc5sc6ccccc6c5c4)cc3)c3ccccc23)cc1 ; c1ccc(-c2c3ccccc3c(-c3cc(-c4ccc5sc6ccccc6c5c4)cc4ccccc34)c3ccccc23)cc1 ; c1cc(-c2cccc3ccccc23)cc(-c2c3ccccc3c(-c3ccc4sc5ccccc5c4c3)c3ccccc23)c1 ; c1ccc(-c2cccc(-c3c4ccccc4c(-c4ccc5sc6ccccc6c5c4)c4ccccc34)c2)cc1]

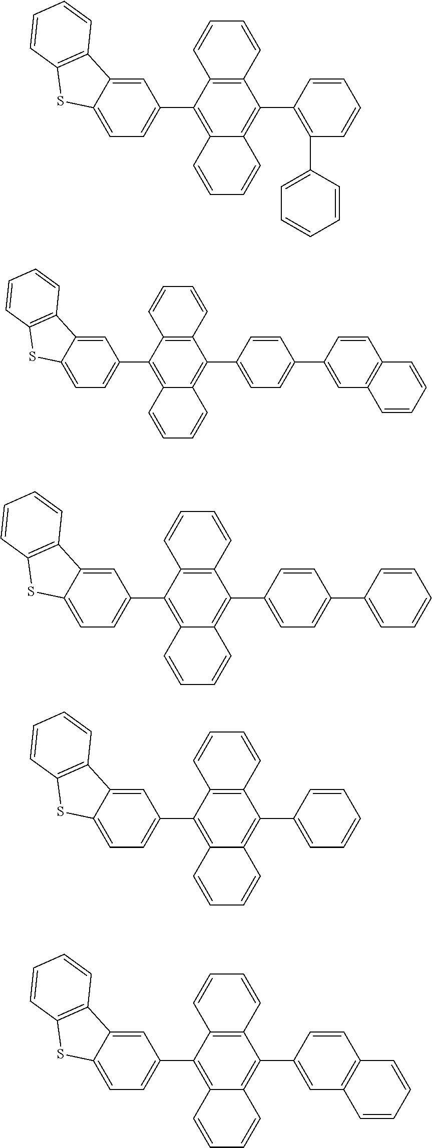 [SMILES: c1ccc(-c2ccccc2-c2c3ccccc3c(-c3ccc4sc5ccccc5c4c3)c3ccccc23)cc1 ; c1ccc2cc(-c3ccc(-c4c5ccccc5c(-c5ccc6sc7ccccc7c6c5)c5ccccc45)cc3)ccc2c1 ; c1ccc(-c2ccc(-c3c4ccccc4c(-c4ccc5sc6ccccc6c5c4)c4ccccc34)cc2)cc1 ; c1ccc(-c2c3ccccc3c(-c3ccc4sc5ccccc5c4c3)c3ccccc23)cc1 ; c1ccc2cc(-c3c4ccccc4c(-c4ccc5sc6ccccc6c5c4)c4ccccc34)ccc2c1]

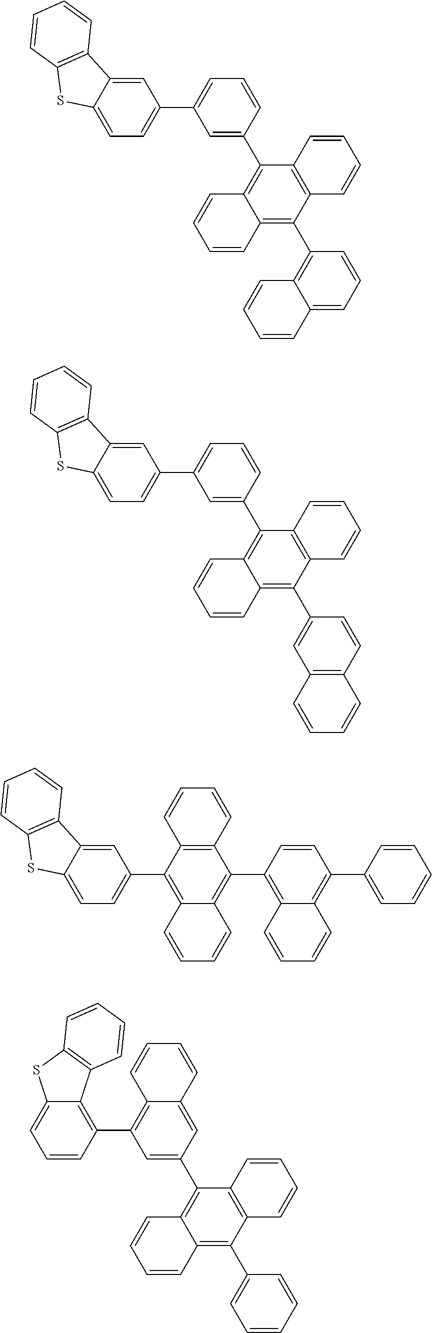 [SMILES: c1cc(-c2ccc3sc4ccccc4c3c2)cc(-c2c3ccccc3c(-c3cccc4ccccc34)c3ccccc23)c1 ; c1cc(-c2ccc3sc4ccccc4c3c2)cc(-c2c3ccccc3c(-c3ccc4ccccc4c3)c3ccccc23)c1 ; c1ccc(-c2ccc(-c3c4ccccc4c(-c4ccc5sc6ccccc6c5c4)c4ccccc34)c3ccccc23)cc1 ; c1ccc(-c2c3ccccc3c(-c3cc(-c4cccc5sc6ccccc6c45)c4ccccc4c3)c3ccccc23)cc1]

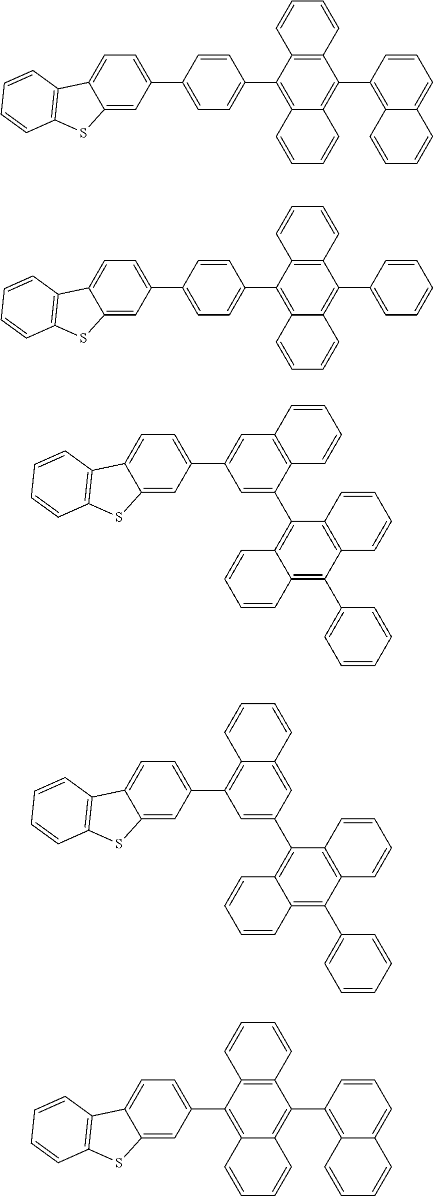

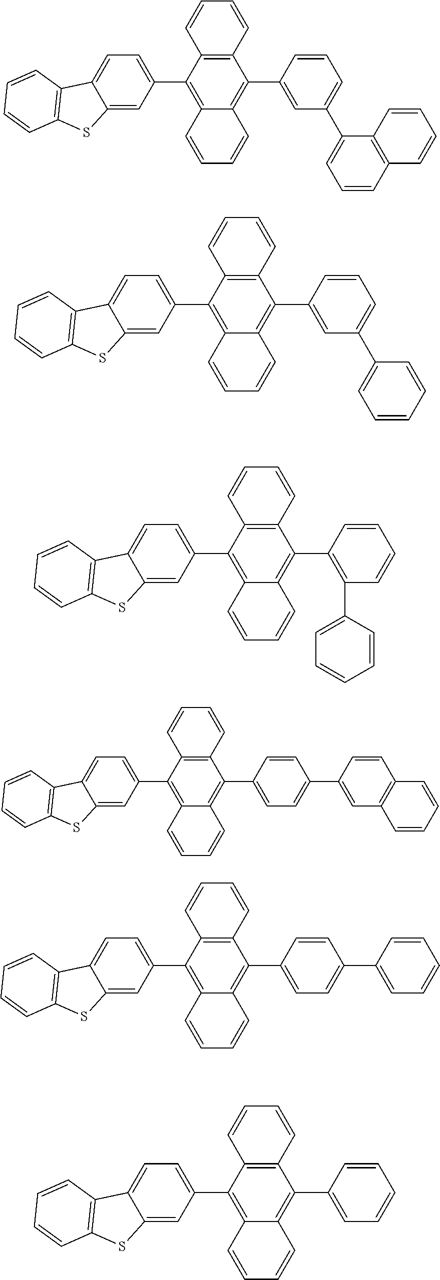

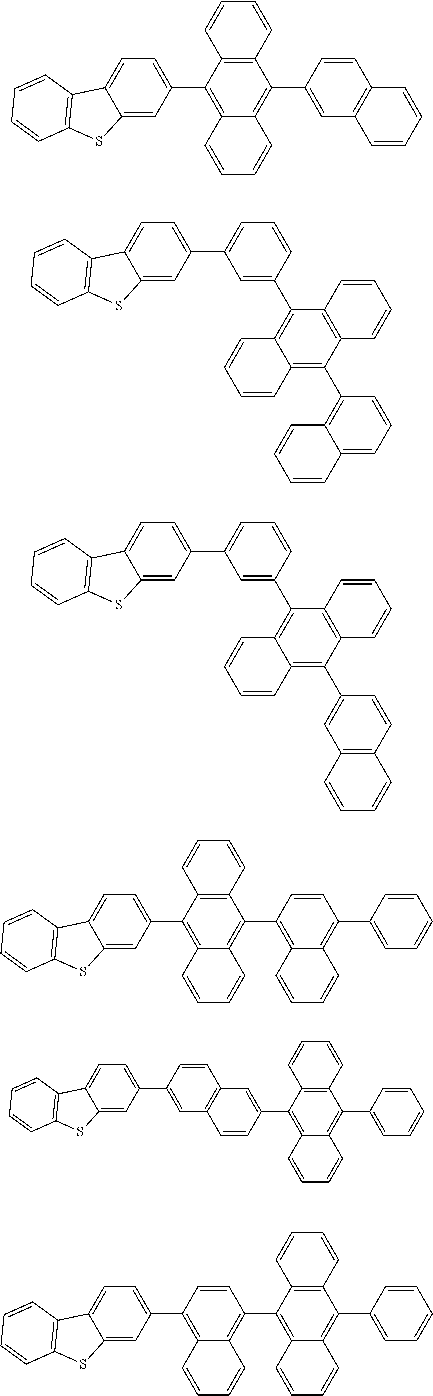

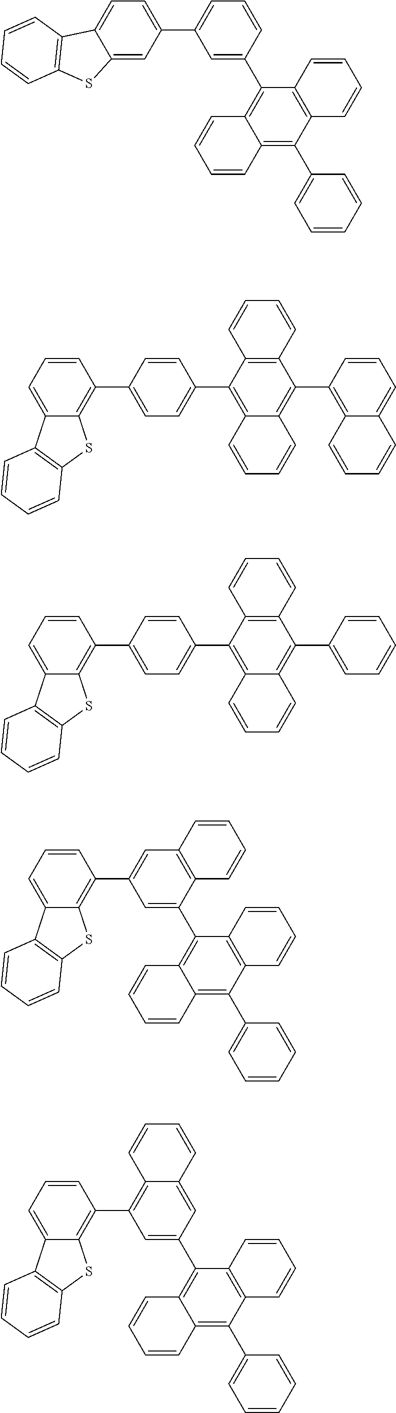 [SMILES: c1ccc(-c2c3ccccc3c(-c3cccc(-c4ccc5c(c4)sc4ccccc45)c3)c3ccccc23)cc1 ; c1ccc2c(-c3c4ccccc4c(-c4ccc(-c5cccc6c5sc5ccccc56)cc4)c4ccccc34)cccc2c1 ; c1ccc(-c2c3ccccc3c(-c3ccc(-c4cccc5c4sc4ccccc45)cc3)c3ccccc23)cc1 ; c1ccc(-c2c3ccccc3c(-c3cc(-c4cccc5c4sc4ccccc45)cc4ccccc34)c3ccccc23)cc1 ; c1ccc(-c2c3ccccc3c(-c3cc(-c4cccc5c4sc4ccccc45)c4ccccc4c3)c3ccccc23)cc1]

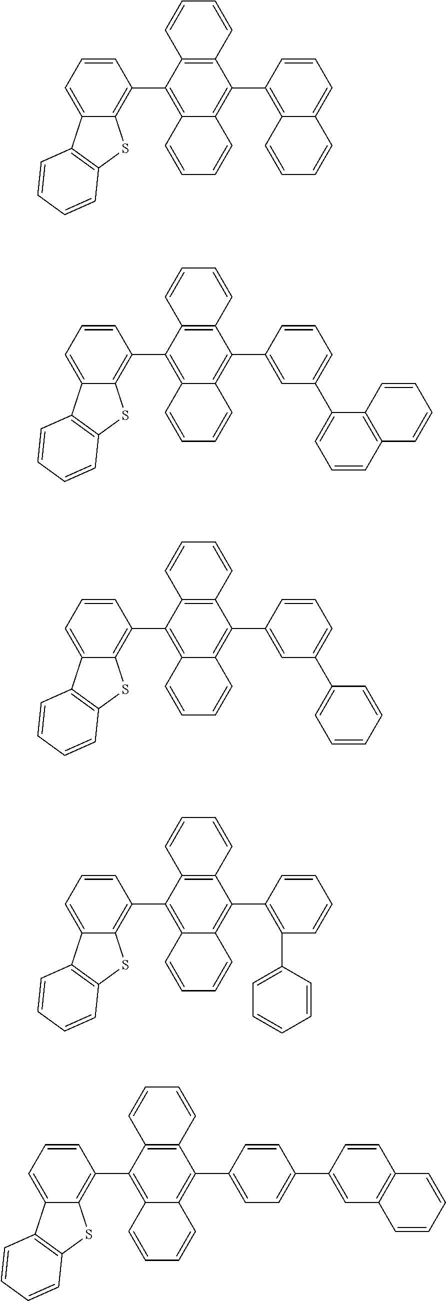 [SMILES: c1ccc2c(-c3c4ccccc4c(-c4cccc5c4sc4ccccc45)c4ccccc34)cccc2c1 ; c1cc(-c2cccc3ccccc23)cc(-c2c3ccccc3c(-c3cccc4c3sc3ccccc34)c3ccccc23)c1 ; c1ccc(-c2cccc(-c3c4ccccc4c(-c4cccc5c4sc4ccccc45)c4ccccc34)c2)cc1 ; c1ccc(-c2ccccc2-c2c3ccccc3c(-c3cccc4c3sc3ccccc34)c3ccccc23)cc1 ; c1ccc2cc(-c3ccc(-c4c5ccccc5c(-c5cccc6c5sc5ccccc56)c5ccccc45)cc3)ccc2c1]

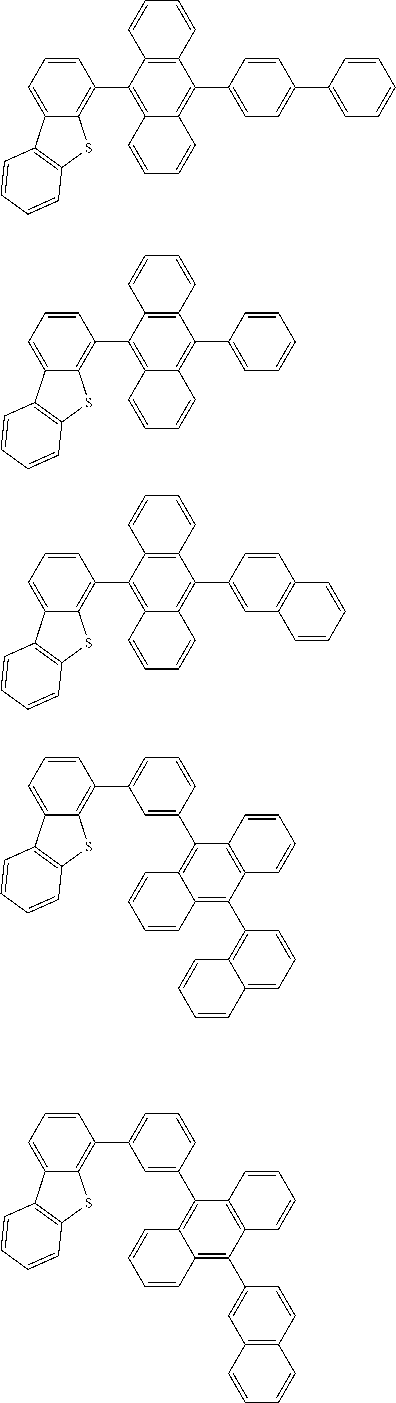 [SMILES: c1ccc(-c2ccc(-c3c4ccccc4c(-c4cccc5c4sc4ccccc45)c4ccccc34)cc2)cc1 ; c1ccc(-c2c3ccccc3c(-c3cccc4c3sc3ccccc34)c3ccccc23)cc1 ; c1ccc2cc(-c3c4ccccc4c(-c4cccc5c4sc4ccccc45)c4ccccc34)ccc2c1 ; c1cc(-c2c3ccccc3c(-c3cccc4ccccc34)c3ccccc23)cc(-c2cccc3c2sc2ccccc23)c1 ; c1cc(-c2c3ccccc3c(-c3ccc4ccccc4c3)c3ccccc23)cc(-c2cccc3c2sc2ccccc23)c1]

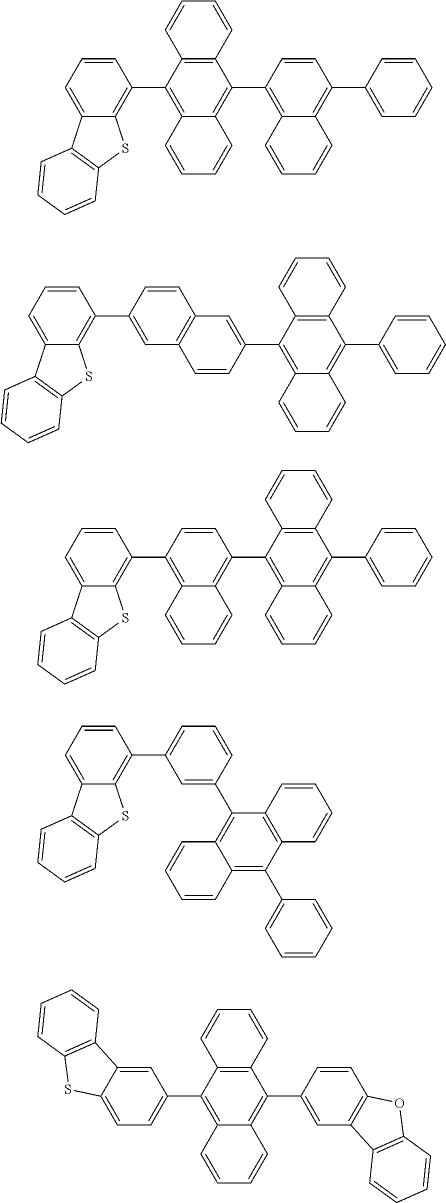

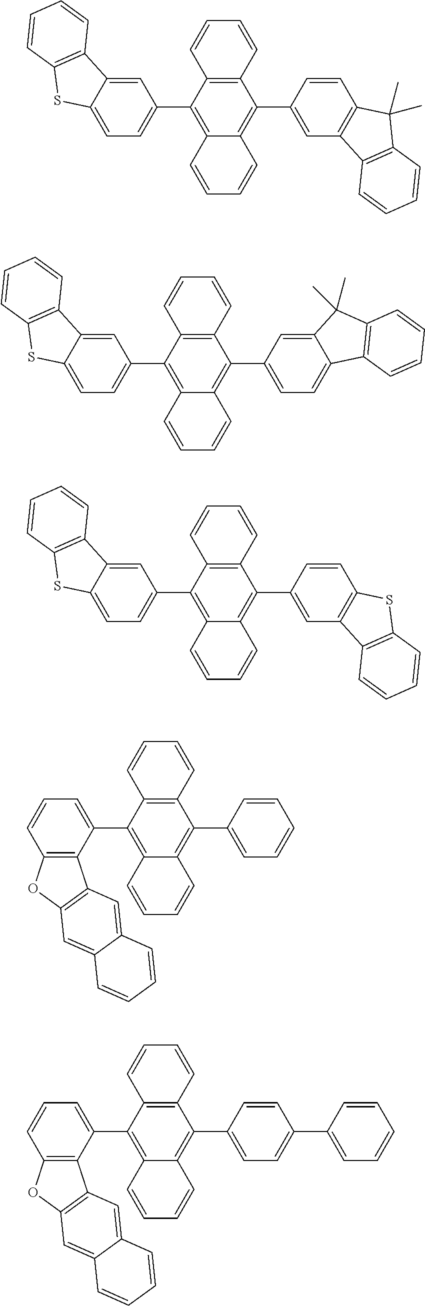

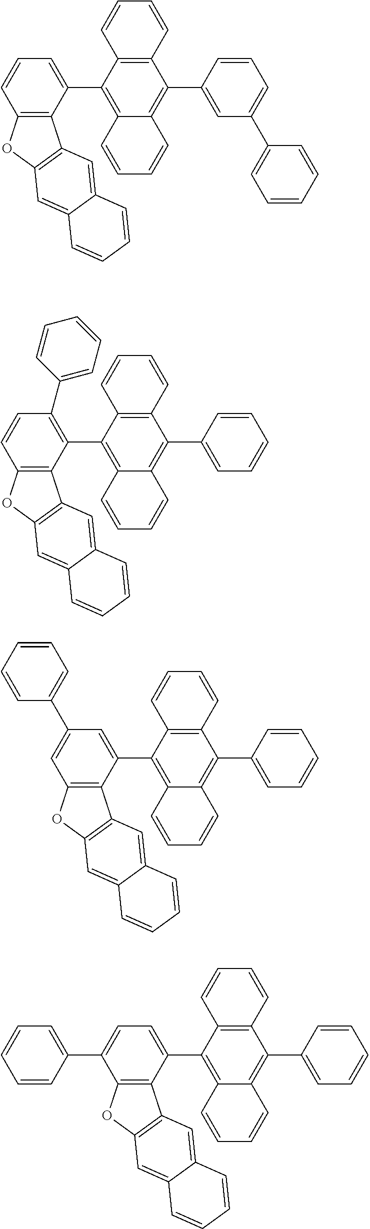

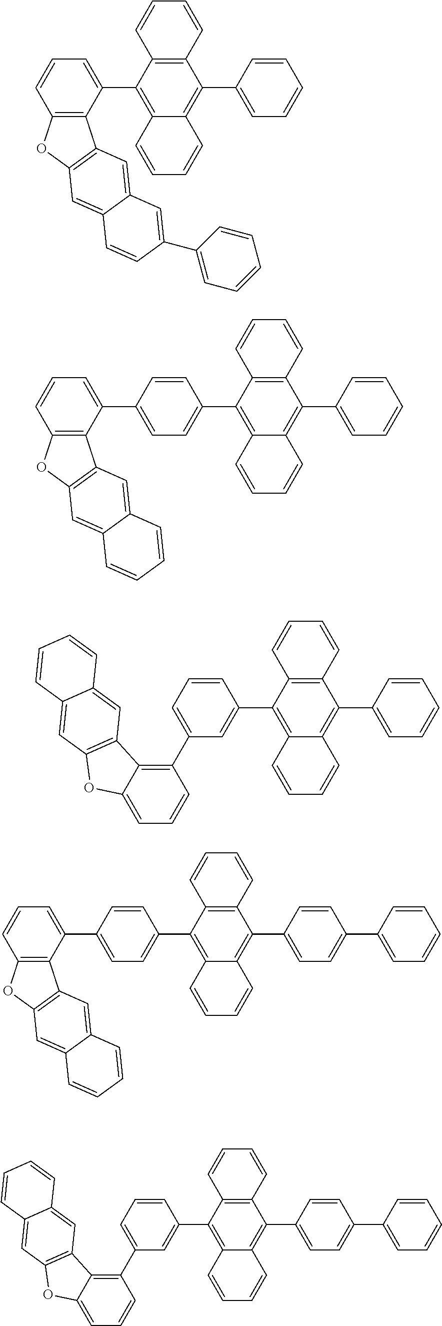

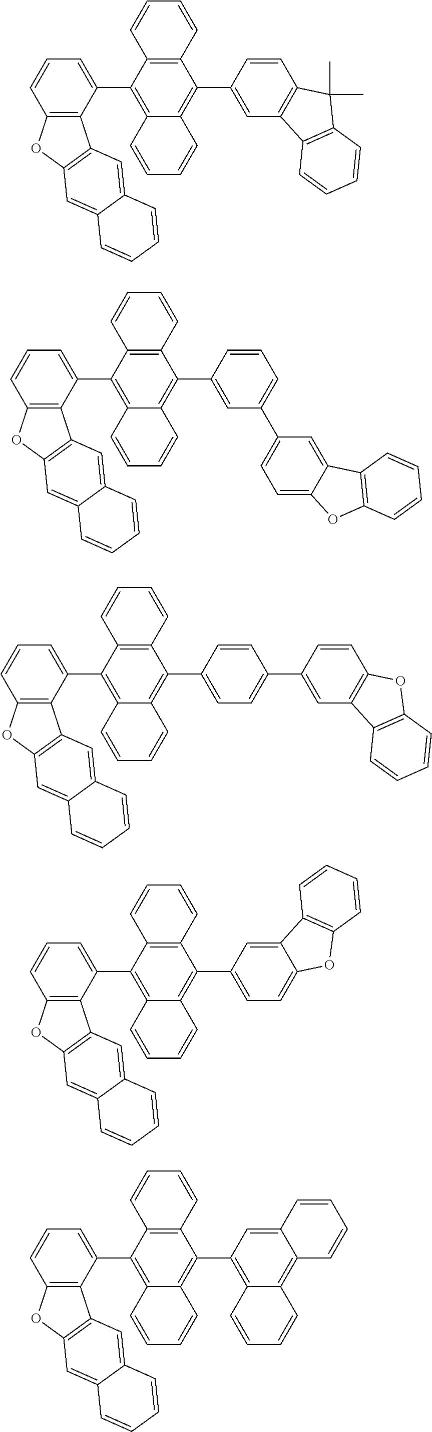

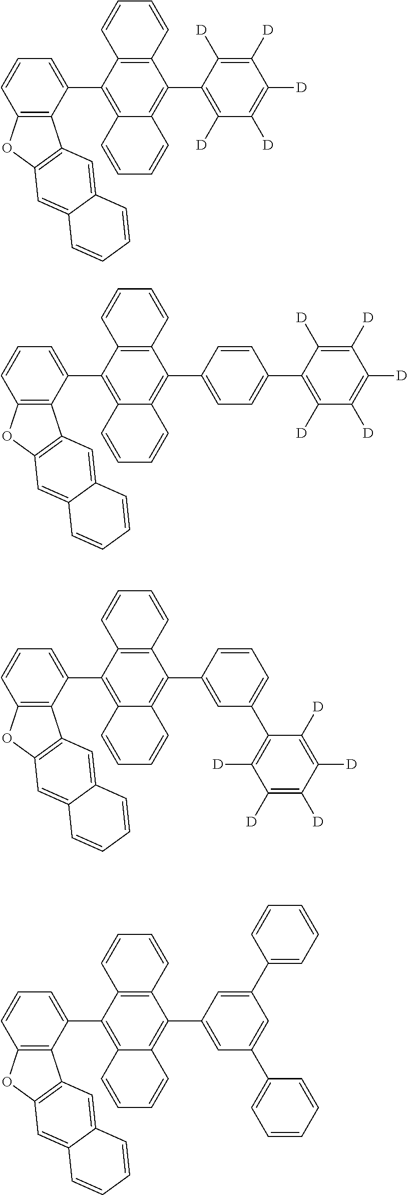

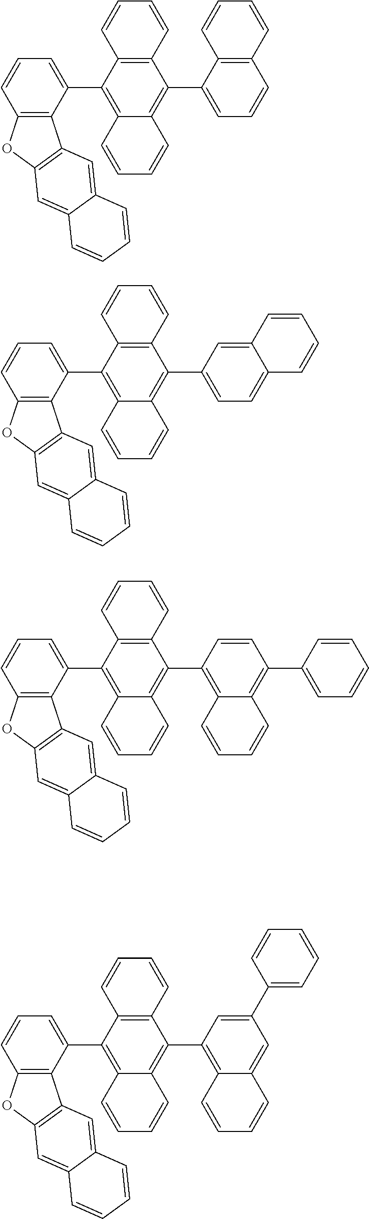

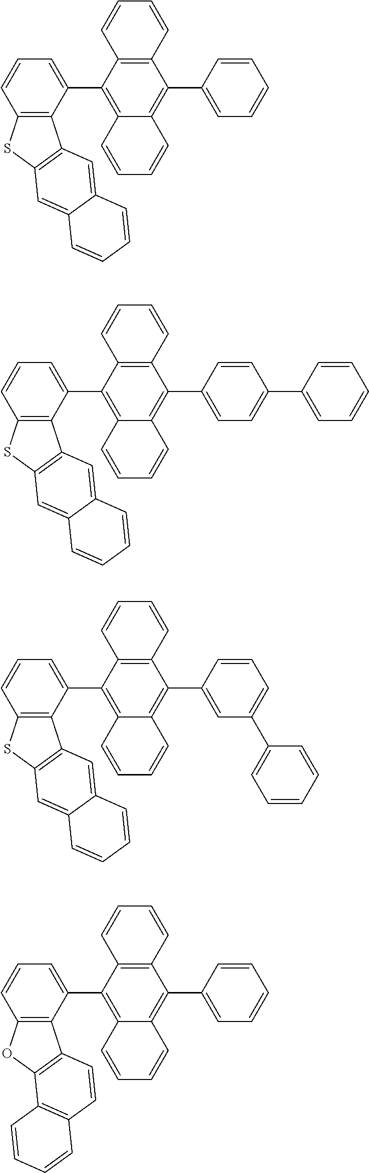

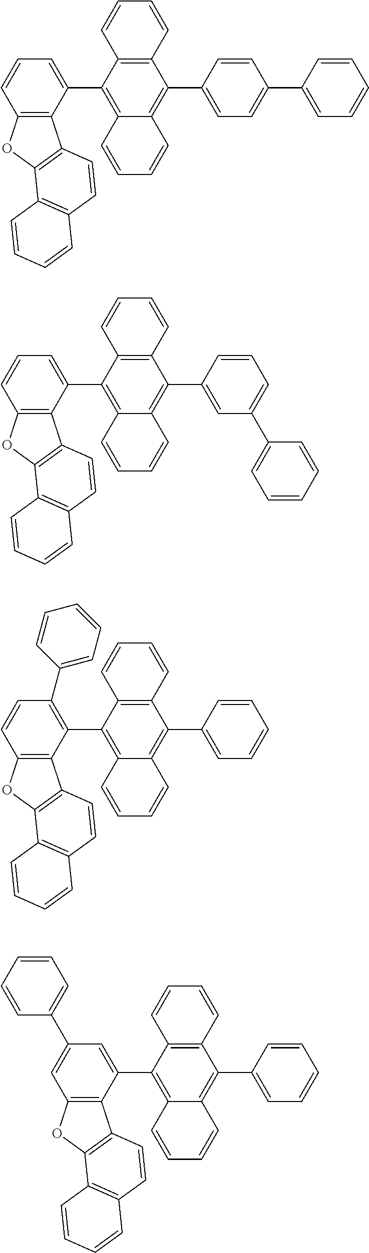

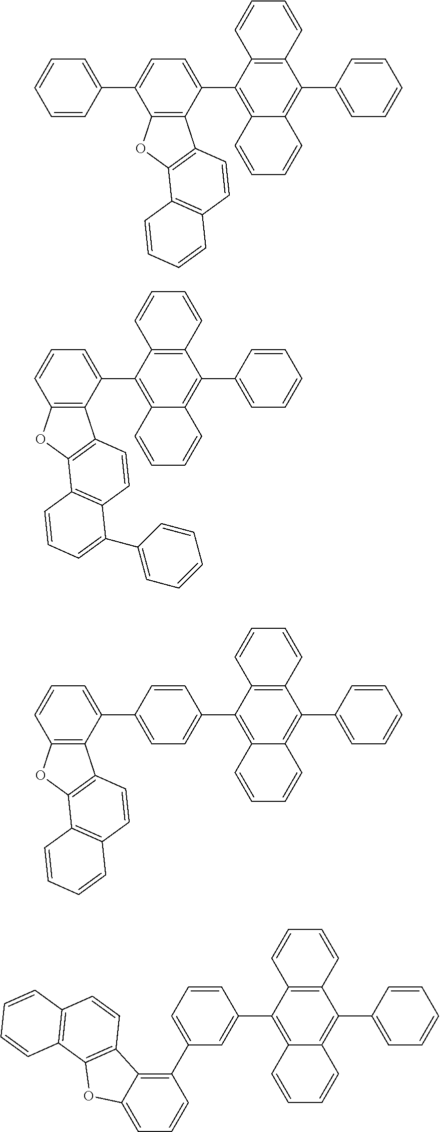

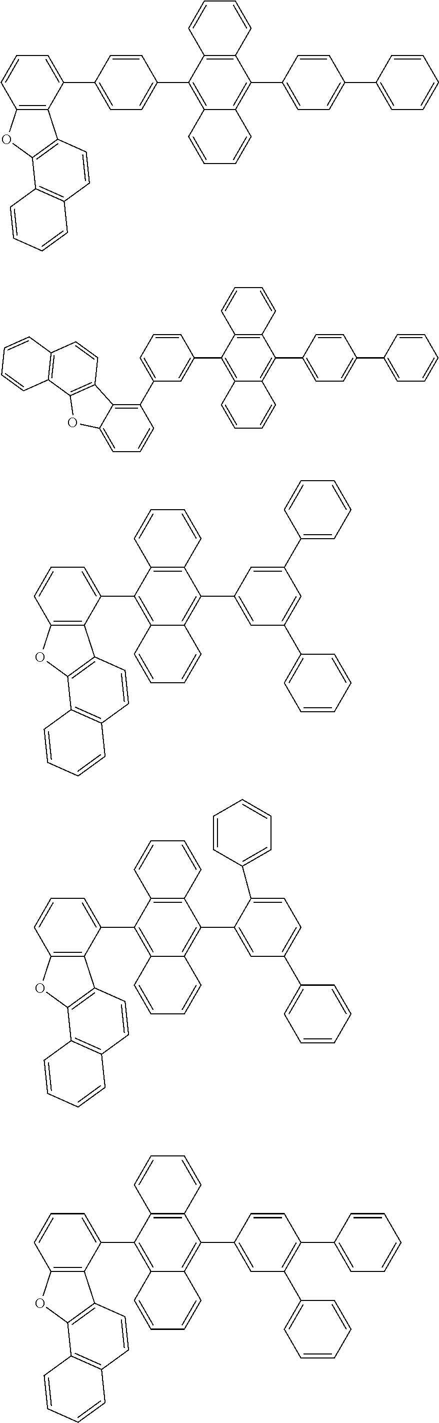

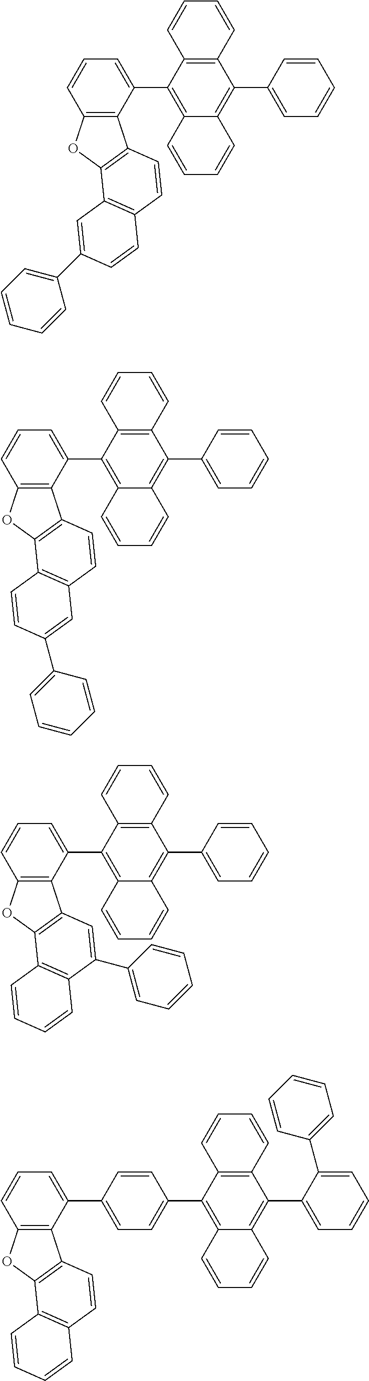 [SMILES: c1ccc(-c2ccc3ccc4c(oc5cccc(-c6c7ccccc7c(-c7ccccc7)c7ccccc67)c54)c3c2)cc1 ; c1ccc(-c2ccc3c(ccc4c3oc3cccc(-c5c6ccccc6c(-c6ccccc6)c6ccccc56)c34)c2)cc1 ; c1ccc(-c2c3ccccc3c(-c3cccc4oc5c6ccccc6c(-c6ccccc6)cc5c34)c3ccccc23)cc1 ; c1ccc(-c2ccccc2-c2c3ccccc3c(-c3ccc(-c4cccc5oc6c7ccccc7ccc6c45)cc3)c3ccccc23)cc1]

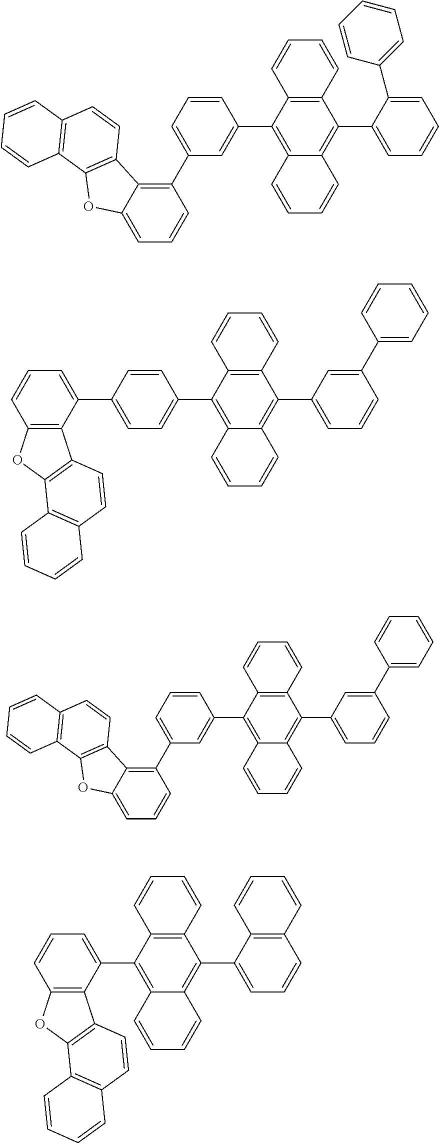 [SMILES: c1ccc(-c2ccccc2-c2c3ccccc3c(-c3cccc(-c4cccc5oc6c7ccccc7ccc6c45)c3)c3ccccc23)cc1 ; c1ccc(-c2cccc(-c3c4ccccc4c(-c4ccc(-c5cccc6oc7c8ccccc8ccc7c56)cc4)c4ccccc34)c2)cc1 ; c1ccc(-c2cccc(-c3c4ccccc4c(-c4cccc(-c5cccc6oc7c8ccccc8ccc7c56)c4)c4ccccc34)c2)cc1 ; c1ccc2c(-c3c4ccccc4c(-c4cccc5oc6c7ccccc7ccc6c45)c4ccccc34)cccc2c1]

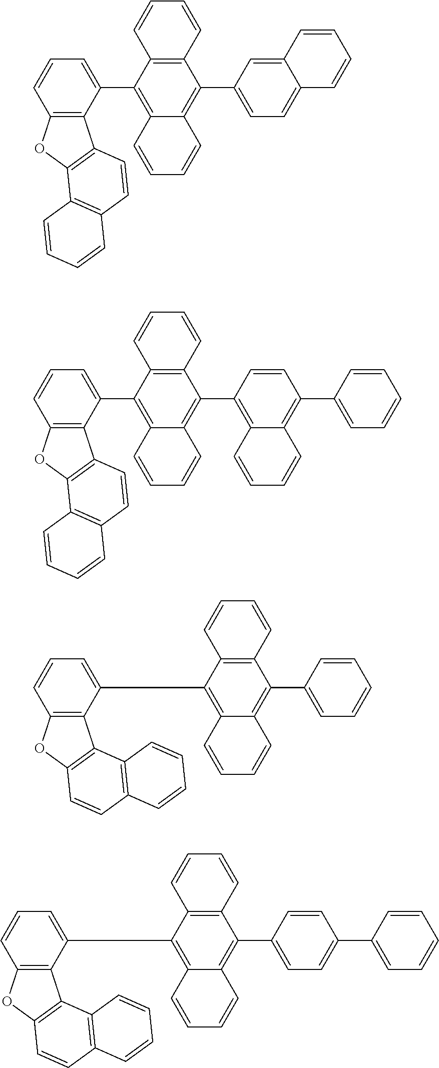 [SMILES: c1ccc2cc(-c3c4ccccc4c(-c4cccc5oc6c7ccccc7ccc6c45)c4ccccc34)ccc2c1 ; c1ccc(-c2ccc(-c3c4ccccc4c(-c4cccc5oc6c7ccccc7ccc6c45)c4ccccc34)c3ccccc23)cc1 ; c1ccc(-c2c3ccccc3c(-c3cccc4oc5ccc6ccccc6c5c34)c3ccccc23)cc1 ; c1ccc(-c2ccc(-c3c4ccccc4c(-c4cccc5oc6ccc7ccccc7c6c45)c4ccccc34)cc2)cc1]

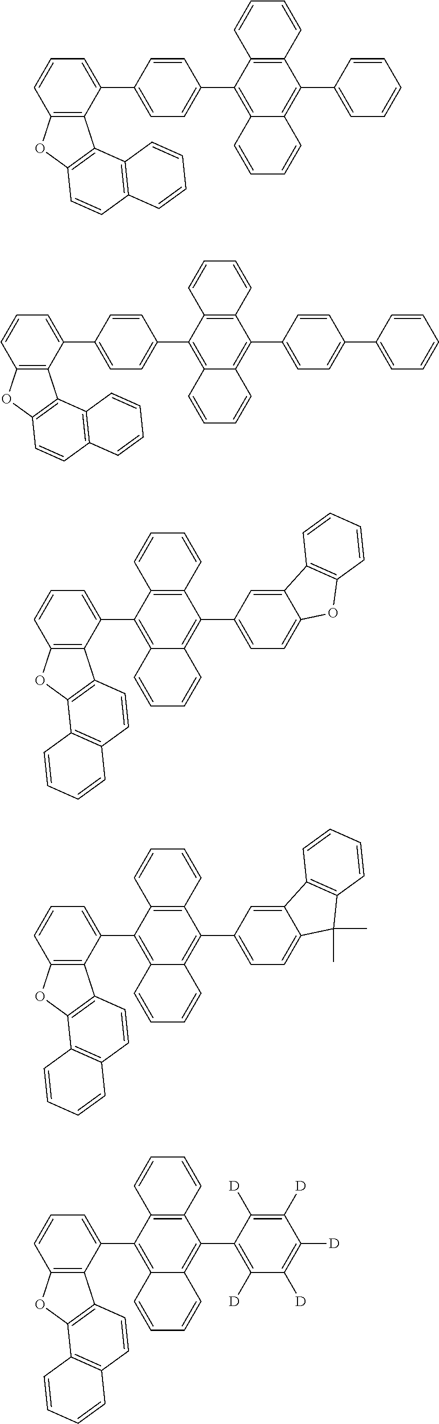 [SMILES: c1ccc(-c2c3ccccc3c(-c3ccc(-c4cccc5oc6ccc7ccccc7c6c45)cc3)c3ccccc23)cc1 ; c1ccc(-c2ccc(-c3c4ccccc4c(-c4ccc(-c5cccc6oc7ccc8ccccc8c7c56)cc4)c4ccccc34)cc2)cc1 ; c1ccc2c(c1)ccc1c2oc2cccc(-c3c4ccccc4c(-c4ccc5oc6ccccc6c5c4)c4ccccc34)c21 ; CC1(C)c2ccccc2-c2cc(-c3c4ccccc4c(-c4cccc5oc6c7ccccc7ccc6c45)c4ccccc34)ccc21 ; [2H]c1c([2H])c([2H])c(-c2c3ccccc3c(-c3cccc4oc5c6ccccc6ccc5c34)c3ccccc23)c([2H])c1[2H]]

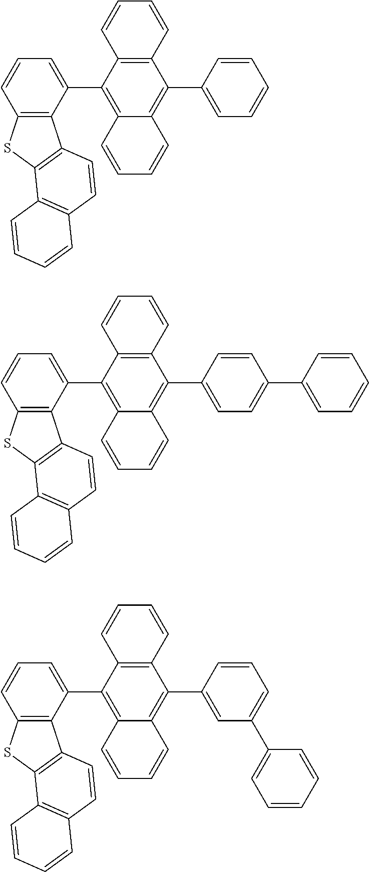 [SMILES: c1ccc(-c2c3ccccc3c(-c3cccc4sc5c6ccccc6ccc5c34)c3ccccc23)cc1 ; c1ccc(-c2ccc(-c3c4ccccc4c(-c4cccc5sc6c7ccccc7ccc6c45)c4ccccc34)cc2)cc1 ; c1ccc(-c2cccc(-c3c4ccccc4c(-c4cccc5sc6c7ccccc7ccc6c45)c4ccccc34)c2)cc1]

The content of the compound represented by the formula (1) in the emitting layer is preferably 1 mass % or more and 20 mass % or less based on the total mass of the emitting layer. The content of the compound represented by the formula (11) in the emitting layer is preferably 80 mass % or more and 99 mass % or less based on the total mass of the emitting layer.

An aspect of the organic EL device of the invention preferably includes a hole-transporting layer between the anode and the emitting layer.

An aspect of the organic EL device of the invention preferably includes an electron-transporting layer between the cathode and the emitting layer.

As the representative device configuration of the organic EL device of the invention, the following layer structures may be given.

(1) an anode/an emitting layer/a cathode,
(2) an anode/a hole-injecting layer/an emitting layer/a cathode,
(3) an anode/an emitting layer/an electron-injecting-transporting layer/a cathode,
(4) an anode/a hole-injecting layer/an emitting layer/an electron-injecting-transporting layer/a cathode,
(5) an anode/an organic semiconductor layer/an emitting layer/a cathode,
(6) an anode/an organic semiconductor layer/an electron barrier layer/an emitting layer/a cathode,
(7) an anode/an organic semiconductor layer/an emitting layer/an adhesion improving layer/a cathode,
(8) an anode/a hole-injecting-transporting layer/an emitting layer/an electron-injecting-transporting layer/a cathode,
(9) an anode/an insulating layer/an emitting layer/an insulating layer/a cathode,
(10) an anode/an inorganic semiconductor layer/an insulating layer/an emitting layer/an insulating layer/a cathode,
(11) an anode/an organic semiconductor layer/an insulating layer/an emitting layer/an insulating layer/a cathode,
(12) an anode/an insulating layer/a hole-injecting-transporting layer/an emitting layer/an insulating layer/a cathode, and
(13) an anode/an insulating layer/a hole-injecting-transporting layer/an emitting layer/an electron-injecting-transporting layer/a cathode.

Among the above-described structures, the configuration of (8) is preferably used, but the device configuration of the organic EL device is not limited thereto.

The emitting layer may be a phosphorescent emitting layer, or a fluorescent emitting layer. The organic EL device may include a plurality of emitting layers. When the organic EL device has a plurality of emitting layers, the organic EL device may have a space layer between the respective emitting layers for the purpose of preventing excitons generated in the phosphorescent emitting layer from diffusing into the fluorescent emitting layer.

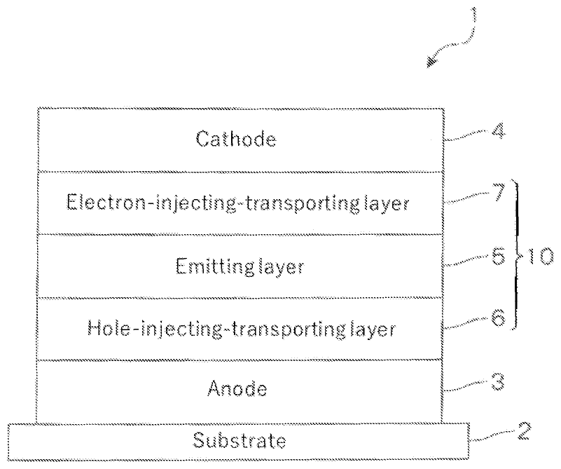

FIG. 1 shows a schematic configuration of one example of the organic EL device in an embodiment of the invention.

The organic EL device 1 includes a transparent substrate 2, an anode 3, a cathode 4, and an organic thin film layer 10 disposed between the anode 3 and the cathode 4.

The organic thin film layer 10 includes the above-mentioned emitting layer 5, but may include a hole-injecting-transporting layer 6 and the like between the emitting layer 5 and the anode 3, and an electron-injecting-transporting layer 7 and the like between the emitting layer 5 and the cathode 4.

Further, the electron barrier layer may be provided on a side of the anode 3 of the emitting layer 5, and a hole barrier layer may be provided on a side of the cathode 4 of the emitting layer 5, respectively.

By these device configurations, electrons and holes can be confined in the emitting layer 5 to enhance the generation probability of the excitons in the emitting layer 5.

The “hole-injecting-transporting layer” in this specification means “at least one of the hole-injecting layer and the hole-transporting layer”, and the “electron-injecting-transporting layer” in this specification means “at least one of the electron-injecting layer and the electron-transporting layer”.

The compound represented by the formula (1) and the compound represented by the formula (11) contained in the emitting layer 5 may be one compound alone or two or more compounds.

A substrate is used as a support of an emitting device. As the substrate, glass, quartz, plastics, and the like can be used, for example. Further, a flexible substrate may be used. The term “flexible substrate” means a bendable (flexible) substrate, and specific examples thereof include a plastic substrate formed of polycarbonate or polyvinyl chloride.

For the anode formed on the substrate, metal, alloy, an electrically conductive compound, a mixture thereof or the like, each having a large work function (specifically 4.0 eV or more), is preferably used. Specific examples include indium oxide-tin oxide (ITO: Indium Tin Oxide), indium oxide-tin oxide containing silicon or silicon oxide, indium oxide-zinc oxide, tungsten oxide, indium oxide containing zinc oxide, graphene, and the like. In addition thereto, specific examples thereof include gold (Au), platinum (Pt), a nitride of a metallic material (for example, titanium nitride), and the like.

The hole-injecting layer is a layer containing a material having high hole-injection properties. As the material having high hole-injection properties, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, an aromatic amine compound, and a condensed polycyclic compound in which a 5-membered ring containing a hetero atom and a benzene ring are continuously fused linearly. such as fluorene derivative, or a polymer compound (oligomer, dendrimer, polymer, or the like) can be used.

The hole-transporting layer is a layer containing a material having high hole-transporting properties. For the hole-transporting layer, an aromatic amine compound, a carbazole derivative, an anthracene derivative, or the like can be used. A polymer compound such as poly(N-vinylcarbazole) (abbreviation: PVK) and poly(4-vinyltriphenylamine) (abbreviation: PVTPA) can also be used. However, a material other than the above-described materials may be used as long as the material has higher transporting properties of holes in comparison with electrons. It should be noted that the layer containing the material having high hole-transporting properties may be formed into not only a monolayer, but also a stacked layer in which two or more layers formed of the above-described materials are stacked.

The electron-transporting layer is a layer containing a material having high electron-transporting properties. For the electron-transporting layer, 1) a metallic complex such as a lithium complex, an aluminum complex, a beryllium complex, or a zinc complex; 2) a heteroaromatic compound such as an imidazole derivative, a benzimidazole derivative, an azine derivative, a carbazole derivative, or a phenanthroline derivative; and 3) a polymer compound can be used.

The electron-injecting layer is a layer containing a material having high electron-injecting properties. For the electron-injecting layer, an alkali metal, an alkaline earth metal, or a compound thereof such as lithium (Li), a lithium complex, lithium fluoride (LiF), cesium fluoride (CsF), calcium fluoride (CaF₂), lithium oxide (LiOₓ), or the like can be used.

For the cathode, a metal, an alloy, an electrically conductive compound, a mixture thereof, or the like, having a small work function (specifically, 3.8 eV or less) is preferably used. Specific examples of such a cathode material include an element belonging to group 1 or group 2 of the periodic table of the elements, namely, alkali metals such as lithium (Li) and cesium (Cs), alkaline earth metal such as magnesium (Mg), and an alloy containing these metals (for example, MgAg and AlLi).

In an aspect of the organic EL device of the invention, a method for forming each layer is not limited. A conventionally-known method for forming each layer according to a vacuum deposition process, a spin coating process or the like can be used. Each layer such as the emitting layer can be formed by a known method such as a vacuum deposition process, a molecular beam deposition process (MBE process), or an application process such as a dipping process, a spin coating process, a casting process, a bar coating process, and a roll coating process, using a solution prepared by dissolving the material in a solvent.

In an aspect of an organic EL device of the invention, the thickness of each layer is not particularly limited, but generally, the thickness of each layer is preferably several nanometers to 1 micrometer in order to suppress defects such as pinholes, suppress applied voltages to be low, and to increase luminous efficiency.

The organic EL device of the invention can be used for a display component such as an organic EL panel module, a display apparatus such as a TV, a cellular phone, or a personal computer, and an electronic appliance such as a light emitting device such as a light, a vehicular lamp, or the like.

### EXAMPLES

Next, the invention will be described in more detail by referring to Synthesis Examples, Examples, and Comparative Examples, but the invention is not limited in any way to the description of these Examples.

The compound represented by the formula (1) used in Examples described later is a novel compound and was synthesized with reference to the methods described in PCT/JP2017/004828. Specifically, the methods are as described in Synthesis Examples as below.

### Synthesis Example 1 (Synthesis of BD-1)

Synthetic scheme of BD-1 is shown below.

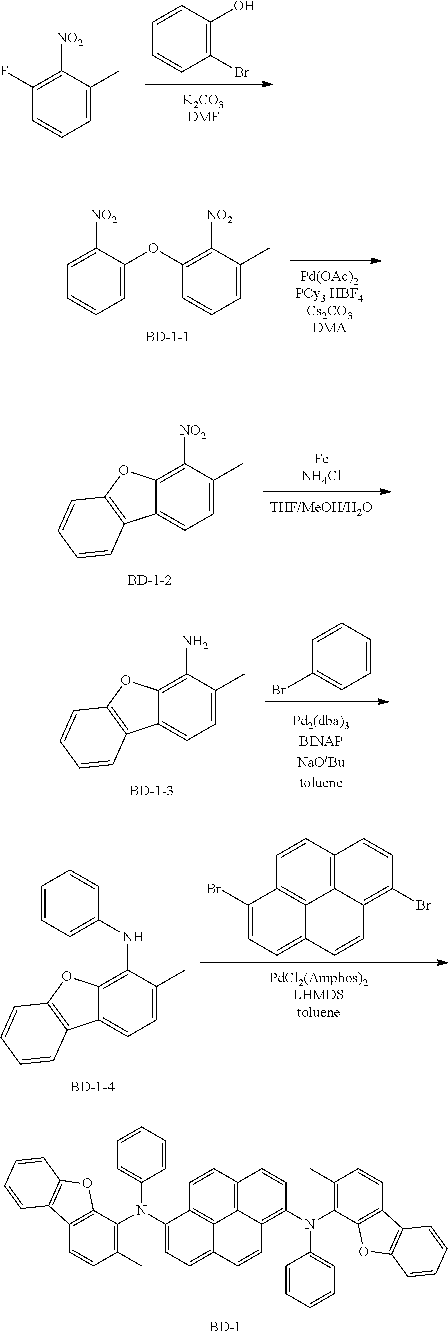

### (1-1) Synthesis of BD-1-1

Under an argon atmosphere, a mixture of 22.8 g of 1-fluoro-3-methyl-2-nitrobenzene, 25.4 g of 2-bromophenol, 40.6 g of potassium carbonate, and 500 mL of dimethylformamide (DMF) was stirred at 120° C. for 2 hours. The obtained reaction solution was cooled to room temperature, and extracted with ethyl acetate, and then the organic phase was washed with water, dried over anhydrous magnesium sulfate, and the solvent was distilled off under reduced pressure. The resulting residue was purified by silica gel column chromatography to obtain 40.3 g of BD-1-1 (yield: 89%).

### (1-2) Synthesis of BD-1-2

Under an argon atmosphere, a mixture of 40.0 g of BD-1-1, 2.91 g of palladium acetate (Pd(OAc)₂), 9.56 g of tricyclohexylphosphine tetrafluoroborate (PCy₃HBF₄), 127 g of cesium carbonate, and 400 mL of dimethylacetamide (DMA) was stirred at 130° C. for 3 hours. The obtained reaction solution was cooled to room temperature, and extracted with ethyl acetate, and then the organic phase was washed with water, dried over anhydrous magnesium sulfate, and the solvent was distilled off under reduced pressure. The obtained residue was purified by silica gel column chromatography to obtain 15.3 g of BD-1-2 (yield: 52%).

### (1-3) Synthesis of BD-1-3

Under an argon atmosphere, a mixture of 7.00 g of BD-1-2, 10.3 g of iron powder, 9.89 g of ammonium chloride, 80 mL of tetrahydrofuran (THF), 40 mL of methanol (MeOH), and 40 mL of water (H₂O) was stirred at 65° C. for 4 hours. The obtained reaction solution was cooled to room temperature, filtered through Celite, and the filtrate was washed with a saturated aqueous solution of sodium hydrogen carbonate, and stirred with hexane, and filtered to obtain 4.48 g of BD-1-3 (yield: 74%).

### (1-4) Synthesis of BD-1-4

Under an argon atmosphere, a mixture of 4.42 g of BD-1-3, 3.20 g of bromobenzene, 0.373 g of trisdibenzylideneacetone dipalladium (Pd₂(dba)₃), 0.508 g of 2,2′-bis(diphenylphosphino)-1,1′-binaphthyl (BINAP), 3.92 g of sodium-t-butoxide (NaO^{t}Bu), and 100 mL of toluene was refluxed under heating for 16 hours. The obtained reaction solution was cooled to room temperature, and extracted with ethyl acetate, and then the organic phase was washed with water, dried over anhydrous magnesium sulfate, and the solvent was distilled off under reduced pressure. The obtained residue was purified by silica gel column chromatography to obtain 4.24 g of BD-1-4 (yield: 76%).

### (1-5) Synthesis of BD-1

Under an argon atmosphere, a mixture of 15 g of BD-1-4 4, 2.60 g of dibromopyrene, 0.102 g of bis(di-t-butyl-4-dimethylaminophenylphosphine)dichloropalladium (Pd(Cl)₂(Amphos)₂), 14.4 mL of lithium bis(trimethylsilyl)amide (LHMDS) (1.0M THF solution), and 50 mL of toluene was refluxed under heating for 3 hours. The obtained reaction solution was cooled to room temperature, followed by filtration, and the obtained residue was purified by silica gel column chromatography to obtain 4.49 g of BD-1 (yield: 83%).

### Synthesis Example 2 (Synthesis of BD-2)

Synthetic scheme of BD-2 is shown below.

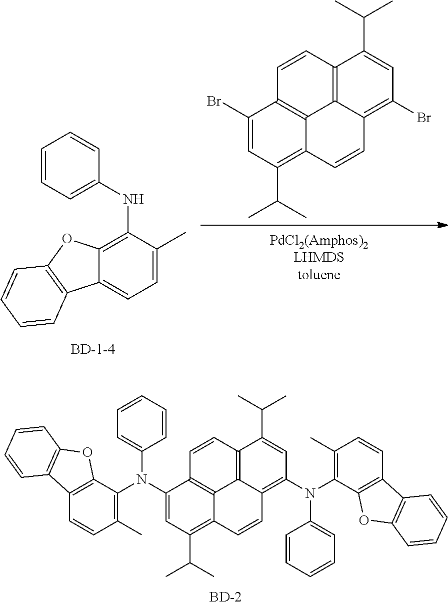

### (2-1) Synthesis of BD-2

Under an argon atmosphere, a mixture of 2.17 g of BD-1-4, 1.70 g of 1,6-dibromo-3,8-bis(1-methylethyl)pyrene, 0.054 g bis(di-t-butyl-4-dimethylaminophenylphosphine)dichloropalladium, 7.56 mL of LHMDS (1.0 M THF solution), and 30 mL of toluene was refluxed under heating for 12 hours. The obtained reaction solution was cooled to room temperature, followed by filteration, and the obtained residue was purified by silica gel column chromatography to obtain 2.29 g of BD-2 (yield: 73%).

### Synthesis Example 3 (Synthesis of BD-3)

Synthetic scheme of BD-3 is shown below.

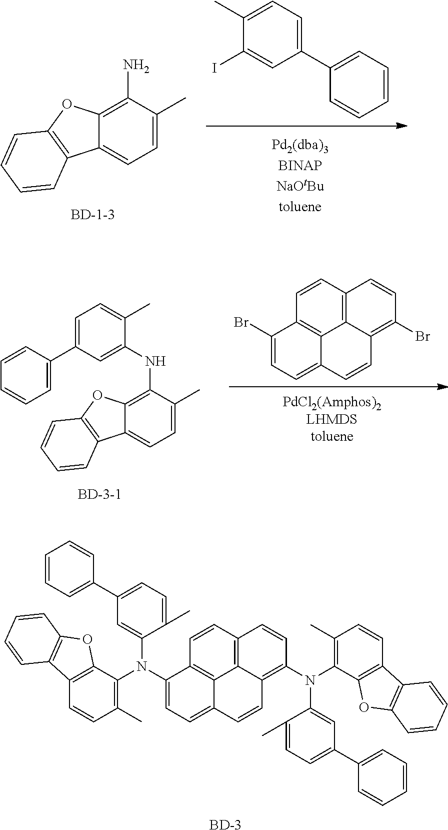 [SMILES: Cc1ccc2c(oc3ccccc32)c1N ; Cc1ccc(-c2ccccc2)cc1I ; Cc1ccc(-c2ccccc2)cc1Nc1c(C)ccc2c1oc1ccccc12 ; Brc1ccc2ccc3c(Br)ccc4ccc1c2c43 ; Cc1ccc(-c2ccccc2)cc1N(c1ccc2ccc3c(N(c4cc(-c5ccccc5)ccc4C)c4c(C)ccc5c4oc4ccccc45)ccc4ccc1c2c43)c1c(C)ccc2c1oc1ccccc12 ; "BD-3"]

### (3-1) Synthesis of BD-3-1

Under an argon atmosphere, a mixture of 5.39 g of BD-1-3, 7.30 g of 3-iodo-4-methyl-1,1′-biphenyl, 0.455 g of trisdibenzylideneacetone dipalladium, 0.618 g of BINAP, 4.77 g of sodium-t-butoxide, and 80 mL of toluene was refluxed under heating for 10 hours. The obtained reaction solution was cooled to room temperature, and extracted with ethyl acetate, and then the organic phase was washed with water, dried over anhydrous magnesium sulfate, and the solvent was distilled off under reduced pressure. The obtained residue was purified by silica gel column chromatography to obtain 7.22 g of BD-3-1 (yield: 70%).

### (3-2) Synthesis of BD-3

Under an argon atmosphere, a mixture of 3.39 g of BD-3-1, 1.60 g of dibromopyrene, 0.0629 g of bis(di-t-butyl-4-dimethylaminophenylphosphine)dichloropalladium, 8.89 mL of LHMDS (1.0 M THF solution), and 30 mL of toluene was refluxed under heating for 16 hours. The obtained reaction solution was cooled to room temperature, followed by filtration, and the obtained residue was purified by silica gel column chromatography to obtain 1.75 g of BD-3 (yield: 43%).

### Synthesis Example 4 (Synthesis of BD-4)

Synthetic scheme of BD-4 is shown below.

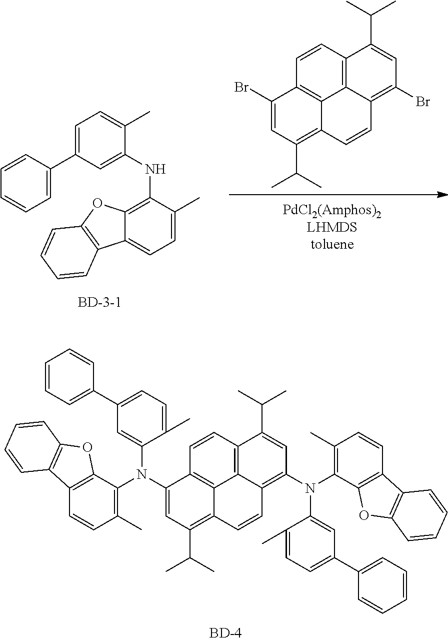 [SMILES: Cc1ccc(-c2ccccc2)cc1Nc1c(C)ccc2c1oc1ccccc12 ; CC(C)c1cc(Br)c2ccc3c(C(C)C)cc(Br)c4ccc1c2c43 ; Cc1ccc(-c2ccccc2)cc1N(c1cc(C(C)C)c2ccc3c(N(c4cc(-c5ccccc5)ccc4C)c4c(C)ccc5c4oc4ccccc45)cc(C(C)C)c4ccc1c2c43)c1c(C)ccc2c1oc1ccccc12 ; "BD-4"]

### (4-1) Synthesis of BD-4

Under an argon atmosphere, 3.44 g of BD-3-1, 2.00 g of 1,6-dibromo-3,8-bis(1-methylethyl)pyrene, 0.0638 g of bis(di-t-butyl-4-dimethylaminophenylphosphine)dichloropalladium, 9.00 mL of LHMDS (1.0 M THF solution), and 30 mL of toluene was refluxed under heating for 20 hours. The obtained reaction solution was cooled to room temperature, followed by filtration, and the obtained residue was purified by silica gel column chromatography to obtain 2.05 g of BD-4 (yield: 45%).

### Example 1

### (Fabrication of Organic EL Device)

A 25 mm×75 mm×1.1 mm-thick glass substrate with an ITO transparent electrode (anode) (manufactured by GEOMATEC Co., Ltd.) was subjected to ultrasonic cleaning in isopropyl alcohol for 5 minutes, and then subjected to UV-ozone cleaning for 30 minutes. The thickness of the ITO film was 130 nm.

The glass substrate with the transparent electrode after being cleaned was mounted onto a substrate holder in a vacuum vapor deposition apparatus. First, a compound HI-1 was deposited on the surface on the side on which the transparent electrode was formed so as to cover the transparent electrode to form a compound HI-1 film having a thickness of 5 nm. The HI-1 film functions as a hole-injecting layer.

Subsequent to the formation of the HI-1 film, a compound HT-1 was deposited thereon to form an HT-1 film having a thickness of 80 nm on the HI-1 film. The HT-1 film functions as a first hole-transporting layer.

Subsequent to the formation of the HT-1 film, a compound HT-2 was deposited thereon to form an HT-2 film having a thickness of 10 nm on the HT-1 film. The HT-2 film functions as a second hole-transporting layer.

BH-1 (host material) and BD-1 (dopant material) were co-deposited on the HT-2 film to be 4% in a proportion (mass ratio) of BD-1 to form an emitting layer having a thickness of 25 nm.

ET-1 was deposited on this emitting layer to form an electron-transporting layer having a thickness of 10 nm. ET-2 as an electron-injecting material was deposited on the electron-transporting layer to form an electron-injecting layer having a thickness of 15 nm. LiF was deposited on the electron-injecting layer to form a LiF film having a thickness of 1 nm. Al metal was deposited on the LiF film to form a metal cathode having a thickness of 80 nm.

As described above, an organic EL device was fabricated. Compounds used in Example 11 are shown below.

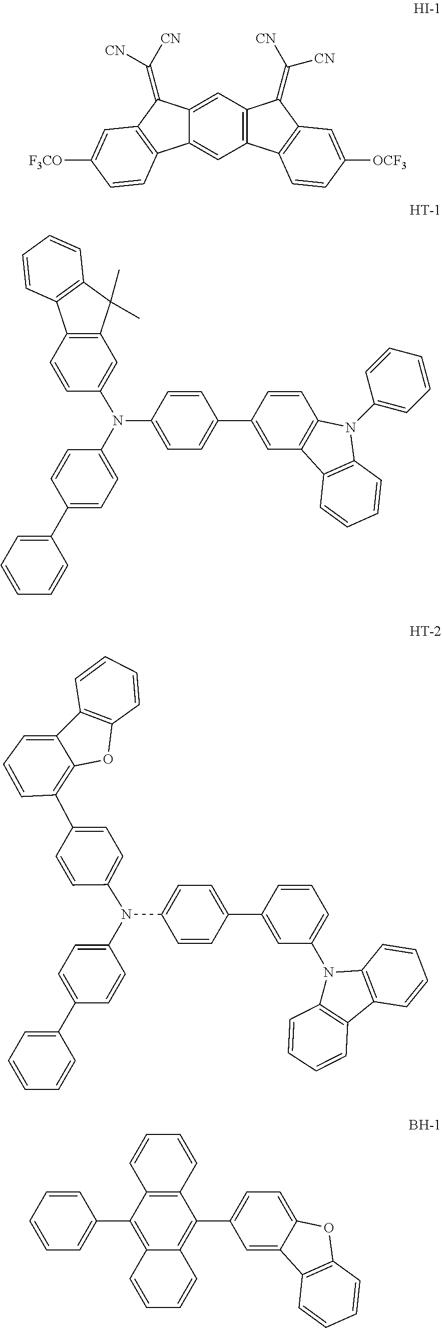

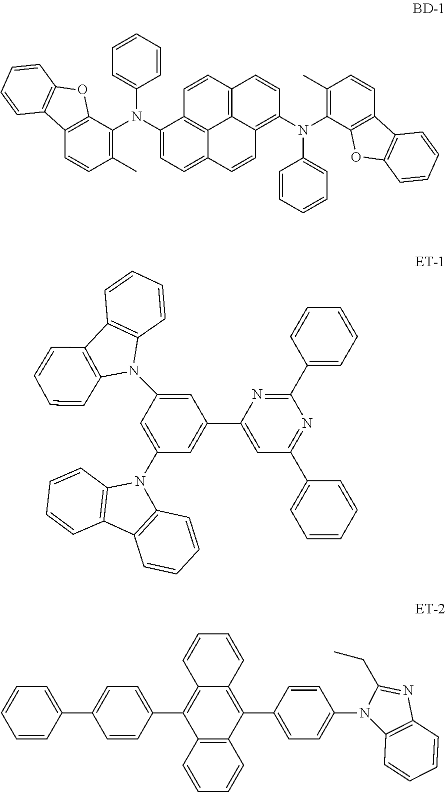 [SMILES: Cc1ccc2c(oc3ccccc32)c1N(c1ccccc1)c1ccc2ccc3c(N(c4ccccc4)c4c(C)ccc5c4oc4ccccc45)ccc4ccc1c2c43 ; c1ccc(-c2cc(-c3cc(-n4c5ccccc5c5ccccc54)cc(-n4c5ccccc5c5ccccc54)c3)nc(-c3ccccc3)n2)cc1 ; CCc1nc2ccccc2n1-c1ccc(-c2c3ccccc3c(-c3ccc(-c4ccccc4)cc3)c3ccccc23)cc1]

### (Evaluation of Organic EL Device)

Initial characteristics of the obtained organic EL device were measured by DC-constant current 10 mA/cm² of DC (direct current) at room temperature. The measurement results of the voltage are shown in Table 1.

Furthermore, a voltage was applied to the organic EL device to be 10 mA/cm² in current density, thereby measuring an EL emission spectrum by using Spectroradiometer CS-1000 (manufactured by Konica Minolta, Inc.). External quantum efficiency (EQE) (%) was calculated from the obtained spectral radiance spectrum. The results are shown in Table 1.

### Examples 2 to 16, Reference Examples 1 to 4, and Comparative Example 1

The organic EL devices were fabricated and evaluated in the same manner as in Example 1 except that the compounds shown in Table 1 were used as the host material and the dopant material of the emitting layer. The results are shown in Table 1.

Compounds of the host material and the dopant material used in Examples 1 to 16, Reference Examples 1 to 4, and Comparative Example 1 will be described below.

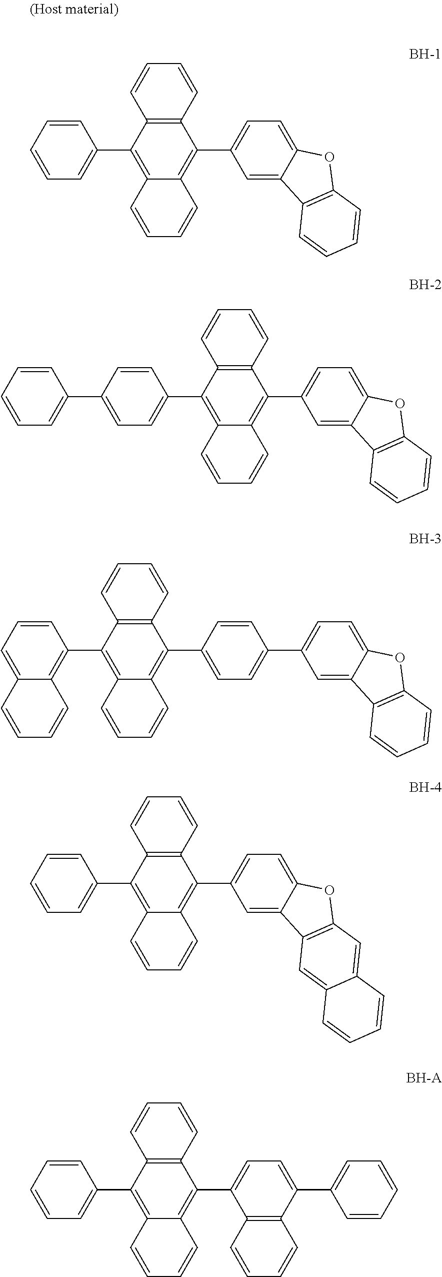

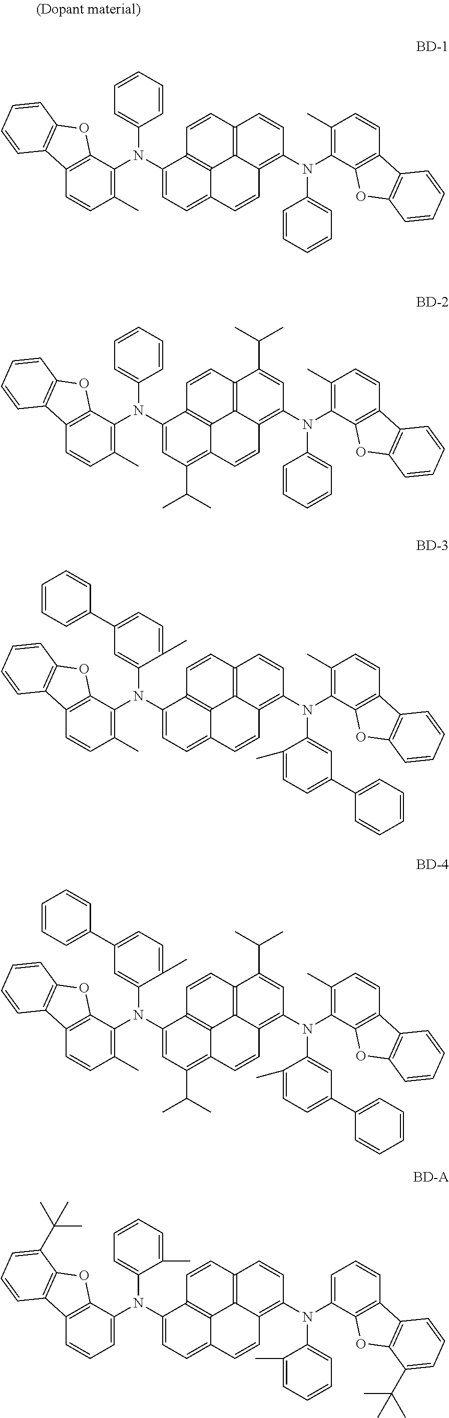

TABLE 1

| | Host | Dopant | Voltage (V) | EQE (%) |
|---|---|---|---|---|
| Example 1 | BH-1 | BD-1 | 3.82 | 8.6 |
| Example 2 | BH-1 | BD-2 | 3.83 | 8.6 |
| Example 3 | BH-1 | BD-3 | 3.81 | 8.6 |
| Example 4 | BH-1 | BD-4 | 3.80 | 8.7 |
| Example 5 | BH-2 | BD-1 | 3.64 | 8.7 |
| Example 6 | BH-2 | BD-2 | 3.64 | 8.8 |
| Example 7 | BH-2 | BD-3 | 3.64 | 8.9 |
| Example 8 | BH-2 | BD-4 | 3.62 | 9.1 |
| Example 9 | BH-3 | BD-1 | 3.62 | 8.7 |
| Example 10 | BH-3 | BD-2 | 3.63 | 8.7 |
| Example 11 | BH-3 | BD-3 | 3.62 | 8.7 |
| Example 12 | BH-3 | BD-4 | 3.61 | 9.0 |
| Example 13 | BH-4 | BD-1 | 3.50 | 8.7 |
| Example 14 | BH-4 | BD-2 | 3.51 | 8.8 |
| Example 15 | BH-4 | BD-3 | 3.51 | 8.8 |
| Example 16 | BH-4 | BD-4 | 3.50 | 8.9 |
| Ref. Ex. 1 | BH-A | BD-1 | 4.22 | 8.2 |
| Ref. Ex. 2 | BH-A | BD-2 | 4.23 | 8.2 |
| Ref. Ex. 3 | BH-A | BD-3 | 4.20 | 8.3 |
| Ref. Ex. 4 | BH-A | BD-4 | 4.22 | 8.4 |
| Comp. Ex. 1 | BH-A | BD-A | 4.21 | 7.9 |

From the results shown in Table 1, it can be seen that in Comparative Example 1 using BH-A as the host material and BD-A as the dopant material, the external quantum efficiency EQE is as low as 7.9% and the driving voltage is as high as 4.21 V. In addition, in Reference Examples 1 to 4 using BD-1 to BD-4 as the dopant material and BH-A as the host material, the external quantum efficiency EQE was 8.2 to 8.4% and the drive voltage was as high as 4.20 to 4.23 V. In contrast, the organic EL devices of Examples 1 to 16 using BH-1 to BH-4 as the host material and BD-1 to BD-4 as the dopant material showed high external quantum efficiency EQE of 8.6 to 9.1% and low drive voltage of 3.50 to 3.83 V.

From the above results, it can be seen that by using the compound represented by the formula (1) as the dopant material, an organic EL device excellent in luminous efficiency can be obtained, and by using the compound represented by the formula (11) in combination as the host material, luminous efficiency can be further increased and the driving voltage can be reduced at the same time.

In particular, it has been found that when the anthracene compound represented by the formula (11) has a dibenzofuranyl group or a naphthobenzofuranyl group, a further reduction in the driving voltage can be achieved while maintaining high luminous efficiency.

### Examples 17 to 25

The organic EL devices were fabricated and evaluated in the same manner as in Example 1 except that the compounds shown in Table 2 were used as the host material and the dopant material of the emitting layer. The results are shown in Table 2.

Compounds of the host material used in Examples 17 to 25 is shown below. Compounds of the dopant material are as previously shown.

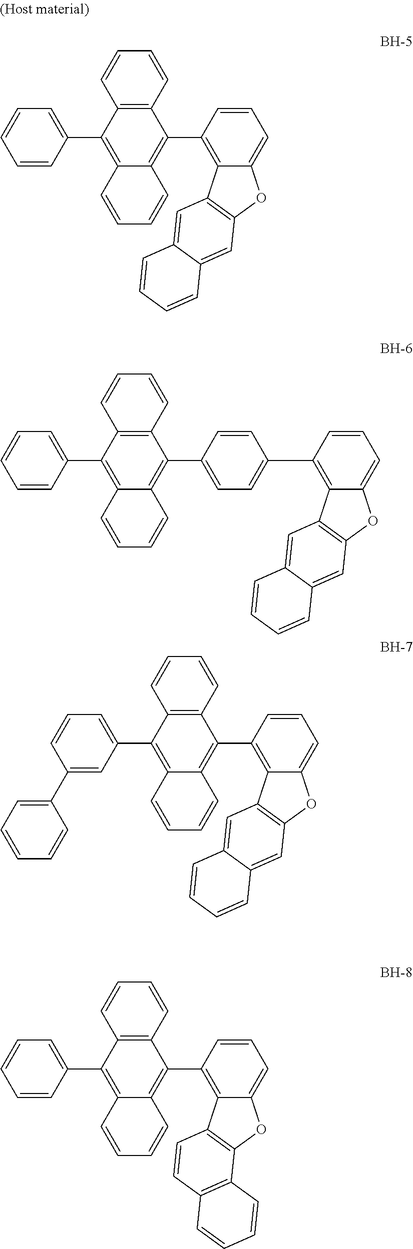 [SMILES: c1ccc(-c2c3ccccc3c(-c3cccc4oc5cc6ccccc6cc5c34)c3ccccc23)cc1 ; c1ccc(-c2c3ccccc3c(-c3ccc(-c4cccc5oc6cc7ccccc7cc6c45)cc3)c3ccccc23)cc1 ; c1ccc(-c2cccc(-c3c4ccccc4c(-c4cccc5oc6cc7ccccc7cc6c45)c4ccccc34)c2)cc1 ; c1ccc(-c2c3ccccc3c(-c3cccc4oc5c6ccccc6ccc5c34)c3ccccc23)cc1]

TABLE 2

| | Host | Dopant | Voltage (V) | EQE (%) |
|---|---|---|---|---|
| Example 17 | BH-5 | BD-1 | 3.39 | 8.6 |
| Example 18 | BH-5 | BD-3 | 3.38 | 8.6 |
| Example 19 | BH-5 | BD-4 | 3.38 | 8.8 |
| Example 20 | BH-6 | BD-1 | 3.40 | 8.8 |
| Example 21 | BH-6 | BD-4 | 3.40 | 9.0 |
| Example 22 | BH-7 | BD-1 | 3.41 | 9.0 |
| Example 23 | BH-7 | BD-4 | 3.40 | 9.3 |
| Example 24 | BH-8 | BD-1 | 3.41 | 9.0 |
| Example 25 | BH-8 | BD-4 | 3.40 | 9.3 |

From the results shown in Table 2, the organic EL devices of Examples 17 to 25 using BH-5 to BH-8 as the host material and BD-1, BD-3 and BD-4 as the dopant material had high external quantum efficiency EQE of 8.6 to 9.3% and low drive voltages of 3.38 to 3.41 V.

From the above results, it can be seen that by using the compound represented by the formula (1) as the dopant material, an organic EL device excellent in luminous efficiency can be obtained, and by using the compound represented by the formula (11) in combination as the host material, luminous efficiency can be further increased and the driving voltage can be reduced at the same time.

Although only some exemplary embodiments and/or examples of this invention have been described in detail above, those skilled in the art will readily appreciate that many modifications are possible in the exemplary embodiments and/or examples without materially departing from the novel teachings and advantages of this invention. Accordingly, many of these modifications are within the scope of the invention.

The documents described in the specification and the specification of Japanese application(s) on the basis of which the present application claims Paris convention priority are incorporated herein by reference in its entirety.

1. An organic electroluminescence device comprising: a cathode, an anode, and an emitting layer disposed between the cathode and the anode, wherein the emitting layer comprises a compound represented by the following formula (1) and a compound represented by the following formula (11): 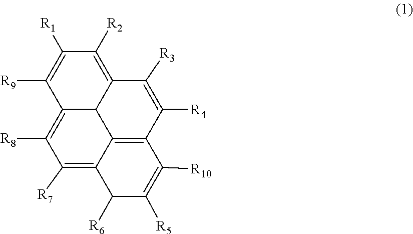
wherein in the formula (1), at least one of R₁ to R₁₀ is a monovalent group represented by the following formula (2);
R₁ to R₁₀ which are not the monovalent group represented by the following formula (2) are independently
a hydrogen atom, a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a substituted or unsubstituted aryl group including 6 to 50 ring carbon atoms, or
a substituted or unsubstituted monovalent heterocyclic group including 5 to 50 ring atoms;
adjacent two or more among R₁ to R₁₀ do not form a ring by bonding with each other;
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and
when two or more of each of R₉₀₁ to R₉₀₇ are present, the two or more of each of R₉₀₁ to R₉₀₇ may be the same or different; 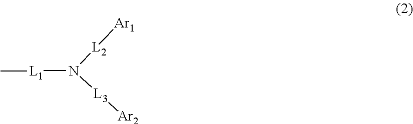
wherein in the formula (2), at least one of Ar₁ and Ar₂ is a group represented by the following formula (3);
Ar₁ or Ar₂ which is not the monovalent group represented by the following formula (3) is
a substituted or unsubstituted aryl group including 6 to 50 ring carbon atoms, or
a substituted or unsubstituted monovalent heterocyclic group including 5 to 50 ring atoms;
L₁, L₂, and L₃ are independently a single bond,
a substituted or unsubstituted arylene group including 6 to 30 ring carbon atoms, or
a substituted or unsubstituted divalent heterocyclic group including 5 to 30 ring atoms; and
when two or more of each of Ar₁, Ar₂, L₁, L₂, and L₃ are present, the two or more of each of Ar₁, Ar₂, L₁, L₂, and L₃ may be the same or different; 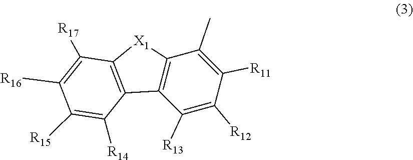
wherein in the formula (3), R₁₁ is a substituted or unsubstituted alkyl group including 1 to 50 carbon atoms,
a substituted or unsubstituted aryl group including 6 to 50 ring carbon atoms, or
a substituted or unsubstituted monovalent heterocyclic group including 5 to 50 ring atoms;
One or more sets of adjacent two or more among R₁₂ to R₁₇ form a substituted or unsubstituted, saturated or unsaturated ring by bonding with each other, or do not form a substituted or unsubstituted, saturated or unsaturated ring;
R₁₂ to R₁₇ which do not form a substituted or unsubstituted, saturated or unsaturated ring are independently
a hydrogen atom, a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are as defined in the formula (1); and
X₁ is an oxygen atom or a sulfur atom; 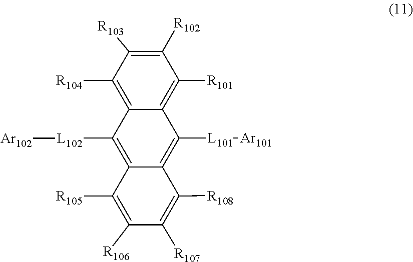
wherein in the formula (11), R₁₀₁ to R₁₀₈ are independently
a hydrogen atom, a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a substituted or unsubstituted aryl group including 6 to 50 ring carbon atoms, or
a substituted or unsubstituted monovalent heterocyclic group including 5 to 50 ring atoms;
adjacent two or more among R₁₀₁ to R₁₀₄, and adjacent two or more among R₁₀₅ to R₁₀₈ do not form a ring by bonding with each other;
R₉₀₁ to R₉₀₇ are as defined in the formula (1);
L₁₀₁ and L₁₀₂ are independently
a single bond,
a substituted or unsubstituted arylene group including 6 to 30 ring carbon atoms, or a substituted or unsubstituted divalent heterocyclic group including 5 to 30 ring atoms;
at least one of Ar₁₀₁ and Ar₁₀₂ is a monovalent group represented by the following formula (12);
Ar₁₀₁ or Ar₁₀₂ which is not the monovalent group represented by the following formula (12) is
a substituted or unsubstituted aryl group including 6 to 50 ring carbon atoms, or
a substituted or unsubstituted monovalent heterocyclic group including 5 to 50 ring atoms; and
when both Ar₁₀₁ and Ar₁₀₂ are the monovalent groups represented by the formula (12), Ar₁₀₁ and Ar₁₀₂ which are the monovalent groups represented by the following formula (12) may be the same as or different from each other; 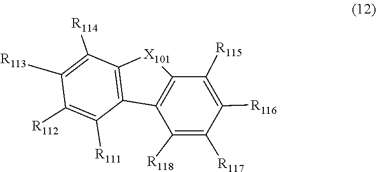
wherein in the formula (12),
X₁₀₁ is an oxygen atom or a sulfur atom; and
one or more sets of adjacent two or more of R₁₁₁ to R₁₁₈ form an unsaturated ring represented by the following formula (20) by bonding with each other, or do not form the unsubstituted ring represented by the following formula (20); 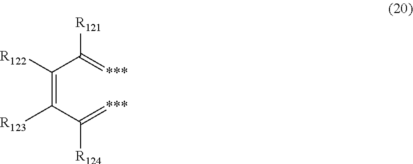
wherein in the formula (20), “***” indicates a position bonding to adjacent two of R₁₁₁ to R₁₁₈;
when one or more sets of adjacent two of R₁₁₁ to R₁₁₈ form the unsaturated ring represented by the formula (20) by bonding with each other, one of R₁₁₁ to R₁₁₈ which do not form the unsaturated ring represented by the formula (20), and one of R₁₂₁ to R₁₂₄ is a single bond bonding with L₁₀₁ or L₁₀₂;
when two or more of the unsaturated rings represented by the formula (20) are formed, a plurality of each of R₁₂₁ to R₁₂₄ may be the same as or different from each other;
when one or more sets of adjacent two of R₁₁₁ to R₁₁₈ do not form the unsaturated ring represented by the formula (20), one of R₁₁₁ to R₁₁₈ is a single bond bonding with L₁₀₁ or L₁₀₂;
when the unsaturated ring represented by the formula (20) is formed and when the unsaturated ring represented by the formula (20) is not formed, one or more sets of adjacent two of R₁₁₁ to R₁₁₈ which do not form the unsaturated ring represented by the formula (20) and are not a single bond bonding with L₁₀₁ or L₁₀₂ form a substituted or unsubstituted, saturated or unsaturated ring other than the unsaturated ring represented by the formula (20) by bonding with each other, or do not form a substituted or unsubstituted, saturated or unsaturated ring;
R₁₁₁ to R₁₁₈ which do not form the unsaturated ring represented by the formula (20), do not form a substituted or unsubstituted, saturated or unsaturated ring other than the unsaturated ring represented by the formula (20), and are not a single bond bonding with L₁₀₁ or L₁₀₂, and R₁₂₁ to R₁₂₄ which are not a single bond bonding with L₁₀₁ or L₁₀₂ are independently
a hydrogen atom, a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a substituted or unsubstituted aryl group including 6 to 50 ring carbon atoms, or
a substituted or unsubstituted monovalent heterocyclic group including 5 to 50 ring atoms; and
R₉₀₁ to R₉₀₇ are as defined in the formula (1).

2. The organic electroluminescence device according to claim 1, wherein X₁ in the formula (3) is an oxygen atom.

3. The organic electroluminescence device according to claim 1, wherein L₁ in the formula (2) is a single bond.

4. The organic electroluminescence device according to claim 1, wherein Ar₁ in the formula (2) is a group represented by the formula (3) and Ar₂ in the formula (2) is a substituted or unsubstituted aryl group including 6 to 50 ring carbon atoms.

5. The organic electroluminescence device according to claim 1, wherein L₂ and L₃ in the formula (2) are single bonds.

6. The organic electroluminescence device according to any claim 1, wherein two among R₁ to R₁₀ in the formula (1) are monovalent groups represented by the formula (2).

7. The organic electroluminescence device according to claim 1, wherein one or more sets of adjacent two or more among R₁₂ to R₁₇ in the formula (3) do not form a substituted or unsubstituted, saturated or unsaturated ring by bonding with each other.

8. The organic electroluminescence device according to claim 1, wherein the compound represented by the formula (1) is a compound represented by the following formula (4A): 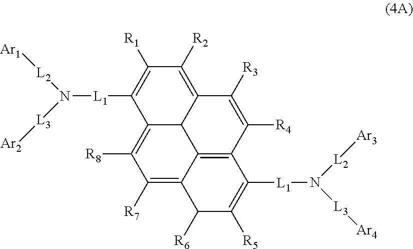
wherein in the formula (4A),
R₁ to R₈ are as defined in the formula (1);
Ar₁, Ar₂, L₁, L₂, and L₃ are as defined in the formula (2);
Ar₃ and Ar₄ are independently a substituted or unsubstituted aryl group including 6 to 50 ring carbon atoms, a substituted or unsubstituted monovalent heterocyclic group including 5 to 50 ring atoms, or a group represented by the formula (3); and
provided that at least one of Ar₃ and Ar₄ is a group represented by the formula (3).

9. The organic electroluminescence device according to claim 1, wherein the compound represented by the formula (1) is a compound represented by the following formula (4B): 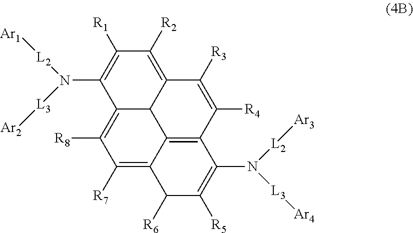
wherein in the formula (4B),
R₁ to R₈ are as defined in the formula (1);
Ar₁, Ar₂, L₂, and L₃ are as defined in the formula (2);
Ar₃ and Ar₄ are independently a substituted or unsubstituted aryl group including 6 to 50 ring carbon atoms, a substituted or unsubstituted monovalent heterocyclic group including 5 to 50 ring atoms, or a group represented by the formula (3); and
provided that at least one of Ar₃ and Ar₄ is a group represented by the formula (3).

10. The organic electroluminescence device according to claim 1, wherein the compound represented by the formula (1) is a compound represented by the following formula (4): 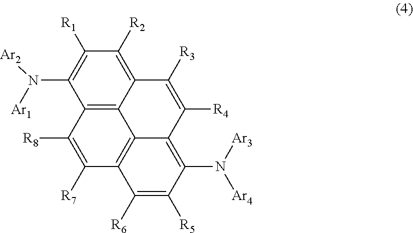
wherein in the formula (4),
R₁ to R₈ is as defined in the formula (1);
Ar₁ and Ar₂ are as defined in the formula (2);
Ar₃ and Ar₄ are independently a substituted or unsubstituted aryl group including 6 to 50 ring carbon atoms, a substituted or unsubstituted monovalent heterocyclic group including 5 to 50 ring atoms, or a group represented by the formula (3); and
provided that at least one of Ar₃ and Ar₄ is a group represented by the formula (3).

11. The organic electroluminescence device according to any claim 8, wherein R₁ to R₈ in the formula (1) are independently a hydrogen atom, or a substituted or unsubstituted alkyl group including 1 to 50 carbon atoms;
Ar₁ in the formula (2), and Ar₃ in the formulas (4A), (4B) and (4) are independently a group represented by the formula (3);
Ar₂ in the formula (2), and Ar₄ in the formula (4A), (4B) and (4) are independently a substituted or unsubstituted aryl group including 6 to 50 ring carbon atoms;
R₁₁ in the formula (3) is a substituted or unsubstituted alkyl group including 1 to 50 carbon atoms, or a substituted or unsubstituted aryl group including 6 to 50 ring carbon atoms; and
R₁₂ to R₁₇ in the formula (3) are independently a hydrogen atom, a substituted or unsubstituted alkyl group including 1 to 50 carbon atoms, or a substituted or unsubstituted aryl group including 6 to 50 ring carbon atoms.

12. The organic electroluminescence device according to claim 8, wherein R₁ to R₈ in the formula (1) are independently a hydrogen atom, or a substituted or unsubstituted alkyl group including 1 to 18 carbon atoms;
Ar₁ in the formula (2), and Ar₃ in the formulas (4A), (4B) and (4) are independently a group represented by the formula (3);
Ar₂ in the formula (2), and Ar₄ in the formula (4A), (4B) and (4) are independently a substituted or unsubstituted aryl group including 6 to 18 ring carbon atoms;
R₁₁ in the formula (3) is a substituted or unsubstituted alkyl group including 1 to 18 carbon atoms, or a substituted or unsubstituted aryl group including 6 to 18 ring carbon atoms; and
R₁₂ to R₁₇ in the formula (3) are independently a hydrogen atom, a substituted or unsubstituted alkyl group including 1 to 18 carbon atoms, or a substituted or unsubstituted aryl group including 6 to 18 ring carbon atoms.

13. The organic electroluminescence device according to claim 11, wherein Ar₂ in the formula (2) and Ar₄ in the formulas (4A), (4B) and (4) are independently a substituted or unsubstituted phenyl group, a substituted or unsubstituted biphenyl group, or a substituted or unsubstituted naphthyl group.

14. The organic electroluminescence device according to any claim 1, wherein R₁₁ in the formula (3) is a substituted or unsubstituted alkyl group including 1 to 8 carbon atoms.

15. The organic electroluminescence device according to claim 1, wherein R₁ to R₈ in the formula (1) are hydrogen atoms.

16. The organic electroluminescence device according to claim 1, wherein at least one of R₁ to R₈ in the formula (1) is a substituted or unsubstituted alkyl group including 1 to 18 carbon atoms, and R₁ to R₈ which are not the substituted or unsubstituted alkyl group including 1 to 18 carbon atoms are hydrogen atoms.

17. The organic electroluminescence device according to claim 1, wherein at least two of R₁ to R₈ in the formula (1) are substituted or unsubstituted alkyl groups including 1 to 18 carbon atoms, and R₁ to R₈ which are not the substituted or unsubstituted alkyl group including 1 to 18 carbon atoms are hydrogen atoms.

18. The organic electroluminescence device according to claim 1, wherein one of Ar₁₀₁ and Ar₁₀₂ in the formula (11) is a monovalent group represented by the formula (12).

19. The organic electroluminescence device according to claim 1, wherein the compound represented by the formula (11) is a compound represented by the following formula (13): 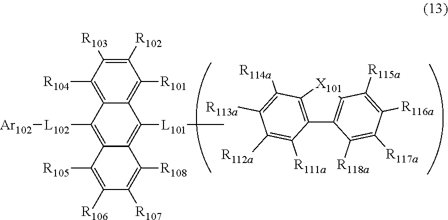
wherein in the formula (13),
R₁₀₁ to R₁₀₈, L₁₀₁, and L₁₀₂ are as defined in the formula (11);
Ar₁₀₂ is a monovalent group represented by the formula (12),
a substituted or unsubstituted aryl group including 6 to 50 ring carbon atoms, or
a substituted or unsubstituted monovalent heterocyclic group including 5 to 50 ring atoms;
X₁₀₁ is as defined in the formula (12);
one of R₁₁₁ₐ and R₁₁₂ₐ is a single bond bonding with L₁₀₁;
one or more sets of adjacent two or more of R₁₁₁ₐ or R₁₁₂ₐ which is not a single bond bonding with L₁₀₁, and R₁₁₃ₐ to R₁₁₈ₐ form an unsaturated ring represented by the formula (20) by bonding with each other, or do not form an unsaturated ring represented by the formula (20);
R₁₁₁ₐ or R₁₁₂ₐ which is not a single bond bonding with L₁₀₁ and which does not form an unsaturated ring represented by the formula (20), and R₁₁₃ₐ to R₁₁₈ₐ which do not form an unsaturated ring represented by the formula (20) are independently
a hydrogen atom, a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a substituted or unsubstituted aryl group including 6 to 50 ring carbon atoms, or
a substituted or unsubstituted monovalent heterocyclic group including 5 to 50 ring atoms; and
R₉₀₁ to R₉₀₇ are as defined in the formula (1).

20. The organic electroluminescence device according to claim 1, wherein the compound represented by the formula (11) is a compound represented by the following formula (14): 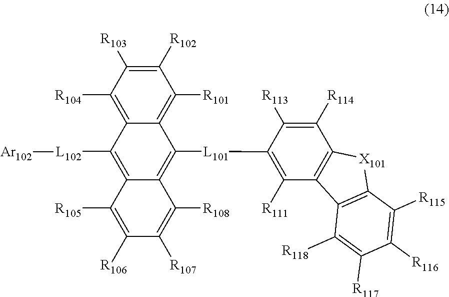
wherein in the formula (14),
R₁₀₁ to R₁₀₈, L₁₀₁, L₁₀₂, X₁₀₁, R₁₁₁, and R₁₁₃ to R₁₁₈ are as defined in the formula (11) and (12); and
Ar₁₀₂ is a monovalent group represented by the formula (12),
a substituted or unsubstituted aryl group including 6 to 50 ring carbon atoms, or
a substituted or unsubstituted monovalent heterocyclic group including 5 to 50 ring atoms.

21. The organic electroluminescence device according to claim 1, wherein the compound represented by the formula (11) is a compound represented by the following formula (14a): 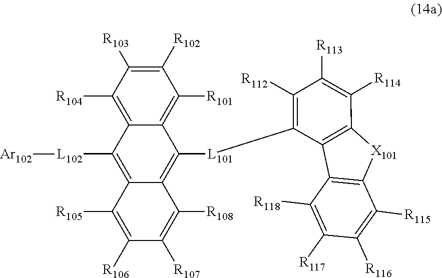
wherein in the formula (14a), R₁₀₁ to R₁₀₈, L₁₀₁, L₁₀₂, X₁₀₁, and R₁₁₂ to R₁₁₈ are as defined in the formula (11) and (12); and
Ar₁₀₂ is a monovalent group represented by the formula (12),
a substituted or unsubstituted aryl group including 6 to 50 ring carbon atoms, or
a substituted or unsubstituted monovalent heterocyclic group including 5 to 50 ring atoms.

22. The organic electroluminescence device according to claim 1, wherein the monovalent group represented by the formula (12) is selected from the monovalent groups represented by the following formulas (12A) to (12F): 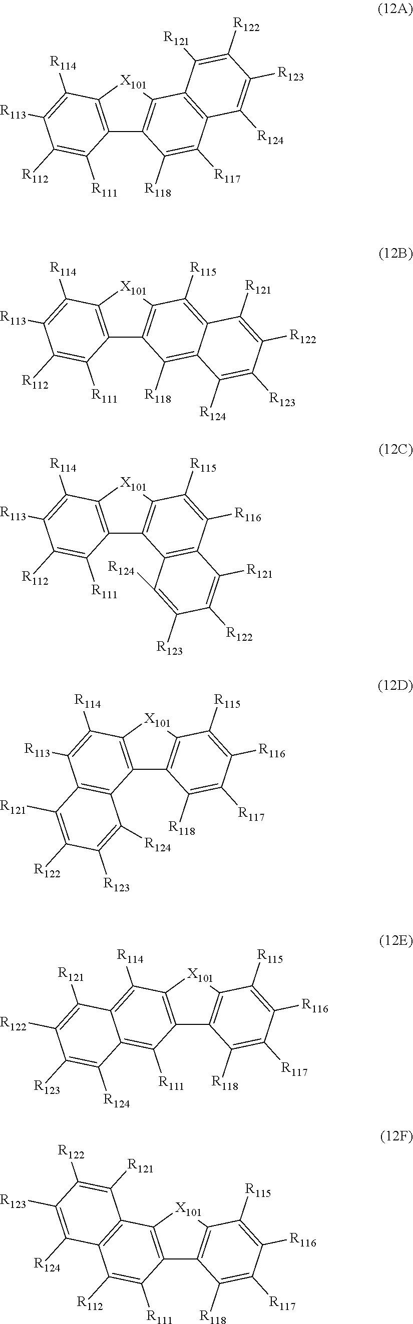
wherein in the formula (12A) to (12F), X₁₀₁, R₁₁₁ to Riis, and R₁₂₁ to R₁₂₄ are as defined in the formula (12).

23. The organic electroluminescence device according to claim 1, wherein one of Ar₁₀₁ and Ar₁₀₂ in the formula (11) is a monovalent group represented by the formula (12), and the other is a substituted or unsubstituted aryl group including 6 to 50 ring carbon atoms.

24. The organic electroluminescence device according to claim 1, wherein Ar₁₀₁ or Ar₁₀₂ in the formula (11) which is not a monovalent group represented by the formula (12) is selected from groups represented by the following formulas (a1) to (a4): 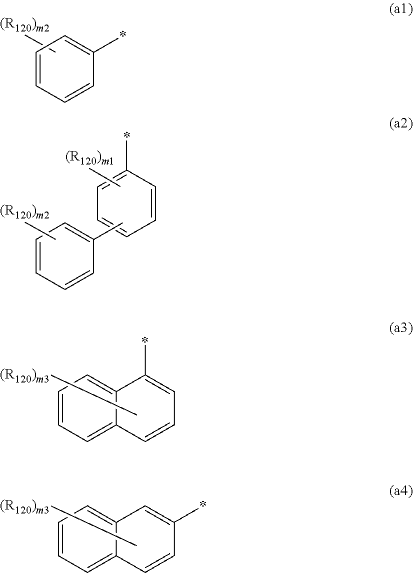
wherein in the formulas (a1) to (a4),
“*” is a single bond bonding with L₁₀₁ or L₁₀₂;
R₁₂₀ is
a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are as defined in the formula (1);
m1 is an integer of 0 to 4;
m2 is an integer of 0 to 5;
m3 is an integer of 0 to 7;
when each of m1 to m3 is 2 or more, a plurality of R₁₂₀'s may be the same as or different from each other; and
when each of m1 to m3 is 2 or more, a plurality of adjacent R₁₂₀'s form a substituted or unsubstituted, saturated or unsaturated ring by bonding with each other, or do not form a substituted or unsubstituted, saturated or unsaturated ring.

25. The organic electroluminescence device according to claim 1, wherein L₁₀₁ and L₁₀₂ in the formula (11) are independently a substituted or unsubstituted arylene group including 6 to 14 ring carbon atoms.

26. The organic electroluminescence device according to claim 1, wherein X₁₀₁ in the formula (12) is an oxygen atom.

27. The organic electroluminescence device according to claim 1, wherein the compound represented by the formula (11) is a compound represented by the following formula (15): 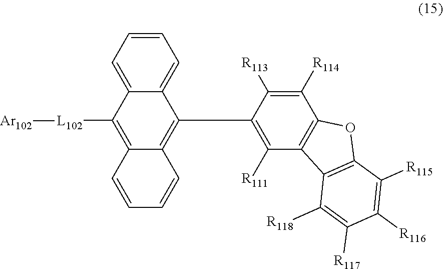
wherein in the formula (15), L₁₀₂ is as defined in the formula (11);
Ar₁₀₂ is a monovalent group represented by the formula (12),
a substituted or unsubstituted aryl group including 6 to 50 ring carbon atoms, or
a substituted or unsubstituted monovalent heterocyclic group including 5 to 50 ring atoms; and
R₁₁₁ and R₁₁₃ to R₁₁₈ are as defined in the formula (12).

28. The organic electroluminescence device according to claim 1, wherein the compound represented by the formula (11) is a compound represented by the following formula (15a): 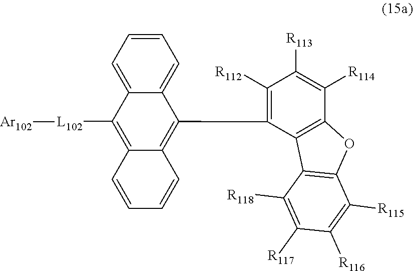
wherein in the formula (15a), L₁₀₂ is as defined in the formula (11);
Ar₁₀₂ is a monovalent group represented by the formula (12),
a substituted or unsubstituted aryl group including 6 to 50 ring carbon atoms, or a substituted or unsubstituted monovalent heterocyclic group including 5 to 50 ring atoms; and
R₁₁₂ to R₁₁₈ are as defined in the formula (12).

29. The organic electroluminescence device according to claim 1, wherein the compound represented by the formula (11) is a compound represented by the following formula (17): 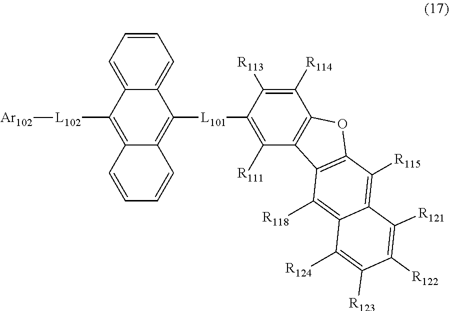
wherein in the formula (17),
L₁₀₁ and L₁₀₂ are as defined in the formula (11);
Ar₁₀₂ is a monovalent group represented by the formula (12),
a substituted or unsubstituted aryl group including 6 to 50 ring carbon atoms, or
a substituted or unsubstituted monovalent heterocyclic group including 5 to 50 ring atoms; and
R₁₁₁, R₁₁₃ to R₁₁₅, Riis, and R₁₂₁ to R₁₂₄ are as defined in the formula (12).

30. The organic electroluminescence device according to claim 1, wherein R₁₀₁ to R₁₀₈ in the formula (11) are hydrogen atoms.

31. The organic electroluminescence device according to claim 1, wherein R₁₁₁ to R₁₁₈ in the formula (12) which are not a single bond bonding with L₁₀₁, and R₁₂₁ to R₁₂₄ which are not a single bond bonding with L₁₀₁ are hydrogen atoms.

32. The organic electroluminescence device according to claim 1, wherein the compound represented by the formula (11) is a compound represented by the following formula (18): 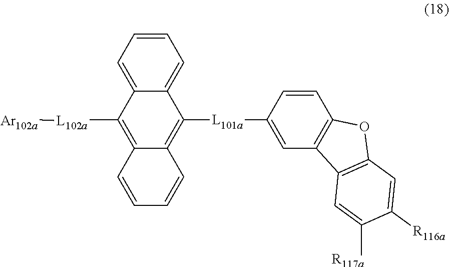
wherein in the formula (18),
L₁₀₁ₐ and L₁₀₂ₐ are independently
a single bond, or
a substituted or unsubstituted arylene group including 6 to 10 ring carbon atoms;
Ar₁₀₂ₐ is a substituted or unsubstituted aryl group including 6 to 10 ring carbon atoms;
R₁₁₆ₐ and R₁₁₇ₐ form a saturated or unsaturated ring by bonding with each other, or do not form a saturated or unsaturated ring;
R₁₁₆ₐ and R₁₁₇ₐ which do not form a saturated or unsaturated ring by bonding with each other are independently
a hydrogen atom, a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅), or
—N(R₉₀₆)(R₉₀₇); and
R₉₀₁ to R₉₀₇ are as defined in the formula (1).

33. The organic electroluminescence device according to claim 1, wherein the compound represented by the formula (11) is a compound represented by the following formula (18a): 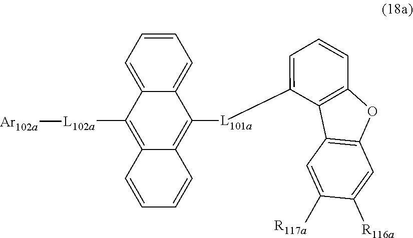
wherein in the formula (18a),
L₁₀₁ₐ and L₁₀₂ₐ are independently
a single bond, or
a substituted or unsubstituted arylene group including 6 to 10 ring carbon atoms;
Ar₁₀₂ₐ is a substituted or unsubstituted aryl group including 6 to 10 ring carbon atoms;
R₁₁₆ₐ and R₁₁₇ₐ form a saturated or unsaturated ring by bonding with each other, or do not form a saturated or unsaturated ring;
R₁₁₆ₐ and R₁₁₇ₐ which do not form a saturated or unsaturated ring by bonding with each other are independently
a hydrogen atom, a halogen atom, a cyano group, a nitro group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅), or
—N(R₉₀₆)(R₉₀₇); and
R₉₀₁ to R₉₀₇ are as defined in the formula (1).

34. An electronic appliance, wherein the organic electroluminescence device according to claim 1 is provided.
